(19) United States
(12) Patent Application Publication
(10) Pub. No.: US 2026/0293417 A1
(43) Pub. Date: Sep. 24, 2026

(54) MIXTURES, FORMULATIONS, AND USES THEREOF IN ORGANIC ELECTRONIC DEVICES

(71) Applicant: Zhejiang Brilliant Optoelectronic Technology Co.,Ltd., Taizhou (CN)

(72) Inventors: Junyou PAN, Taizhou (CN); Hao ZHANG, Taizhou (CN)

(73) Assignee: Zhejiang Brilliant Optoelectronic Technology Co.,Ltd., Taizhou (CN)

(21) Appl. No.: 19/664,641

(22) Filed: Apr. 30, 2026

Related U.S. Application Data
(63) Continuation of application No. PCT/CN2024/088890, filed on Apr. 19, 2024.

(30) Foreign Application Priority Data
Nov. 8, 2023 (CN) 202311478363.1

(51) Int. Cl. H10K 50/11; H10K 50/16; H10K 50/18; H10K 85/60; H10K 101/30

(52) U.S. Cl. CPC H10K 50/11; H10K 50/16; H10K 50/18; H10K 85/622; H10K 85/654; H10K 85/6572; H10K 85/6574; H10K 85/6576; H10K 2101/30

(57) ABSTRACT

A mixture includes a first compound H1 and a second compound H2. An organic electronic device contains a mixture. An organic compound contains a structure of formula (VI). The mixture or organic compound is used as a phosphorescent host material to achieve high device efficiency and long device lifetime. In particular, the organic mixture effectively balances hole and electron transport rates, enabling low efficiency roll-off at high brightness and long lifetime in OLEDs.

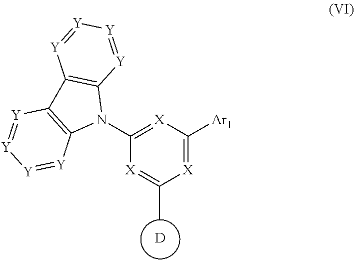

### CROSS-REFERENCE TO RELATED APPLICATIONS

The present application is a continuation of International Application No. PCT/CN2024/088890, filed on Apr. 19, 2024, which claims priority to Chinese Patent Application No. 202311478363.1, filed on Nov. 8, 2023. All of the aforementioned applications are incorporated herein by reference in their entireties.

### TECHNICAL FIELD

The present disclosure relates to the field of electroluminescent material, and in particular to a mixture, and the applications thereof in organic electronic devices, particularly in organic electroluminescent devices.

### BACKGROUND

Due to the structural diversity, the low manufacturing cost, and the excellent optical and electrical properties, the organic light-emitting diodes (OLEDs) show great potential for optoelectronic device applications, such as flat-panel displays and lighting.

The organic electroluminescent phenomenon refers to a phenomenon of converting electrical energy to photonic energy with organic substances. An organic electroluminescent element utilizing the organic electroluminescent phenomenon usually has a structure comprising an anode, a cathode, and an organic layer therebetween. In order to improve the efficiency and lifetime of the organic electroluminescent element, the organic layer has a multi-layer structure, and each layer comprises different organic substances. Specifically, each layer can be a hole-injection layer, a hole-transport layer, an emissive layer, an electron-transport layer, an electron-injection layer, etc. When a voltage is applied between the two electrodes of such an organic electroluminescent element, holes are injected into the organic layer from the anode, and electrons are injected into the organic layer from the cathode; and an exciton is formed when an injected hole and an injected electron recombine in the emissive layer. The exciton emits light when it transitions back to the ground state. The organic electroluminescent element has characteristics of self-emission, high luminance, high efficiency, low driving voltage, wide viewing angle, high contrast, high responsivity, etc.

Various organic optoelectronic material systems have been extensively developed to enhance the luminous performance of the OLEDs and advance their large-scale industrialization. The host materials play a decisive role in the luminescence efficiency, lifetime, and other aspects of OLED devices. For example, a triazine or pyrimidine derivative as a host material have been disclosed in patent application publications such as US2016141505A1, WO2015111848A1, and KR2022139504A, but the resulting device performance, especially their lifetime, still requires further improvement.

In order to meet practical requirements, it is necessary to further develop high-performance OLED materials, particularly host materials.

### SUMMARY

In one aspect, the present disclosure provides a mixture comprising a first compound H1 and a second compound H2, where min(LUMO(H1)−HOMO(H2), LUMO(H2)−HOMO(H1))≤min(E_{T1}(H1), E_{T1}(H2))+0.1 eV, LUMO(H1), HOMO(H1), and E_{T1}(H1) respectively represent the lowest unoccupied orbital energy level, the highest occupied orbital energy level, and the triplet energy level of the first compound H1; LUMO(H2), HOMO(H2), and E_{T1}(H2) respectively represent the lowest unoccupied orbital energy level, the highest occupied orbital energy level, and the triplet energy level of the second compound H2; the first compound H1 comprises a structure of formula (I):

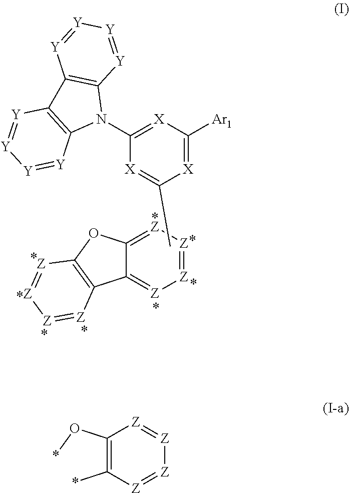

Where two adjacent * in formula (I) are bonded to two * in formula (I-a); each X is independently N or CR₁, and at least one X is N atom; Y at each occurrence is independently CR₂ or N; where no more than two Ys in each ring are N atoms; each Z is independently N or CR₃; Ar₁ is independently selected from a substituted/unsubstituted aromatic or heteroaromatic group containing 5 to 40 ring atoms, an aryloxy or heteroaryloxy group containing 5 to 40 ring atoms, or any combination thereof, where one or more Aris may form a monocyclic or polycyclic aliphatic or aromatic ring system with each other and/or with the rings bonded thereto; each of R₁ to R₃ is independently selected from —H, -D, a substituted/unsubstituted C₁-C₁₀ alkyl group, a substituted/unsubstituted aromatic or heteroaromatic group containing 5 to 60 ring atoms, or any combination thereof.

In another aspect, the present disclosure also provides a formulation comprising a mixture as described herein, and at least one organic solvent.

In yet another aspect, the present disclosure further provides an organic electronic device comprising a mixture as described herein.

In yet another aspect, the present disclosure further provides an organic compound comprising a structure of formula (VI):

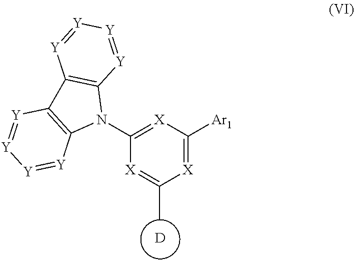

Where each X is independently N or CR₁, and at least one X is N atom; Y at each occurrence is independently CR₂ or N; where no more than two Ys in each ring are N atoms; Ar₁ is independently selected from a substituted/unsubstituted aromatic or heteroaromatic group containing 5 to 40 ring atoms, an aryloxy or heteroaryloxy group containing 5 to 40 ring atoms, or any combination thereof, where one or more Aris may form a monocyclic or polycyclic aliphatic or aromatic ring system with each other and/or with the rings bonded thereto;

D ring is selected from the group consisting of:

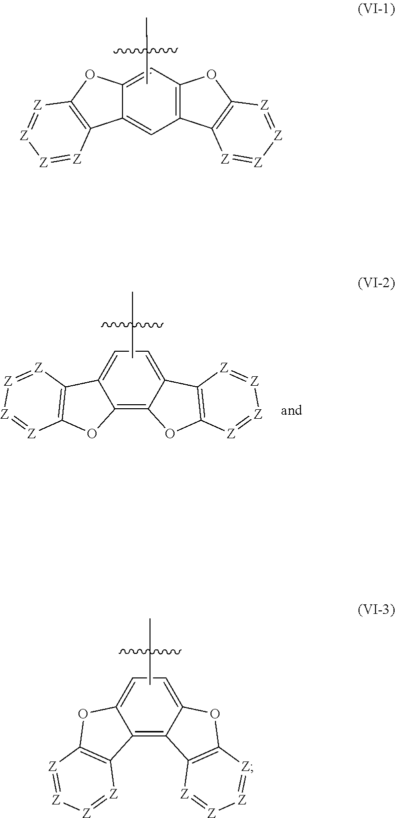

Where each Z is independently N or CR₃; each of R₁ to R₃ is independently selected from —H, -D, a substituted/unsubstituted C₁-C₁₀ alkyl group, a substituted/unsubstituted aromatic or heteroaromatic group containing 5 to 60 ring atoms, or any combination thereof.

The mixture as described herein comprises a first compound H1 and a second compound H2 capable of forming an exiplex, and the first compound H1 and the second compound H2 can form type II heterojunction energy structure, and have high stability. The mixture or organic compound as described herein can be used as an electrophosphorescent host material or co-host material, which can improve the device luminescence efficiency and lifetime by matching a suitable dopant material, thereby providing materials for high-efficiency and long-lifetime light-emitting devices.

### DETAILED DESCRIPTION OF THE EMBODIMENTS

The present disclosure provides a mixture, and the applications thereof in organic electronic devices, aiming to solve the problems of poor stability and short lifetime in the existing organic electronic devices.

The present disclosure provides a mixture, a formulation, and the applications thereof in organic electronic devices. In order to make the objects, the technical solutions and the effects of the present disclosure more clear and definite, the present disclosure is further described in detail below. It should be understood that the embodiments described herein are only intended to explain the present disclosure and are not intended to limit the present disclosure.

As used herein, the terms “formulation”, “printing ink”, and “ink” have the same meaning, and they are interchangeable with each other.

As used herein, the terms “host material”, “matrix material” have the same meaning, and they are interchangeable with each other.

As used herein, the terms “metal organic clathrate”, “metal organic complex”, and “organometallic complex” have the same meaning, and they are interchangeable with each other.

As used herein, the heterojunction refers to an interface region formed by contacting two different semiconductors. According to the alignment of the conduction band (LUMO) and the valence band (HOMO) of the two materials in the heterojunction, the heterojunction can be classified into type I heterojunction and type II heterojunction, while the basic features of the type II heterojunction are the separation of electrons and hole spatially near the intersection and the localization in the self-consistent quantum well. In addition, the overlap of wave functions near the interface leads to a reduction of the optical matrix elements, resulting in longer radiative lifetime and less exciton binding energy.

In one aspect, the present disclosure provides a mixture comprising a first compound H1 and a second compound H2, where min(LUMO(H1)−HOMO(H2), LUMO(H2)−HOMO(H1))≤min(E_{T1}(H1), E_{T1}(H2))+0.1 eV, LUMO(H1), HOMO(H1), and E_{T1}(H1) respectively represent the lowest unoccupied orbital energy level, the highest occupied orbital energy level, and the triplet energy level of the first compound H1; LUMO(H2), HOMO(H2), and E_{T1}(H2) respectively represent the lowest unoccupied orbital energy level, the highest occupied orbital energy level, and the triplet energy level of the second compound H2; the first compound H1 comprises a structure of formula (I):

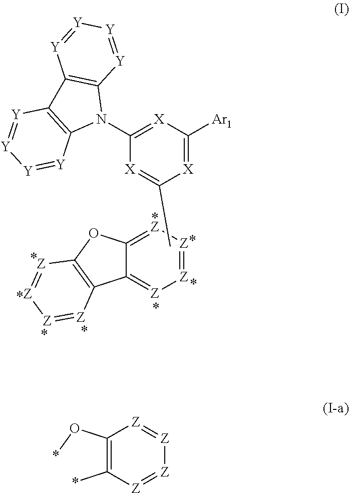

Where two adjacent * in formula (I) are bonded to two * in formula (I-a); each X is independently N or CR₁, and at least one X is N atom; Y at each occurrence is independently CR₂ or N; where no more than two Ys in each ring are N atoms; each Z is independently N or CR₃; Ar₁ is independently selected from a substituted/unsubstituted aromatic or heteroaromatic group containing 5 to 40 ring atoms, an aryloxy or heteroaryloxy group containing 5 to 40 ring atoms, or any combination thereof, where one or more Aris may form a monocyclic or polycyclic aliphatic or aromatic ring system with each other and/or with the rings bonded thereto; each of R₁ to R₃ is independently selected from —H, -D, a substituted/unsubstituted C₁-C₁₀ alkyl group, a substituted/unsubstituted aromatic or heteroaromatic group containing 5 to 60 ring atoms, or any combination thereof.

In some embodiments, Ar₁ is a substituted/unsubstituted aromatic or heteroaromatic group containing 5 to 30 ring atoms. In some embodiments, Ar₁ is a substituted/unsubstituted aromatic or heteroaromatic group containing 5 to 20 ring atoms.

In some embodiments, each of R₁ to R₃ is independently —H or -D.

In some embodiments, each of R₁ to R₃ is independently a substituted/unsubstituted aromatic or heteroaromatic group containing 5 to 50 ring atoms, or any combination thereof. In some embodiments, each of R₁ to R₃ is independently a substituted/unsubstituted aromatic or heteroaromatic group containing 5 to 40 ring atoms, or any combination thereof. In some embodiments, each of R₁ to R₃ is independently a substituted/unsubstituted aromatic or heteroaromatic group containing 5 to 30 ring atoms, or any combination thereof.

In some embodiments, the aromatic ring system contains 5 to 25 ring atoms in the ring system, the heteroaromatic ring system contains 5 to 25 ring atoms and at least one heteroatom in the ring system, provided that the total number of carbon atoms and heteroatoms is at least 5. In some embodiments, the aromatic ring system contains 5 to 20 ring atoms in the ring system, the heteroaromatic ring system contains 5 to 20 ring atoms and at least one heteroatom in the ring system. In some embodiments, the aromatic ring system contains 5 to 16 ring atoms in the ring system, the heteroaromatic ring system contains 5 to 16 ring atoms and at least one heteroatom in the ring system. The heteroatoms are preferably selected from Si, N, P, O, S and/or Ge, particularly preferably selected from Si, N, P, O and/or S.

The term “aromatic ring system” or “aromatic group” refers to a hydrocarbon group consisting of an aromatic ring, including monocyclic groups and polycyclic systems. The term “heteroaromatic ring system” or “heteroaromatic group” refers to a hydrocarbon group (containing a heteroatom) consisting of at least one heteroaromatic ring, including monocyclic groups and polycyclic systems. The polycyclic systems contain two or more rings, in which two carbon atoms are shared by the adjacent two rings, i.e., fused rings. Specifically, at least one of the rings in the polycyclic rings are aromatic or heteroaromatic. For the purposes of the present disclosure, the aromatic or heteroaromatic ring systems contain not only aromatic or heteroaromatic groups, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic ring systems for the purposes of this disclosure.

Specifically, examples of the aromatic groups include: benzene, naphthalene, anthracene, phenanthrene, perylene, naphthacene, pyrene, benzpyrene, triphenylene, acenaphthene, fluorene, and derivatives thereof.

Specifically, examples of heteroaromatic groups include: furan, benzofuran, thiophene, 1-benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazanaphthalene, quinoxaline, phenanthridine, perimidine, quinazoline, benzoyleneurea, and derivatives thereof.

In some embodiments, the first compound H1 is selected from the group consisting of:

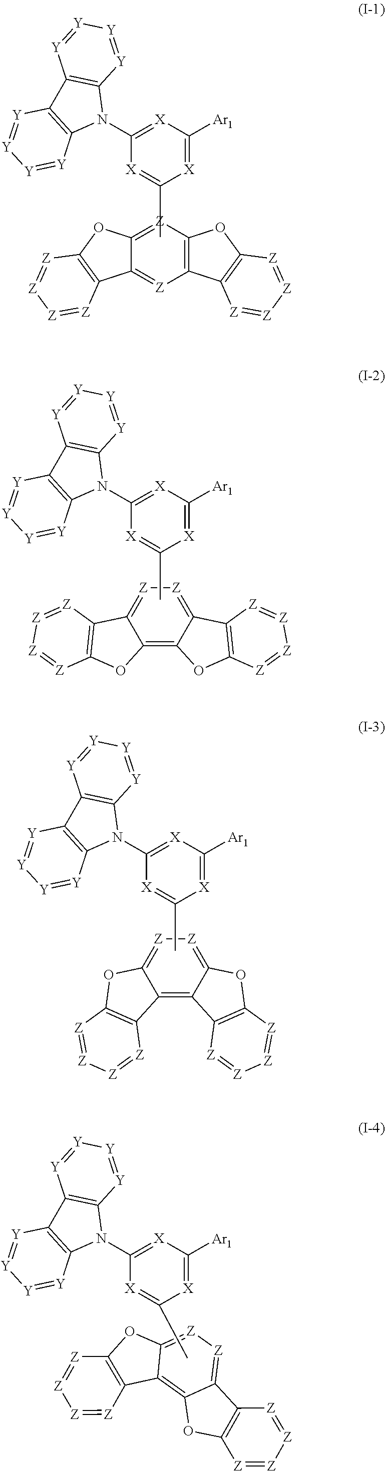

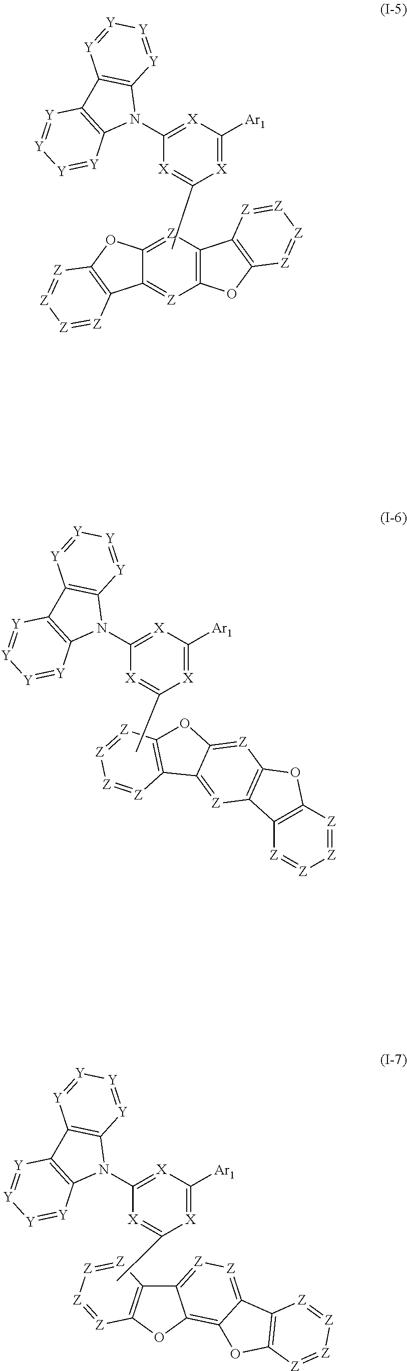

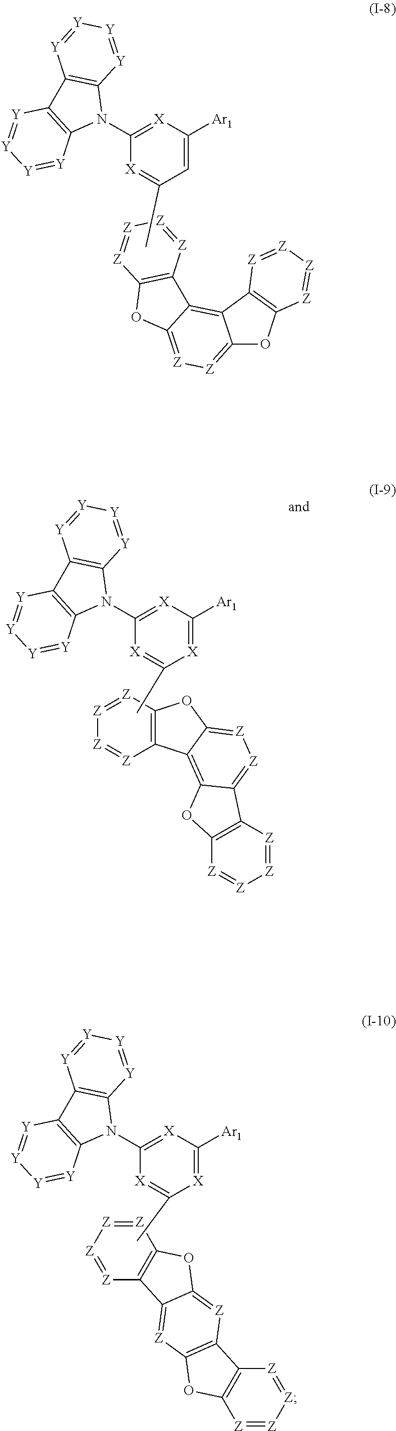

Where X, Y, Ar₁, and Z are defined identically as described herein;
Where each Ar₁ in formula (I-5) and formula (I-10) is independently selected from the following group:

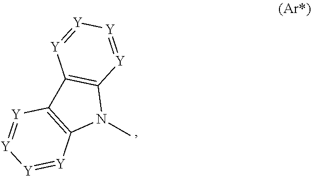

each Y is defined identically as described herein.

In some embodiments, in the mixture as described herein, each Ar₁ in the first compound H1 is independently selected from one of the following groups or any combination thereof:

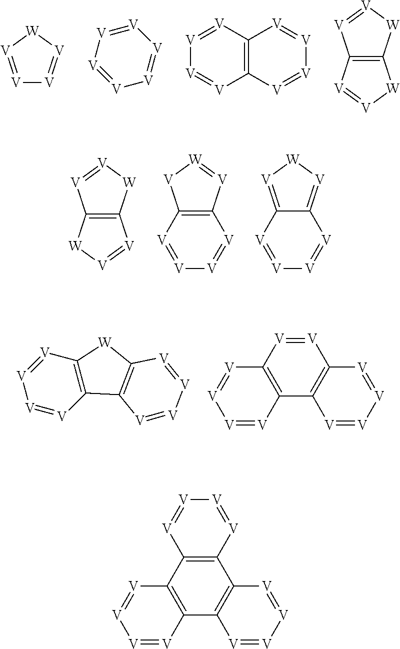

V, in multiple occurrences, is independently selected from CR₄ or N;
W, in multiple occurrences, is independently selected from CR₅R₆, SiR₇R₈, NR₉, C(═O), S, or O;
each of R₄ to R₉ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, where one or more R₄-R₉ may form a monocyclic or polycyclic aliphatic or aromatic ring system with each other and/or with the rings bonded thereto.

In some embodiments, each Ar₁ is independently selected from the following structural formulas and may be substituted with 0, 1, 2, or 3 substituents, and the substituents are each independently selected from -D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group:

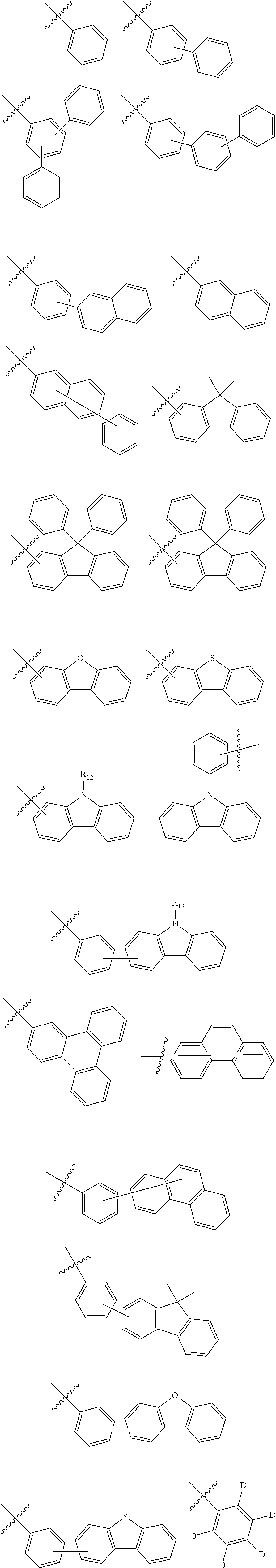

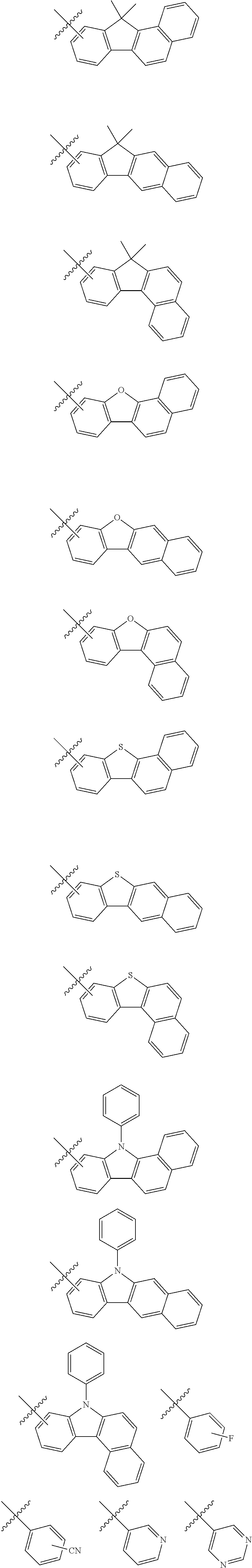

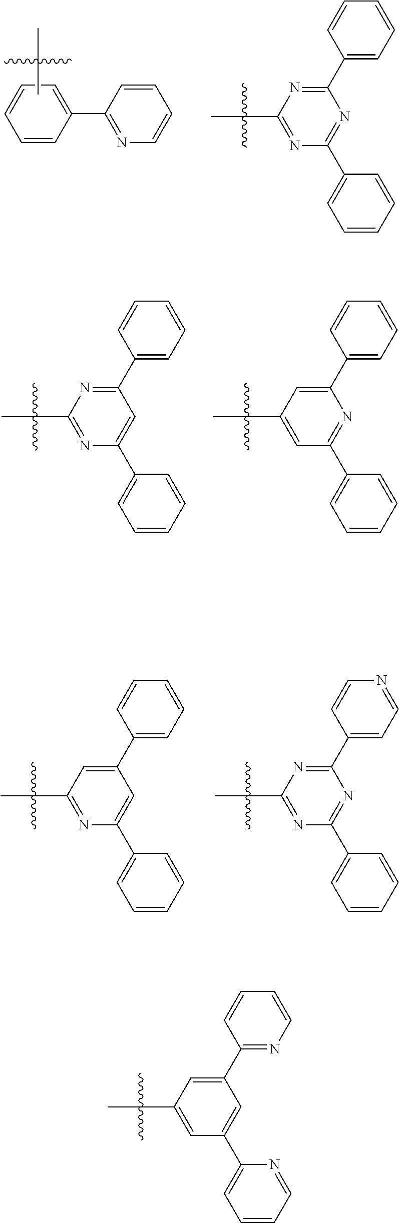

Where R₁₂ and R₁₃ are defined identically as the above-mentioned R₄.

In some embodiments, no more than three Zs are N atoms, preferably two Zs are N atoms, more preferably one Z is N atom, and most preferably each Z is CH.

In some embodiments, in the mixture as described herein, the first compound H1 and/or the second compound H2 have a high triplet energy level E_{T1}, generally ≥2.2 eV, preferably ≥2.4 eV, more preferably ≥2.5 eV, further preferably ≥2.6 eV, and most preferably ≥2.8 eV.

In some embodiments, in the mixture as described herein, the first compound H1 and/or the second compound H2 are partially deuterated; preferably 10% or more of total H, more preferably 20% or more of total H, further preferably 30% or more of total H, and most preferably 40% or more of total H, are deuterated.

In some embodiments, the mixture as described herein facilitates a thermally activated delayed fluorescence effect. And the triplet exciton of the first compound H1 and/or the second compound H2 can be converted to a singlet exciton by reverse internal conversion when ΔE(E_{S1}-E_{T1}) is sufficiently small, thus achieving high luminescence efficiency. In general, such material is obtained by connecting electron-donating group (Donor) to electron-deficient or electron-accepting (Acceptor) groups, having obvious D-A structures.

The preferred examples of the first compound H1s as described herein are shown in the following structures. These structures may be substituted at all possible substitution sites.

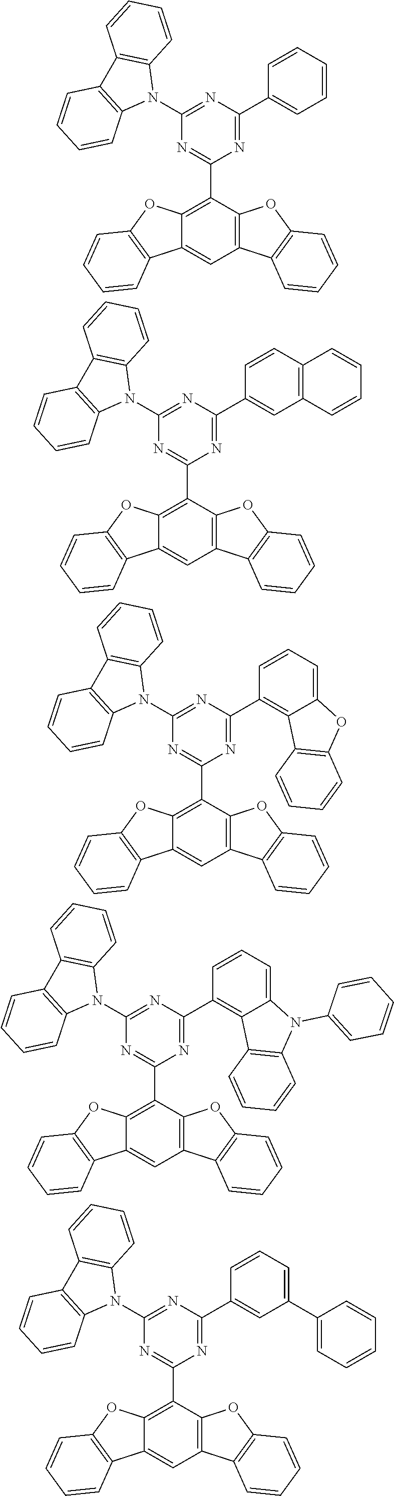

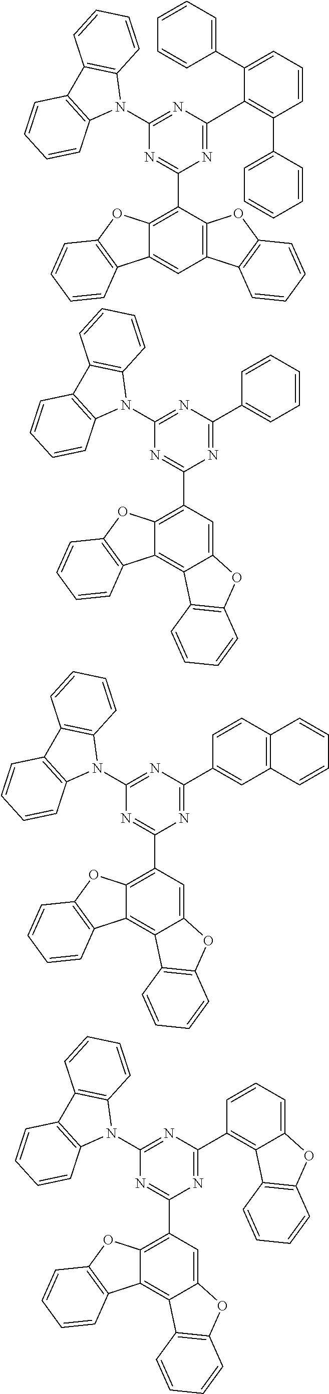

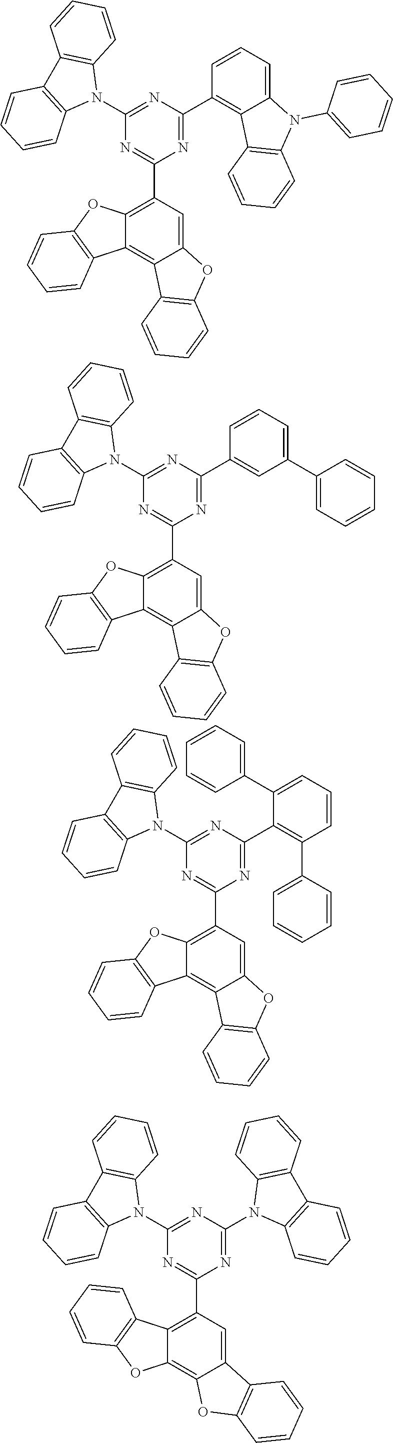

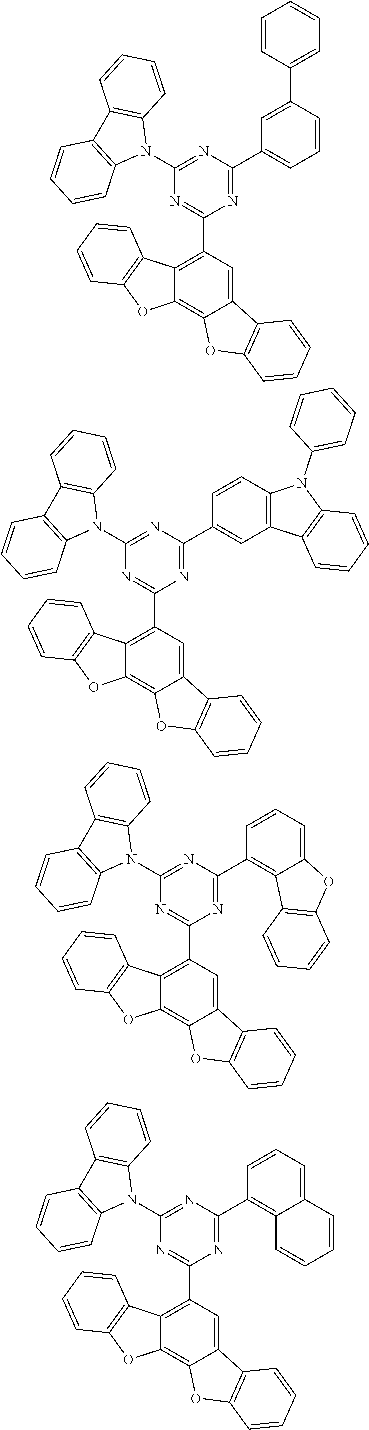

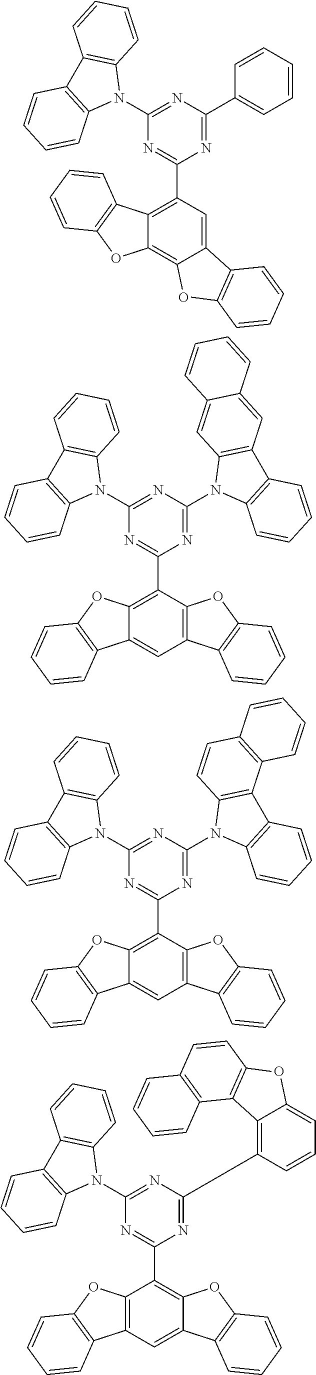

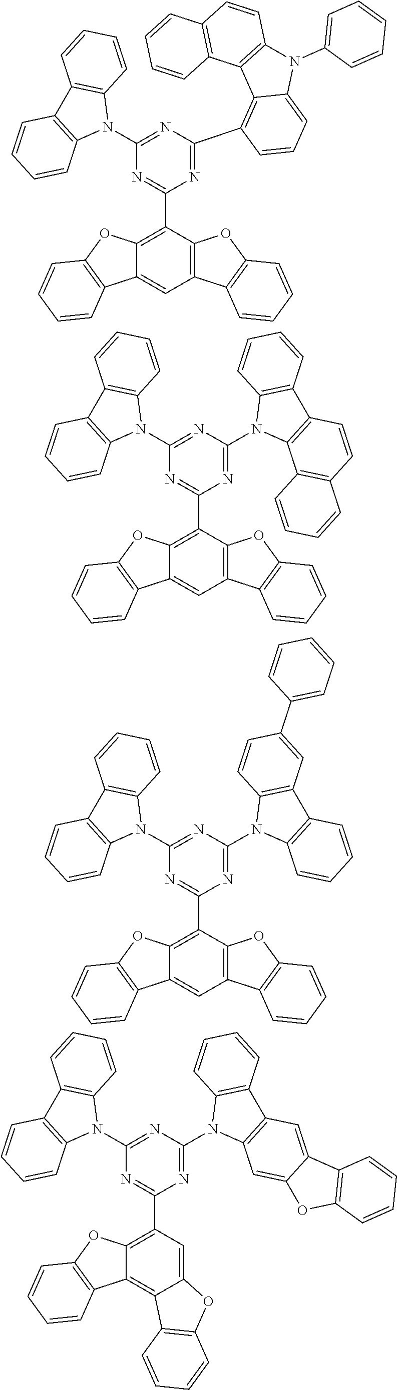

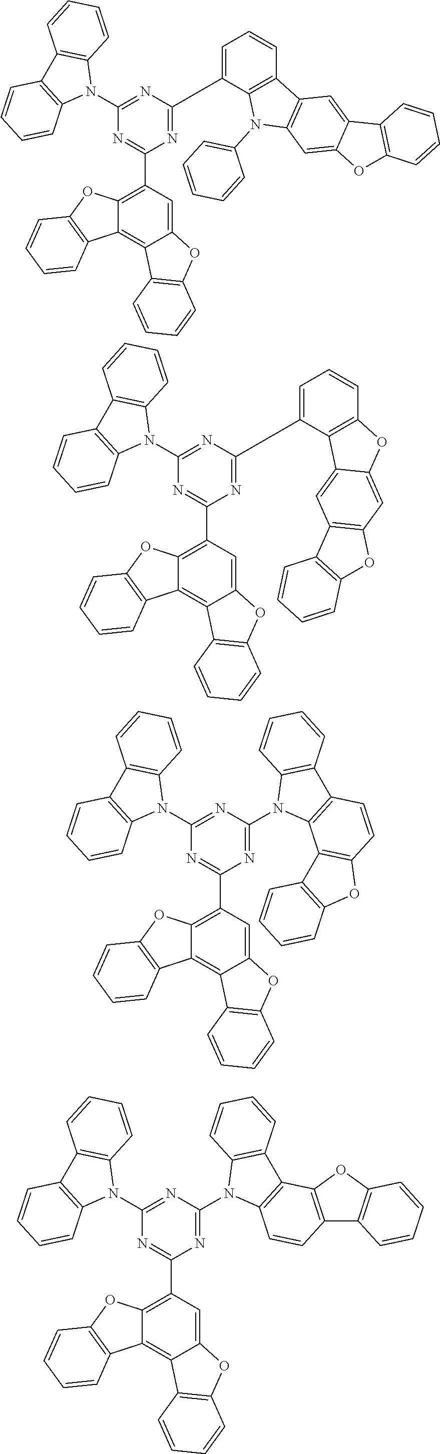

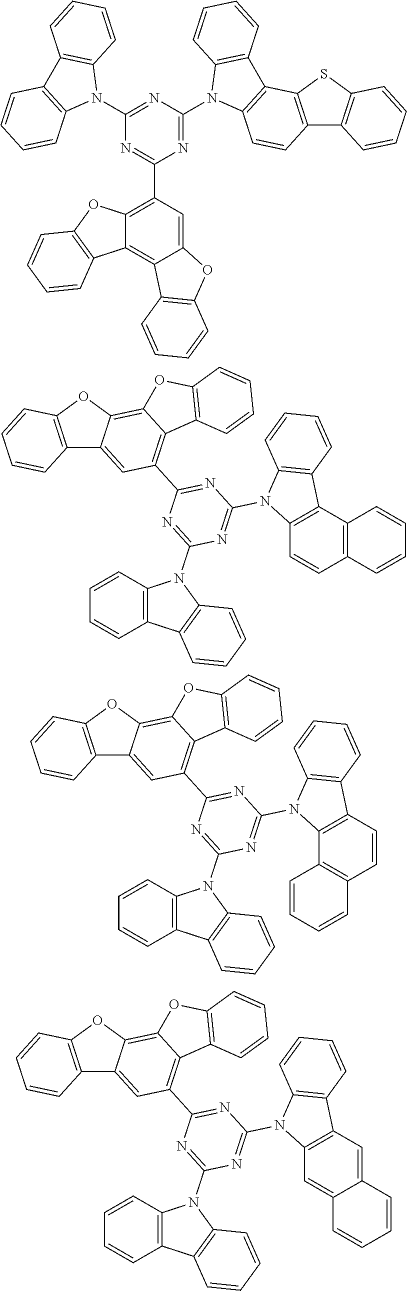

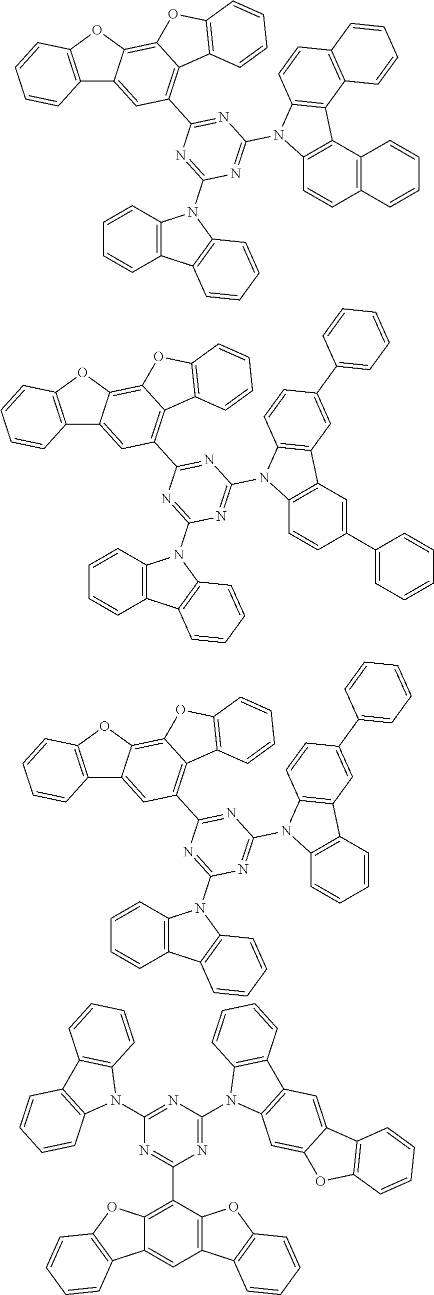

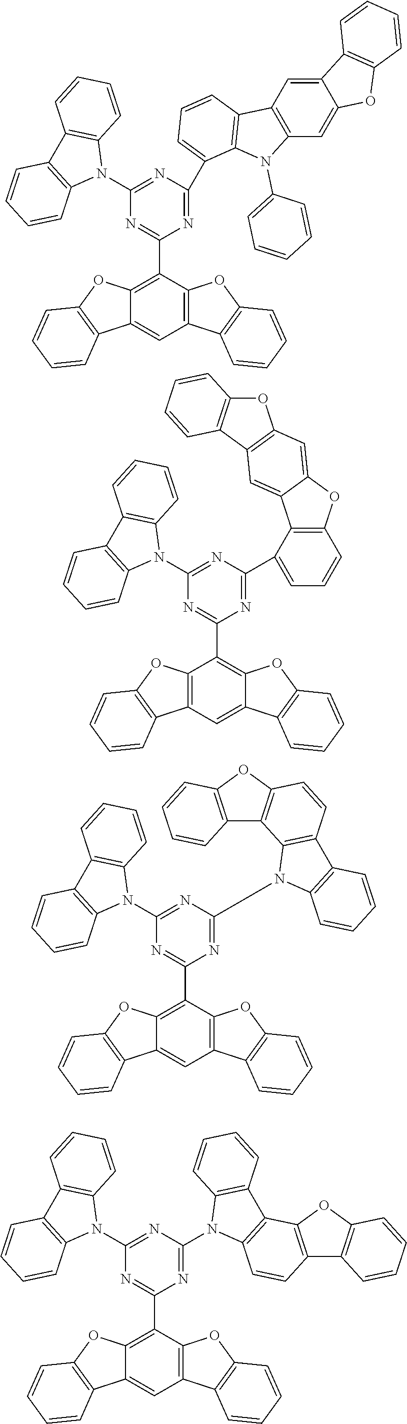

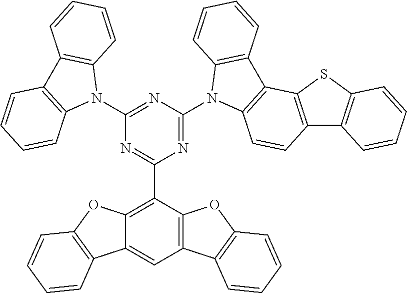 [SMILES: c1ccc2c(c1)oc1c(-c3nc(-n4c5ccccc5c5ccccc54)nc(-n4c5ccccc5c5c6sc7ccccc7c6ccc54)n3)c3oc4ccccc4c3cc12]

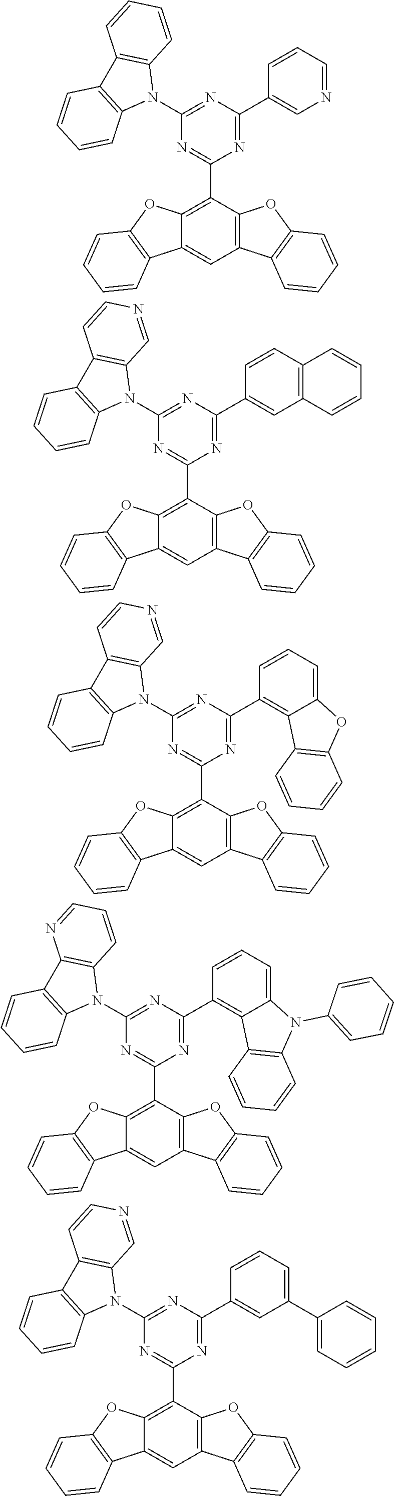

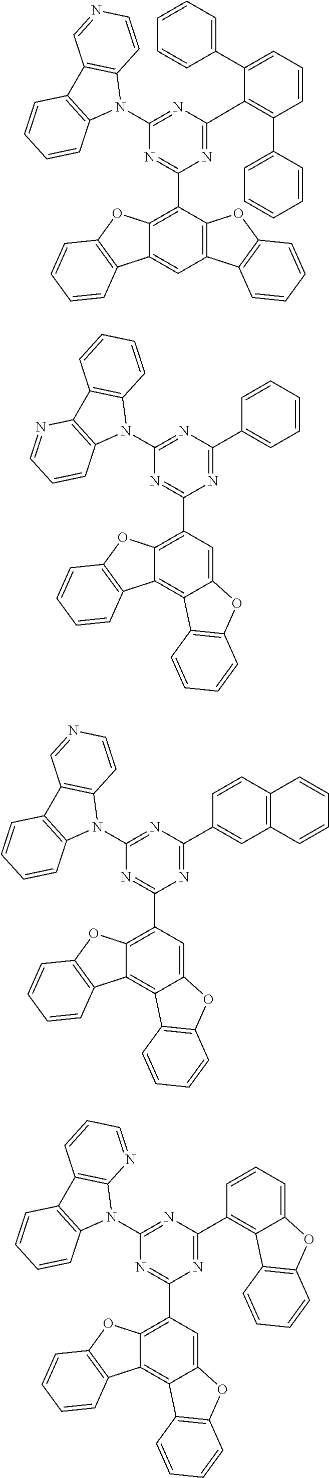

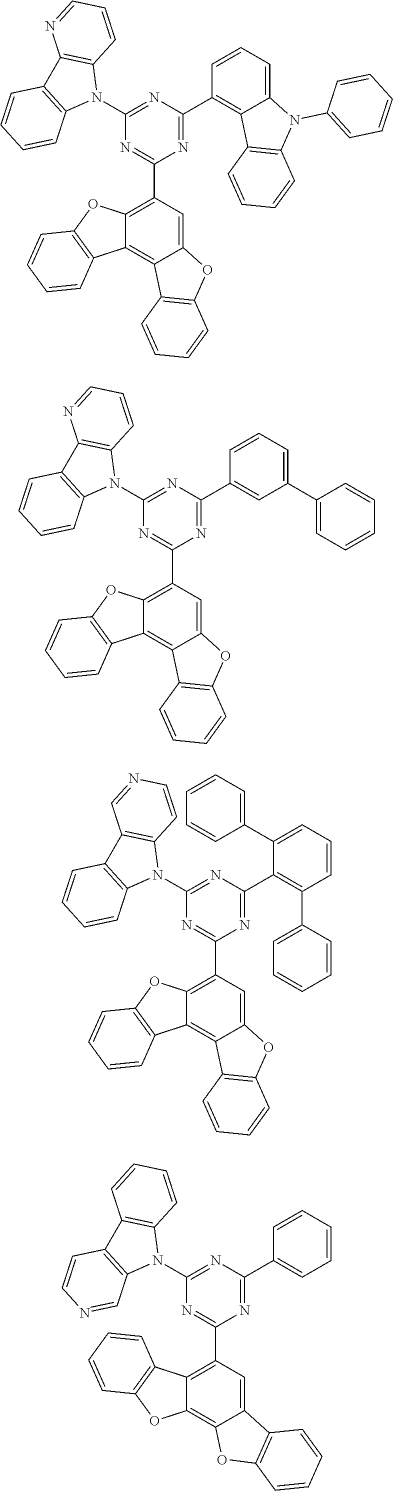

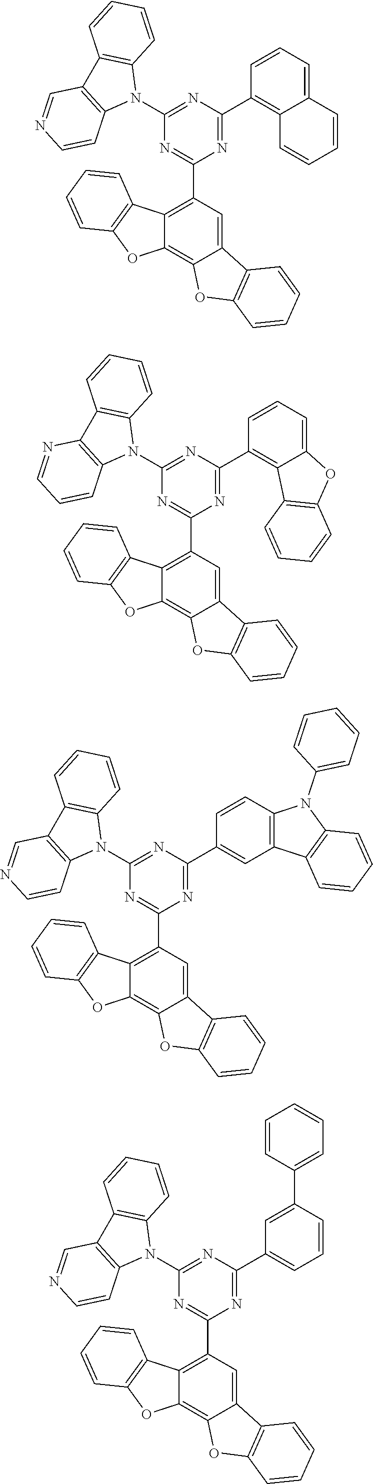

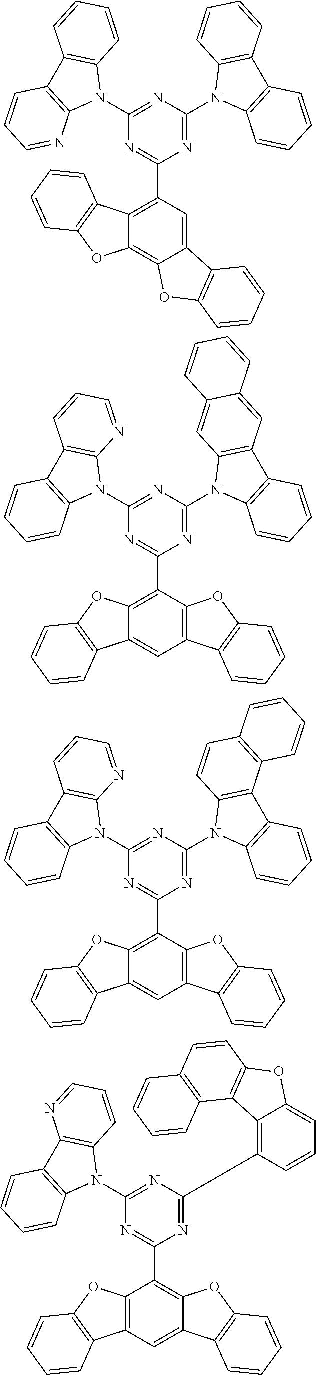

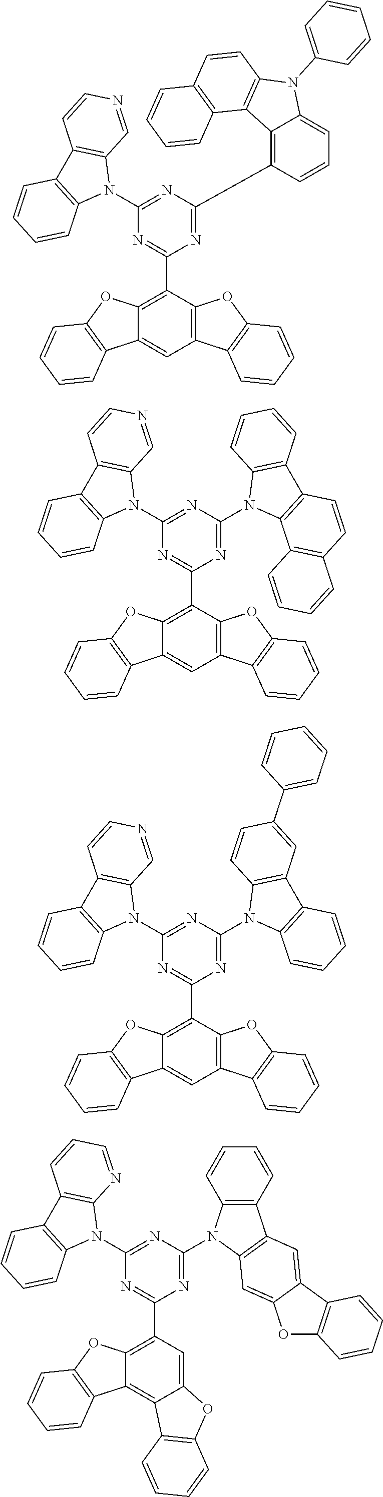

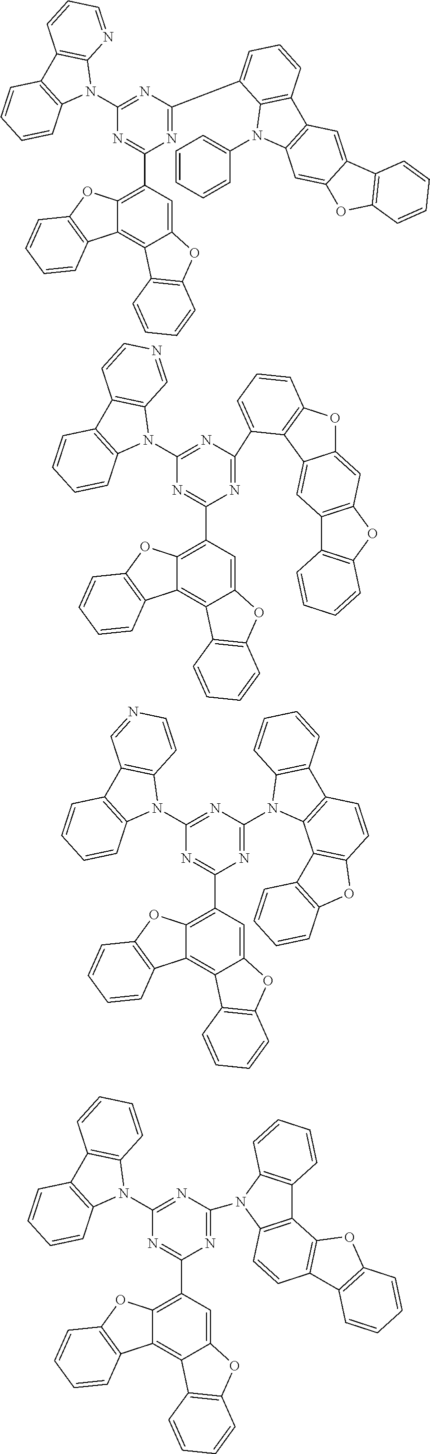

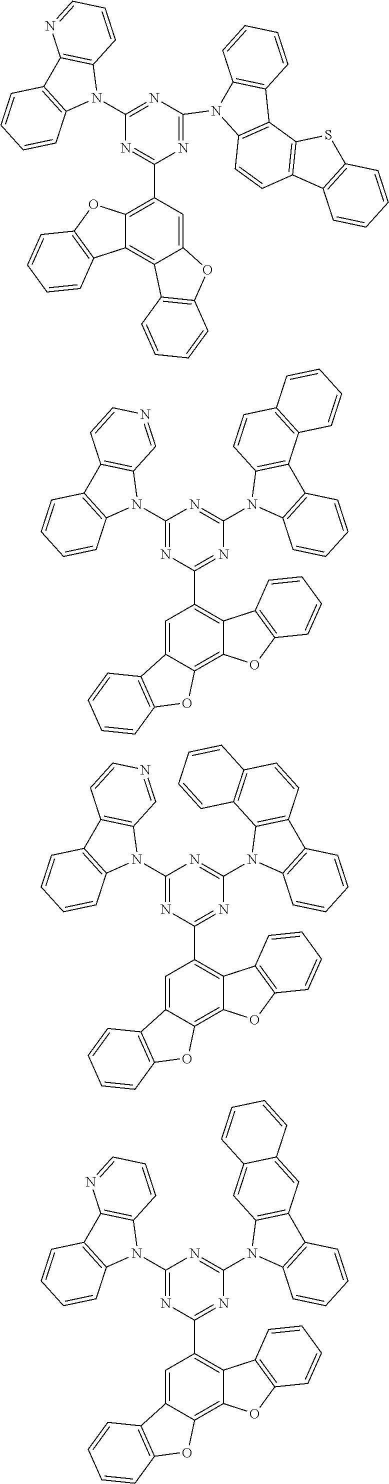

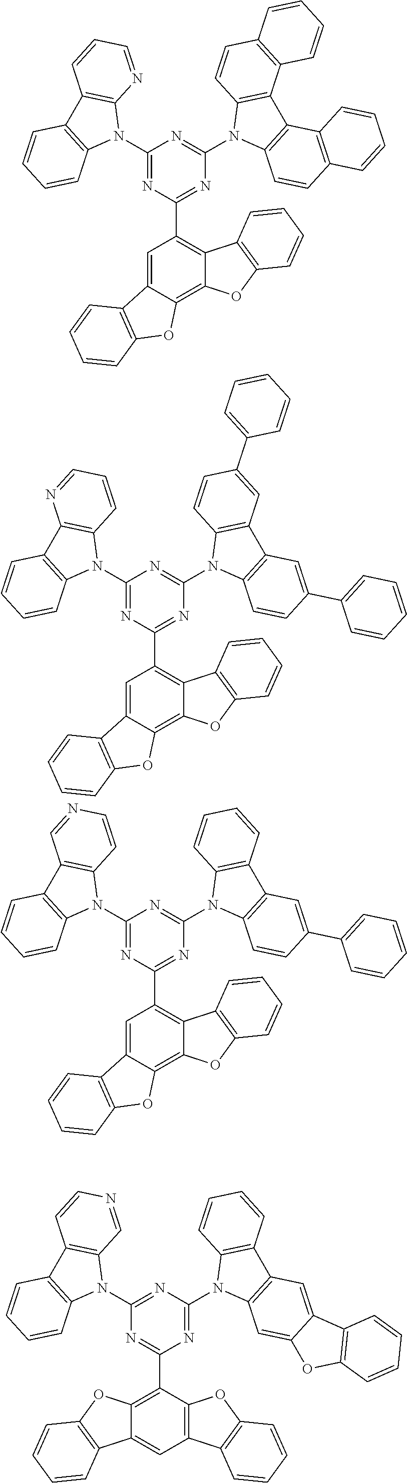

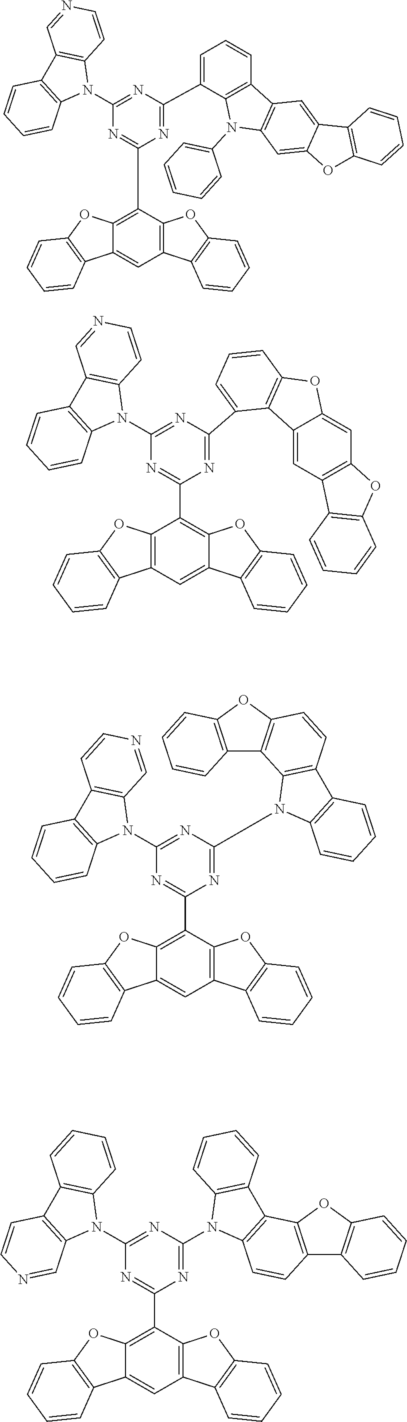

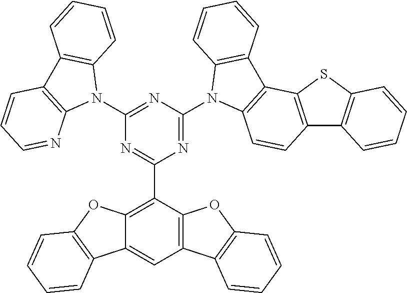 [SMILES: c1ccc2c(c1)oc1c(-c3nc(-n4c5ccccc5c5c6sc7ccccc7c6ccc54)nc(-n4c5ccccc5c5cccnc54)n3)c3oc4ccccc4c3cc12]

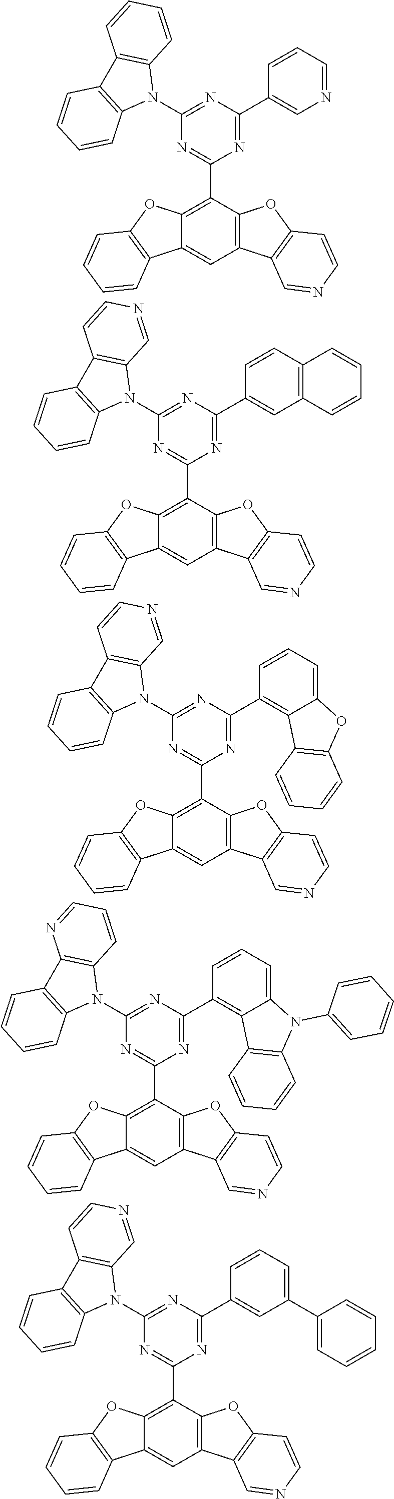

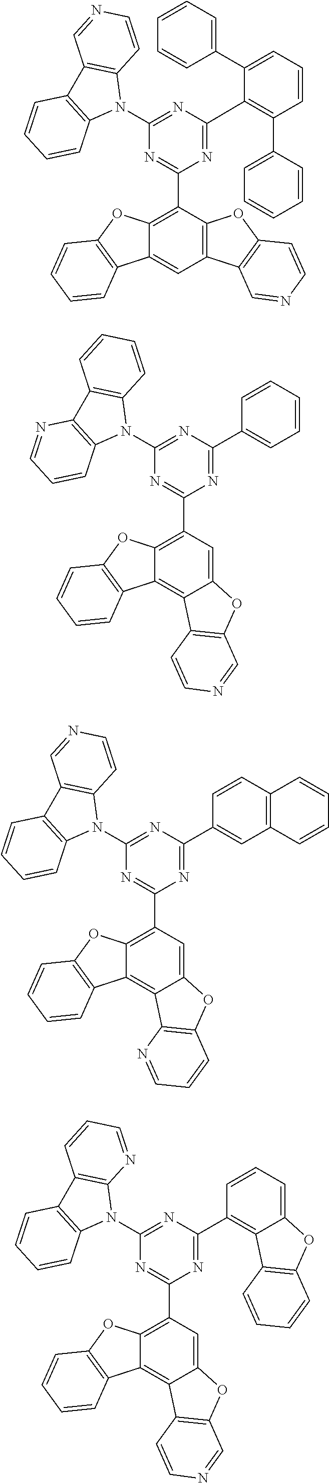

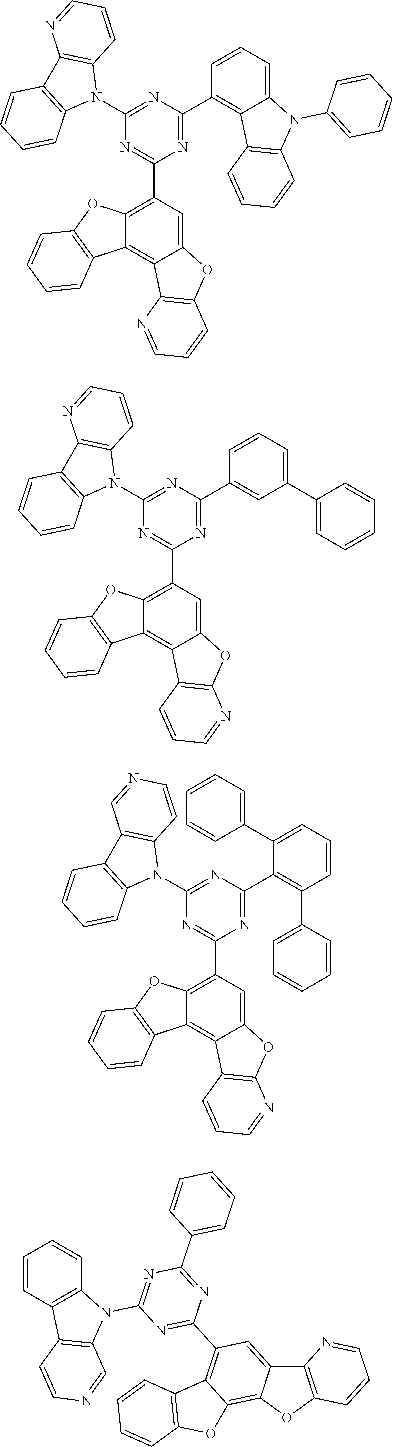

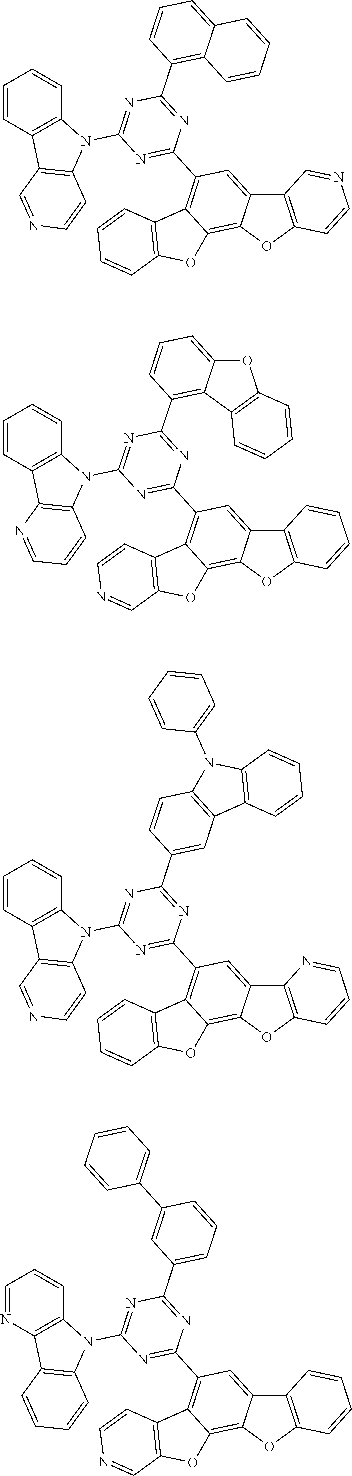 [SMILES: c1ccc2c(-c3nc(-c4cc5c6cnccc6oc5c5oc6ccccc6c45)nc(-n4c5ccccc5c5cnccc54)n3)cccc2c1 ; c1ccc2c(c1)oc1c2cc(-c2nc(-c3cccc4oc5ccccc5c34)nc(-n3c4ccccc4c4ncccc43)n2)c2c3ccncc3oc12 ; c1ccc(-n2c3ccccc3c3cc(-c4nc(-c5cc6c7ncccc7oc6c6oc7ccccc7c56)nc(-n5c6ccccc6c6cnccc65)n4)ccc32)cc1 ; c1ccc(-c2cccc(-c3nc(-c4cc5c6ccccc6oc5c5oc6cnccc6c45)nc(-n4c5ccccc5c5ncccc54)n3)c2)cc1]

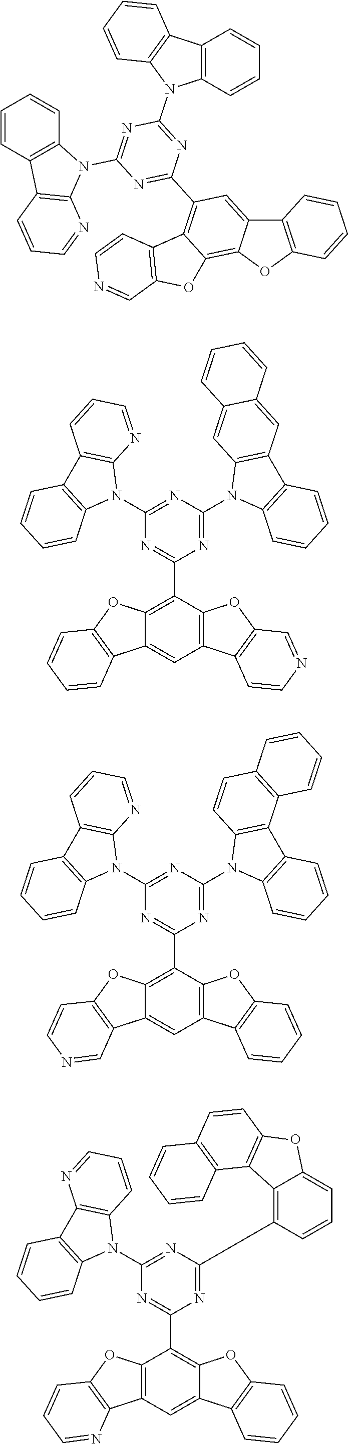

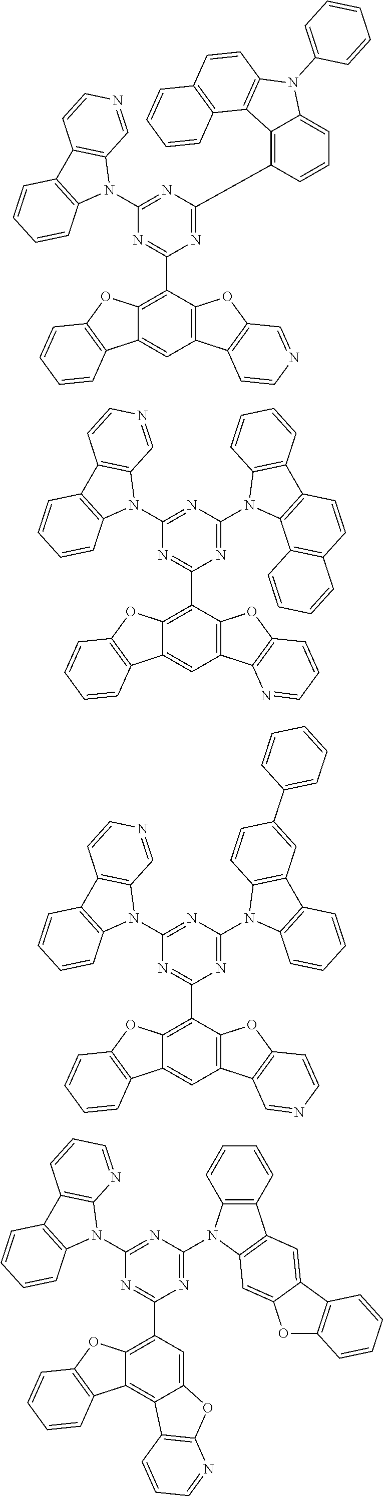

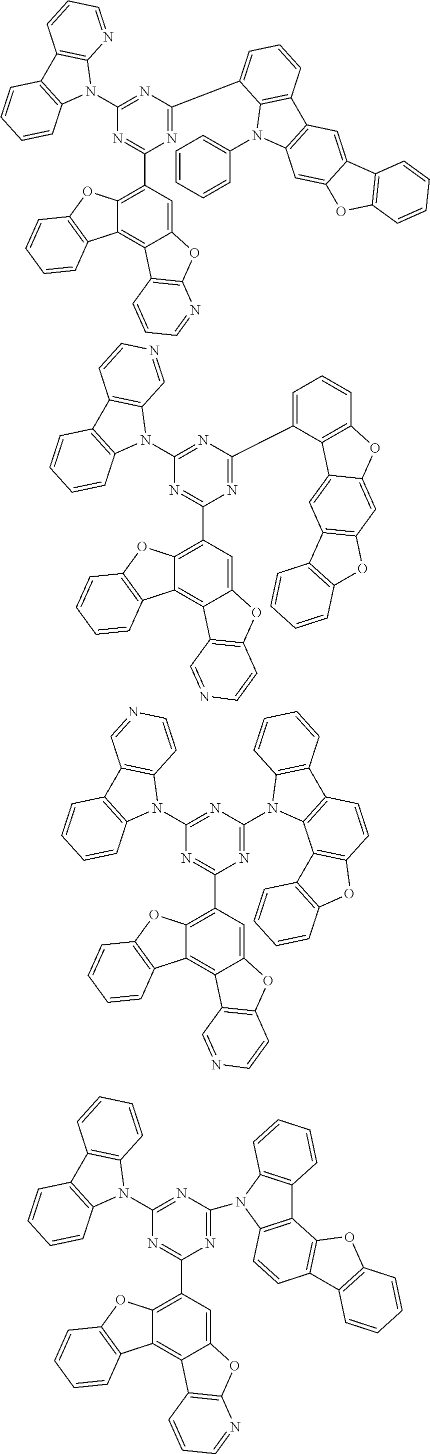

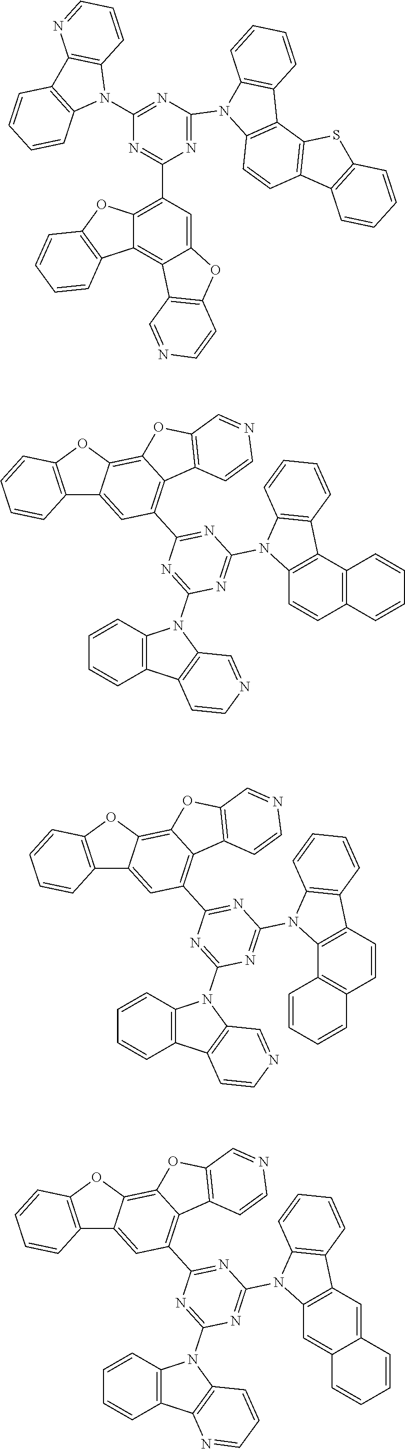 [SMILES: c1ccc2c(c1)oc1c(-c3nc(-n4c5ccccc5c5ncccc54)nc(-n4c5ccccc5c5c6sc7ccccc7c6ccc54)n3)cc3oc4ccncc4c3c12 ; c1ccc2c(c1)ccc1c2c2ccccc2n1-c1nc(-c2cc3c4ccccc4oc3c3oc4cnccc4c23)nc(-n2c3ccccc3c3ccncc32)n1 ; c1ccc2c(c1)ccc1c3ccccc3n(-c3nc(-c4cc5c6ccccc6oc5c5oc6cnccc6c45)nc(-n4c5ccccc5c5ccncc54)n3)c21 ; c1ccc2cc3c(cc2c1)c1ccccc1n3-c1nc(-c2cc3c4ccccc4oc3c3oc4cnccc4c23)nc(-n2c3ccccc3c3ncccc32)n1]

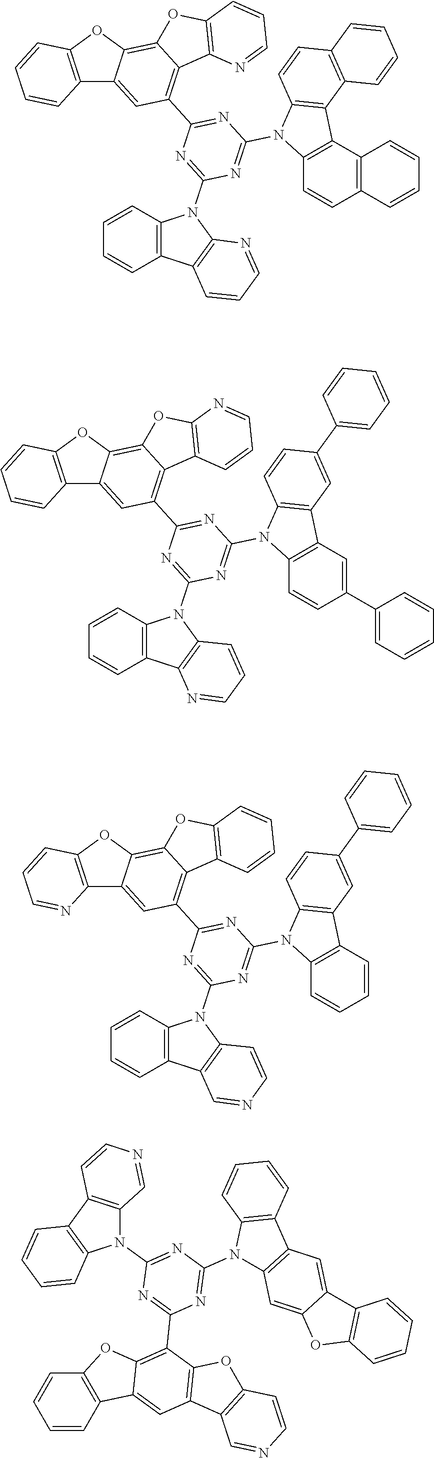 [SMILES: c1ccc2c(c1)ccc1c2c2c3ccccc3ccc2n1-c1nc(-c2cc3c4ccccc4oc3c3oc4cccnc4c23)nc(-n2c3ccccc3c3cccnc32)n1 ; c1ccc(-c2ccc3c(c2)c2cc(-c4ccccc4)ccc2n3-c2nc(-c3cc4c5ccccc5oc4c4oc5ncccc5c34)nc(-n3c4ccccc4c4ncccc43)n2)cc1 ; c1ccc(-c2ccc3c(c2)c2ccccc2n3-c2nc(-c3cc4c5ncccc5oc4c4oc5ccccc5c34)nc(-n3c4ccccc4c4cnccc43)n2)cc1 ; c1ccc2c(c1)oc1cc3c(cc12)c1ccccc1n3-c1nc(-c2c3oc4ccccc4c3cc3c2oc2ccncc23)nc(-n2c3ccccc3c3ccncc32)n1]

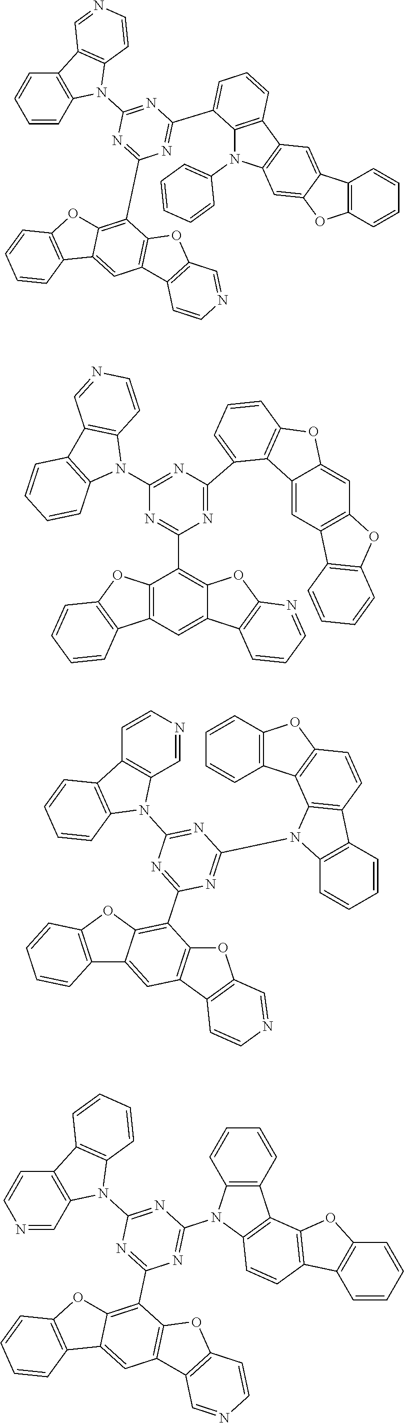 [SMILES: c1ccc(-n2c3cc4oc5ccccc5c4cc3c3cccc(-c4nc(-c5c6oc7ccccc7c6cc6c5oc5cnccc56)nc(-n5c6ccccc6c6cnccc65)n4)c32)cc1 ; c1ccc2c(c1)oc1cc3oc4cccc(-c5nc(-c6c7oc8ccccc8c7cc7c6oc6ncccc67)nc(-n6c7ccccc7c7cnccc76)n5)c4c3cc12 ; c1ccc2c(c1)oc1c(-c3nc(-n4c5ccccc5c5ccncc54)nc(-n4c5ccccc5c5ccc6oc7ccccc7c6c54)n3)c3oc4cnccc4c3cc12 ; c1ccc2c(c1)oc1c(-c3nc(-n4c5ccccc5c5ccncc54)nc(-n4c5ccccc5c5c6oc7ccccc7c6ccc54)n3)c3oc4ccncc4c3cc12]

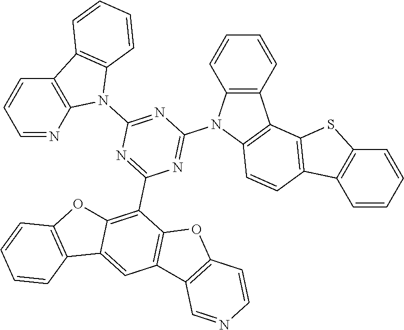 [SMILES: c1ccc2c(c1)oc1c(-c3nc(-n4c5ccccc5c5c6sc7ccccc7c6ccc54)nc(-n4c5ccccc5c5cccnc54)n3)c3oc4ccncc4c3cc12]

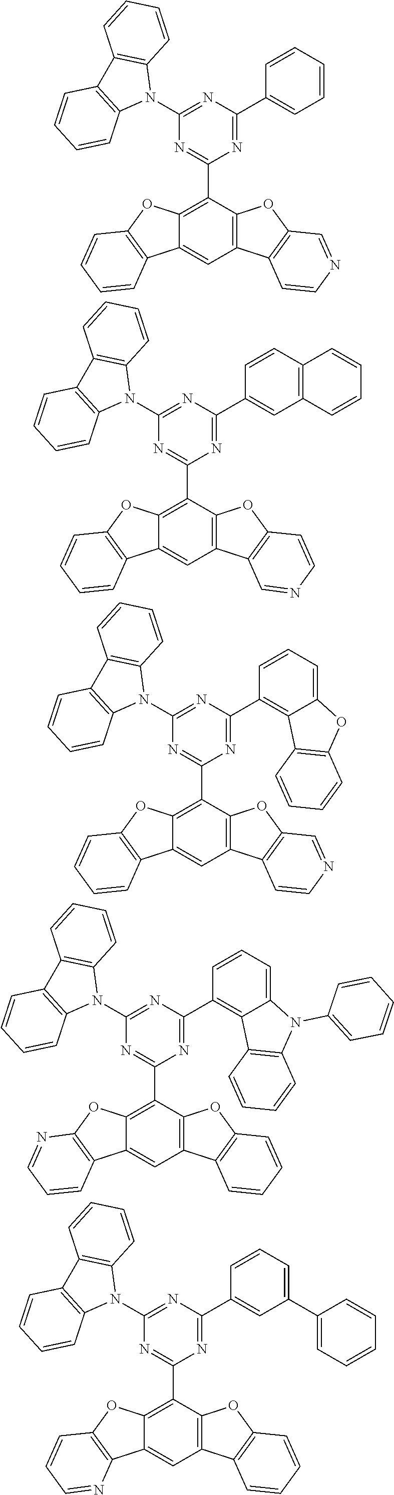

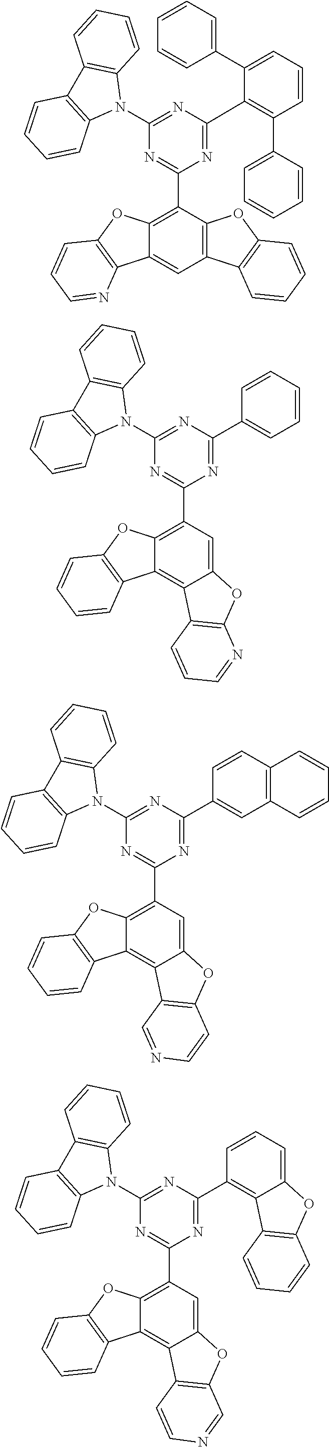

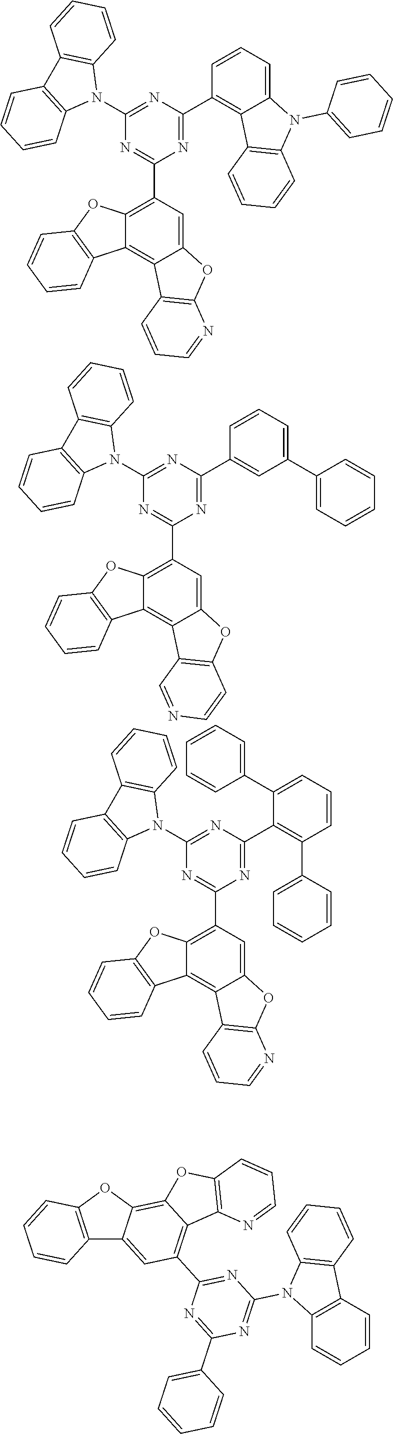

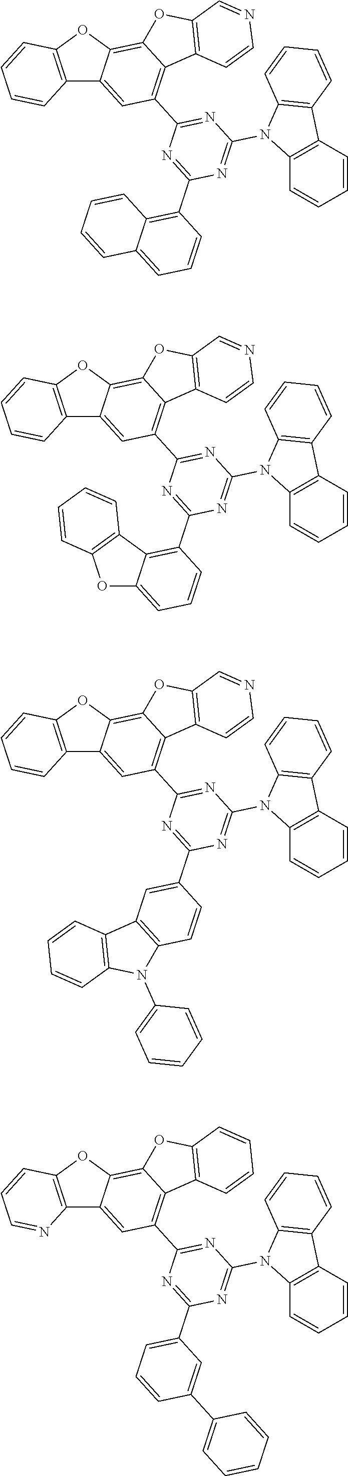 [SMILES: c1ccc2c(-c3nc(-c4cc5c6ccccc6oc5c5oc6cnccc6c45)nc(-n4c5ccccc5c5ccccc54)n3)cccc2c1 ; c1ccc2c(c1)oc1c2cc(-c2nc(-c3cccc4oc5ccccc5c34)nc(-n3c4ccccc4c4ccccc43)n2)c2c3ccncc3oc12 ; c1ccc(-n2c3ccccc3c3cc(-c4nc(-c5cc6c7ccccc7oc6c6oc7cnccc7c56)nc(-n5c6ccccc6c6ccccc65)n4)ccc32)cc1 ; c1ccc(-c2cccc(-c3nc(-c4cc5c6ncccc6oc5c5oc6ccccc6c45)nc(-n4c5ccccc5c5ccccc54)n3)c2)cc1]

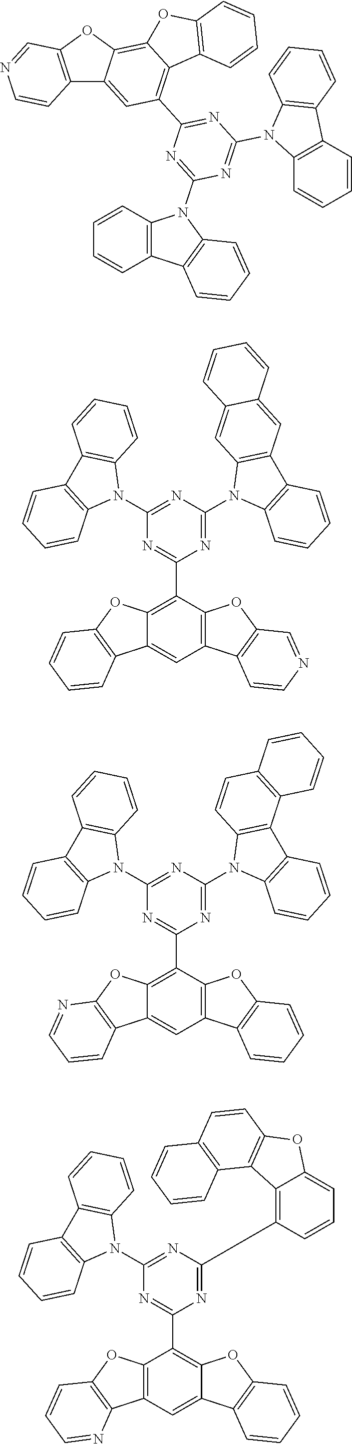

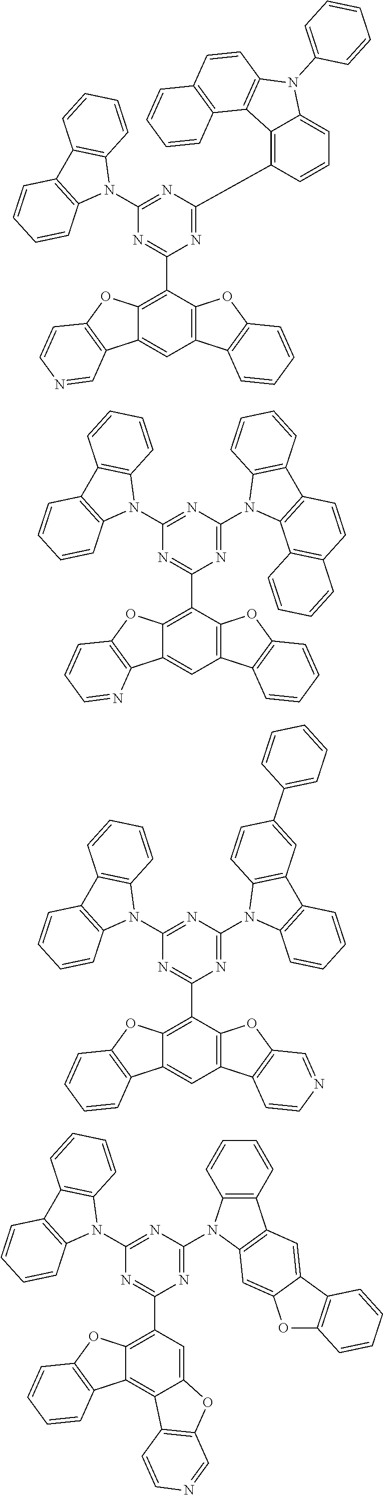

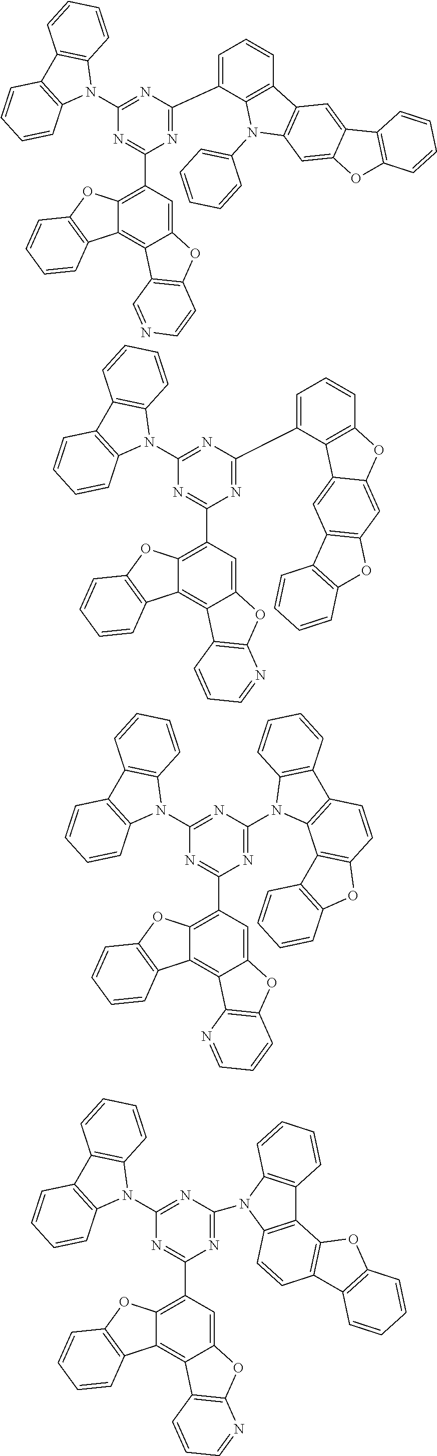

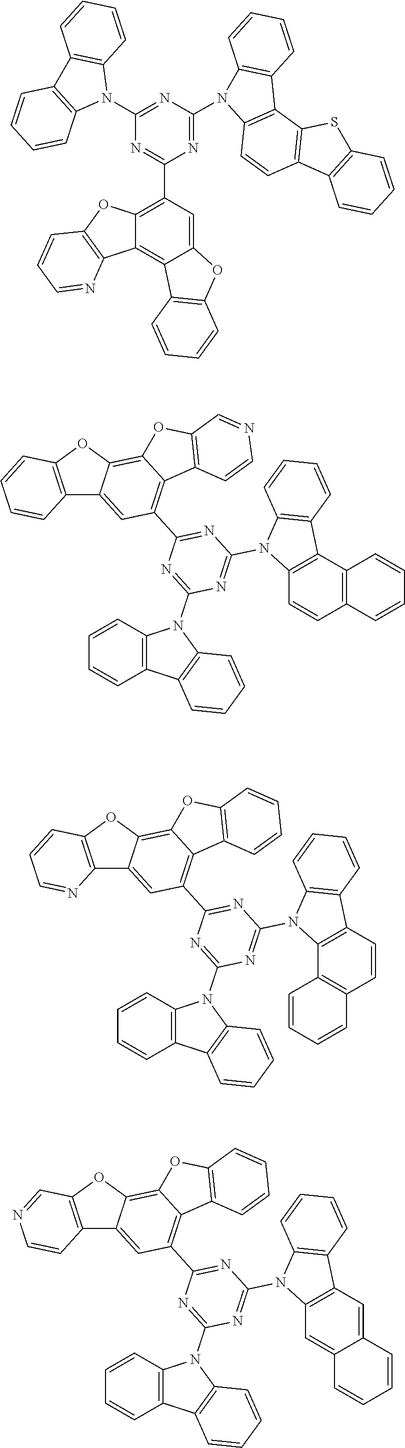 [SMILES: c1ccc2c(c1)oc1cc(-c3nc(-n4c5ccccc5c5ccccc54)nc(-n4c5ccccc5c5c6sc7ccccc7c6ccc54)n3)c3oc4cccnc4c3c12 ; c1ccc2c(c1)ccc1c2c2ccccc2n1-c1nc(-c2cc3c4ccccc4oc3c3oc4cnccc4c23)nc(-n2c3ccccc3c3ccccc32)n1 ; c1ccc2c(c1)ccc1c3ccccc3n(-c3nc(-c4cc5c6ncccc6oc5c5oc6ccccc6c45)nc(-n4c5ccccc5c5ccccc54)n3)c21 ; c1ccc2cc3c(cc2c1)c1ccccc1n3-c1nc(-c2cc3c4ccncc4oc3c3oc4ccccc4c23)nc(-n2c3ccccc3c3ccccc32)n1]

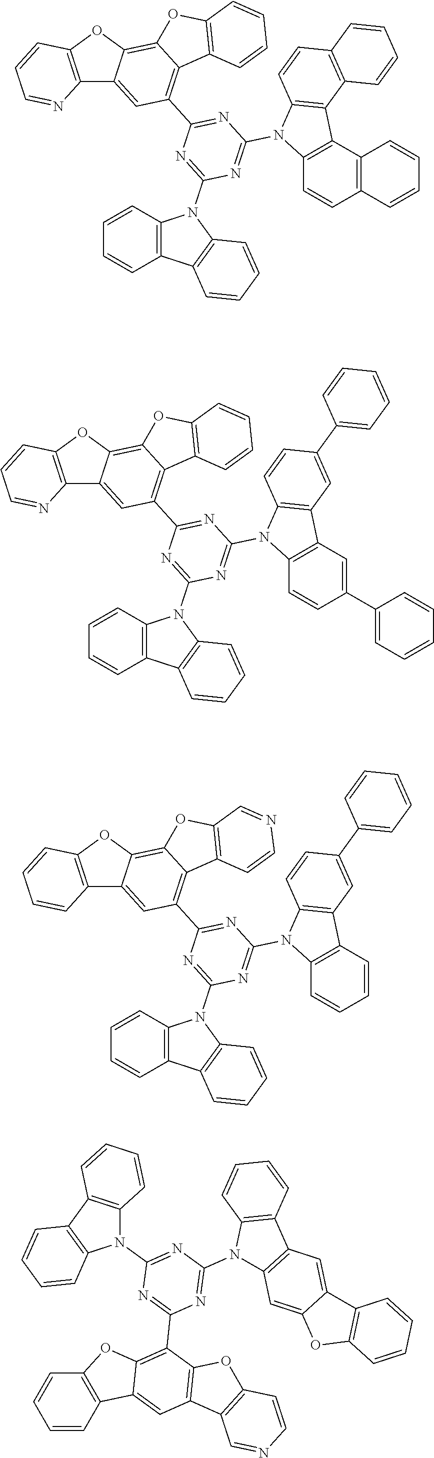 [SMILES: c1ccc2c(c1)ccc1c2c2c3ccccc3ccc2n1-c1nc(-c2cc3c4ncccc4oc3c3oc4ccccc4c23)nc(-n2c3ccccc3c3ccccc32)n1 ; c1ccc(-c2ccc3c(c2)c2cc(-c4ccccc4)ccc2n3-c2nc(-c3cc4c5ncccc5oc4c4oc5ccccc5c34)nc(-n3c4ccccc4c4ccccc43)n2)cc1 ; c1ccc(-c2ccc3c(c2)c2ccccc2n3-c2nc(-c3cc4c5ccccc5oc4c4oc5cnccc5c34)nc(-n3c4ccccc4c4ccccc43)n2)cc1 ; c1ccc2c(c1)oc1cc3c(cc12)c1ccccc1n3-c1nc(-c2c3oc4ccccc4c3cc3c2oc2ccncc23)nc(-n2c3ccccc3c3ccccc32)n1]

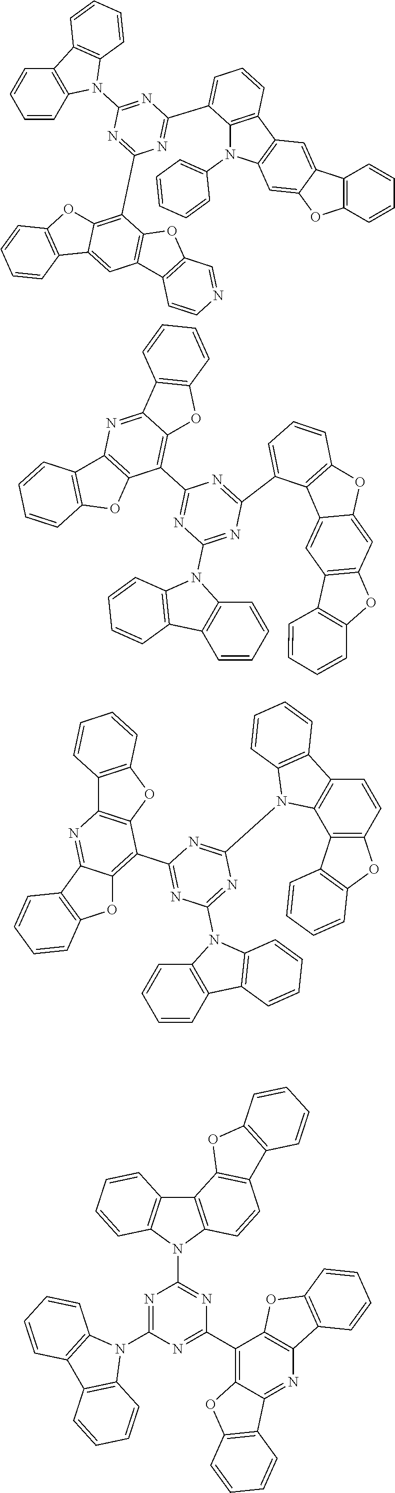

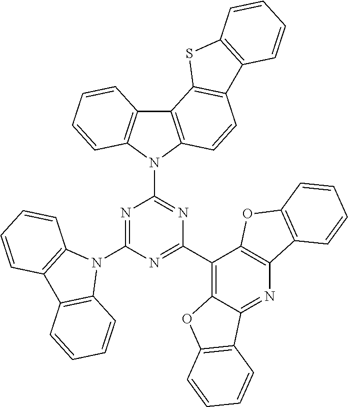 [SMILES: c1ccc2c(c1)oc1c(-c3nc(-n4c5ccccc5c5ccccc54)nc(-n4c5ccccc5c5c6sc7ccccc7c6ccc54)n3)c3oc4ccccc4c3nc12]

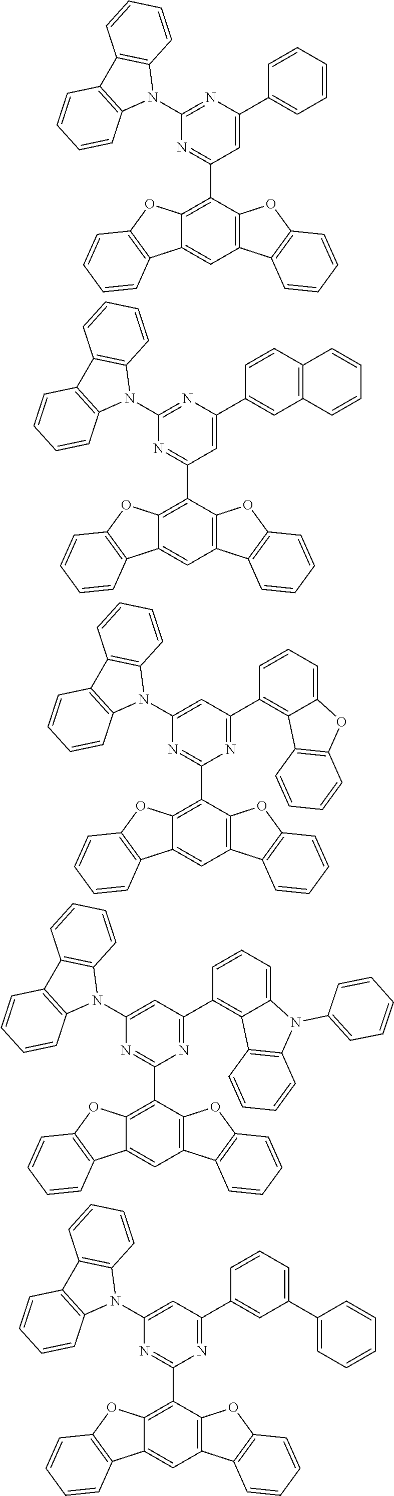

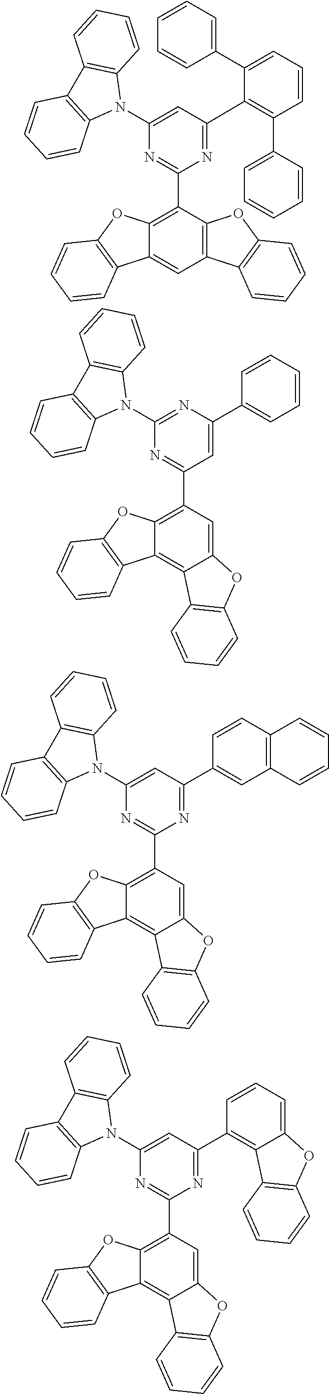

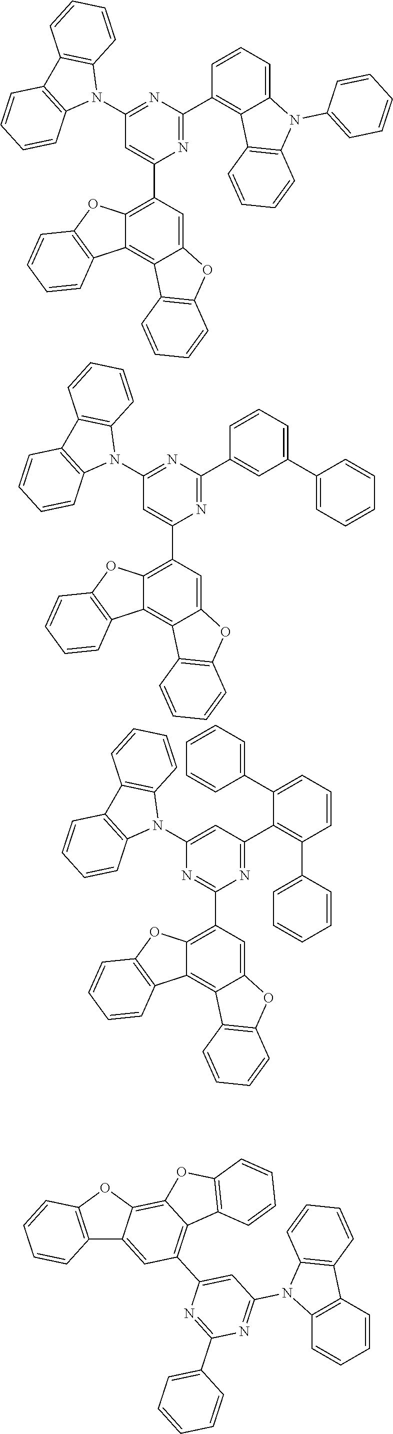

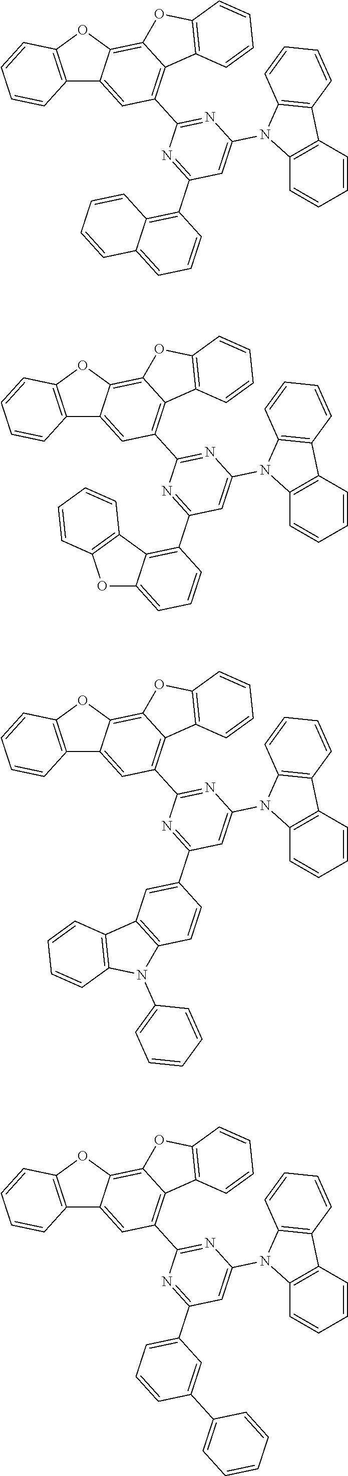 [SMILES: c1ccc2c(-c3cc(-n4c5ccccc5c5ccccc54)nc(-c4cc5c6ccccc6oc5c5oc6ccccc6c45)n3)cccc2c1 ; c1ccc2c(c1)oc1c2cc(-c2nc(-c3cccc4oc5ccccc5c34)cc(-n3c4ccccc4c4ccccc43)n2)c2c3ccccc3oc12 ; c1ccc(-n2c3ccccc3c3cc(-c4cc(-n5c6ccccc6c6ccccc65)nc(-c5cc6c7ccccc7oc6c6oc7ccccc7c56)n4)ccc32)cc1 ; c1ccc(-c2cccc(-c3cc(-n4c5ccccc5c5ccccc54)nc(-c4cc5c6ccccc6oc5c5oc6ccccc6c45)n3)c2)cc1]

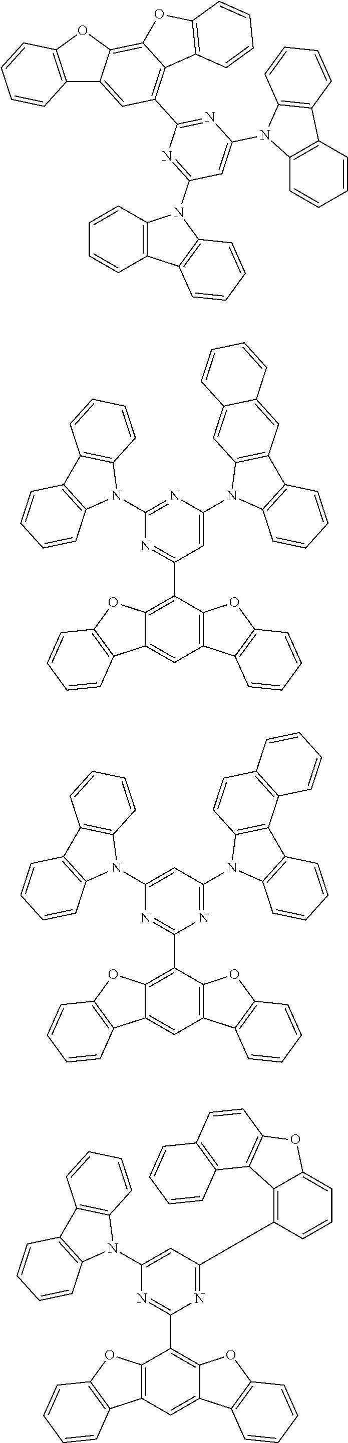

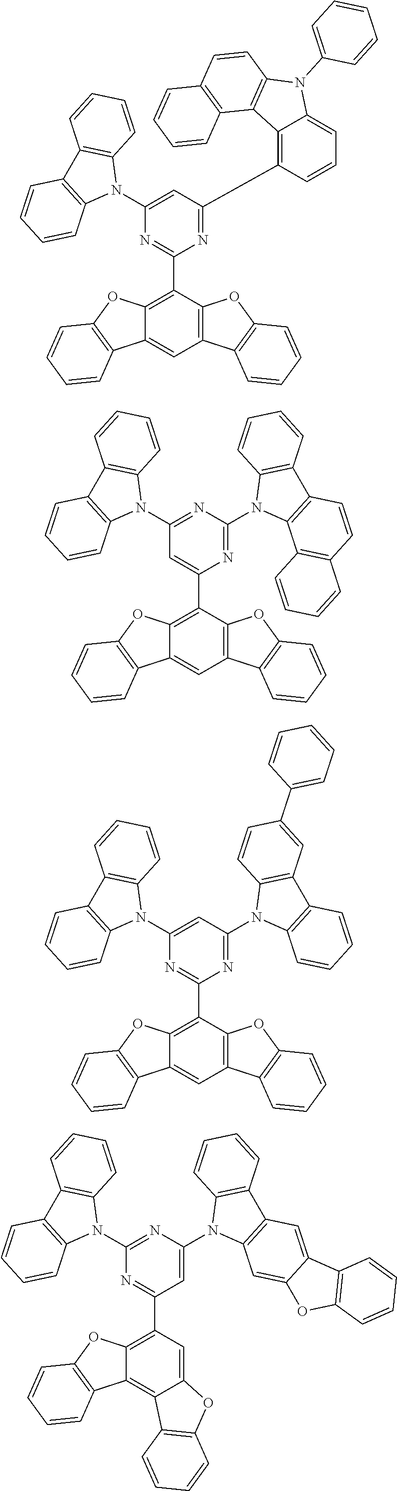

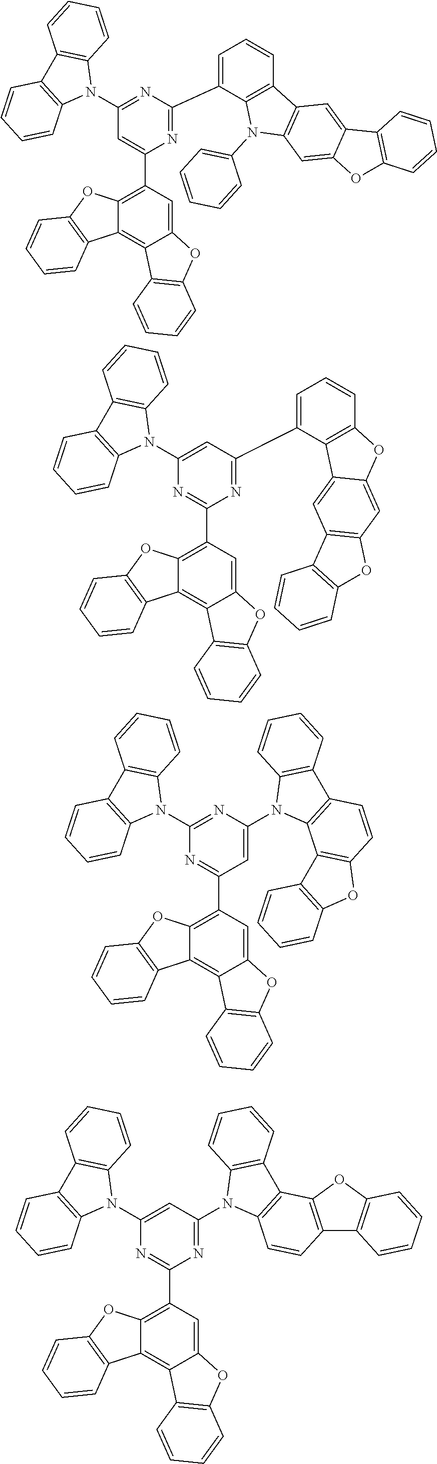

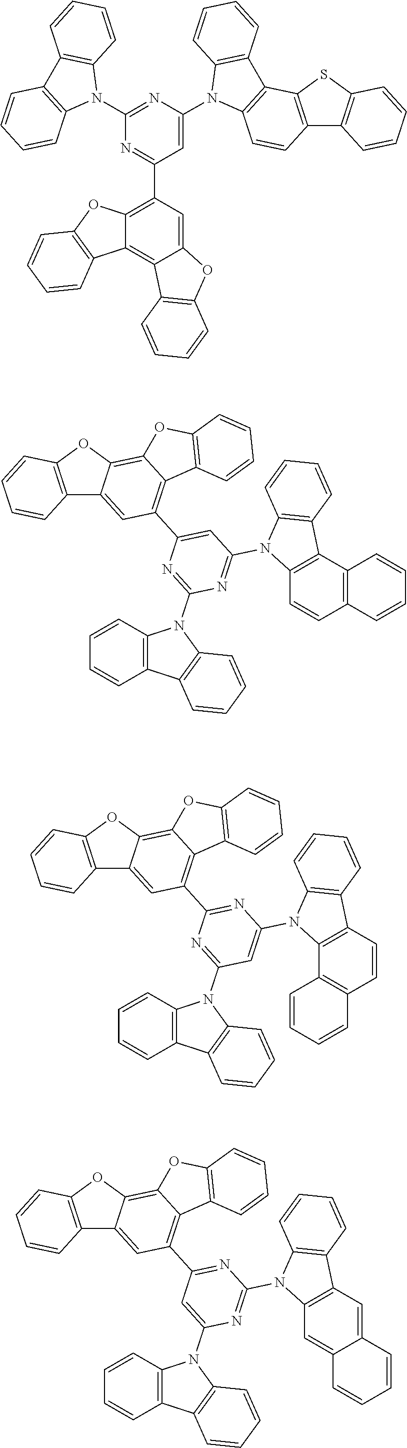 [SMILES: c1ccc2c(c1)oc1cc(-c3cc(-n4c5ccccc5c5c6sc7ccccc7c6ccc54)nc(-n4c5ccccc5c5ccccc54)n3)c3oc4ccccc4c3c12 ; c1ccc2c(c1)ccc1c2c2ccccc2n1-c1cc(-c2cc3c4ccccc4oc3c3oc4ccccc4c23)nc(-n2c3ccccc3c3ccccc32)n1 ; c1ccc2c(c1)ccc1c3ccccc3n(-c3cc(-n4c5ccccc5c5ccccc54)nc(-c4cc5c6ccccc6oc5c5oc6ccccc6c45)n3)c21 ; c1ccc2cc3c(cc2c1)c1ccccc1n3-c1nc(-c2cc3c4ccccc4oc3c3oc4ccccc4c23)cc(-n2c3ccccc3c3ccccc32)n1]

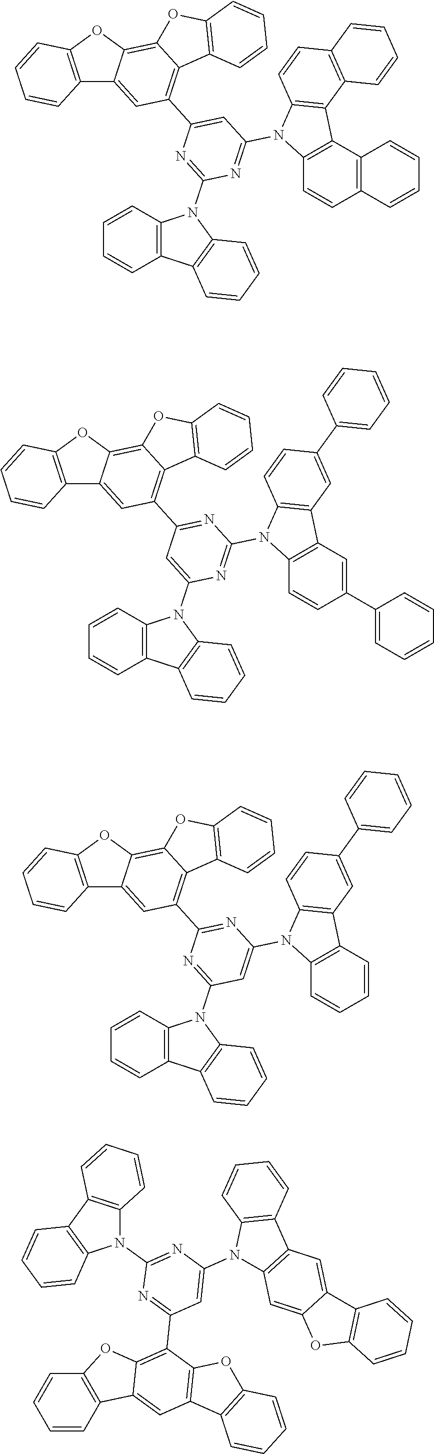 [SMILES: c1ccc2c(c1)ccc1c2c2c3ccccc3ccc2n1-c1cc(-c2cc3c4ccccc4oc3c3oc4ccccc4c23)nc(-n2c3ccccc3c3ccccc32)n1 ; c1ccc(-c2ccc3c(c2)c2cc(-c4ccccc4)ccc2n3-c2nc(-c3cc4c5ccccc5oc4c4oc5ccccc5c34)cc(-n3c4ccccc4c4ccccc43)n2)cc1 ; c1ccc(-c2ccc3c(c2)c2ccccc2n3-c2cc(-n3c4ccccc4c4ccccc43)nc(-c3cc4c5ccccc5oc4c4oc5ccccc5c34)n2)cc1 ; c1ccc2c(c1)oc1cc3c(cc12)c1ccccc1n3-c1cc(-c2c3oc4ccccc4c3cc3c2oc2ccccc23)nc(-n2c3ccccc3c3ccccc32)n1]

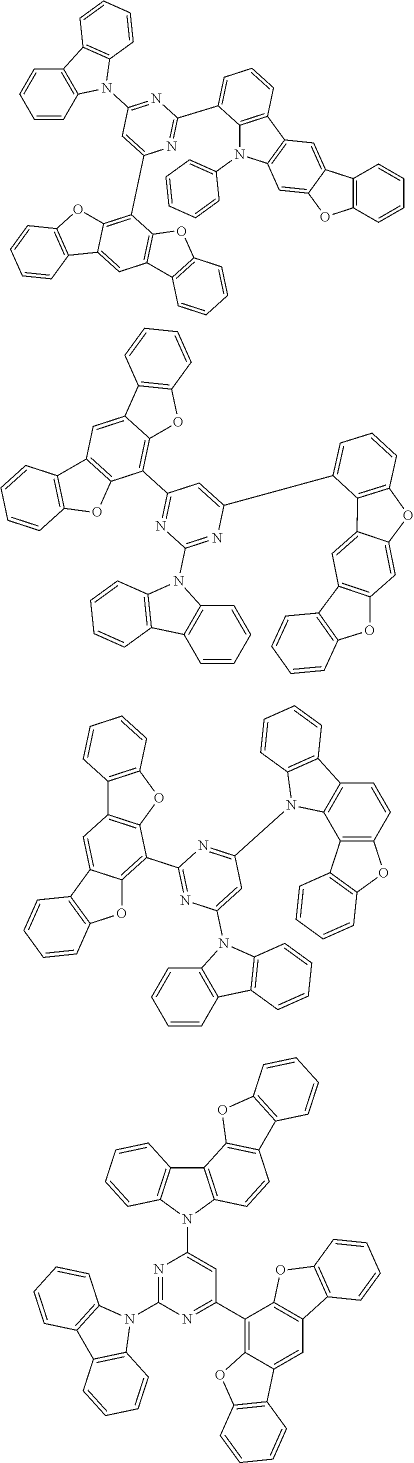

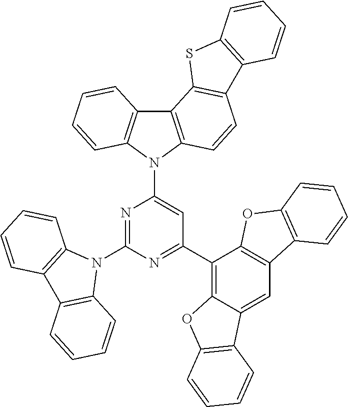 [SMILES: c1ccc2c(c1)oc1c(-c3cc(-n4c5ccccc5c5c6sc7ccccc7c6ccc54)nc(-n4c5ccccc5c5ccccc54)n3)c3oc4ccccc4c3cc12]

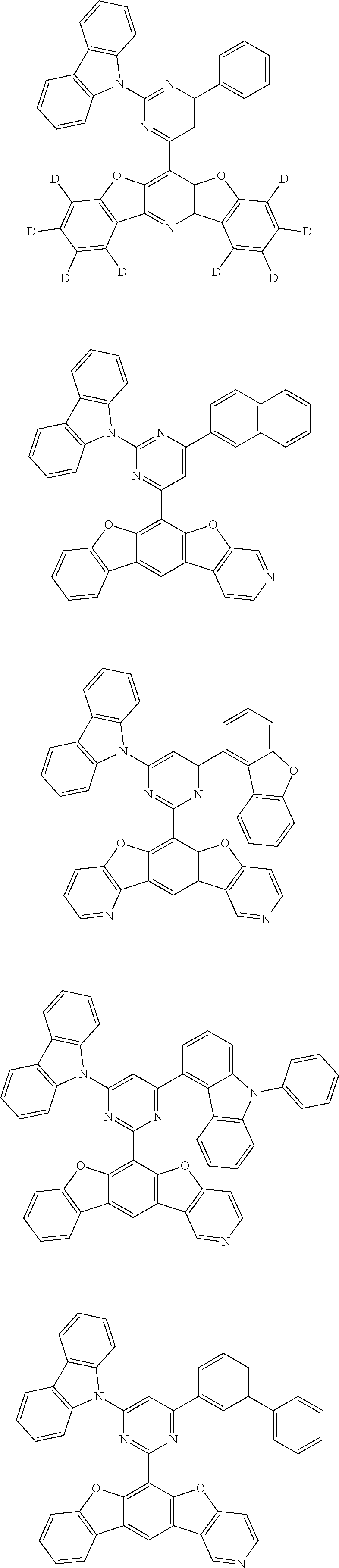 [SMILES: [2H]c1c([2H])c([2H])c2c(oc3c(-c4cc(-c5ccccc5)nc(-n5c6ccccc6c6ccccc65)n4)c4oc5c([2H])c([2H])c([2H])c([2H])c5c4nc32)c1[2H] ; c1ccc2cc(-c3cc(-c4c5oc6ccccc6c5cc5c4oc4cnccc45)nc(-n4c5ccccc5c5ccccc54)n3)ccc2c1 ; c1ccc2c(c1)oc1cccc(-c3cc(-n4c5ccccc5c5ccccc54)nc(-c4c5oc6ccncc6c5cc5c4oc4cccnc45)n3)c12 ; c1ccc(-n2c3ccccc3c3c(-c4cc(-n5c6ccccc6c6ccccc65)nc(-c5c6oc7ccccc7c6cc6c5oc5ccncc56)n4)cccc32)cc1 ; c1ccc(-c2cccc(-c3cc(-n4c5ccccc5c5ccccc54)nc(-c4c5oc6ccccc6c5cc5c4oc4ccncc45)n3)c2)cc1]

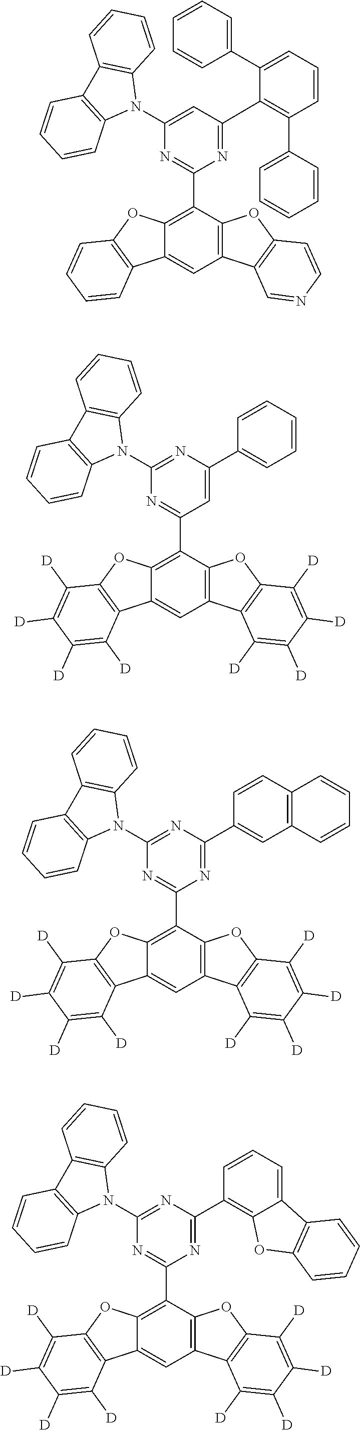 [SMILES: c1ccc(-c2cccc(-c3ccccc3)c2-c2cc(-n3c4ccccc4c4ccccc43)nc(-c3c4oc5ccccc5c4cc4c3oc3ccncc34)n2)cc1 ; [2H]c1c([2H])c([2H])c2c(oc3c(-c4cc(-c5ccccc5)nc(-n5c6ccccc6c6ccccc65)n4)c4oc5c([2H])c([2H])c([2H])c([2H])c5c4cc32)c1[2H] ; [2H]c1c([2H])c([2H])c2c(oc3c(-c4nc(-c5ccc6ccccc6c5)nc(-n5c6ccccc6c6ccccc65)n4)c4oc5c([2H])c([2H])c([2H])c([2H])c5c4cc32)c1[2H] ; [2H]c1c([2H])c([2H])c2c(oc3c(-c4nc(-c5cccc6c5oc5ccccc56)nc(-n5c6ccccc6c6ccccc65)n4)c4oc5c([2H])c([2H])c([2H])c([2H])c5c4cc32)c1[2H]]

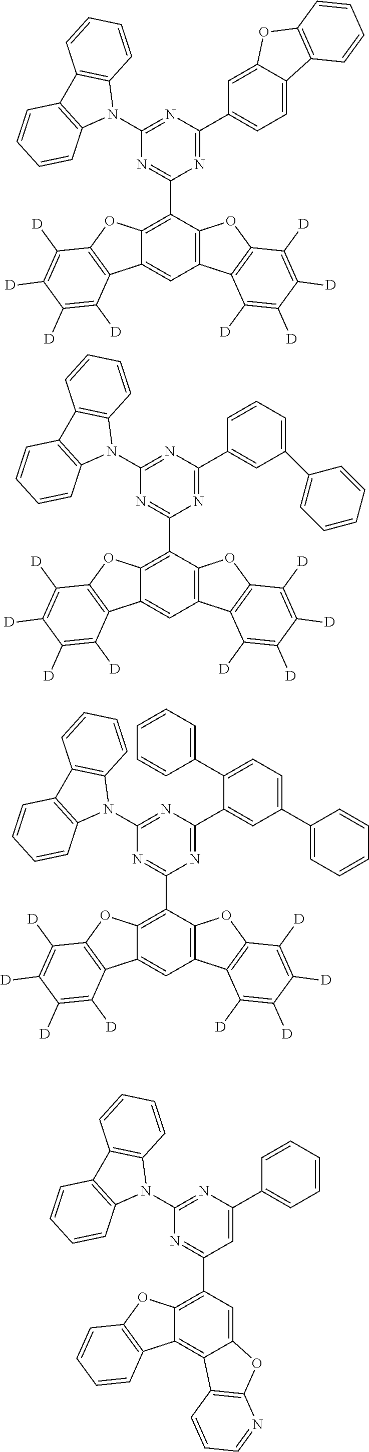

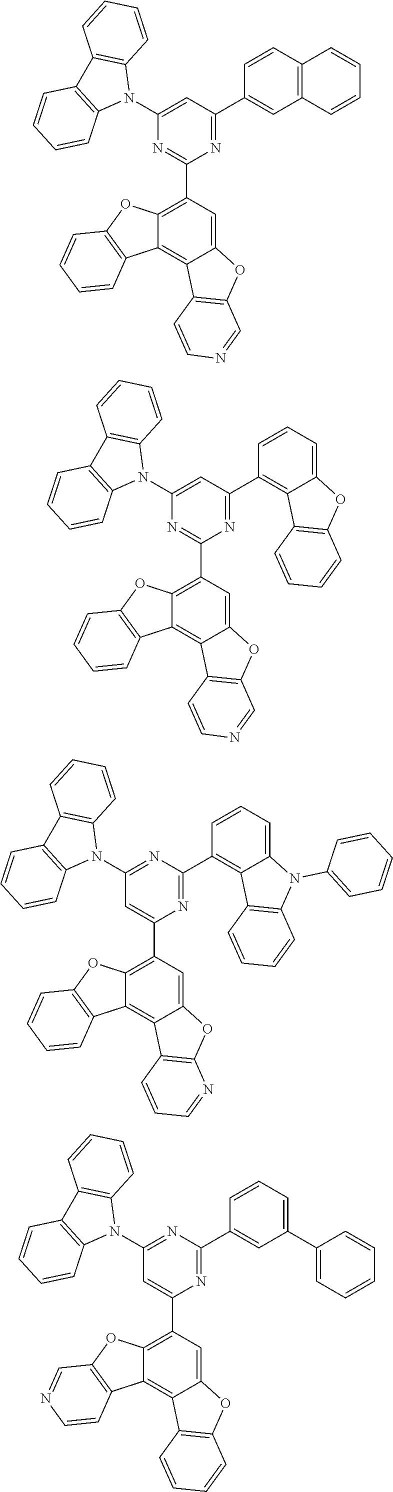

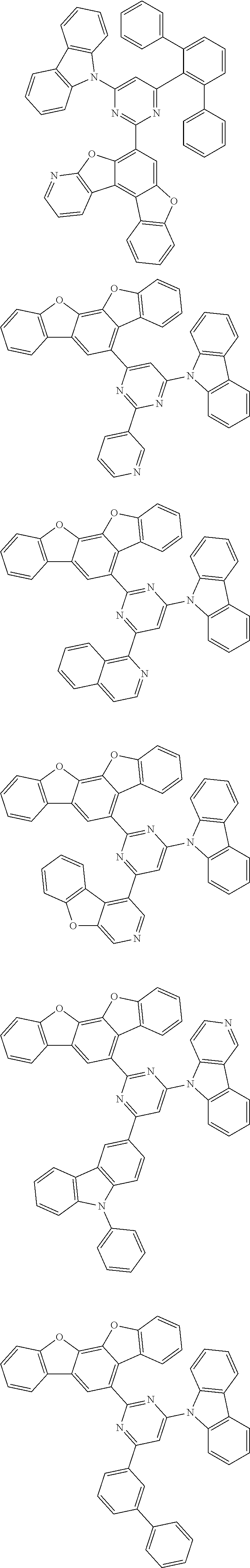

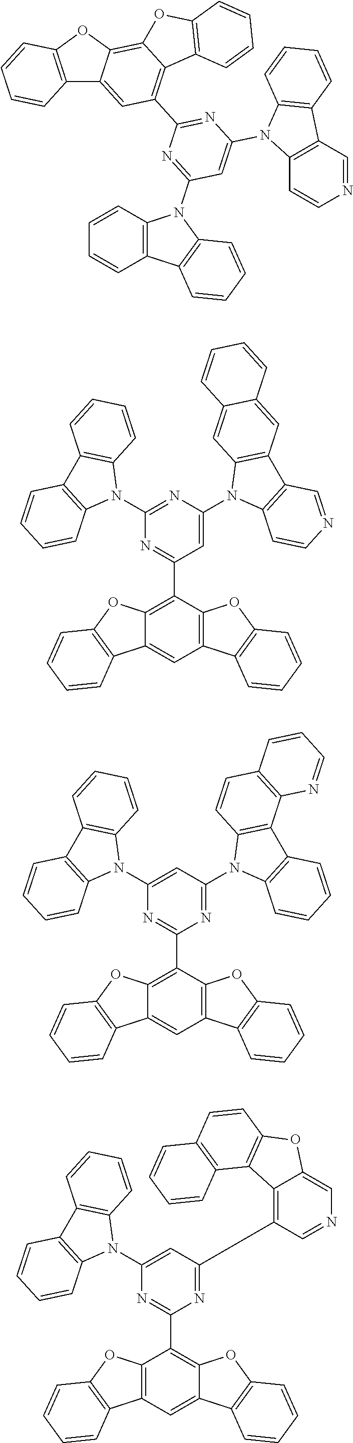

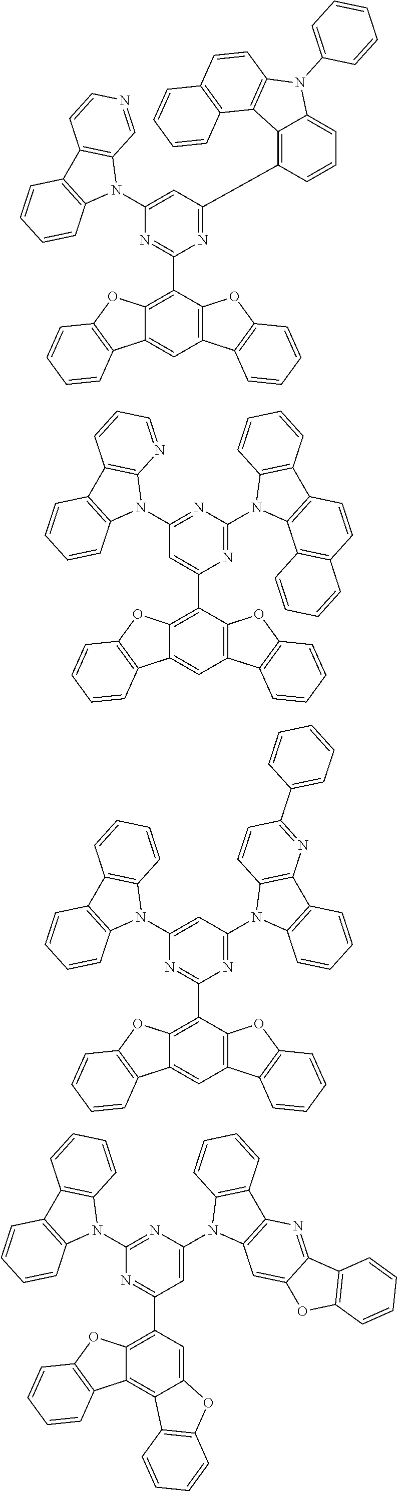

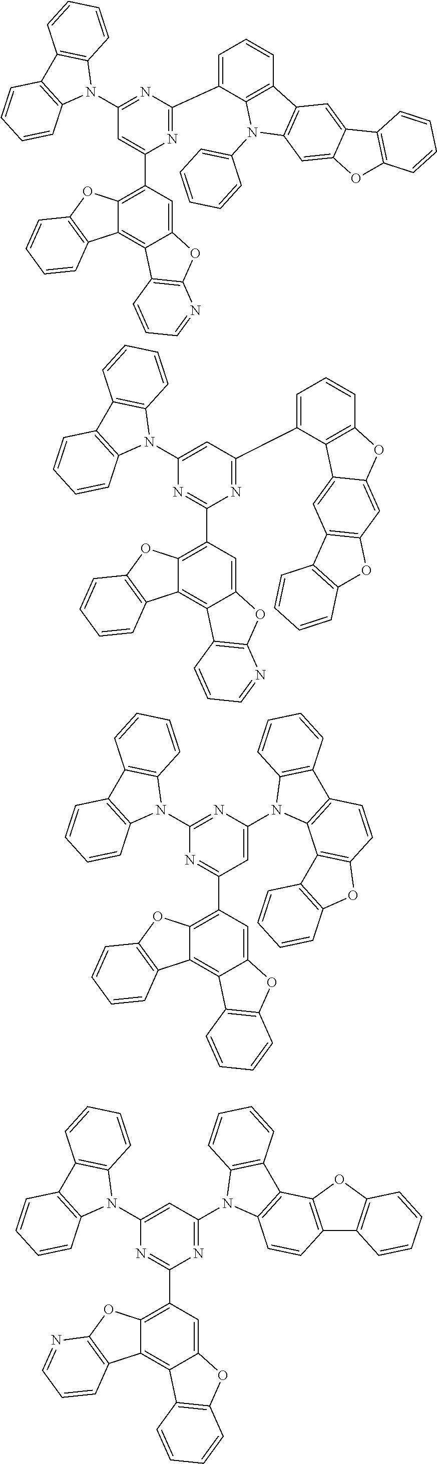

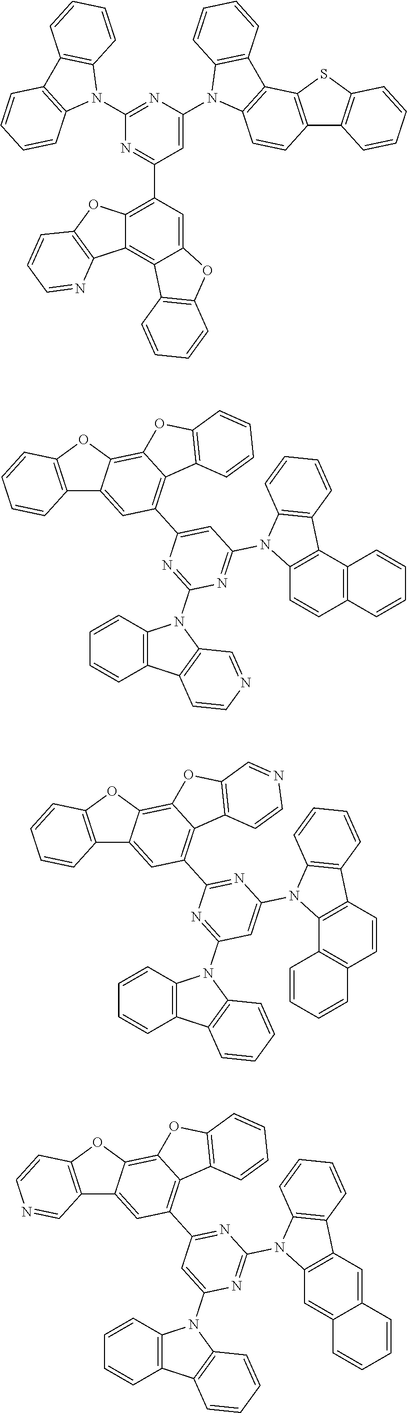 [SMILES: c1ccc2c(c1)oc1cc(-c3cc(-n4c5ccccc5c5c6sc7ccccc7c6ccc54)nc(-n4c5ccccc5c5ccccc54)n3)c3oc4cccnc4c3c12 ; c1ccc2c(c1)ccc1c2c2ccccc2n1-c1cc(-c2cc3c4ccccc4oc3c3oc4ccccc4c23)nc(-n2c3ccccc3c3ccncc32)n1 ; c1ccc2c(c1)ccc1c3ccccc3n(-c3cc(-n4c5ccccc5c5ccccc54)nc(-c4cc5c6ccccc6oc5c5oc6cnccc6c45)n3)c21 ; c1ccc2cc3c(cc2c1)c1ccccc1n3-c1nc(-c2cc3c4cnccc4oc3c3oc4ccccc4c23)cc(-n2c3ccccc3c3ccccc32)n1]

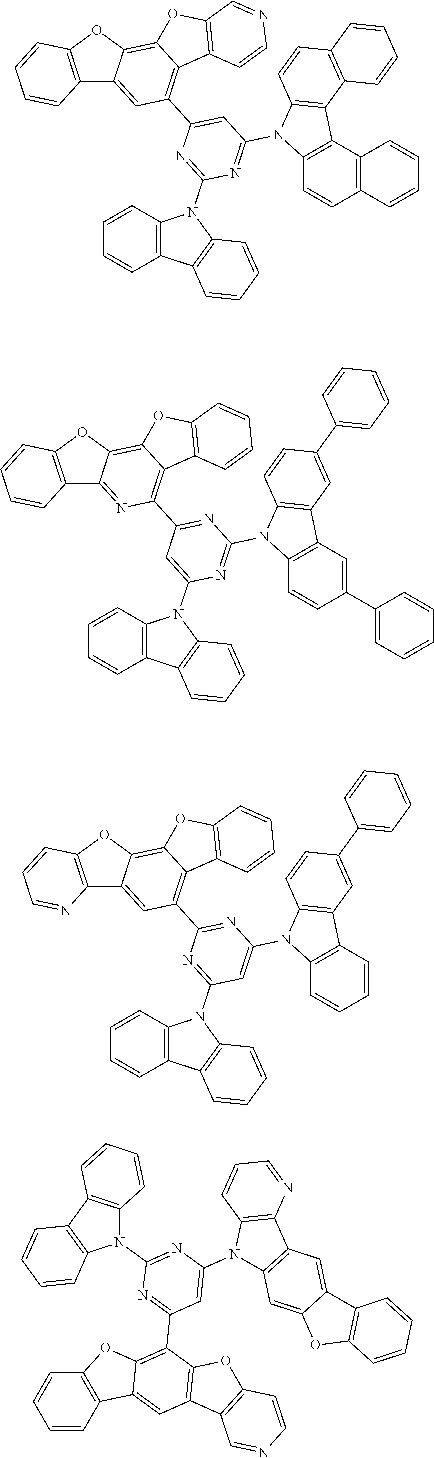 [SMILES: c1ccc2c(c1)ccc1c2c2c3ccccc3ccc2n1-c1cc(-c2cc3c4ccccc4oc3c3oc4cnccc4c23)nc(-n2c3ccccc3c3ccccc32)n1 ; c1ccc(-c2ccc3c(c2)c2cc(-c4ccccc4)ccc2n3-c2nc(-c3nc4c5ccccc5oc4c4oc5ccccc5c34)cc(-n3c4ccccc4c4ccccc43)n2)cc1 ; c1ccc(-c2ccc3c(c2)c2ccccc2n3-c2cc(-n3c4ccccc4c4ccccc43)nc(-c3cc4c5ncccc5oc4c4oc5ccccc5c34)n2)cc1 ; c1ccc2c(c1)oc1cc3c(cc12)c1ncccc1n3-c1cc(-c2c3oc4ccccc4c3cc3c2oc2ccncc23)nc(-n2c3ccccc3c3ccccc32)n1]

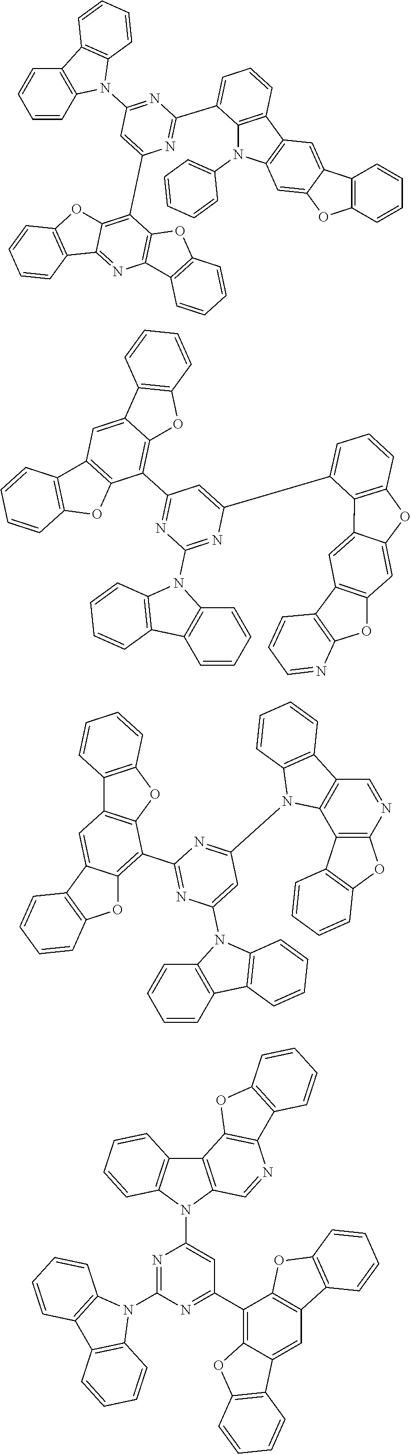

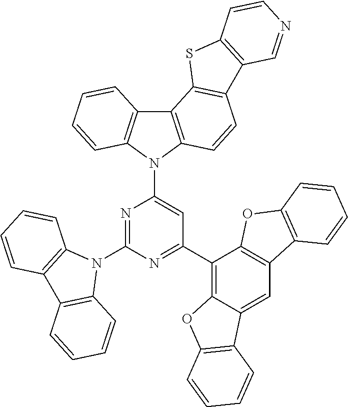 [SMILES: c1ccc2c(c1)oc1c(-c3cc(-n4c5ccccc5c5c6sc7ccncc7c6ccc54)nc(-n4c5ccccc5c5ccccc54)n3)c3oc4ccccc4c3cc12]

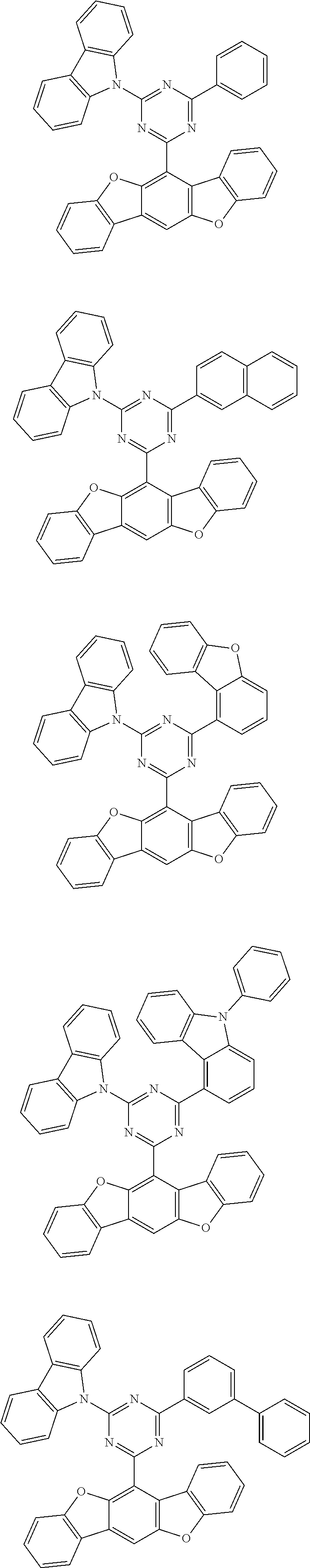 [SMILES: c1ccc(-c2nc(-c3c4oc5ccccc5c4cc4oc5ccccc5c34)nc(-n3c4ccccc4c4ccccc43)n2)cc1 ; c1ccc2cc(-c3nc(-c4c5oc6ccccc6c5cc5oc6ccccc6c45)nc(-n4c5ccccc5c5ccccc54)n3)ccc2c1 ; c1ccc2c(c1)oc1c(-c3nc(-c4cccc5oc6ccccc6c45)nc(-n4c5ccccc5c5ccccc54)n3)c3c(cc12)oc1ccccc13 ; c1ccc(-n2c3ccccc3c3c(-c4nc(-c5c6oc7ccccc7c6cc6oc7ccccc7c56)nc(-n5c6ccccc6c6ccccc65)n4)cccc32)cc1 ; c1ccc(-c2cccc(-c3nc(-c4c5oc6ccccc6c5cc5oc6ccccc6c45)nc(-n4c5ccccc5c5ccccc54)n3)c2)cc1]

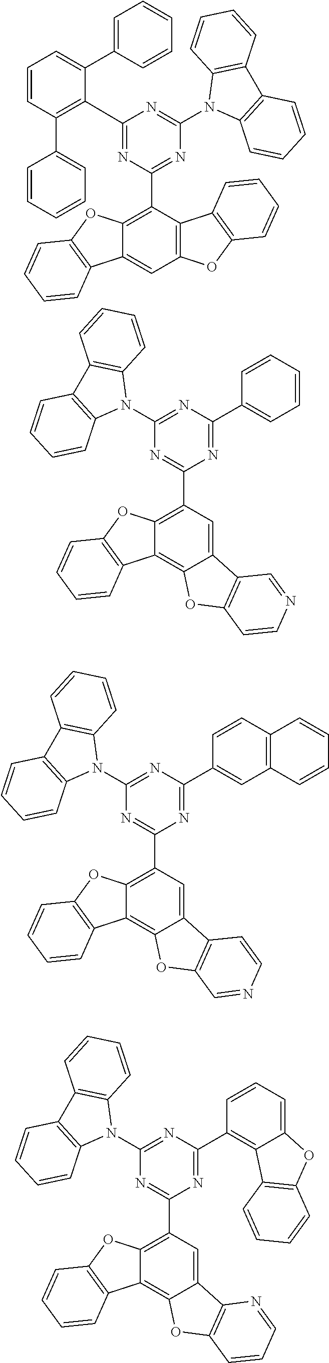

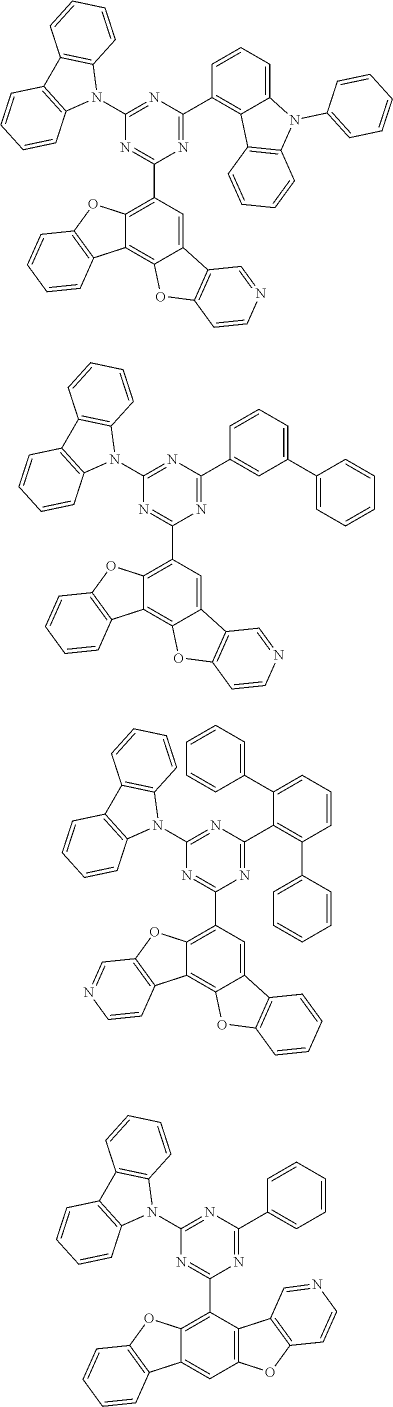

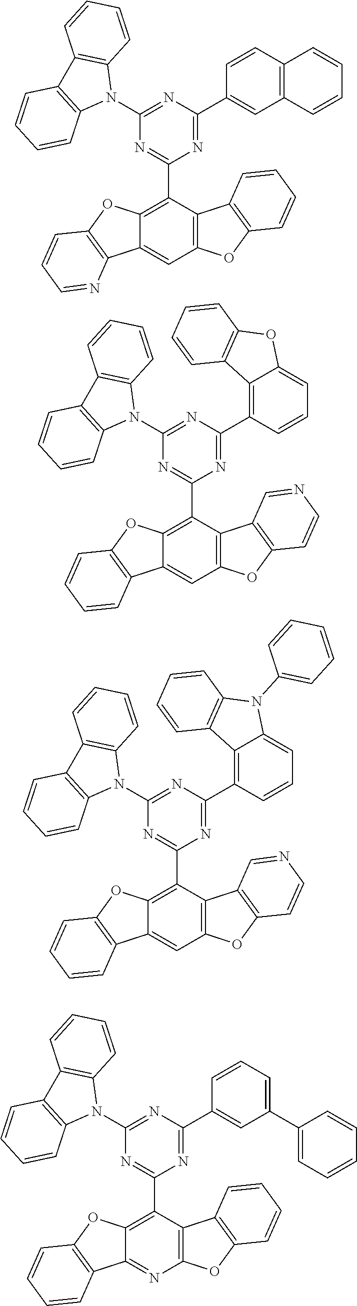

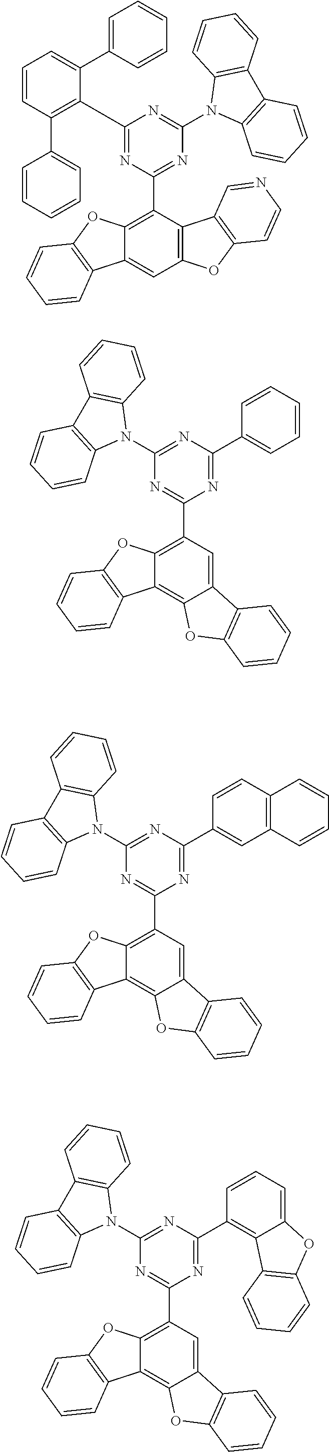

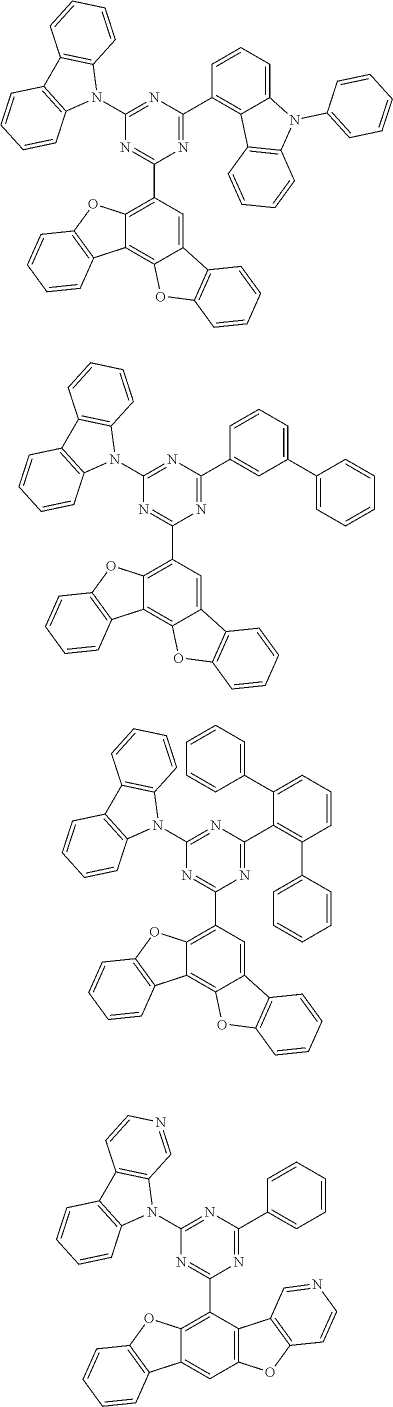

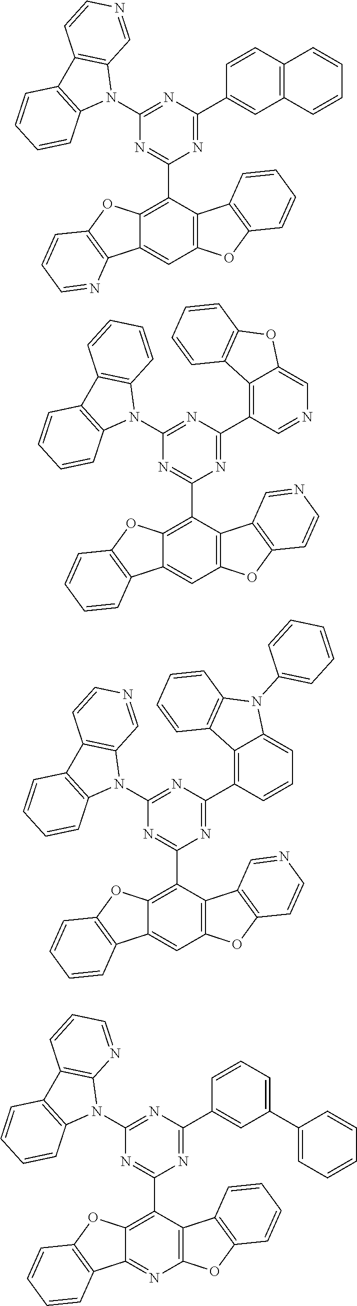

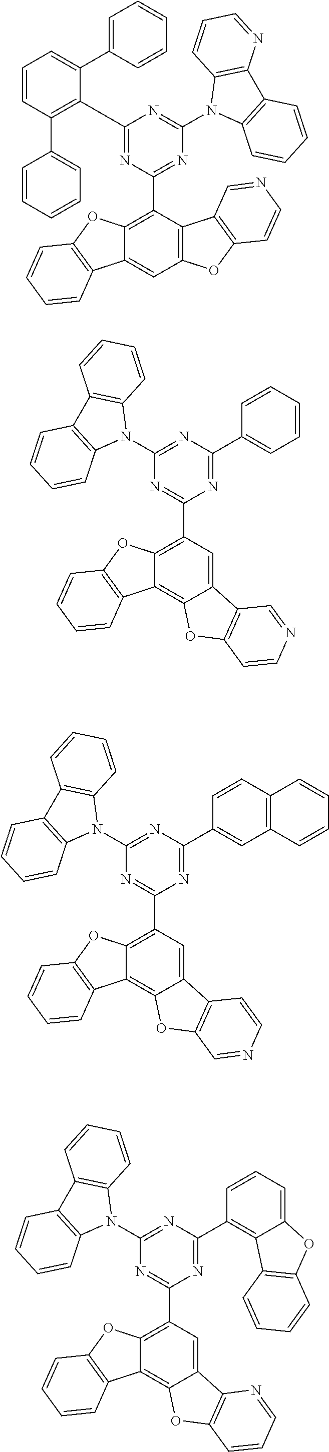

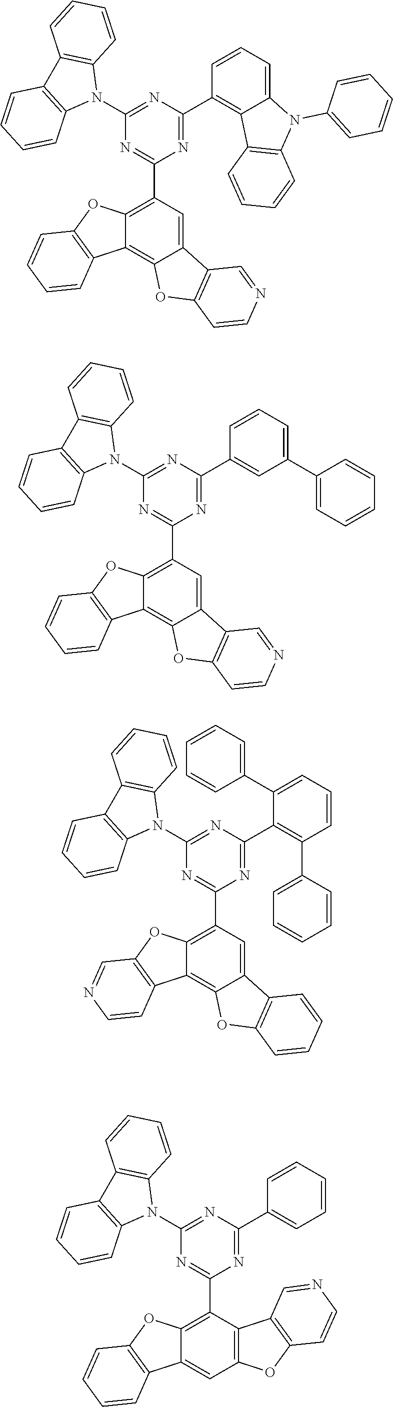

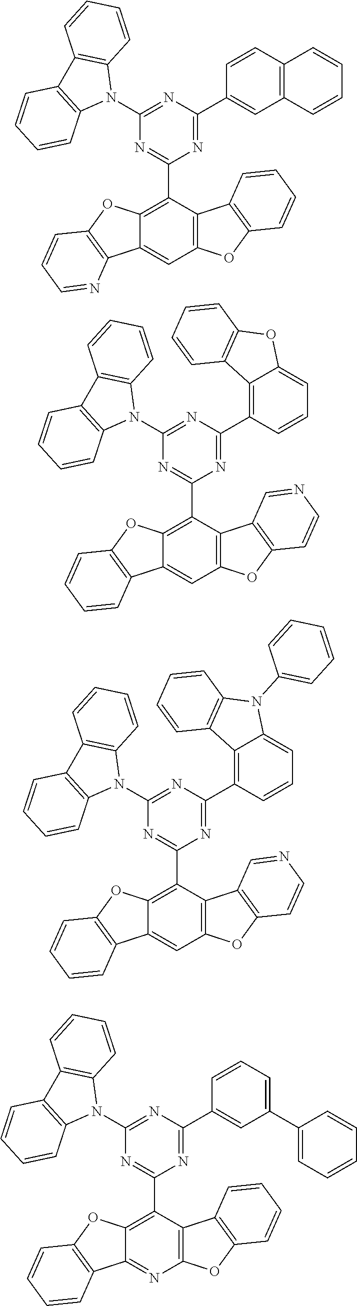

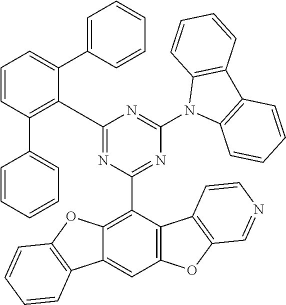 [SMILES: c1ccc(-c2cccc(-c3ccccc3)c2-c2nc(-c3c4oc5ccccc5c4cc4oc5cnccc5c34)nc(-n3c4ccccc4c4ccccc43)n2)cc1]

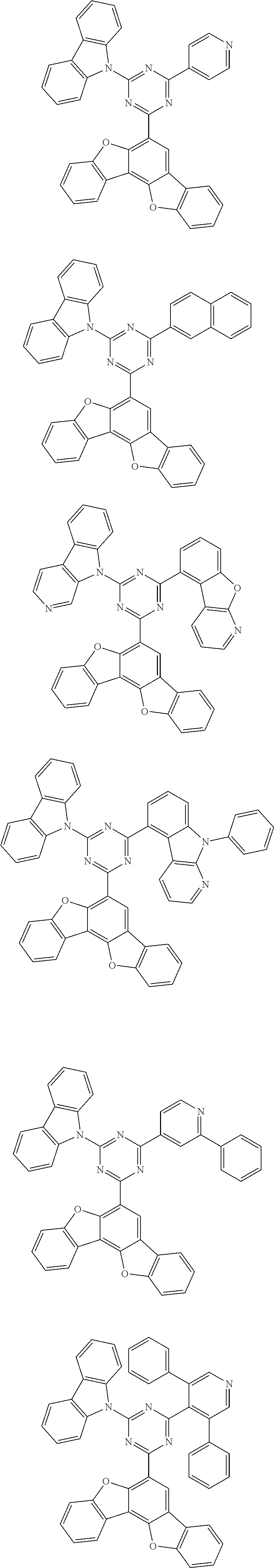

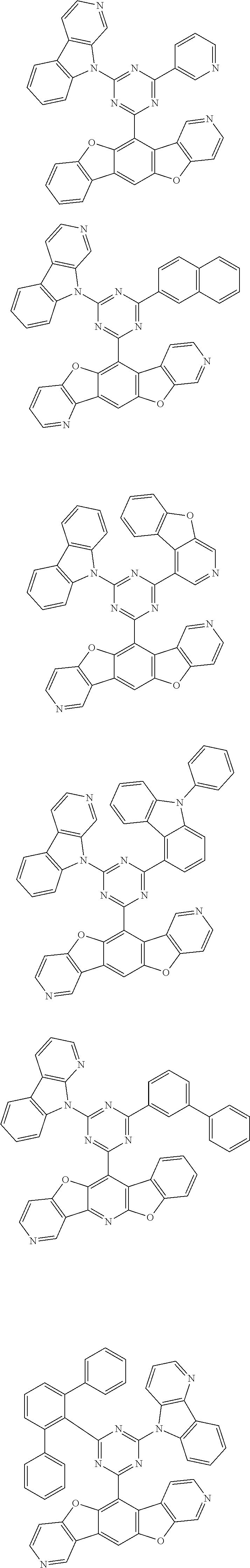

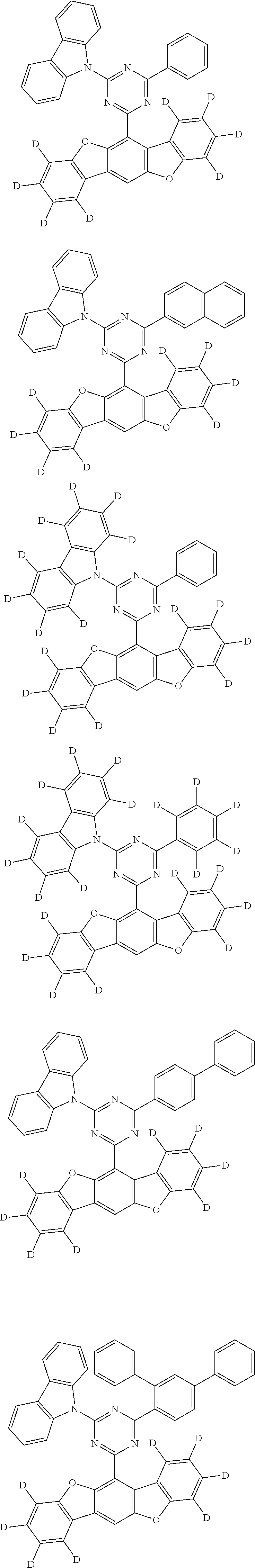

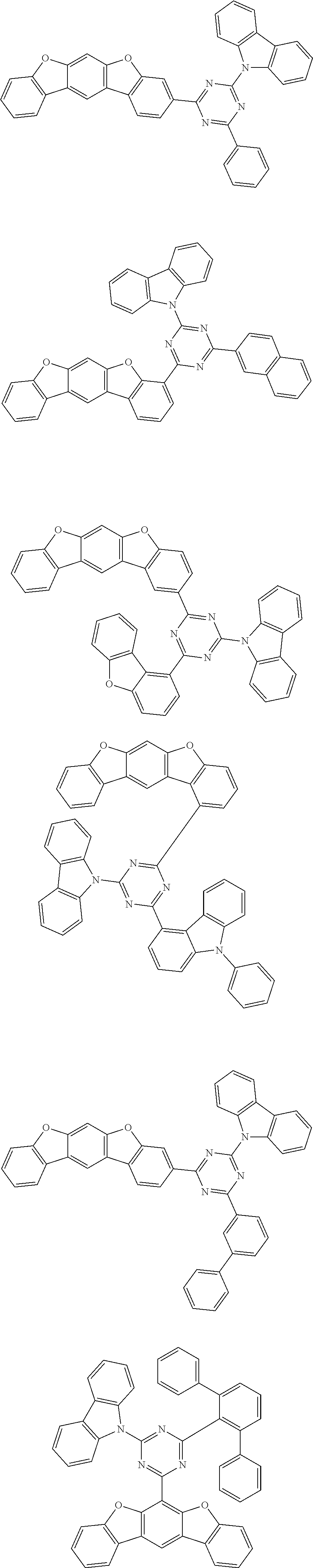

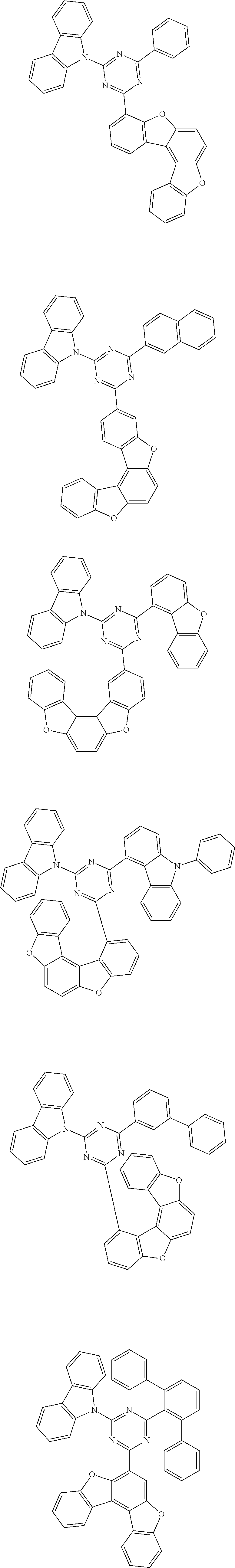

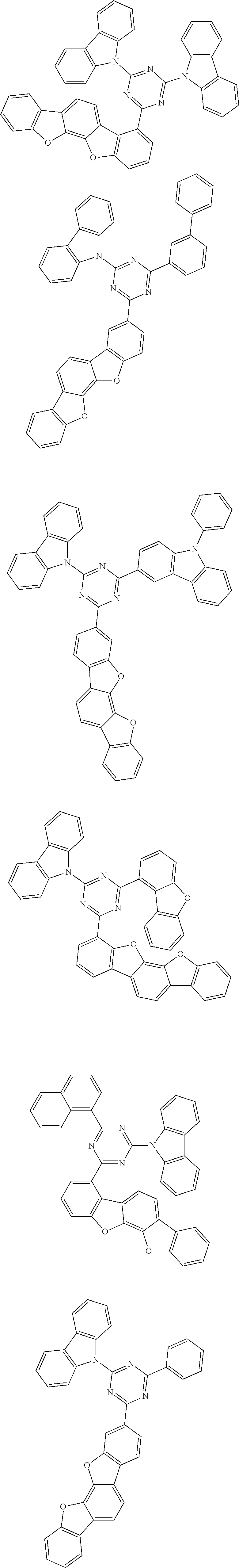

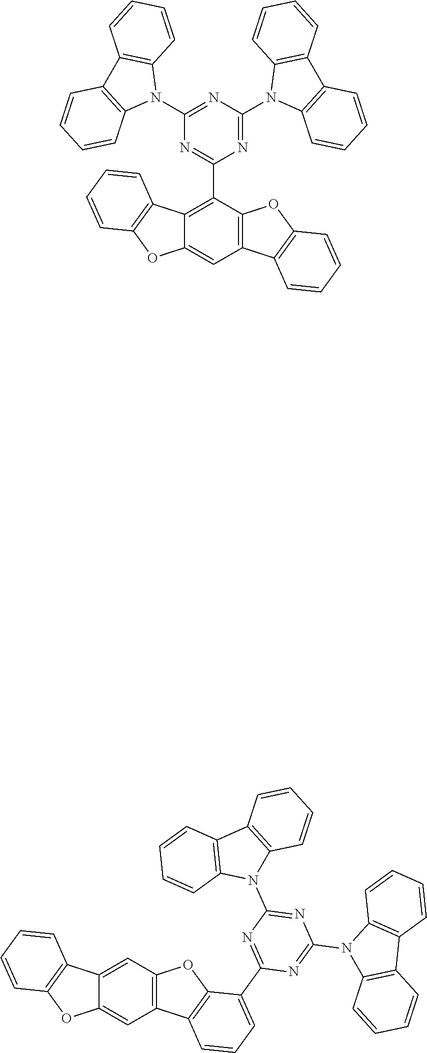 [SMILES: c1ccc2c(c1)oc1c(-c3nc(-n4c5ccccc5c5ccccc54)nc(-n4c5ccccc5c5ccccc54)n3)c3c(cc12)oc1ccccc13 ; c1ccc2c(c1)oc1cc3c(cc12)oc1c(-c2nc(-n4c5ccccc5c5ccccc54)nc(-n4c5ccccc5c5ccccc54)n2)cccc13]

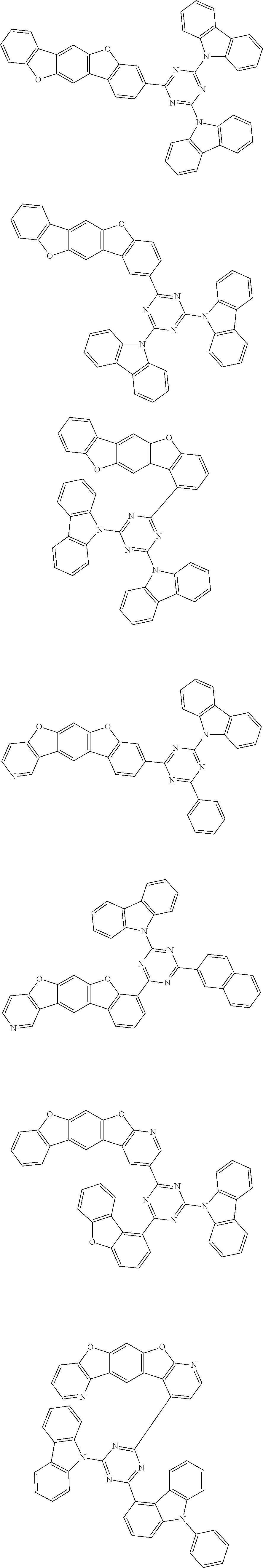

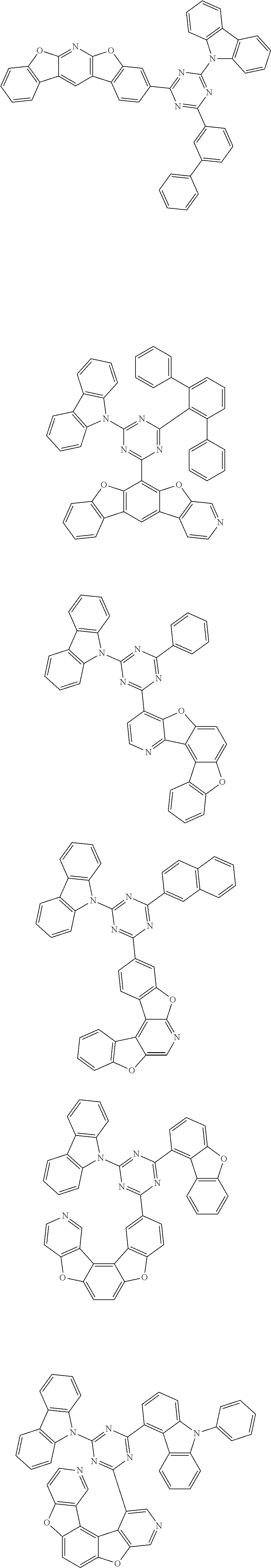

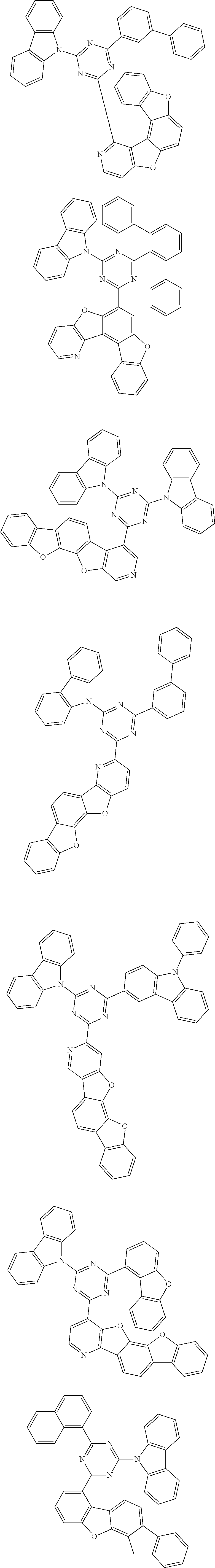

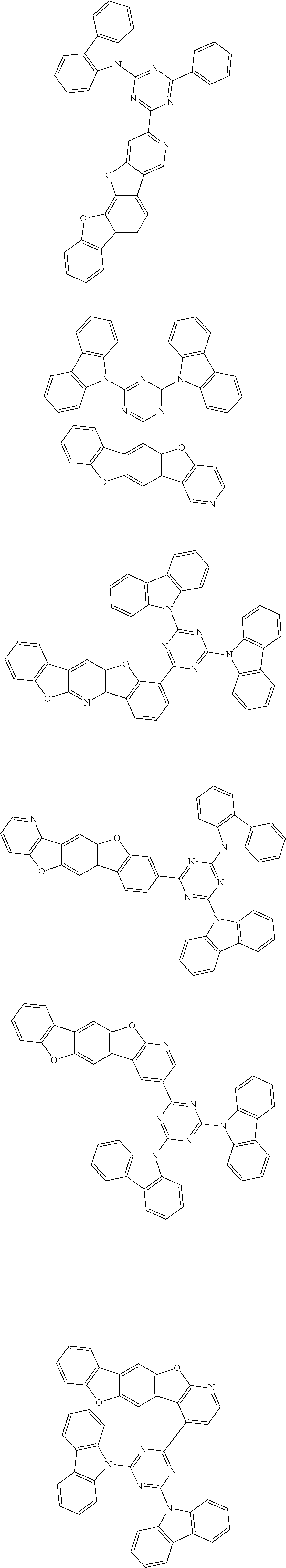

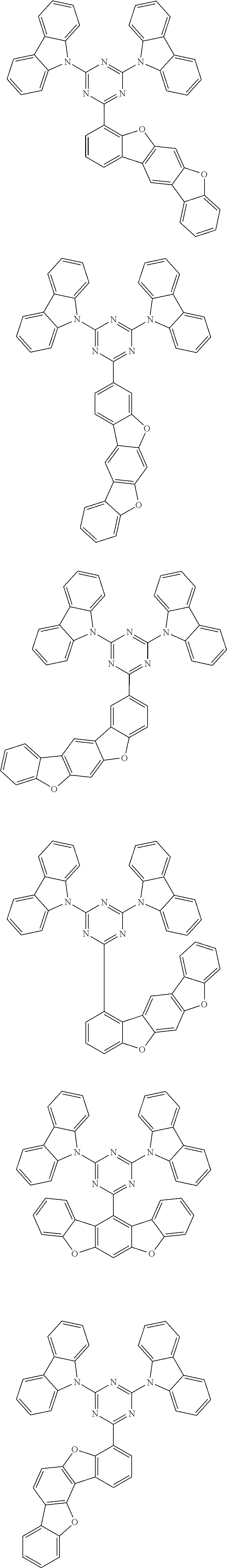

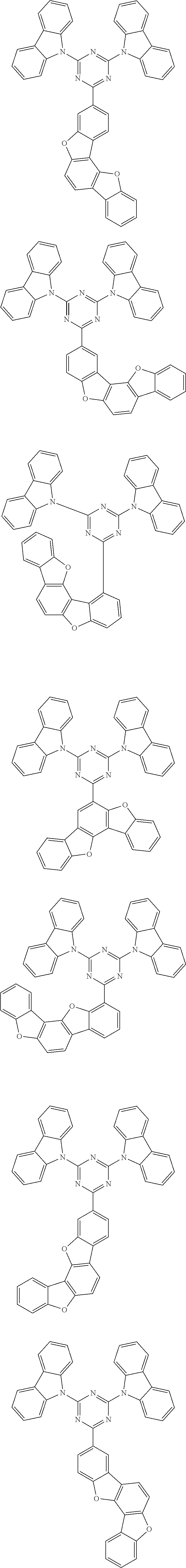

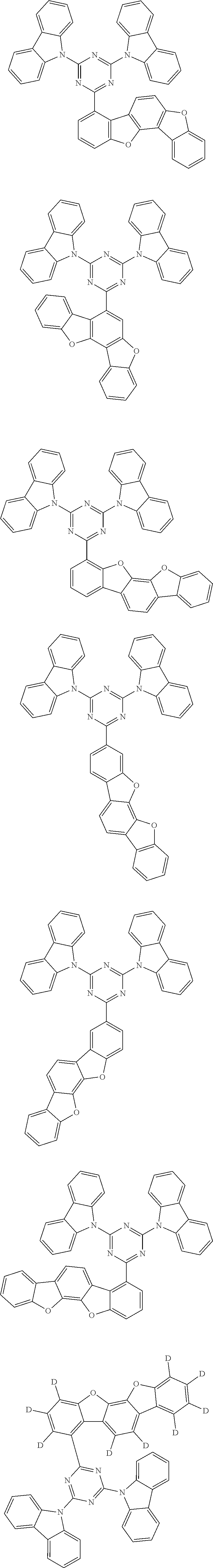

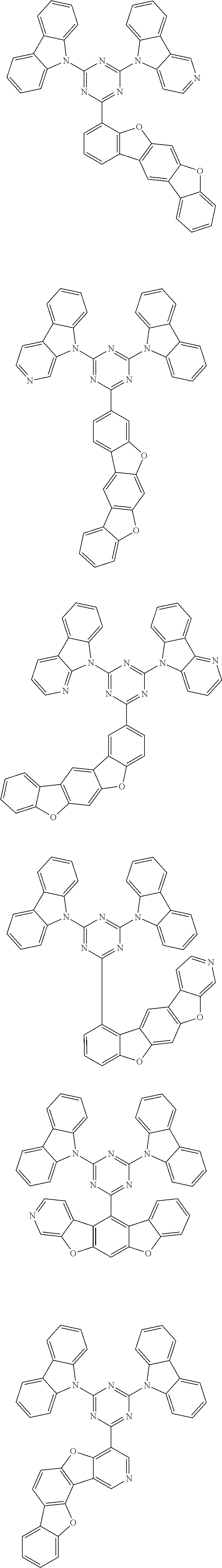

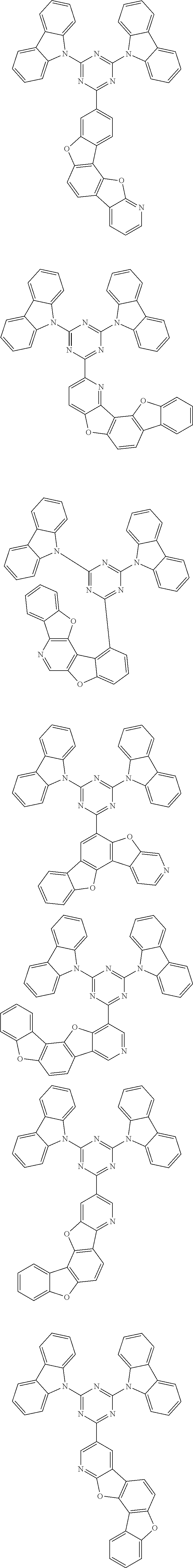

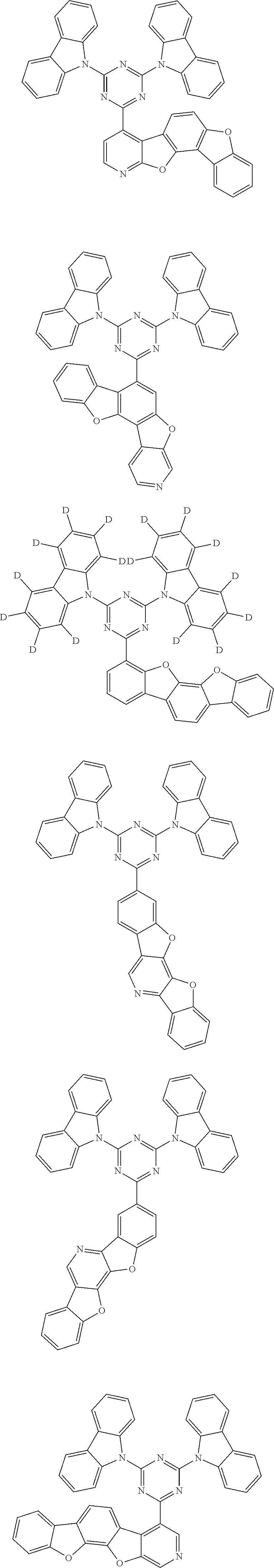

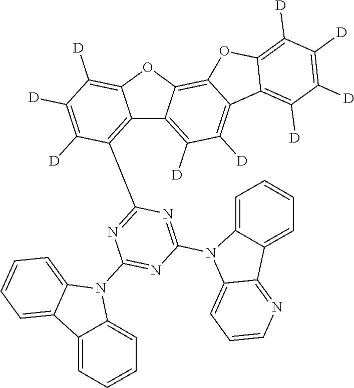 [SMILES: [2H]c1c([2H])c([2H])c2c(oc3c4oc5c([2H])c([2H])c([2H])c(-c6nc(-n7c8ccccc8c8ccccc87)nc(-n7c8ccccc8c8ncccc87)n6)c5c4c([2H])c([2H])c32)c1[2H]]

In some embodiments, the mixture as described herein are used for evaporation-based OLEDs. For this purpose, the first compound H1 and/or the second compound H2 of the mixture as described herein have a molecular weight ≤1000 g/mol, preferably ≤900 g/mol, more preferably ≤850 g/mol, further preferably ≤800 g/mol, and most preferably ≤750 g/mol.

In some embodiments, the first compound H1 and the second compound H2 of the mixture form a type II heterojunction energy structure.

In some embodiments, in the mixture as described herein, min(LUMO(H1)−HOMO(H2), LUMO(H2)−HOMO(H1))≤min(E_{T1}(H1), E_{T1}(H2))+0.05 eV.

In some embodiments, in the mixture as described herein, min(LUMO(H1)−HOMO(H2), LUMO(H2)−HOMO(H1))≤min(E_{T1}(H1), E_{T1}(H2)).

In some embodiments, in the mixture as described herein, min(LUMO(H1)−HOMO(H2), LUMO(H2)−HOMO(H1))≤min(E_{T1}(H1), E_{T1}(H2))−0.05 eV.

In some embodiments, in the mixture as described herein, min(LUMO(H1)−HOMO(H2), LUMO(H2)−HOMO(H1))≤min(E_{T1}(H1), E_{T1}(H2))−0.1 eV.

In some embodiments, in the mixture as described herein, min(LUMO(H1)−HOMO(H2), LUMO(H2)−HOMO(H1))≤min(E_{T1}(H1), E_{T1}(H2))−0.15 eV.

In some embodiments, in the mixture as described herein, min(LUMO(H1)−HOMO(H2), LUMO(H2)−HOMO(H1))≤min(E_{T1}(H1), E_{T1}(H2))−0.2 eV.

In some embodiments, in the mixture as described herein, the first compound H1 and the second compound H2 form a type I heterojunction energy structure; preferably, the band gap of the second compound H2 in the mixture is greater than that of the first compound H1. In some embodiments, the ΔE(E_{S1}-E_{T1}) of the first compound H1≤0.3 eV, preferably ≤0.2 eV, and most preferably ≤0.1 eV.

In embodiments of the present disclosure, the energy level structure of the organic materials, singlet energy level (E_{S1}), triplet energy level (E_{T1}), highest occupied molecular orbital (HOMO), and lowest unoccupied molecular orbital (LUMO) play key roles. The determination of these energy levels is introduced as follows.

HOMO and LUMO energy levels can be measured by optoelectronic effect, for example, by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT), are becoming effective methods for calculating the molecular orbital energy levels.

The singlet energy level E_{S1} of the organic materials can be determined by the emission spectrum. The triplet energy level E_{T1} of the organic materials can be measured by low-temperature time-resolved spectroscopy. E_{S1} and E_{T1} can also be calculated by quantum simulation (for example, by time-dependent DFT), for instance with the commercial software Gaussian 09W (Gaussian Inc.), the specific simulation method can be found in CN110892543B or as described in the following embodiments.

It should be noted that the absolute values of HOMO, LUMO, E_{S1} and E_{T1} may depend on the measurement method or calculation method used. Even for the same method, different ways of evaluation, for example, using either the onset or peak value of a CV curve as reference, may result in different HOMO/LUMO values. Therefore, reasonable and meaningful comparison should be carried out by employing the same measurement and evaluation methods. In the embodiments of the present disclosure, the values of HOMO, LUMO, E_{S1} and E_{T1} are based on the time-dependent DFT simulation, which however should not exclude the applications of other measurement or calculation methods.

A possible benefit of the mixture as described herein is that the excited state of the system will preferentially occupy the lowest-energy composite excited state, or facilitate energy transfer from the triplet excited state of either the first compound H1 or the second compound H2 to the composite excited state, thereby increasing the concentration of the composite excited state.

In some embodiments, the mixture as described herein can be used as a phosphorescent host material.

In the disclosure, (HOMO−1) is defined as the energy level of the second highest occupied molecular orbital, (HOMO−2) is defined as the energy level of the third highest occupied molecular orbital, and so on. (LUMO+1) is defined as the energy level of the second lowest unoccupied molecular orbital, (LUMO+2) is defined as the energy level of the third lowest occupied molecular orbital, and so on.

In some embodiments, in the mixture as described herein, min((LUMO_{H1}−HOMO_{H2}), (LUMO_{H2}−HOMO_{H1})) is less than or equal to the triplet excited energy level of the first compound H1, while at the same time being less than or equal to the triplet excited energy level of the second compound H2. The energy of the composite excited state formed by the first compound H1 and the second compound H2 depends on min((LUMO_{H1}−HOMO_{H2}), (LUMO_{H2}−HOMO_{H1})).

In some embodiments, in the mixture as described herein, at least one of the first compound H1 or the second compound H2 has (HOMO−(HOMO−1))≥0.2 eV, preferably ≥0.25 eV, more preferably ≥0.3 eV, even more preferably ≥0.35 eV, further preferably ≥0.4 eV, and most preferably ≥0.45 eV.

In some embodiments, the second compound H2 of the mixture comprises an organic compound containing an electron-donating group. One possible benefit of this combination is that the first compound H1 and the second compound H2 are easy to form a type II semiconductor heterojunction.

In some embodiments, the second compound H2 of the mixture has (HOMO−(HOMO−1))≥0.2 eV, preferably ≥0.25 eV, more preferably ≥0.3 eV, even more preferably ≥0.35 eV, further preferably ≥0.4 eV, and most preferably ≥0.45 eV.

In some embodiments, the second compound H2 of the mixture is selected from the group consisting of:

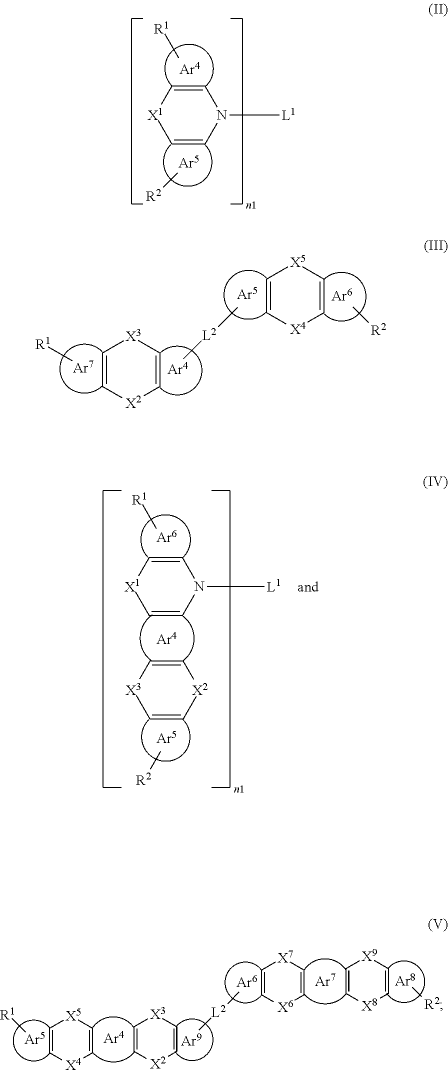

Where each L¹ independently represents an aromatic or a heteroaromatic group containing 5 to 60 ring atoms; each L² independently represents a single bond, an aromatic or a heteroaromatic group containing 5 to 30 ring atoms, an attachment position of L¹ or L² can be on any carbon atom of the benzene ring; each of Ar⁴ to Ar⁹ is independently selected from an aromatic or a heteroaromatic group containing 5 to 30 ring atoms; each X¹ independently represents a single bond, N(R), C(R)₂, Si(R)₂, O, C═N(R), C═C(R)₂, P(R), P(═O)R, S, S═O, or SO₂; each of X² to X⁹ is independently selected from a single bond, N(R), C(R)₂, Si(R)₂, O, C═N(R), C═C(R)₂, P(R), P(═O)R, S, S═O, or SO₂; but X² and X³ are not simultaneously a single bond, X⁴ and X⁵ are not simultaneously a single bond, X⁶ and X⁷ are not simultaneously a single bond, X⁸ and X⁹ are not simultaneously a single bond; R¹, R², and R are each independently selected from —H, -D, —F, —CN, an alkenyl group, an alkynyl group, a nitrile group, an amine group, a nitro group, an acyl group, an alkoxy group, a carbonyl group, a sulfone group, a C₁-C₃₀ alkyl group, a C₃-C₃₀ cycloalkyl group, an aromatic hydrocarbon group containing 5 to 60 ring atoms, or an aromatic heterocyclic group containing 5 to 60 ring atoms, where the attachment position of R¹ or R² can be on any carbon atom of the fused ring, and there can be any number of carbon atoms substituted with R¹ and R²; n1 is an integer from 1 to 4.

In some embodiments, the second compound H2 is selected from the group consisting of:

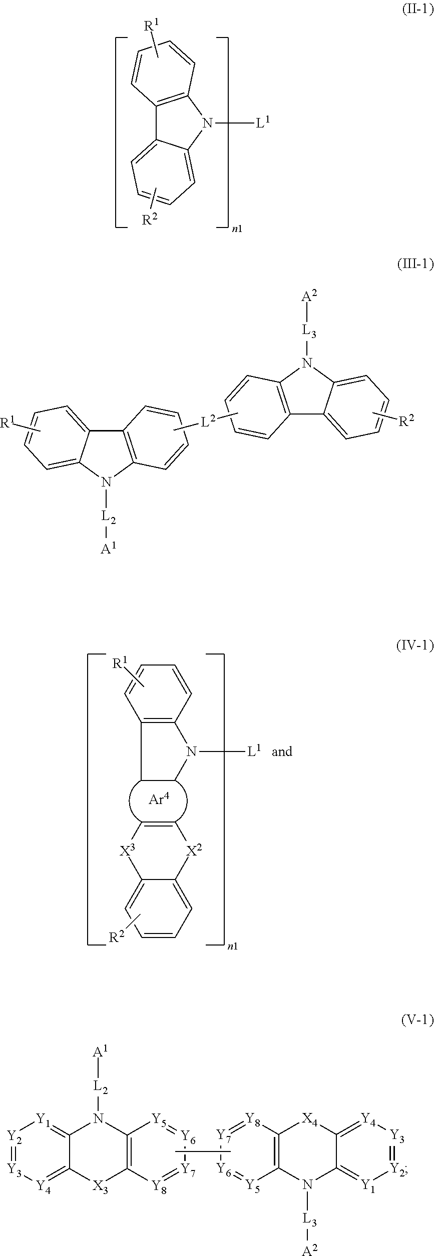

Where X²-X⁴, L¹, R¹, R², and n1 are defined identically as described herein; each of L₂ and L₃ is defined identically as the above-mentioned L¹; each of A¹ and A² is independently selected from an aromatic or a heteroaromatic group containing 5 to 30 ring atoms; each of Y₁ to Y₈ is independently N or CR, and any two adjacent substituents of Y₁-Y₈ cannot be N at the same time.

Preferably, the second compound H2 according to formulas (II-1)-(V-1) is selected from the group consisting of:

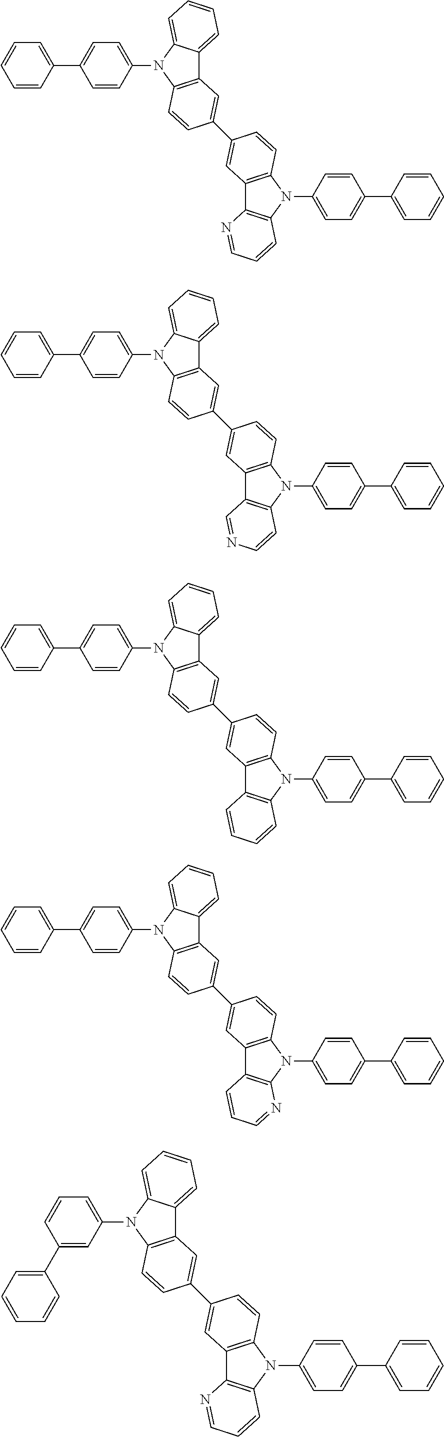 [SMILES: c1ccc(-c2ccc(-n3c4ccccc4c4cc(-c5ccc6c(c5)c5ncccc5n6-c5ccc(-c6ccccc6)cc5)ccc43)cc2)cc1 ; c1ccc(-c2ccc(-n3c4ccccc4c4cc(-c5ccc6c(c5)c5cnccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5cccnc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4ncccc43)cc2)cc1]

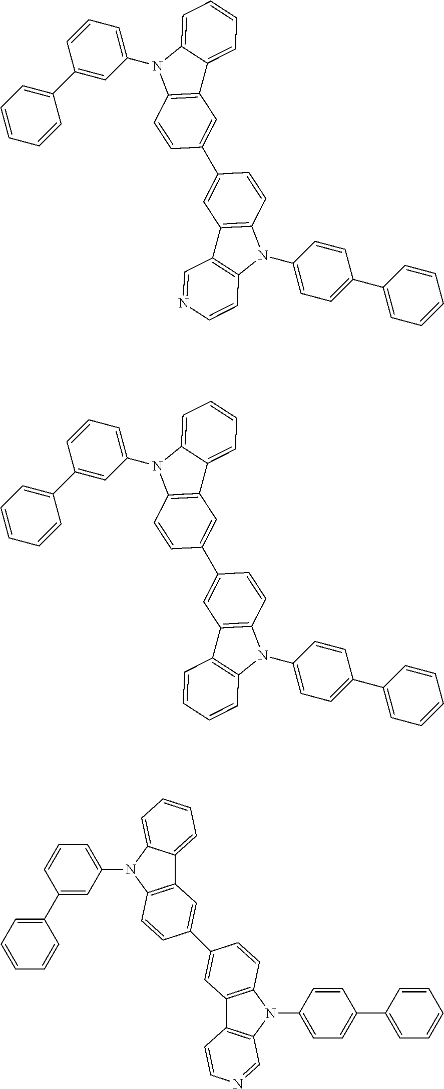 [SMILES: c1ccc(-c2ccc(-n3c4ccncc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccc(-c5ccc6c(c5)c5ccccc5n6-c5cccc(-c6ccccc6)c5)cc4c4ccncc43)cc2)cc1]

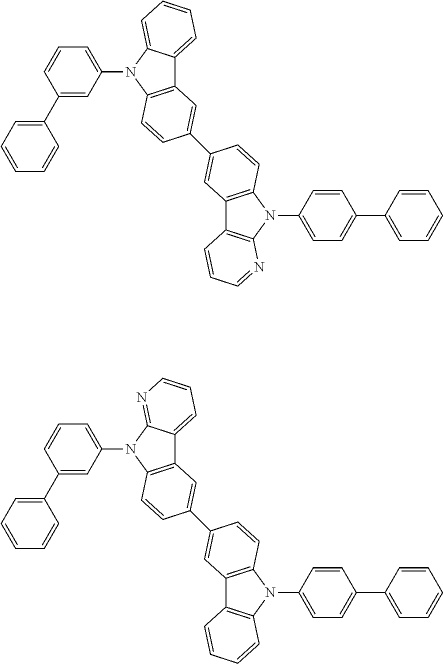 [SMILES: c1ccc(-c2ccc(-n3c4ccc(-c5ccc6c(c5)c5ccccc5n6-c5cccc(-c6ccccc6)c5)cc4c4cccnc43)cc2)cc1 ; c1ccc(-c2ccc(-n3c4ccccc4c4cc(-c5ccc6c(c5)c5cccnc5n6-c5cccc(-c6ccccc6)c5)ccc43)cc2)cc1]

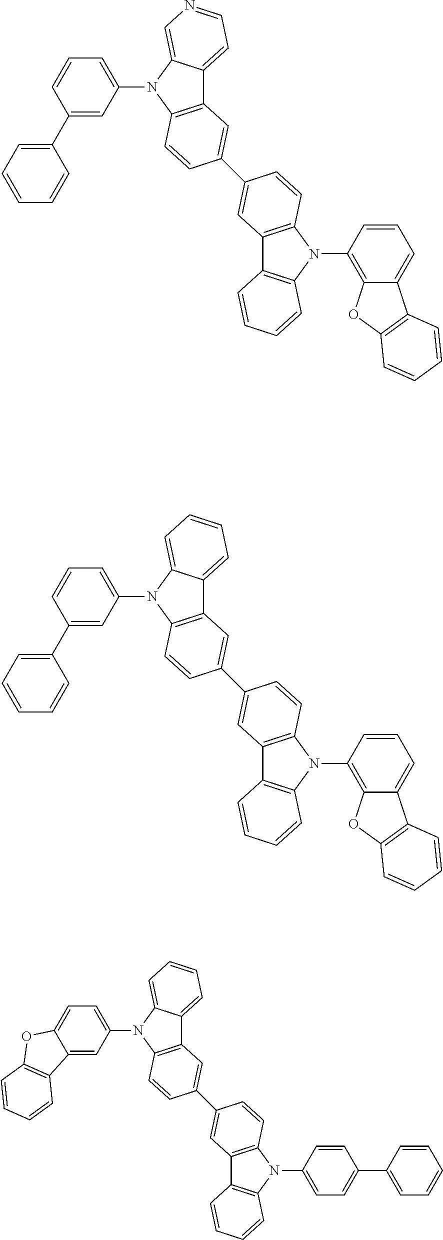 [SMILES: c1ccc(-c2cccc(-n3c4ccc(-c5ccc6c(c5)c5ccccc5n6-c5cccc6c5oc5ccccc56)cc4c4ccncc43)c2)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5ccc6oc7ccccc7c6c5)ccc43)cc2)cc1]

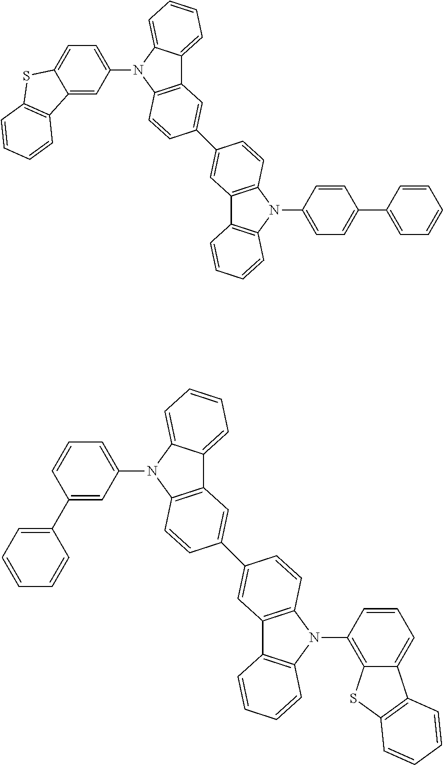 [SMILES: c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cccc(-n3c4ccccc4c4cc(-c5ccc6c(c5)c5ccccc5n6-c5cccc6c5sc5ccccc56)ccc43)c2)cc1]

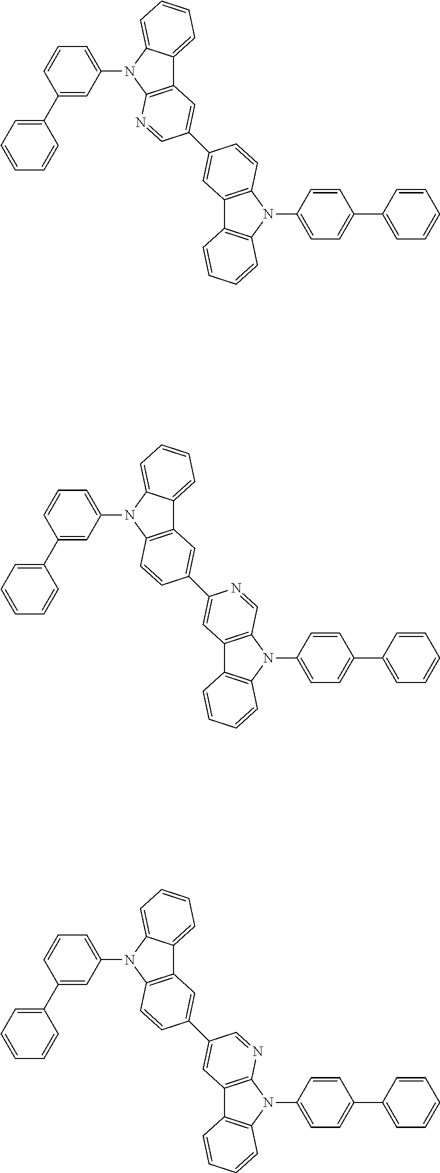 [SMILES: c1ccc(-c2ccc(-n3c4ccccc4c4cc(-c5cnc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ncc43)cc2)cc1 ; c1ccc(-c2ccc(-n3c4ccccc4c4cc(-c5ccc6c(c5)c5ccccc5n6-c5cccc(-c6ccccc6)c5)cnc43)cc2)cc1]

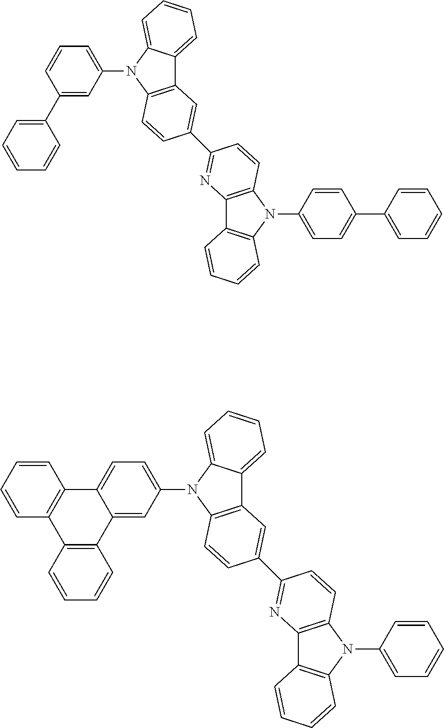 [SMILES: c1ccc(-c2ccc(-n3c4ccccc4c4nc(-c5ccc6c(c5)c5ccccc5n6-c5cccc(-c6ccccc6)c5)ccc43)cc2)cc1 ; c1ccc(-n2c3ccccc3c3nc(-c4ccc5c(c4)c4ccccc4n5-c4ccc5c6ccccc6c6ccccc6c5c4)ccc32)cc1]

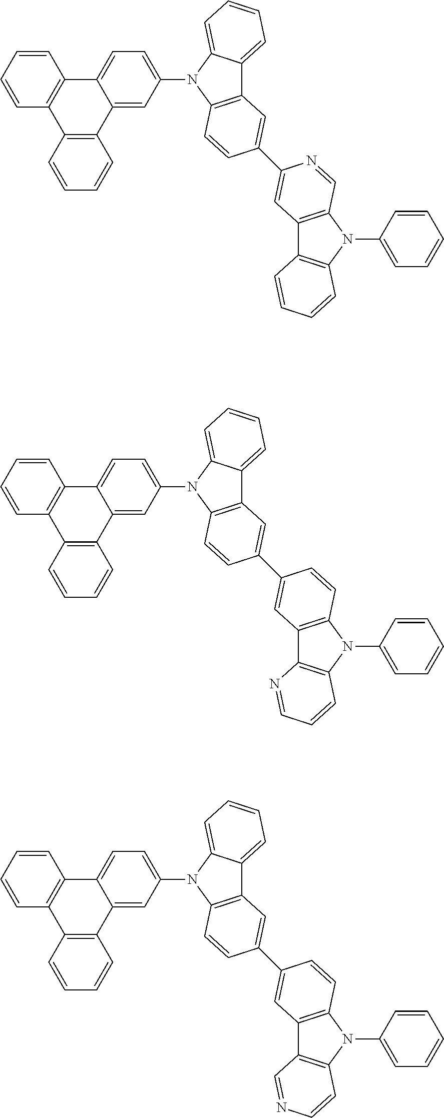 [SMILES: c1ccc(-n2c3ccccc3c3cc(-c4ccc5c(c4)c4ccccc4n5-c4ccc5c6ccccc6c6ccccc6c5c4)ncc32)cc1 ; c1ccc(-n2c3ccc(-c4ccc5c(c4)c4ccccc4n5-c4ccc5c6ccccc6c6ccccc6c5c4)cc3c3ncccc32)cc1 ; c1ccc(-n2c3ccncc3c3cc(-c4ccc5c(c4)c4ccccc4n5-c4ccc5c6ccccc6c6ccccc6c5c4)ccc32)cc1]

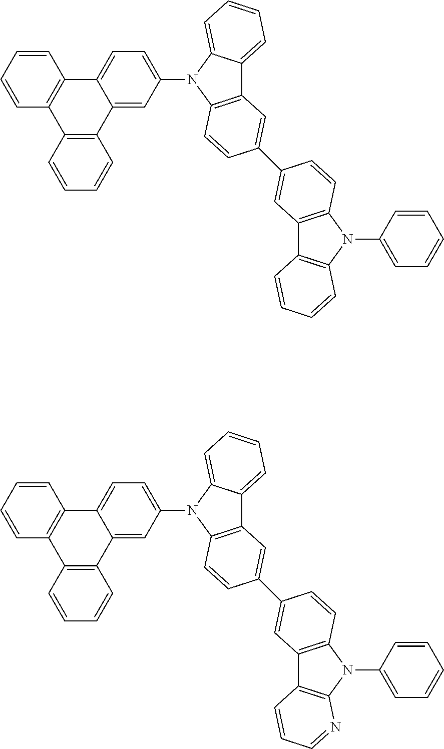 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccc(-c4ccc5c(c4)c4ccccc4n5-c4ccc5c6ccccc6c6ccccc6c5c4)cc3c3cccnc32)cc1]

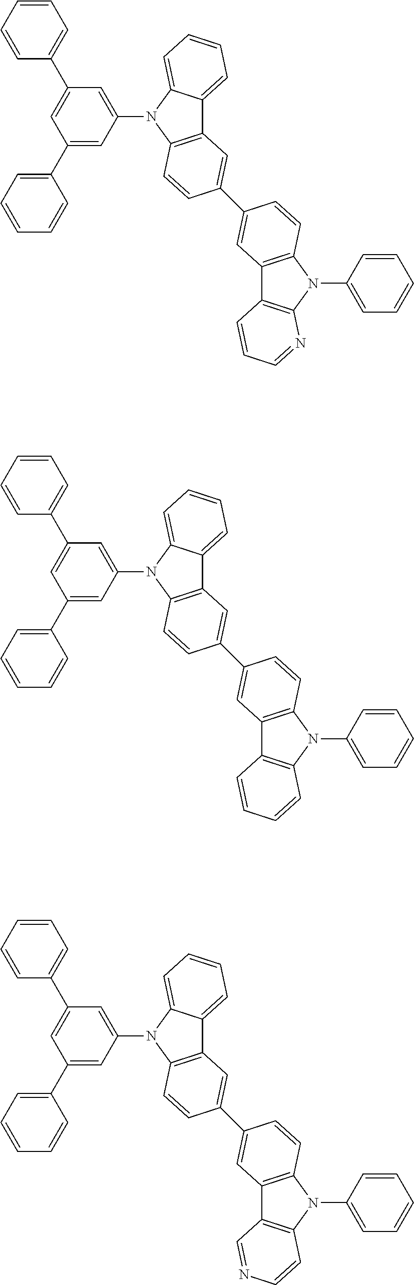 [SMILES: c1ccc(-c2cc(-c3ccccc3)cc(-n3c4ccccc4c4cc(-c5ccc6c(c5)c5cccnc5n6-c5ccccc5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cc(-c3ccccc3)cc(-n3c4ccccc4c4cc(-c5ccc6c(c5)c5cnccc5n6-c5ccccc5)ccc43)c2)cc1]

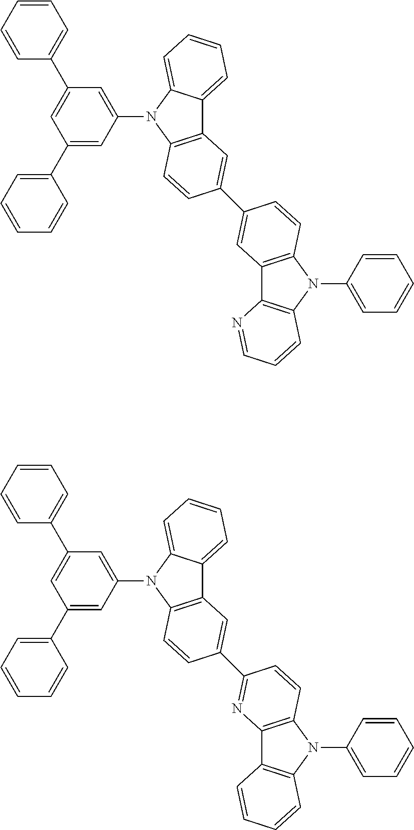 [SMILES: c1ccc(-c2cc(-c3ccccc3)cc(-n3c4ccccc4c4cc(-c5ccc6c(c5)c5ncccc5n6-c5ccccc5)ccc43)c2)cc1 ; c1ccc(-c2cc(-c3ccccc3)cc(-n3c4ccccc4c4cc(-c5ccc6c(n5)c5ccccc5n6-c5ccccc5)ccc43)c2)cc1]

In some embodiments, the mixture as described herein can be used as a host material of the emissive layer in an organic electroluminescent device, where min((LUMO_{H1}−HOMO_{H2}), (LUMO_{H2}−HOMO_{H1})) is less than or equal to the triplet excited energy level of the first compound H1/the second compound H2.

When a single material with either electron-biased or hole-biased characteristics is used to form the emissive layer, relatively more excitons can form at the interface between the emissive layer and the electron-transport layer or the hole-transport layer. Therefore, the excitons of the emissive layer may interact with the interfacial charge of the electron-transport layer or the hole-transport layer, thus causing a sharp roll-off in the device efficiency at high luminance and shortening device lifetime. In order to solve this problem, the first compound H1 and the second compound H2 are mixed and introduced into the emissive layer to balance the hole and electron mobility of the emissive layer, so that the middle region of the emissive layer emits light, improving device efficiency and extending device lifetime. Mixing the first compound H1 and the second compound H2 to form a mixture, the range of mass ratios that can be used is preferably from 2:8 to 8:2, more preferably from 3:7 to 7:3, even more preferably from 4:6 to 6:4, further preferably from 4.5:5.5 to 5.5:4.5, and most preferably 5:5.

In some embodiments, the first compound H1/the second compound H2 as described herein is a small molecular material, and the mixture as described herein is also a small-molecule mixture.

The term “small molecule” herein refers to a molecule that is no one of following: a polymer, an oligomer, a dendrimer, or a blend. In particular, there are no repeating structures in the small molecule. The molecular weight of the small molecule ≤4000 g/mol, preferably ≤3000 g/mol, more preferably ≤2000 g/mol, and most preferably ≤1500 g/mol.

The term “polymer” comprises homopolymer, copolymer, and block copolymer. Also in the present disclosure, the term “polymer” comprises dendrimer. For the synthesis and application of the dendrimers please refer to [Dendrimers and Dendrons, Wiley-VCH Verlag GmbH & Co. KGaA, 2002, Ed. George R. Newkome, Charles N. Moorefield, Fritz Vogtle.].

The term “conjugated polymer” refers to a polymer with backbone mainly comprising sp² hybrid orbitals of carbon atoms, well-known examples are polyacetylene and poly(phenylene vinylene). The carbon atoms on the backbones can also be substituted with other non-carbon atoms. Moreover, the above-mentioned structure should still be considered as a conjugated polymer when the sp² hybridization on the backbone is interrupted by natural defects. Also in the present disclosure, the backbone of the conjugated polymer comprises aryl amines, aryl phosphines and other heteroarmotics, organometallic complexes, etc.

In some embodiments, in order to improve the material evaporation efficiency and material utilization, as well as to simplify the material evaporation process, the molecular weight difference between the first compound H1 and the second compound H2 is ≤100 Dalton, preferably ≤90 Dalton, more preferably ≤70 Dalton, even more preferably ≤60 Dalton, further preferably ≤50 Dalton, and most preferably ≤20 Dalton.

In some embodiments, in the mixture as described herein, the sublimation temperature difference between the first compound H1 and the second compound H2≤40 K, preferably ≤30 K, more preferably ≤25 K, even more preferably ≤20 K, further preferably ≤18 K, and most preferably ≤15 K.

The examples of the second compound H2s as described herein are shown in the following structures. These structures may be substituted at all possible substitution sites.

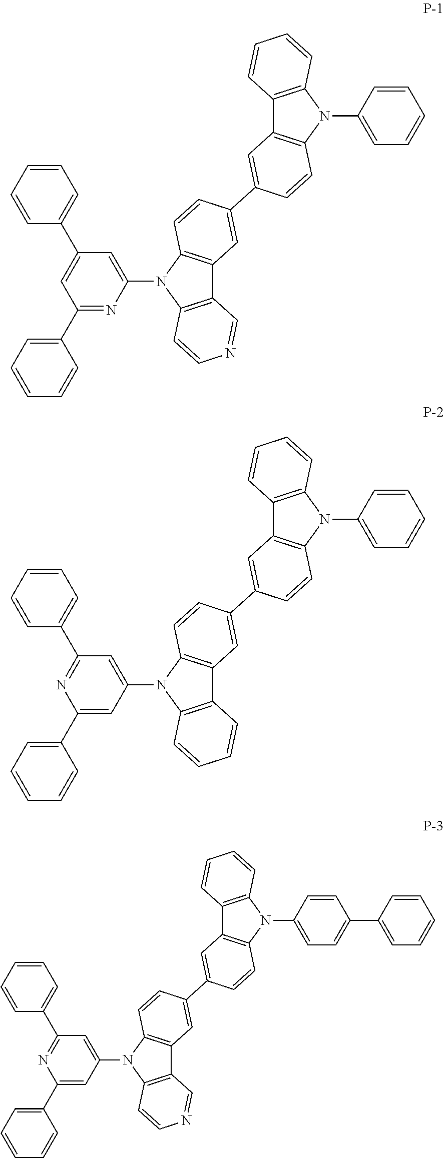 [SMILES: c1ccc(-c2cc(-c3ccccc3)nc(-n3c4ccncc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5cnccc5n6-c5cc(-c6ccccc6)nc(-c6ccccc6)c5)ccc43)cc2)cc1]

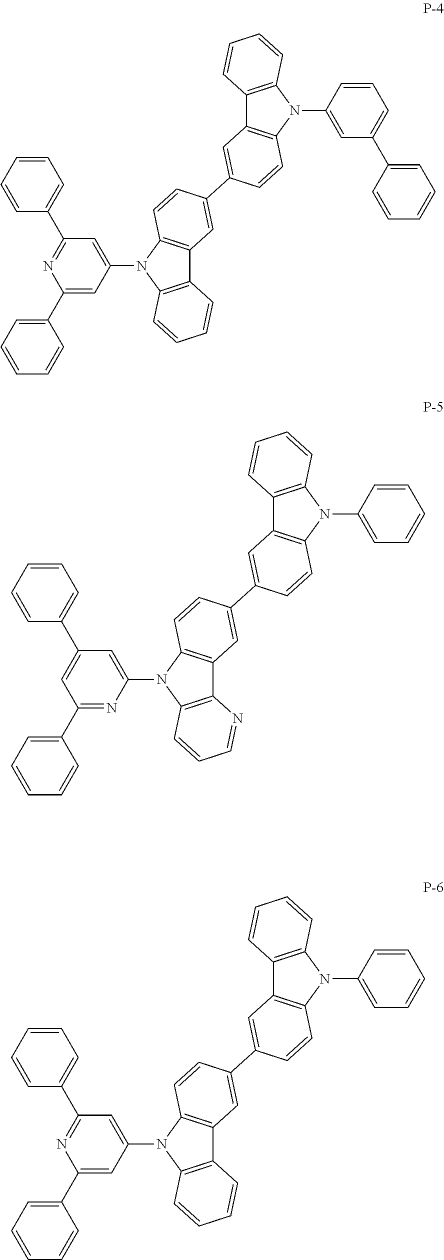 [SMILES: c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccc(-c5ccc6c(c5)c5ccccc5n6-c5ccccc5)cc4c4ncccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1]

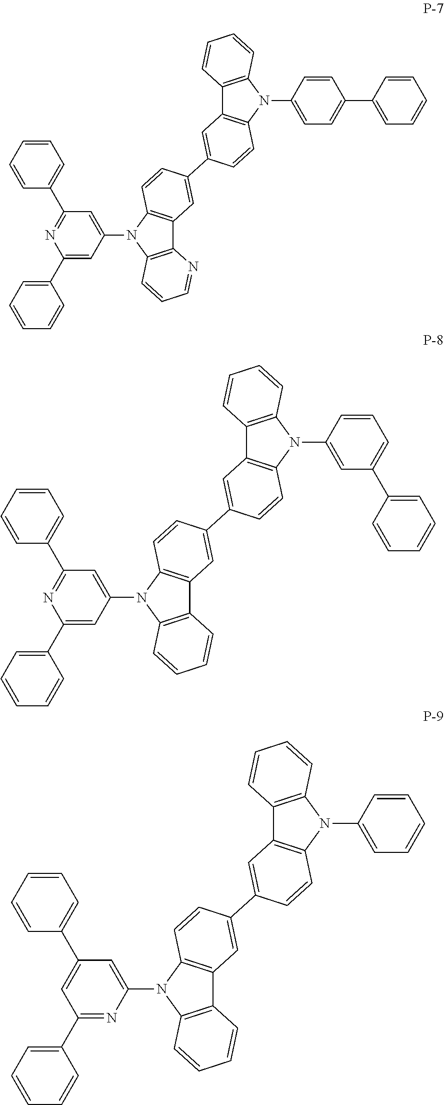 [SMILES: c1ccc(-c2ccc(-n3c4ccccc4c4cc(-c5ccc6c(c5)c5ncccc5n6-c5cc(-c6ccccc6)nc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5ccccc5)ccc43)c2)cc1]

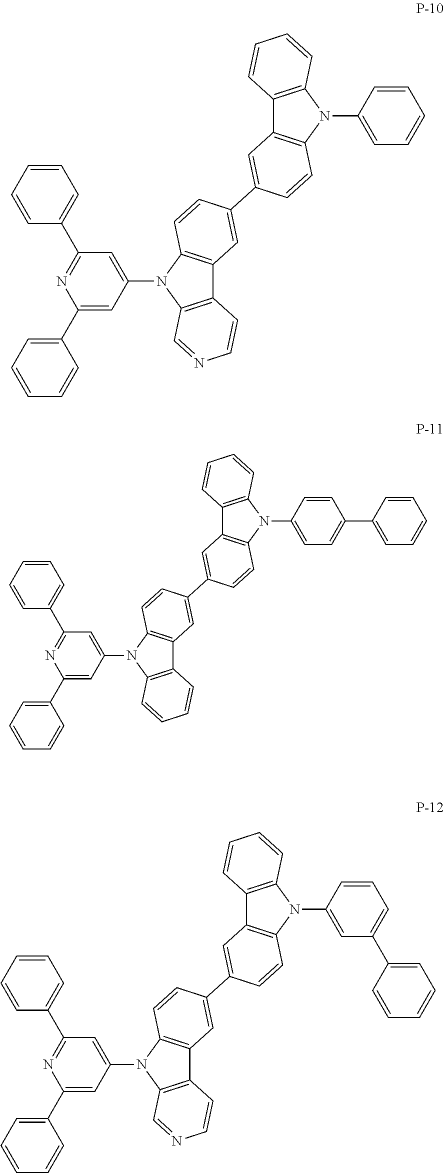 [SMILES: c1ccc(-c2cc(-n3c4ccc(-c5ccc6c(c5)c5ccccc5n6-c5ccccc5)cc4c4ccncc43)cc(-c3ccccc3)n2)cc1 ; c1ccc(-c2ccc(-n3c4ccccc4c4cc(-c5ccc6c(c5)c5ccccc5n6-c5cc(-c6ccccc6)nc(-c6ccccc6)c5)ccc43)cc2)cc1 ; c1ccc(-c2cccc(-n3c4ccccc4c4cc(-c5ccc6c(c5)c5ccncc5n6-c5cc(-c6ccccc6)nc(-c6ccccc6)c5)ccc43)c2)cc1]

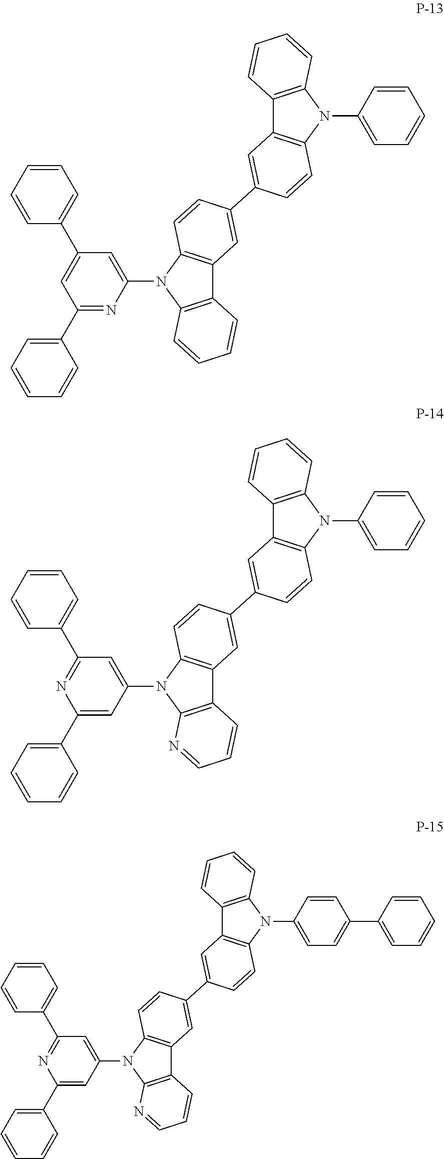 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccc(-c5ccc6c(c5)c5ccccc5n6-c5ccccc5)cc4c4cccnc43)cc(-c3ccccc3)n2)cc1 ; c1ccc(-c2ccc(-n3c4ccccc4c4cc(-c5ccc6c(c5)c5cccnc5n6-c5cc(-c6ccccc6)nc(-c6ccccc6)c5)ccc43)cc2)cc1]

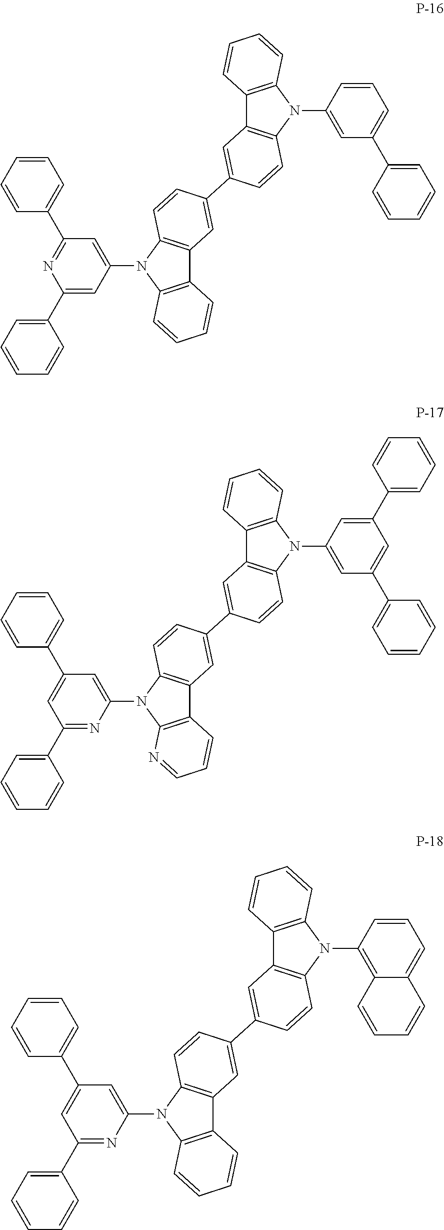 [SMILES: c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)cc(-n3c4ccccc4c4cc(-c5ccc6c(c5)c5cccnc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

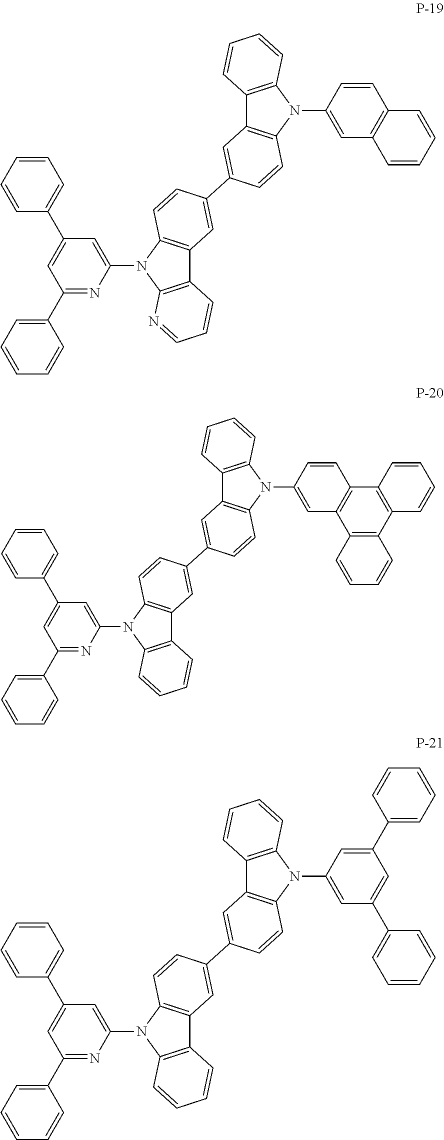 [SMILES: c1ccc(-c2cc(-c3ccccc3)nc(-n3c4ccc(-c5ccc6c(c5)c5ccccc5n6-c5ccc6ccccc6c5)cc4c4cccnc43)c2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

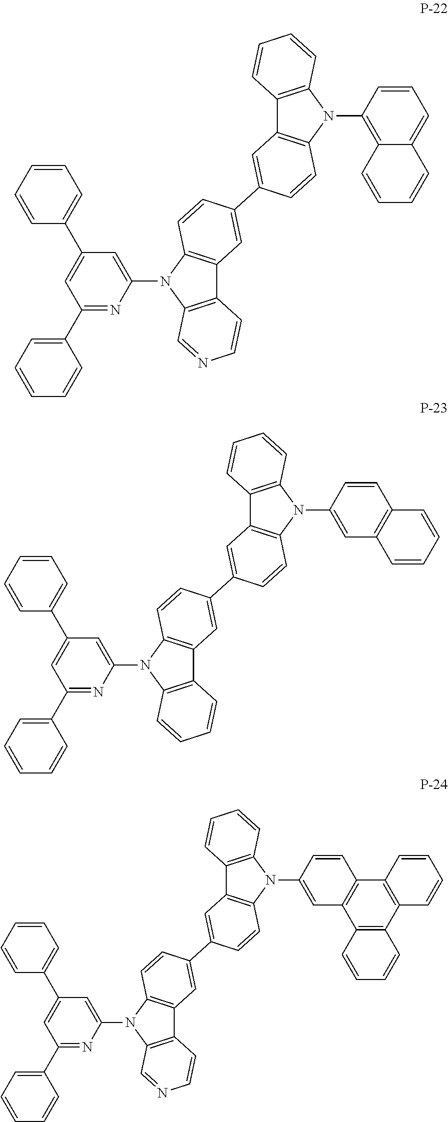 [SMILES: c1ccc(-c2cc(-c3ccccc3)nc(-n3c4ccc(-c5ccc6c(c5)c5ccccc5n6-c5cccc6ccccc56)cc4c4ccncc43)c2)cc1 ; c1ccc(-c2cc(-c3ccccc3)nc(-n3c4ccccc4c4cc(-c5ccc6c(c5)c5ccccc5n6-c5ccc6ccccc6c5)ccc43)c2)cc1 ; c1ccc(-c2cc(-c3ccccc3)nc(-n3c4ccc(-c5ccc6c(c5)c5ccccc5n6-c5ccc6c7ccccc7c7ccccc7c6c5)cc4c4ccncc43)c2)cc1]

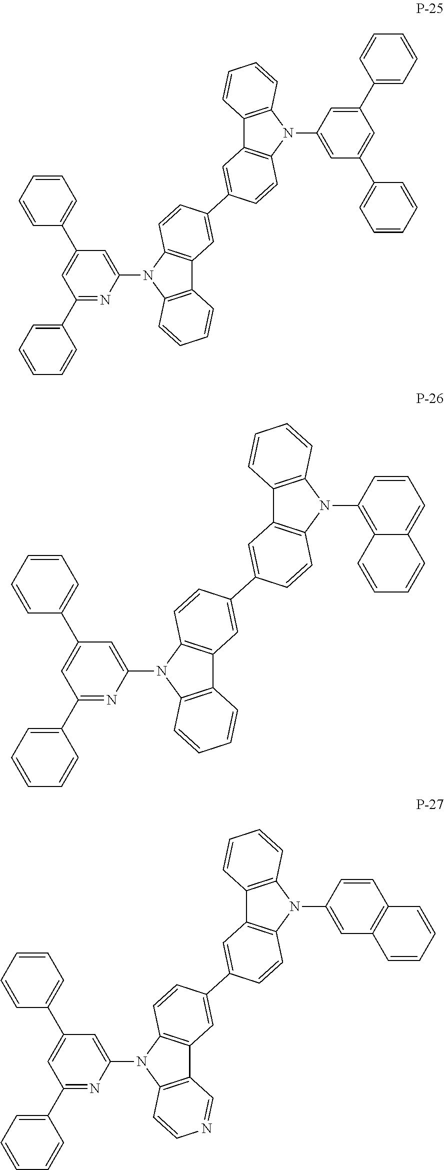 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1 ; c1ccc(-c2cc(-c3ccccc3)nc(-n3c4ccncc4c4cc(-c5ccc6c(c5)c5ccccc5n6-c5ccc6ccccc6c5)ccc43)c2)cc1]

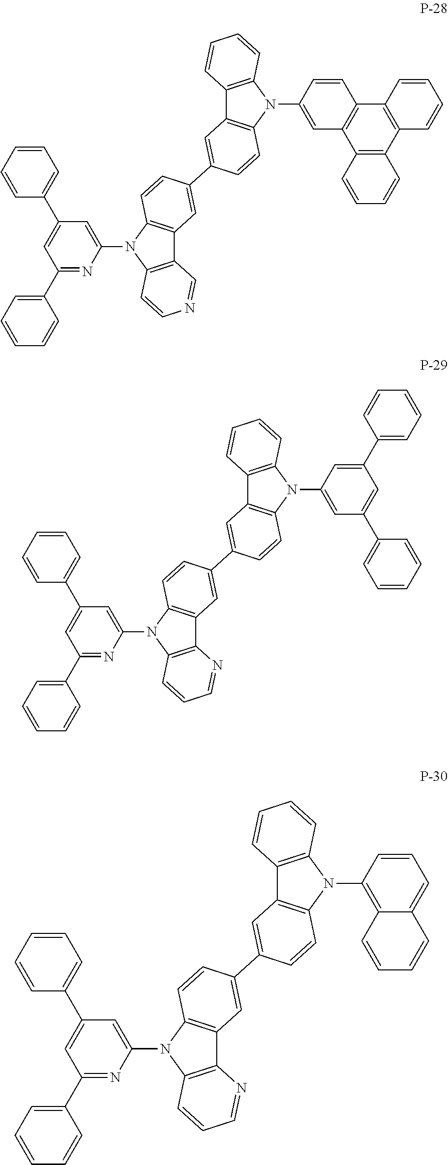 [SMILES: c1ccc(-c2cc(-c3ccccc3)nc(-n3c4ccncc4c4cc(-c5ccc6c(c5)c5ccccc5n6-c5ccc6c7ccccc7c7ccccc7c6c5)ccc43)c2)cc1 ; c1ccc(-c2cc(-c3ccccc3)cc(-n3c4ccccc4c4cc(-c5ccc6c(c5)c5ncccc5n6-c5cc(-c6ccccc6)cc(-c6ccccc6)n5)ccc43)c2)cc1 ; c1ccc(-c2cc(-c3ccccc3)nc(-n3c4ccc(-c5ccc6c(c5)c5ccccc5n6-c5cccc6ccccc56)cc4c4ncccc43)c2)cc1]

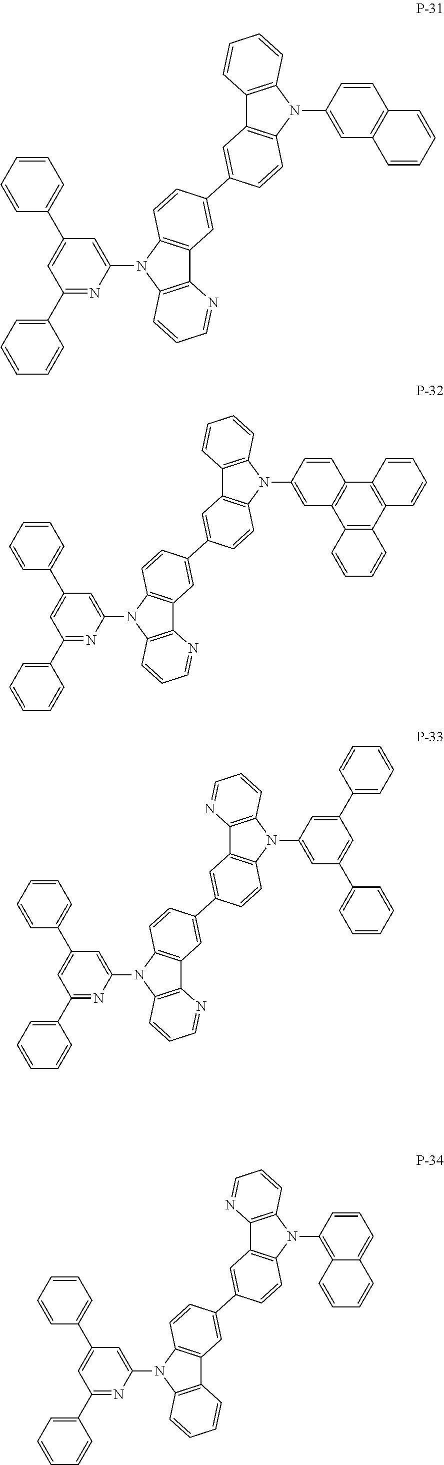 [SMILES: c1ccc(-c2cc(-c3ccccc3)nc(-n3c4ccc(-c5ccc6c(c5)c5ccccc5n6-c5ccc6ccccc6c5)cc4c4ncccc43)c2)cc1 ; c1ccc(-c2cc(-c3ccccc3)nc(-n3c4ccc(-c5ccc6c(c5)c5ccccc5n6-c5ccc6c7ccccc7c7ccccc7c6c5)cc4c4ncccc43)c2)cc1 ; c1ccc(-c2cc(-c3ccccc3)cc(-n3c4ccc(-c5ccc6c(c5)c5ncccc5n6-c5cc(-c6ccccc6)cc(-c6ccccc6)n5)cc4c4ncccc43)c2)cc1 ; c1ccc(-c2cc(-c3ccccc3)nc(-n3c4ccccc4c4cc(-c5ccc6c(c5)c5ncccc5n6-c5cccc6ccccc56)ccc43)c2)cc1]

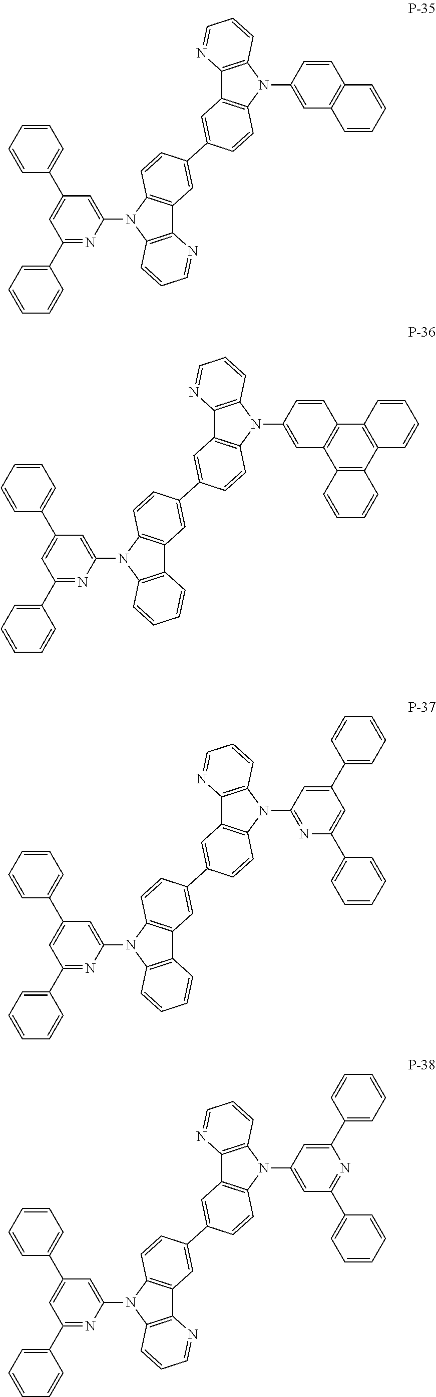 [SMILES: c1ccc(-c2cc(-c3ccccc3)nc(-n3c4ccc(-c5ccc6c(c5)c5ncccc5n6-c5ccc6ccccc6c5)cc4c4ncccc43)c2)cc1 ; c1ccc(-c2cc(-c3ccccc3)nc(-n3c4ccccc4c4cc(-c5ccc6c(c5)c5ncccc5n6-c5ccc6c7ccccc7c7ccccc7c6c5)ccc43)c2)cc1 ; c1ccc(-c2cc(-c3ccccc3)nc(-n3c4ccccc4c4cc(-c5ccc6c(c5)c5ncccc5n6-c5cc(-c6ccccc6)cc(-c6ccccc6)n5)ccc43)c2)cc1 ; c1ccc(-c2cc(-c3ccccc3)nc(-n3c4ccc(-c5ccc6c(c5)c5ncccc5n6-c5cc(-c6ccccc6)nc(-c6ccccc6)c5)cc4c4ncccc43)c2)cc1]

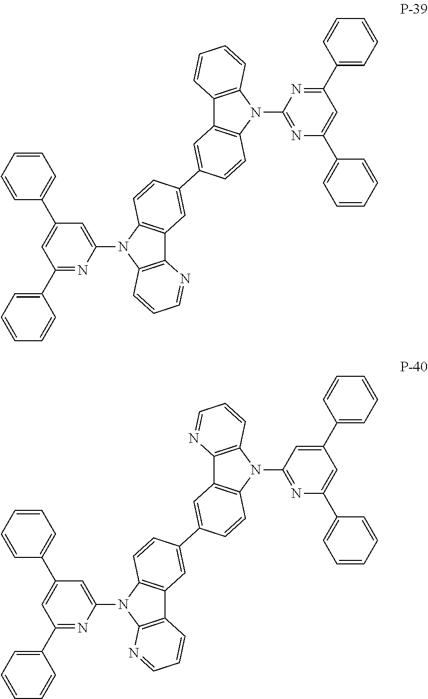 [SMILES: c1ccc(-c2cc(-c3ccccc3)nc(-n3c4ccc(-c5ccc6c(c5)c5ccccc5n6-c5nc(-c6ccccc6)cc(-c6ccccc6)n5)cc4c4ncccc43)c2)cc1 ; c1ccc(-c2cc(-c3ccccc3)nc(-n3c4ccc(-c5ccc6c(c5)c5cccnc5n6-c5cc(-c6ccccc6)cc(-c6ccccc6)n5)cc4c4ncccc43)c2)cc1]

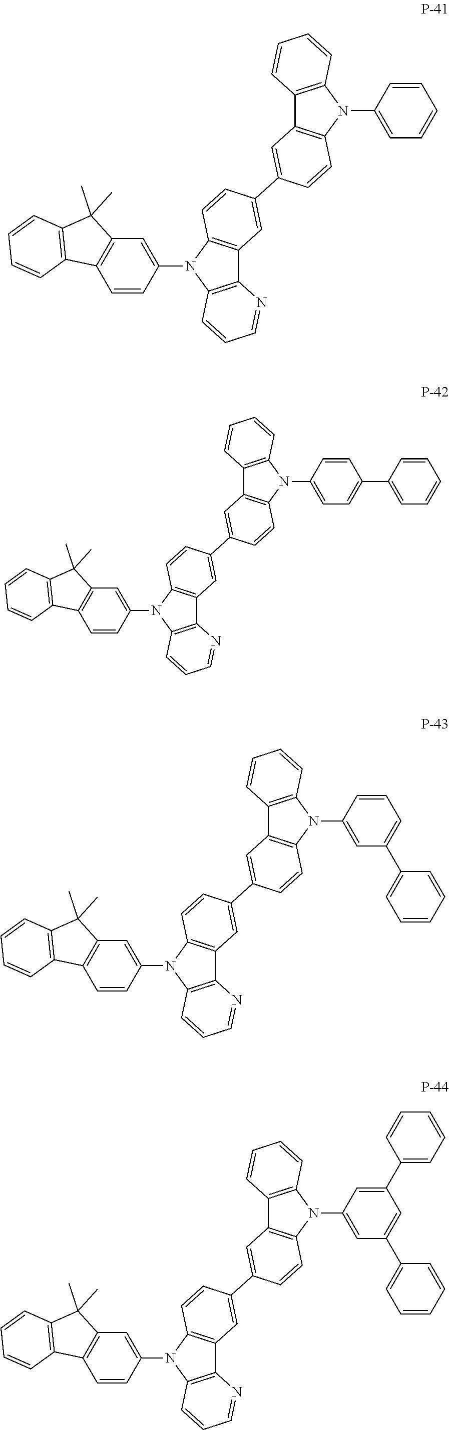 [SMILES: CC1(C)c2ccccc2-c2ccc(-n3c4ccc(-c5ccc6c(c5)c5ccccc5n6-c5ccccc5)cc4c4ncccc43)cc21 ; CC1(C)c2ccccc2-c2ccc(-n3c4ccc(-c5ccc6c(c5)c5ccccc5n6-c5ccc(-c6ccccc6)cc5)cc4c4ncccc43)cc21 ; CC1(C)c2ccccc2-c2ccc(-n3c4ccc(-c5ccc6c(c5)c5ccccc5n6-c5cccc(-c6ccccc6)c5)cc4c4ncccc43)cc21 ; CC1(C)c2ccccc2-c2ccc(-n3c4ccc(-c5ccc6c(c5)c5ccccc5n6-c5cc(-c6ccccc6)cc(-c6ccccc6)c5)cc4c4ncccc43)cc21]

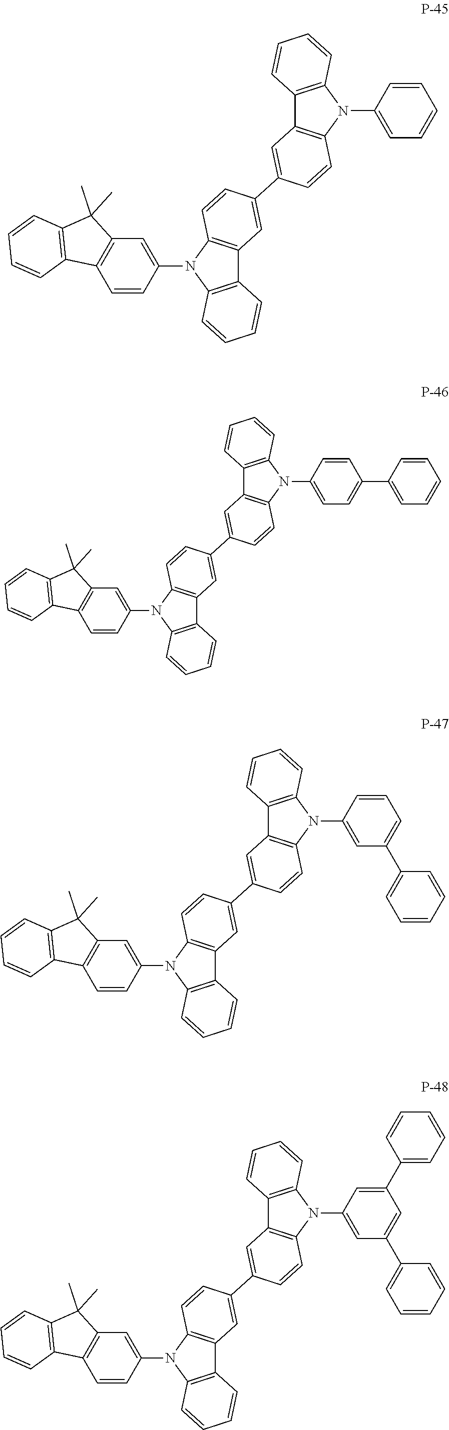 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cccc(-c6ccccc6)c5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

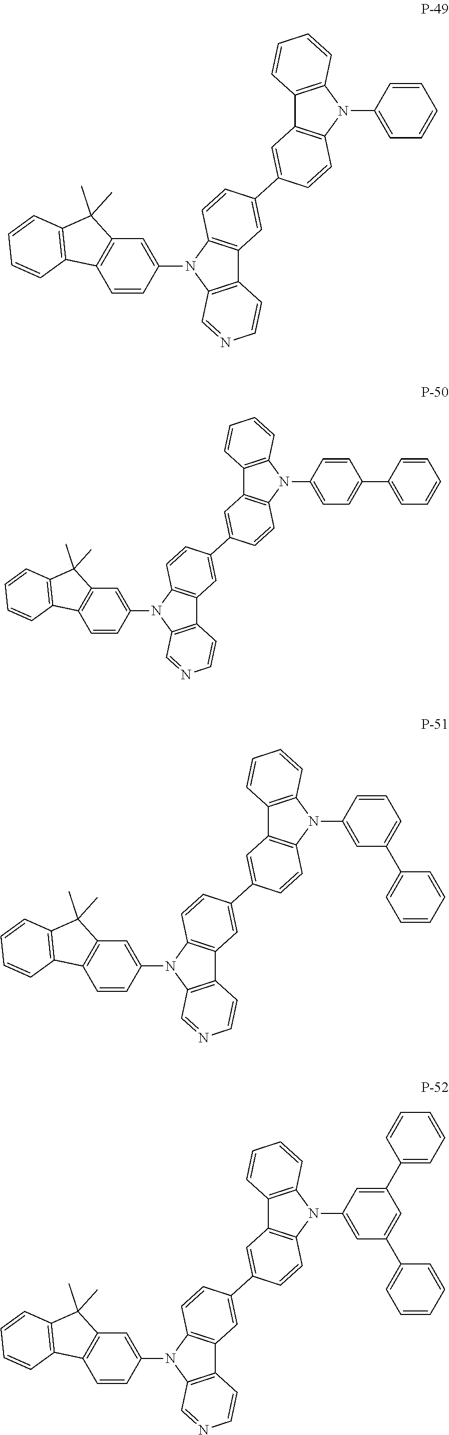 [SMILES: CC1(C)c2ccccc2-c2ccc(-n3c4ccc(-c5ccc6c(c5)c5ccccc5n6-c5ccccc5)cc4c4ccncc43)cc21 ; CC1(C)c2ccccc2-c2ccc(-n3c4ccc(-c5ccc6c(c5)c5ccccc5n6-c5ccc(-c6ccccc6)cc5)cc4c4ccncc43)cc21 ; CC1(C)c2ccccc2-c2ccc(-n3c4ccc(-c5ccc6c(c5)c5ccccc5n6-c5cccc(-c6ccccc6)c5)cc4c4ccncc43)cc21 ; CC1(C)c2ccccc2-c2ccc(-n3c4ccc(-c5ccc6c(c5)c5ccccc5n6-c5cc(-c6ccccc6)cc(-c6ccccc6)c5)cc4c4ccncc43)cc21]

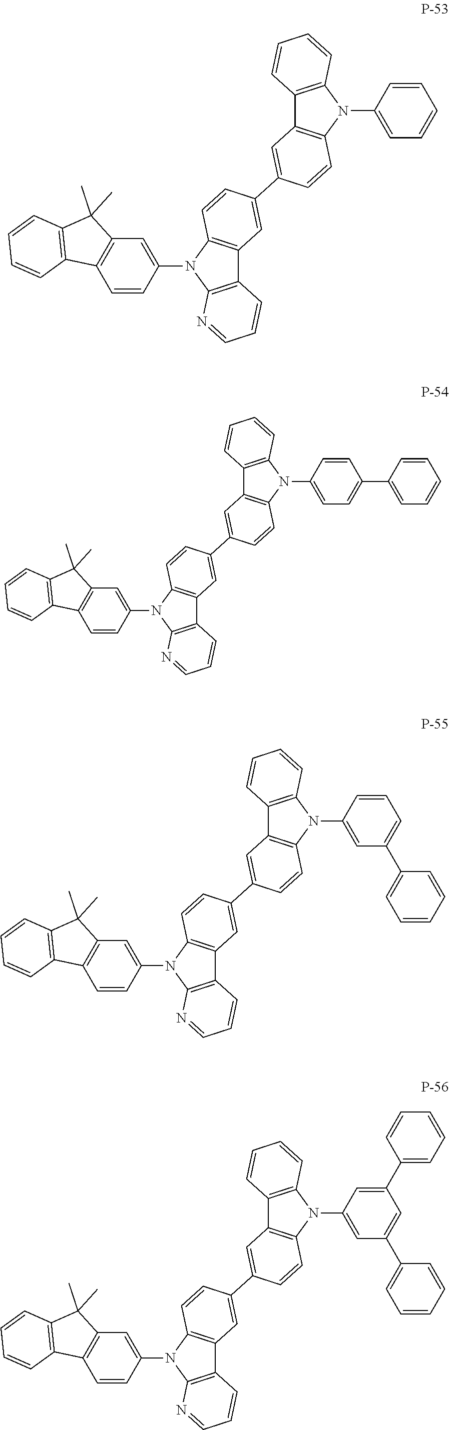 [SMILES: CC1(C)c2ccccc2-c2ccc(-n3c4ccc(-c5ccc6c(c5)c5ccccc5n6-c5ccccc5)cc4c4cccnc43)cc21 ; CC1(C)c2ccccc2-c2ccc(-n3c4ccc(-c5ccc6c(c5)c5ccccc5n6-c5ccc(-c6ccccc6)cc5)cc4c4cccnc43)cc21 ; CC1(C)c2ccccc2-c2ccc(-n3c4ccc(-c5ccc6c(c5)c5ccccc5n6-c5cccc(-c6ccccc6)c5)cc4c4cccnc43)cc21 ; CC1(C)c2ccccc2-c2ccc(-n3c4ccc(-c5ccc6c(c5)c5ccccc5n6-c5cc(-c6ccccc6)cc(-c6ccccc6)c5)cc4c4cccnc43)cc21]

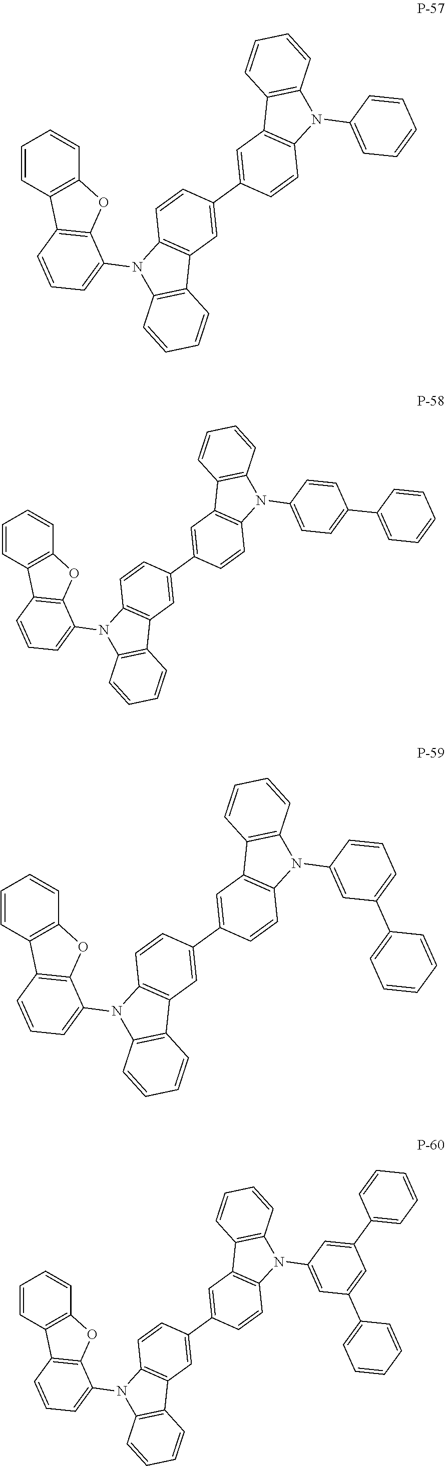 [SMILES: c1ccc(-n2c3ccccc3c3cc(-c4ccc5c(c4)c4ccccc4n5-c4cccc5c4oc4ccccc45)ccc32)cc1 ; c1ccc(-c2ccc(-n3c4ccccc4c4cc(-c5ccc6c(c5)c5ccccc5n6-c5cccc6c5oc5ccccc56)ccc43)cc2)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

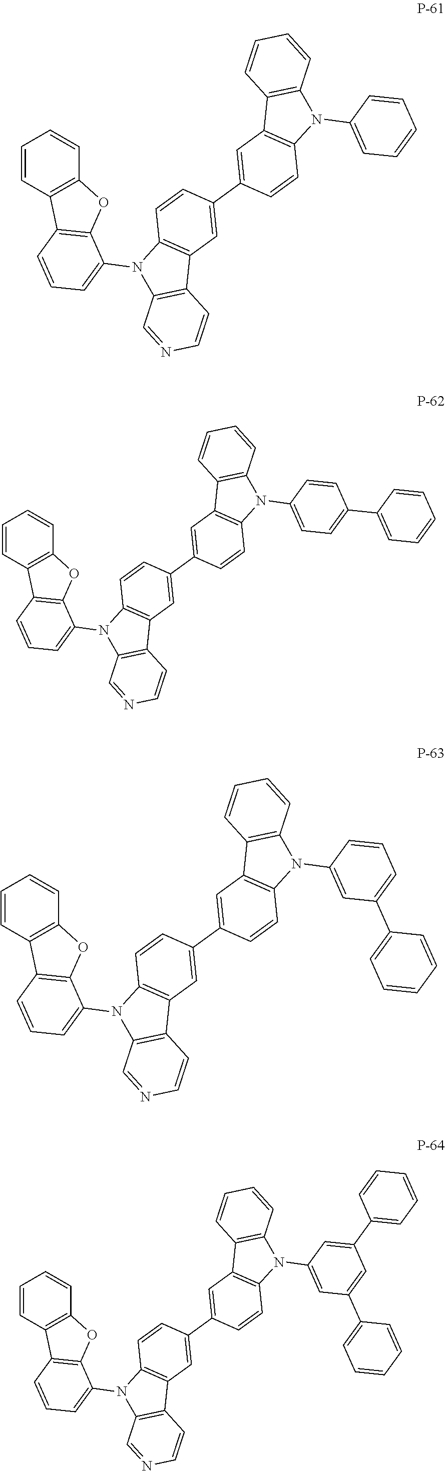 [SMILES: c1ccc(-n2c3ccccc3c3cc(-c4ccc5c(c4)c4ccncc4n5-c4cccc5c4oc4ccccc45)ccc32)cc1 ; c1ccc(-c2ccc(-n3c4ccccc4c4cc(-c5ccc6c(c5)c5ccncc5n6-c5cccc6c5oc5ccccc56)ccc43)cc2)cc1 ; c1ccc(-c2cccc(-n3c4ccccc4c4cc(-c5ccc6c(c5)c5ccncc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccncc5n6-c5cccc6c5oc5ccccc56)ccc43)c2)cc1]

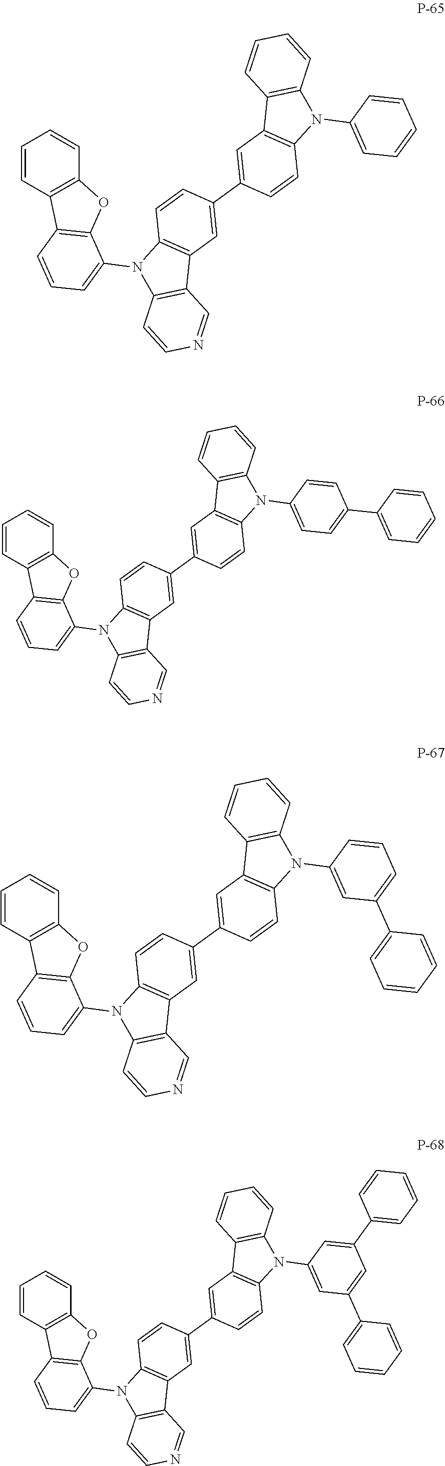 [SMILES: c1ccc(-n2c3ccccc3c3cc(-c4ccc5c(c4)c4cnccc4n5-c4cccc5c4oc4ccccc45)ccc32)cc1 ; c1ccc(-c2ccc(-n3c4ccccc4c4cc(-c5ccc6c(c5)c5cnccc5n6-c5cccc6c5oc5ccccc56)ccc43)cc2)cc1 ; c1ccc(-c2cccc(-n3c4ccccc4c4cc(-c5ccc6c(c5)c5cnccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5cnccc5n6-c5cccc6c5oc5ccccc56)ccc43)c2)cc1]

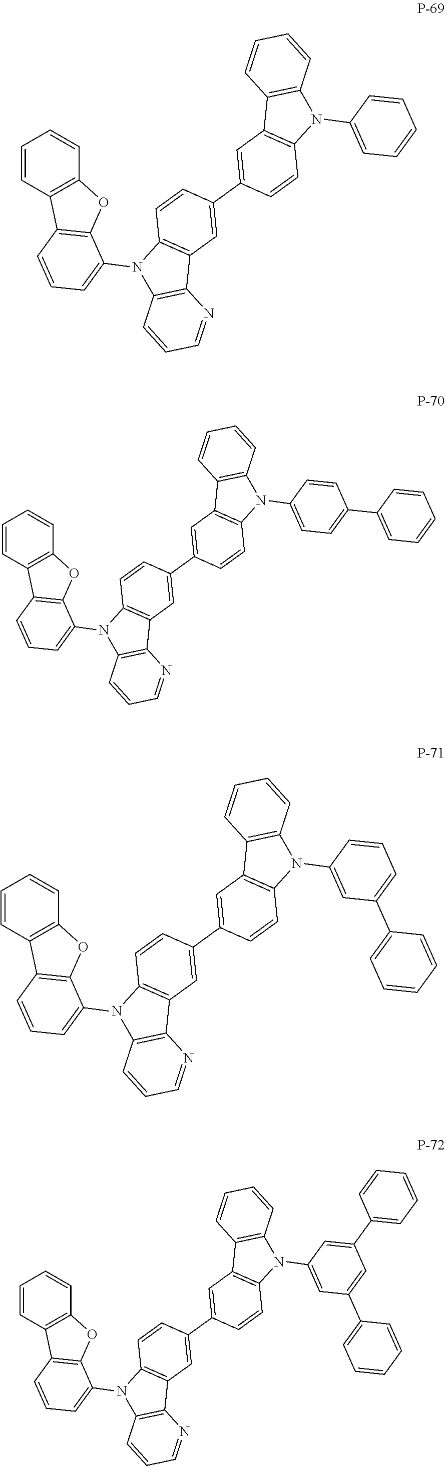 [SMILES: c1ccc(-n2c3ccccc3c3cc(-c4ccc5c(c4)c4ncccc4n5-c4cccc5c4oc4ccccc45)ccc32)cc1 ; c1ccc(-c2ccc(-n3c4ccccc4c4cc(-c5ccc6c(c5)c5ncccc5n6-c5cccc6c5oc5ccccc56)ccc43)cc2)cc1 ; c1ccc(-c2cccc(-n3c4ccccc4c4cc(-c5ccc6c(c5)c5ncccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ncccc5n6-c5cccc6c5oc5ccccc56)ccc43)c2)cc1]

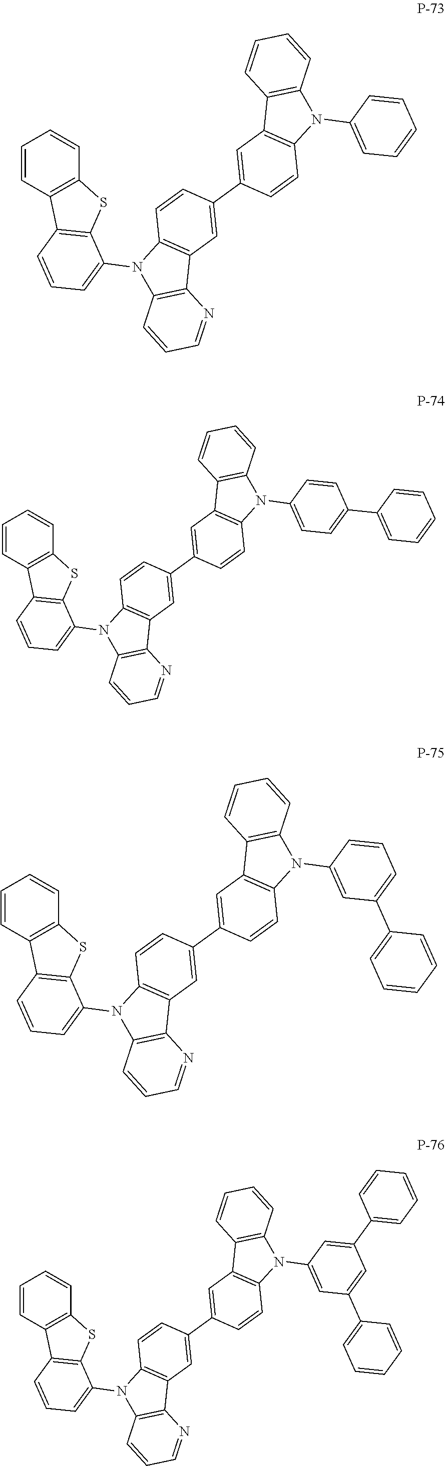 [SMILES: c1ccc(-n2c3ccccc3c3cc(-c4ccc5c(c4)c4ncccc4n5-c4cccc5c4sc4ccccc45)ccc32)cc1 ; c1ccc(-c2ccc(-n3c4ccccc4c4cc(-c5ccc6c(c5)c5ncccc5n6-c5cccc6c5sc5ccccc56)ccc43)cc2)cc1 ; c1ccc(-c2cccc(-n3c4ccccc4c4cc(-c5ccc6c(c5)c5ncccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ncccc5n6-c5cccc6c5sc5ccccc56)ccc43)c2)cc1]

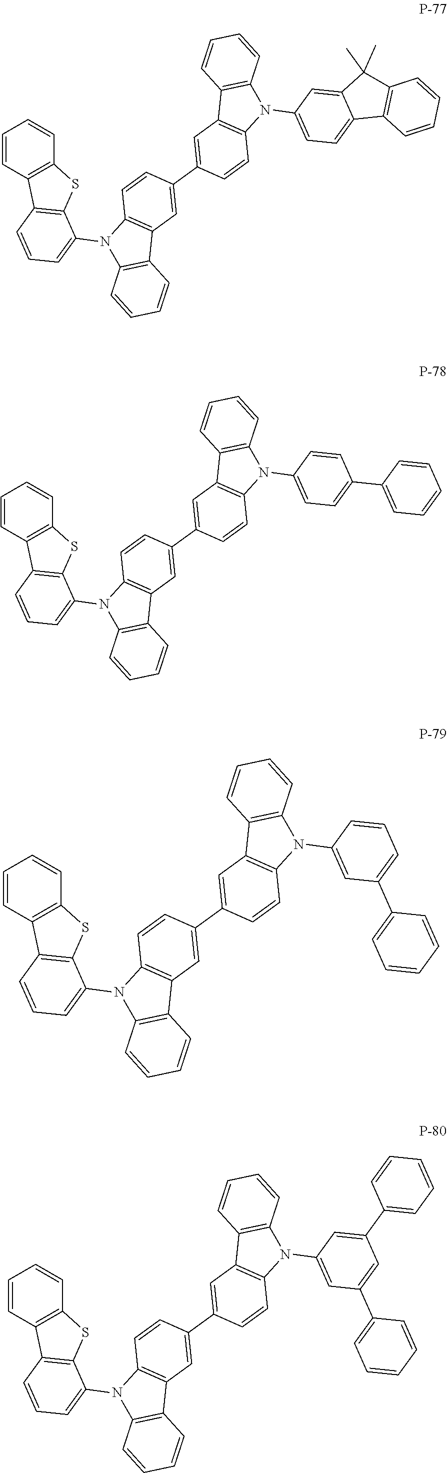 [SMILES: CC1(C)c2ccccc2-c2ccc(-n3c4ccccc4c4cc(-c5ccc6c(c5)c5ccccc5n6-c5cccc6c5sc5ccccc56)ccc43)cc21 ; c1ccc(-c2ccc(-n3c4ccccc4c4cc(-c5ccc6c(c5)c5ccccc5n6-c5cccc6c5sc5ccccc56)ccc43)cc2)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

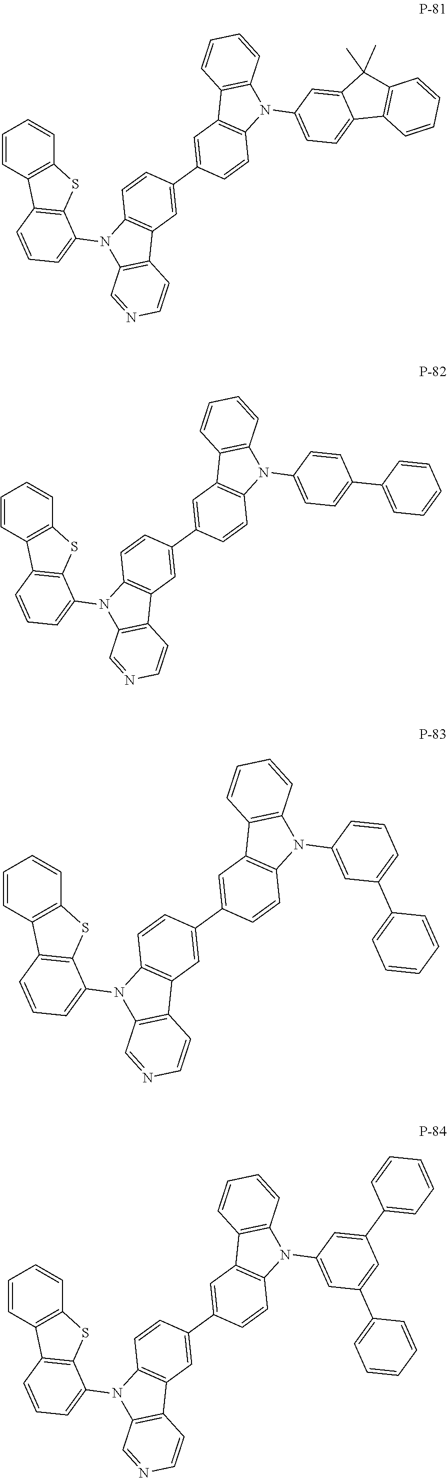 [SMILES: CC1(C)c2ccccc2-c2ccc(-n3c4ccccc4c4cc(-c5ccc6c(c5)c5ccncc5n6-c5cccc6c5sc5ccccc56)ccc43)cc21 ; c1ccc(-c2ccc(-n3c4ccccc4c4cc(-c5ccc6c(c5)c5ccncc5n6-c5cccc6c5sc5ccccc56)ccc43)cc2)cc1 ; c1ccc(-c2cccc(-n3c4ccccc4c4cc(-c5ccc6c(c5)c5ccncc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccncc5n6-c5cccc6c5sc5ccccc56)ccc43)c2)cc1]

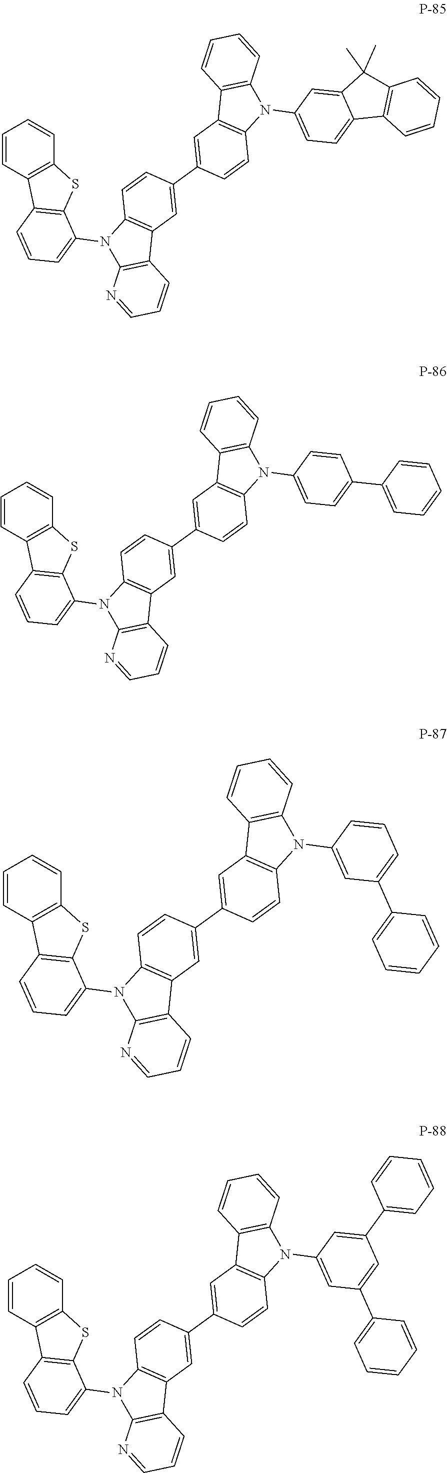 [SMILES: CC1(C)c2ccccc2-c2ccc(-n3c4ccccc4c4cc(-c5ccc6c(c5)c5cccnc5n6-c5cccc6c5sc5ccccc56)ccc43)cc21 ; c1ccc(-c2ccc(-n3c4ccccc4c4cc(-c5ccc6c(c5)c5cccnc5n6-c5cccc6c5sc5ccccc56)ccc43)cc2)cc1 ; c1ccc(-c2cccc(-n3c4ccccc4c4cc(-c5ccc6c(c5)c5cccnc5n6-c5cccc6c5sc5ccccc56)ccc43)c2)cc1 ; c1ccc(-c2cc(-c3ccccc3)cc(-n3c4ccccc4c4cc(-c5ccc6c(c5)c5cccnc5n6-c5cccc6c5sc5ccccc56)ccc43)c2)cc1]

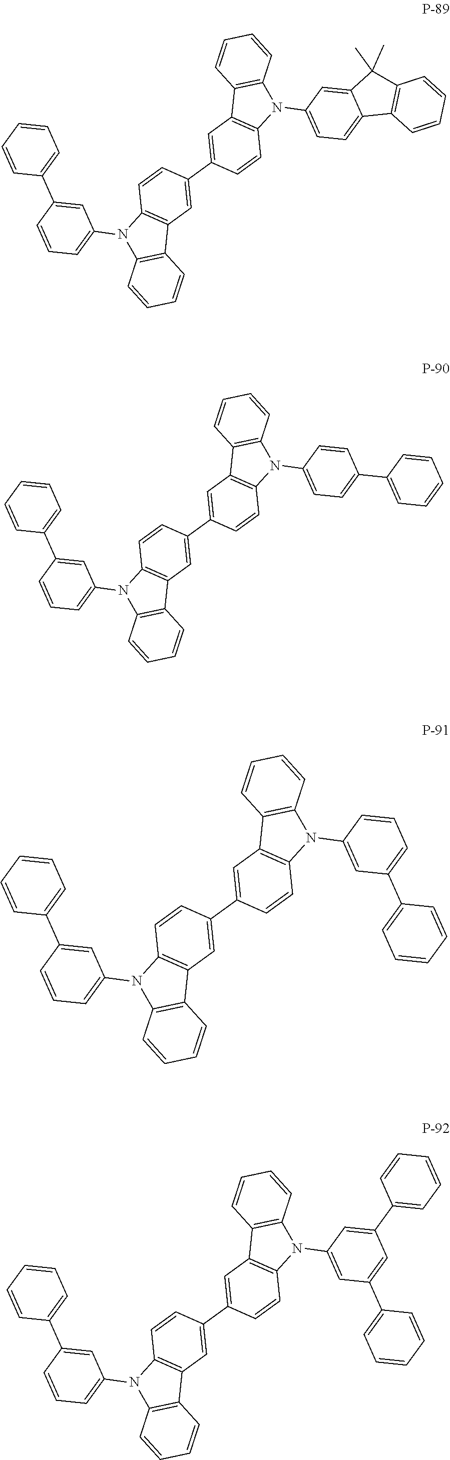 [SMILES: CC1(C)c2ccccc2-c2ccc(-n3c4ccccc4c4cc(-c5ccc6c(c5)c5ccccc5n6-c5cccc(-c6ccccc6)c5)ccc43)cc21 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

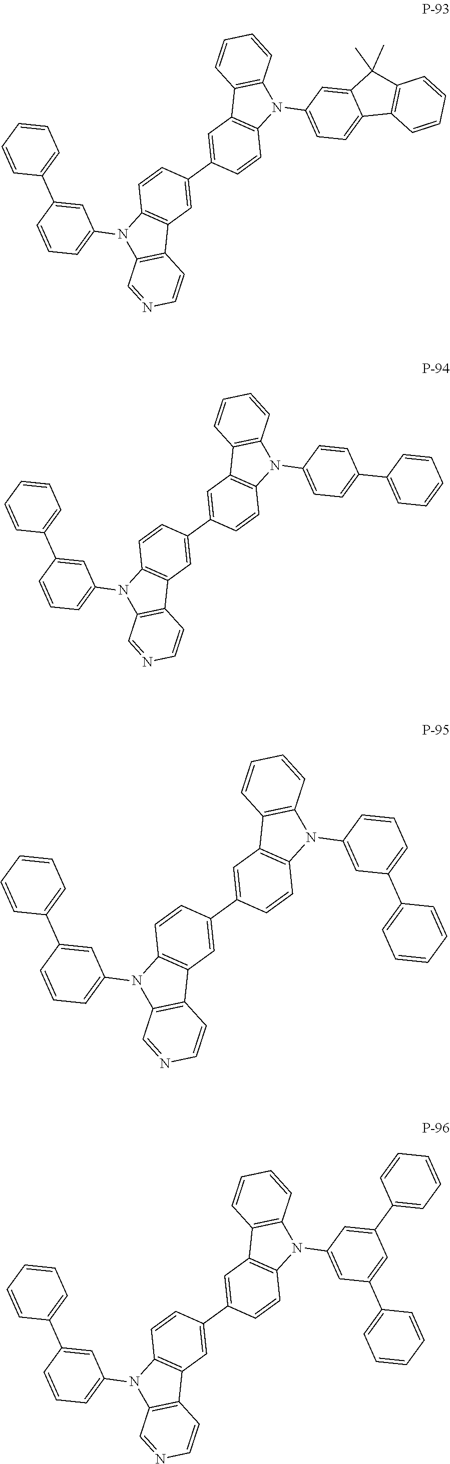 [SMILES: CC1(C)c2ccccc2-c2ccc(-n3c4ccccc4c4cc(-c5ccc6c(c5)c5ccncc5n6-c5cccc(-c6ccccc6)c5)ccc43)cc21 ; c1ccc(-c2ccc(-n3c4ccccc4c4cc(-c5ccc6c(c5)c5ccncc5n6-c5cccc(-c6ccccc6)c5)ccc43)cc2)cc1 ; c1ccc(-c2cccc(-n3c4ccccc4c4cc(-c5ccc6c(c5)c5ccncc5n6-c5cccc(-c6ccccc6)c5)ccc43)c2)cc1 ; c1ccc(-c2cccc(-n3c4ccc(-c5ccc6c(c5)c5ccccc5n6-c5cc(-c6ccccc6)cc(-c6ccccc6)c5)cc4c4ccncc43)c2)cc1]

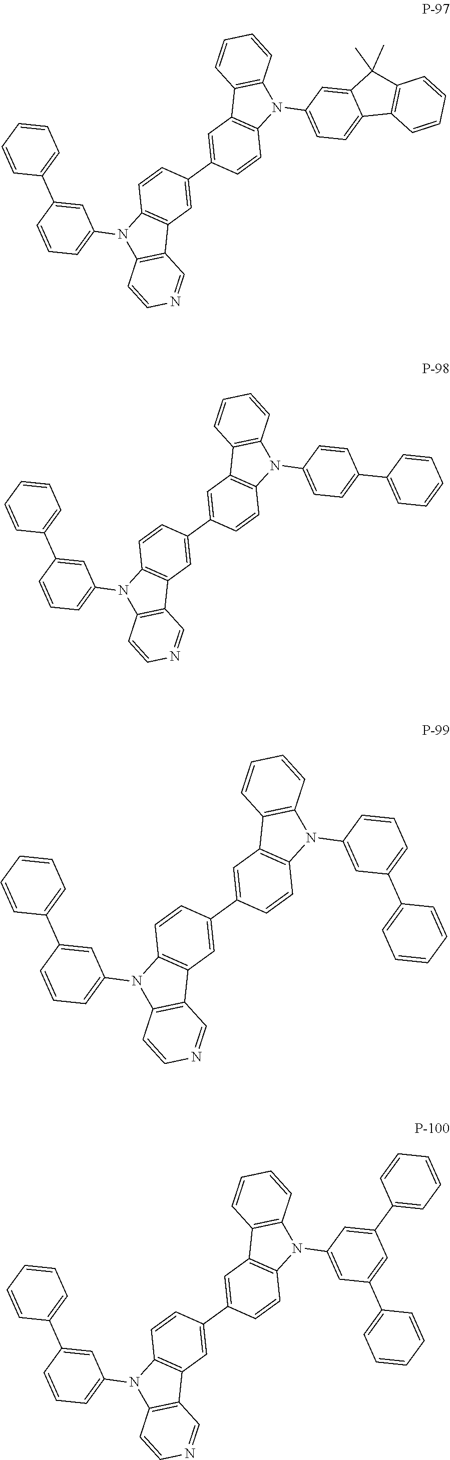 [SMILES: CC1(C)c2ccccc2-c2ccc(-n3c4ccccc4c4cc(-c5ccc6c(c5)c5cnccc5n6-c5cccc(-c6ccccc6)c5)ccc43)cc21 ; c1ccc(-c2ccc(-n3c4ccccc4c4cc(-c5ccc6c(c5)c5cnccc5n6-c5cccc(-c6ccccc6)c5)ccc43)cc2)cc1 ; c1ccc(-c2cccc(-n3c4ccccc4c4cc(-c5ccc6c(c5)c5cnccc5n6-c5cccc(-c6ccccc6)c5)ccc43)c2)cc1 ; c1ccc(-c2cccc(-n3c4ccncc4c4cc(-c5ccc6c(c5)c5ccccc5n6-c5cc(-c6ccccc6)cc(-c6ccccc6)c5)ccc43)c2)cc1]

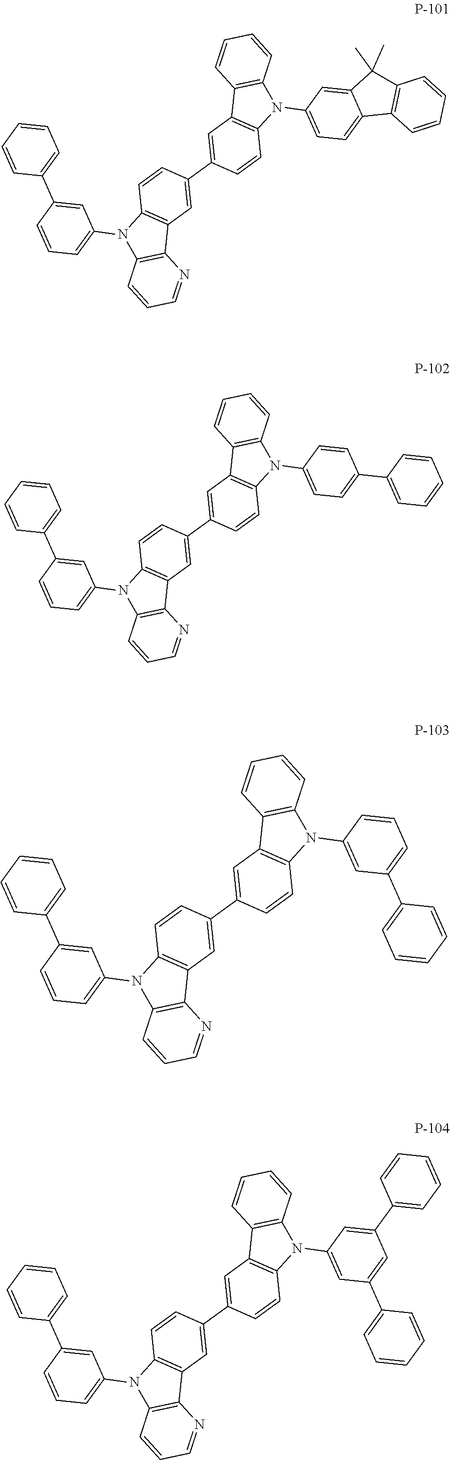 [SMILES: CC1(C)c2ccccc2-c2ccc(-n3c4ccccc4c4cc(-c5ccc6c(c5)c5ncccc5n6-c5cccc(-c6ccccc6)c5)ccc43)cc21 ; c1ccc(-c2ccc(-n3c4ccccc4c4cc(-c5ccc6c(c5)c5ncccc5n6-c5cccc(-c6ccccc6)c5)ccc43)cc2)cc1 ; c1ccc(-c2cccc(-n3c4ccccc4c4cc(-c5ccc6c(c5)c5ncccc5n6-c5cccc(-c6ccccc6)c5)ccc43)c2)cc1 ; c1ccc(-c2cc(-c3ccccc3)cc(-n3c4ccccc4c4cc(-c5ccc6c(c5)c5ncccc5n6-c5cccc(-c6ccccc6)c5)ccc43)c2)cc1]

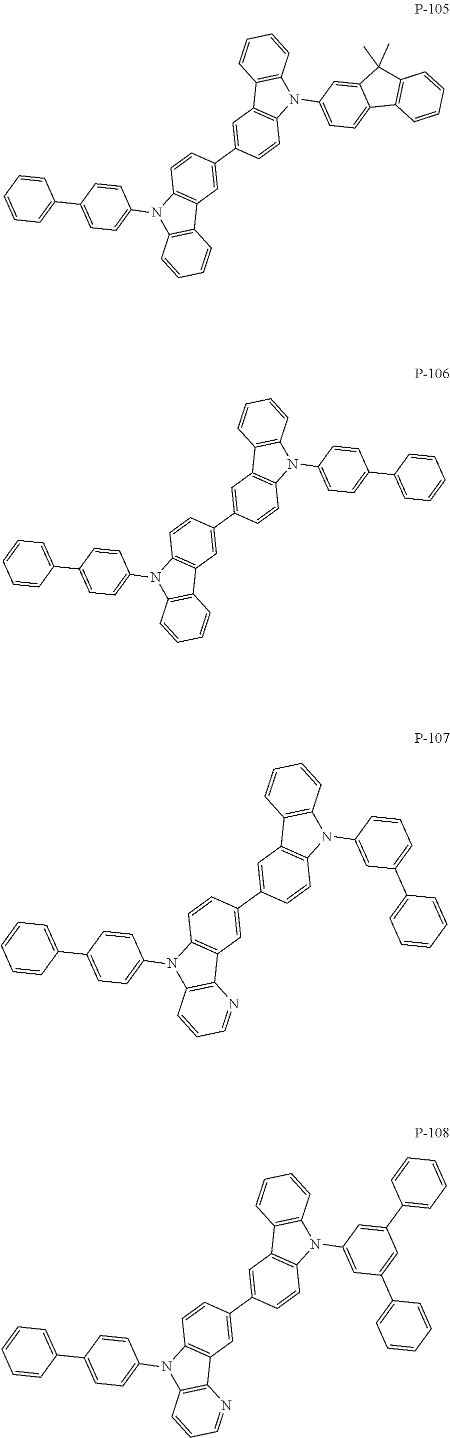 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4ncccc43)cc2)cc1 ; c1ccc(-c2ccc(-n3c4ccc(-c5ccc6c(c5)c5ccccc5n6-c5cc(-c6ccccc6)cc(-c6ccccc6)c5)cc4c4ncccc43)cc2)cc1]

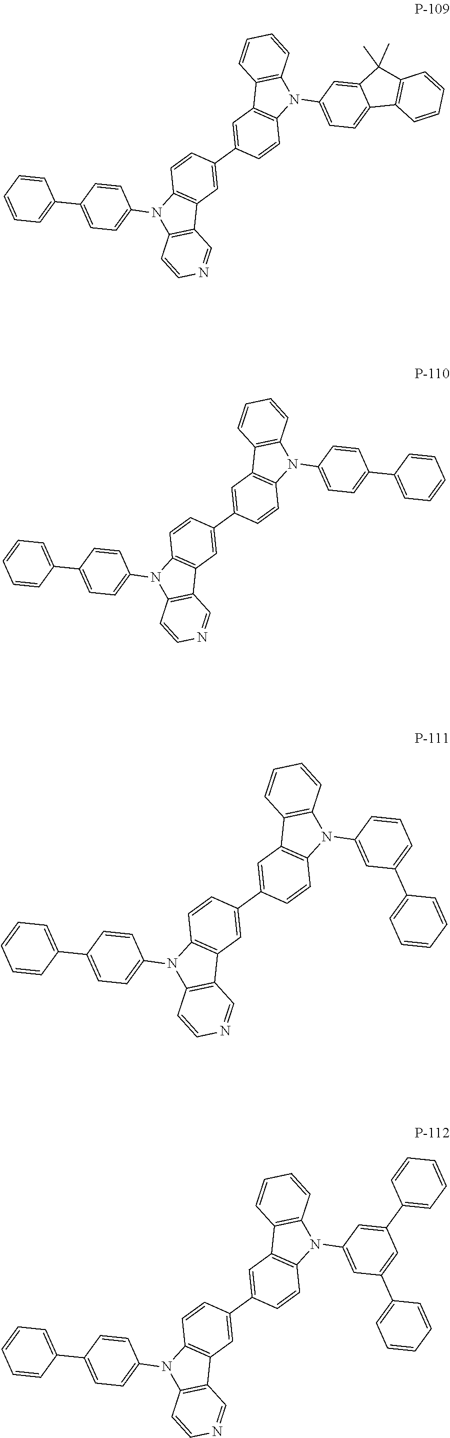 [SMILES: CC1(C)c2ccccc2-c2ccc(-n3c4ccccc4c4cc(-c5ccc6c(c5)c5cnccc5n6-c5ccc(-c6ccccc6)cc5)ccc43)cc21 ; c1ccc(-c2ccc(-n3c4ccccc4c4cc(-c5ccc6c(c5)c5cnccc5n6-c5ccc(-c6ccccc6)cc5)ccc43)cc2)cc1 ; c1ccc(-c2ccc(-n3c4ccncc4c4cc(-c5ccc6c(c5)c5ccccc5n6-c5cccc(-c6ccccc6)c5)ccc43)cc2)cc1 ; c1ccc(-c2ccc(-n3c4ccncc4c4cc(-c5ccc6c(c5)c5ccccc5n6-c5cc(-c6ccccc6)cc(-c6ccccc6)c5)ccc43)cc2)cc1]

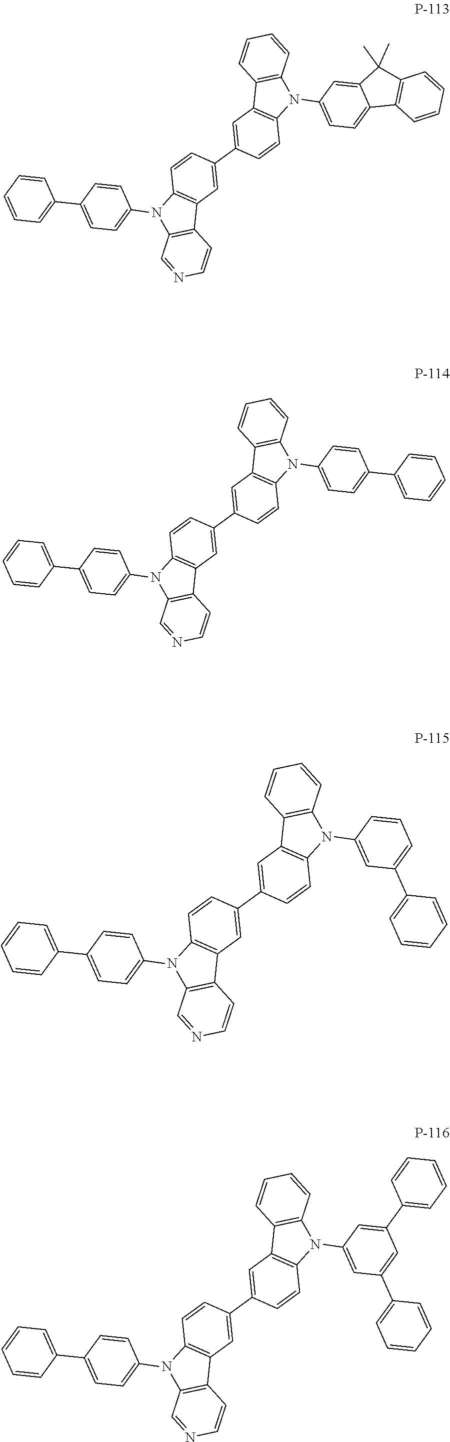 [SMILES: CC1(C)c2ccccc2-c2ccc(-n3c4ccccc4c4cc(-c5ccc6c(c5)c5ccncc5n6-c5ccc(-c6ccccc6)cc5)ccc43)cc21 ; c1ccc(-c2ccc(-n3c4ccccc4c4cc(-c5ccc6c(c5)c5ccncc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4ccncc43)cc2)cc1 ; c1ccc(-c2ccc(-n3c4ccc(-c5ccc6c(c5)c5ccccc5n6-c5cc(-c6ccccc6)cc(-c6ccccc6)c5)cc4c4ccncc43)cc2)cc1]

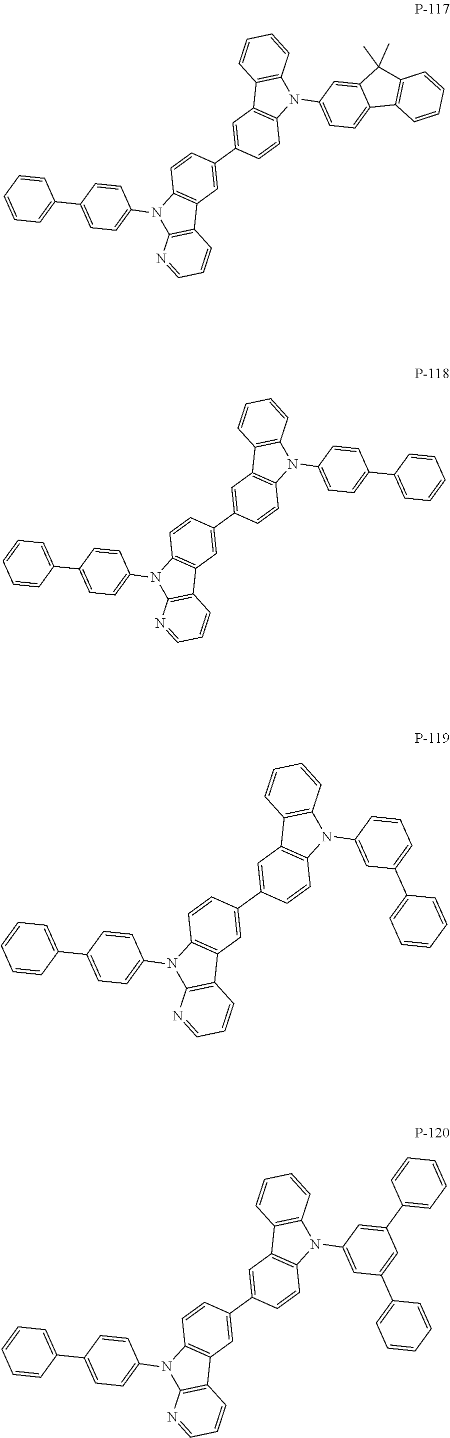 [SMILES: CC1(C)c2ccccc2-c2ccc(-n3c4ccccc4c4cc(-c5ccc6c(c5)c5cccnc5n6-c5ccc(-c6ccccc6)cc5)ccc43)cc21 ; c1ccc(-c2ccc(-n3c4ccccc4c4cc(-c5ccc6c(c5)c5cccnc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4cccnc43)cc2)cc1 ; c1ccc(-c2ccc(-n3c4ccc(-c5ccc6c(c5)c5ccccc5n6-c5cc(-c6ccccc6)cc(-c6ccccc6)c5)cc4c4cccnc43)cc2)cc1]

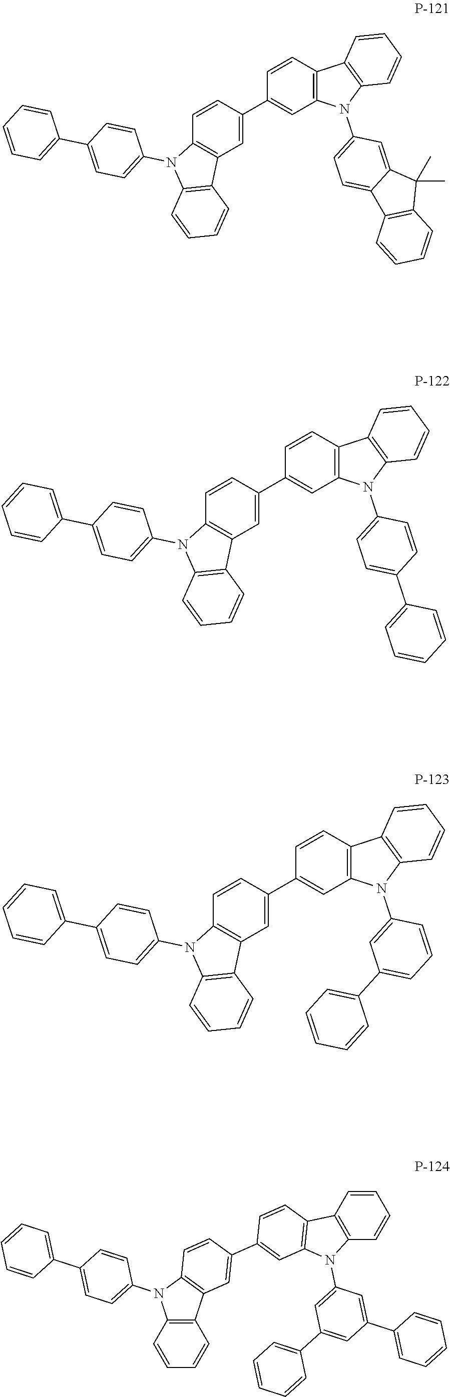 [SMILES: CC1(C)c2ccccc2-c2ccc(-n3c4ccccc4c4ccc(-c5ccc6c(c5)c5ccccc5n6-c5ccc(-c6ccccc6)cc5)cc43)cc21 ; c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1]

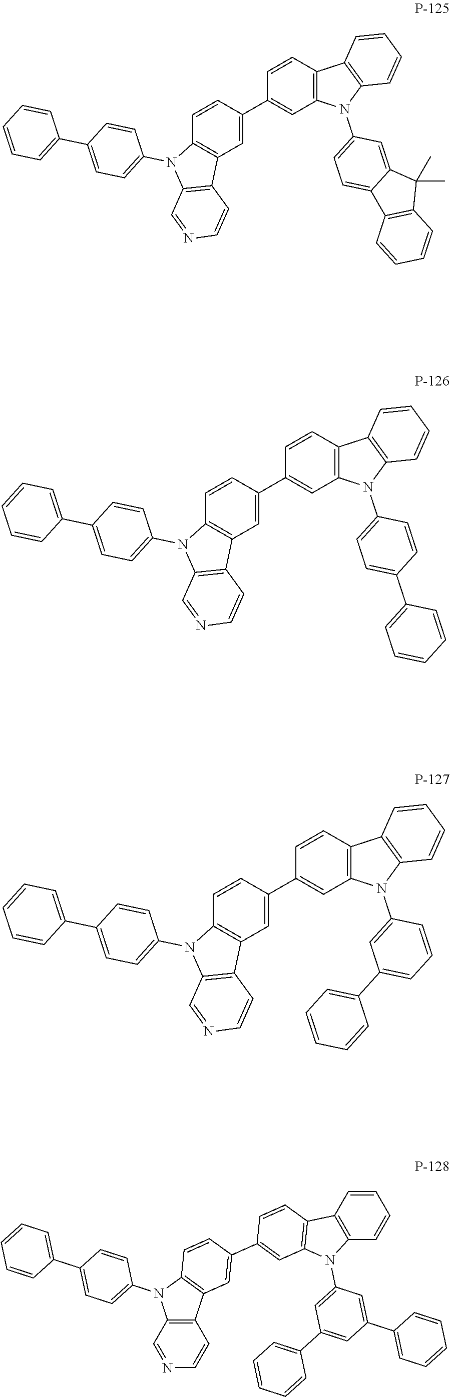 [SMILES: CC1(C)c2ccccc2-c2ccc(-n3c4ccccc4c4ccc(-c5ccc6c(c5)c5ccncc5n6-c5ccc(-c6ccccc6)cc5)cc43)cc21 ; c1ccc(-c2ccc(-n3c4ccc(-c5ccc6c7ccccc7n(-c7ccc(-c8ccccc8)cc7)c6c5)cc4c4ccncc43)cc2)cc1 ; c1ccc(-c2ccc(-n3c4ccc(-c5ccc6c7ccccc7n(-c7cccc(-c8ccccc8)c7)c6c5)cc4c4ccncc43)cc2)cc1 ; c1ccc(-c2ccc(-n3c4ccc(-c5ccc6c7ccccc7n(-c7cc(-c8ccccc8)cc(-c8ccccc8)c7)c6c5)cc4c4ccncc43)cc2)cc1]

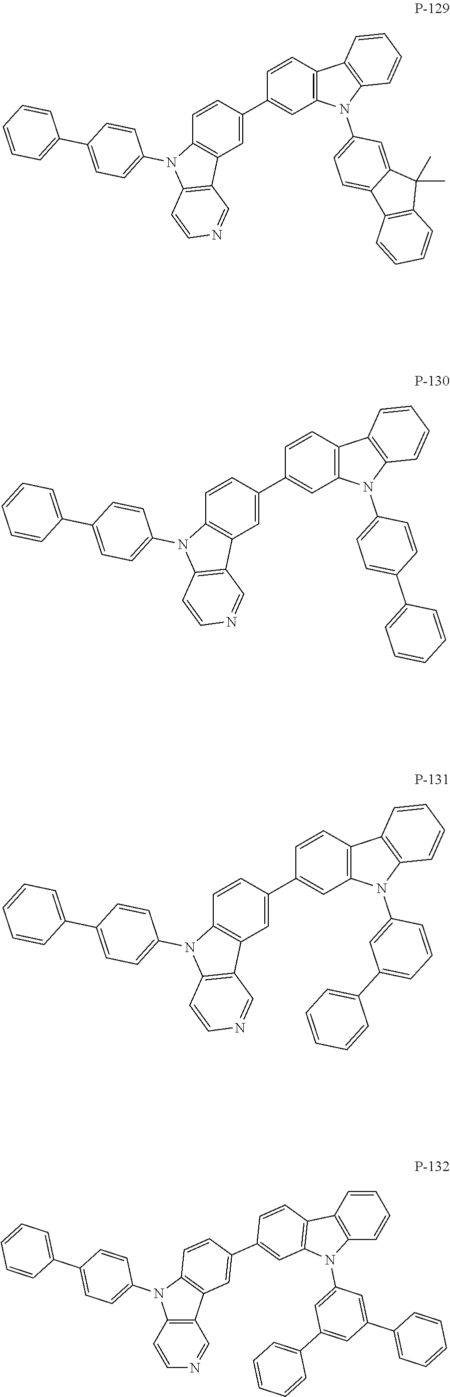 [SMILES: CC1(C)c2ccccc2-c2ccc(-n3c4ccccc4c4ccc(-c5ccc6c(c5)c5cnccc5n6-c5ccc(-c6ccccc6)cc5)cc43)cc21 ; c1ccc(-c2ccc(-n3c4ccncc4c4cc(-c5ccc6c7ccccc7n(-c7ccc(-c8ccccc8)cc7)c6c5)ccc43)cc2)cc1 ; c1ccc(-c2ccc(-n3c4ccncc4c4cc(-c5ccc6c7ccccc7n(-c7cccc(-c8ccccc8)c7)c6c5)ccc43)cc2)cc1 ; c1ccc(-c2ccc(-n3c4ccncc4c4cc(-c5ccc6c7ccccc7n(-c7cc(-c8ccccc8)cc(-c8ccccc8)c7)c6c5)ccc43)cc2)cc1]

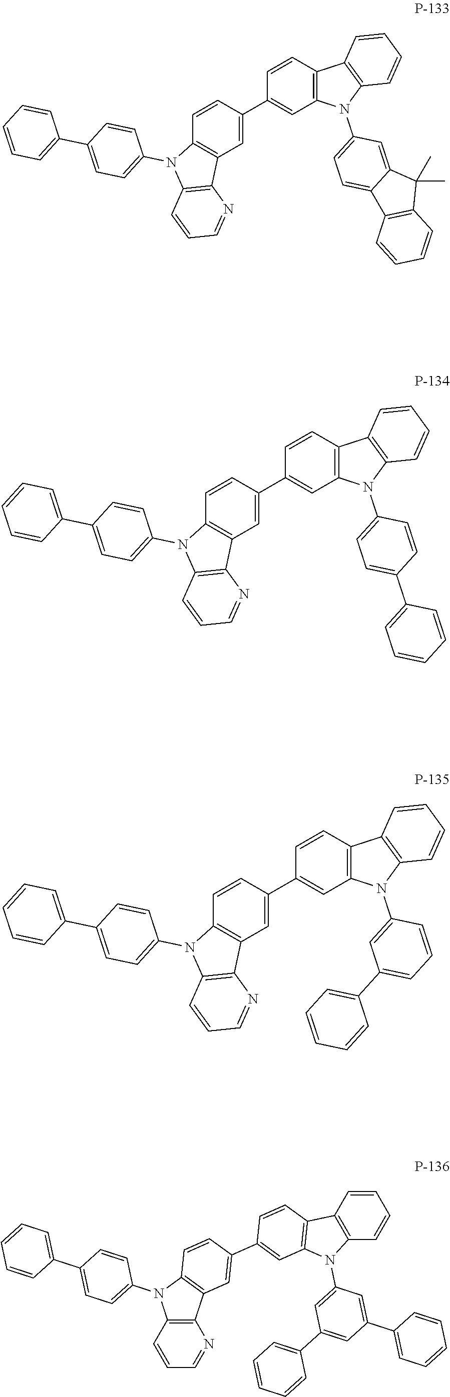 [SMILES: CC1(C)c2ccccc2-c2ccc(-n3c4ccccc4c4ccc(-c5ccc6c(c5)c5ncccc5n6-c5ccc(-c6ccccc6)cc5)cc43)cc21 ; c1ccc(-c2ccc(-n3c4ccccc4c4ccc(-c5ccc6c(c5)c5ncccc5n6-c5ccc(-c6ccccc6)cc5)cc43)cc2)cc1 ; c1ccc(-c2ccc(-n3c4ccc(-c5ccc6c7ccccc7n(-c7cccc(-c8ccccc8)c7)c6c5)cc4c4ncccc43)cc2)cc1 ; c1ccc(-c2ccc(-n3c4ccc(-c5ccc6c7ccccc7n(-c7cc(-c8ccccc8)cc(-c8ccccc8)c7)c6c5)cc4c4ncccc43)cc2)cc1]

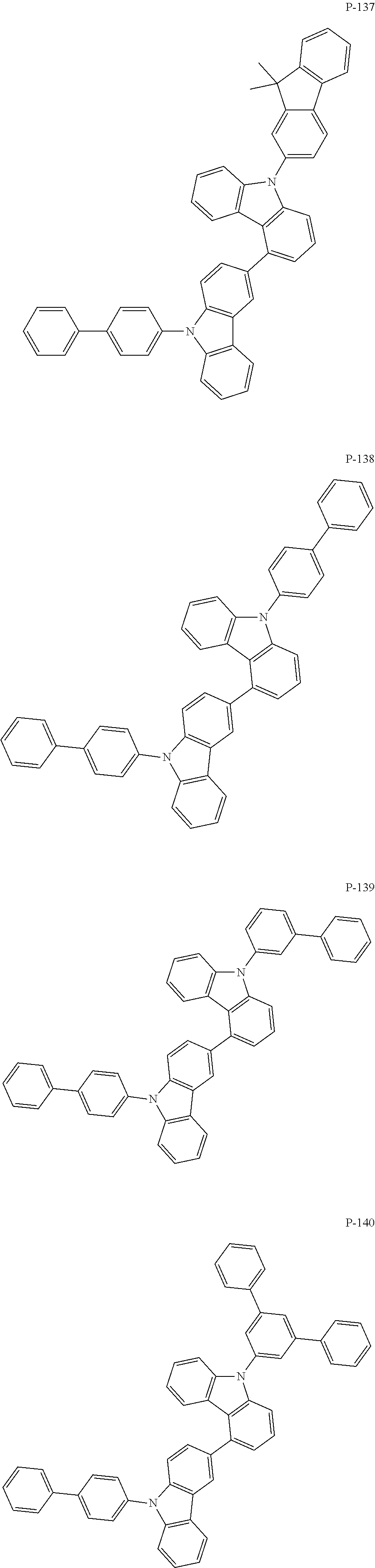 [SMILES: CC1(C)c2ccccc2-c2ccc(-n3c4ccccc4c4c(-c5ccc6c(c5)c5ccccc5n6-c5ccc(-c6ccccc6)cc5)cccc43)cc21 ; c1ccc(-c2ccc(-n3c4ccccc4c4cc(-c5cccc6c5c5ccccc5n6-c5ccc(-c6ccccc6)cc5)ccc43)cc2)cc1 ; c1ccc(-c2ccc(-n3c4ccccc4c4cc(-c5cccc6c5c5ccccc5n6-c5cccc(-c6ccccc6)c5)ccc43)cc2)cc1 ; c1ccc(-c2ccc(-n3c4ccccc4c4cc(-c5cccc6c5c5ccccc5n6-c5cc(-c6ccccc6)cc(-c6ccccc6)c5)ccc43)cc2)cc1]

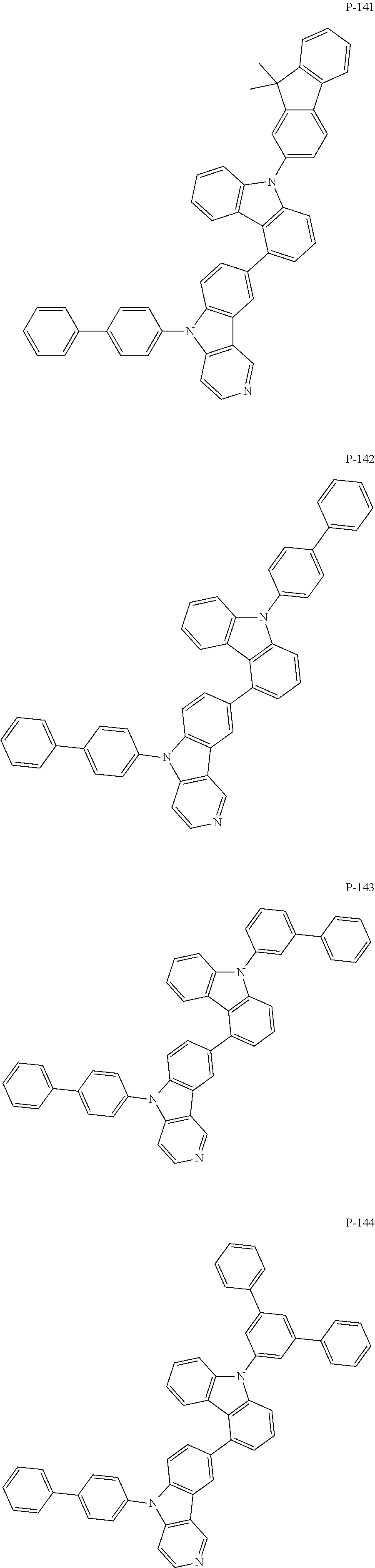 [SMILES: CC1(C)c2ccccc2-c2ccc(-n3c4ccccc4c4c(-c5ccc6c(c5)c5cnccc5n6-c5ccc(-c6ccccc6)cc5)cccc43)cc21 ; c1ccc(-c2ccc(-n3c4ccncc4c4cc(-c5cccc6c5c5ccccc5n6-c5ccc(-c6ccccc6)cc5)ccc43)cc2)cc1 ; c1ccc(-c2ccc(-n3c4ccncc4c4cc(-c5cccc6c5c5ccccc5n6-c5cccc(-c6ccccc6)c5)ccc43)cc2)cc1 ; c1ccc(-c2ccc(-n3c4ccncc4c4cc(-c5cccc6c5c5ccccc5n6-c5cc(-c6ccccc6)cc(-c6ccccc6)c5)ccc43)cc2)cc1]

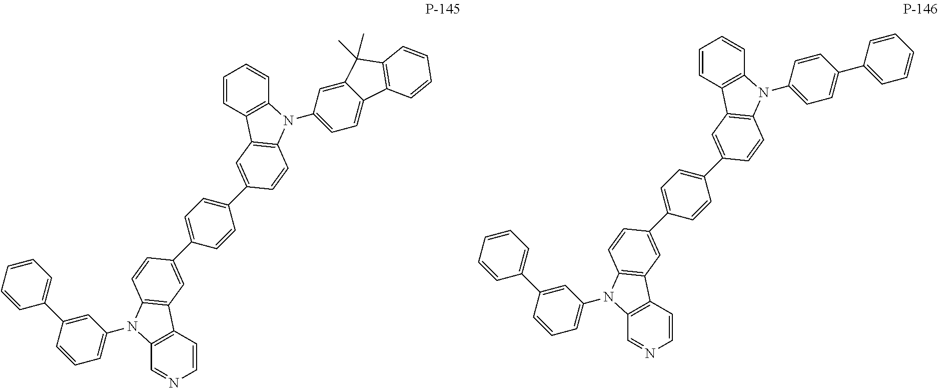 [SMILES: CC1(C)c2ccccc2-c2ccc(-n3c4ccccc4c4cc(-c5ccc(-c6ccc7c(c6)c6ccncc6n7-c6cccc(-c7ccccc7)c6)cc5)ccc43)cc21 ; c1ccc(-c2ccc(-n3c4ccccc4c4cc(-c5ccc(-c6ccc7c(c6)c6ccncc6n7-c6cccc(-c7ccccc7)c6)cc5)ccc43)cc2)cc1]

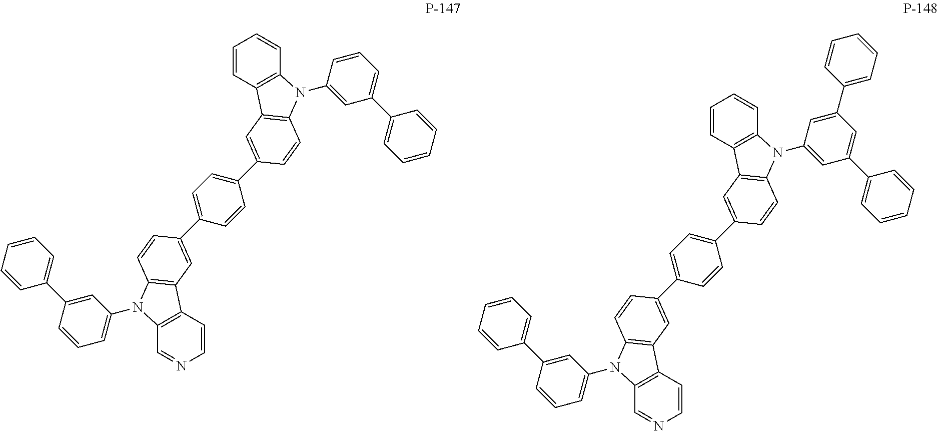 [SMILES: c1ccc(-c2cccc(-n3c4ccccc4c4cc(-c5ccc(-c6ccc7c(c6)c6ccncc6n7-c6cccc(-c7ccccc7)c6)cc5)ccc43)c2)cc1 ; c1ccc(-c2cccc(-n3c4ccc(-c5ccc(-c6ccc7c(c6)c6ccccc6n7-c6cc(-c7ccccc7)cc(-c7ccccc7)c6)cc5)cc4c4ccncc43)c2)cc1]

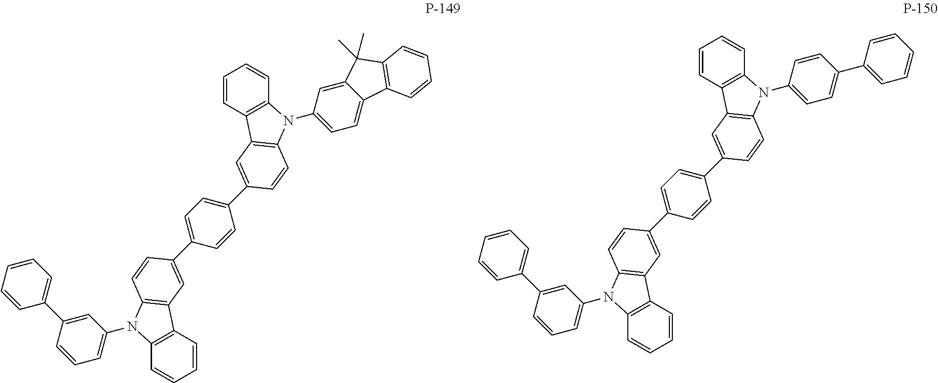 [SMILES: CC1(C)c2ccccc2-c2ccc(-n3c4ccccc4c4cc(-c5ccc(-c6ccc7c(c6)c6ccccc6n7-c6cccc(-c7ccccc7)c6)cc5)ccc43)cc21 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

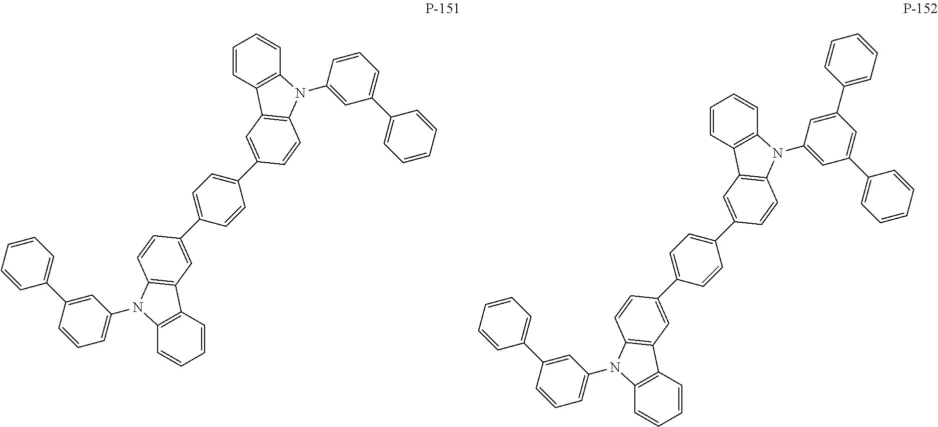 [SMILES: c1ccc(-c2cccc(-n3c4ccccc4c4cc(-c5ccc(-c6ccc7c(c6)c6ccccc6n7-c6cccc(-c7ccccc7)c6)cc5)ccc43)c2)cc1 ; c1ccc(-c2cccc(-n3c4ccccc4c4cc(-c5ccc(-c6ccc7c(c6)c6ccccc6n7-c6cc(-c7ccccc7)cc(-c7ccccc7)c6)cc5)ccc43)c2)cc1]

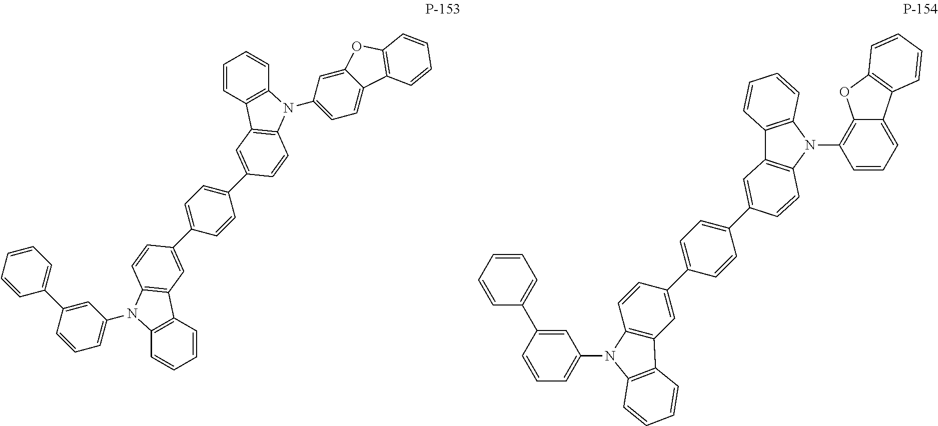 [SMILES: c1ccc(-c2cccc(-n3c4ccccc4c4cc(-c5ccc(-c6ccc7c(c6)c6ccccc6n7-c6ccc7c(c6)oc6ccccc67)cc5)ccc43)c2)cc1 ; c1ccc(-c2cccc(-n3c4ccccc4c4cc(-c5ccc(-c6ccc7c(c6)c6ccccc6n7-c6cccc7c6oc6ccccc67)cc5)ccc43)c2)cc1]

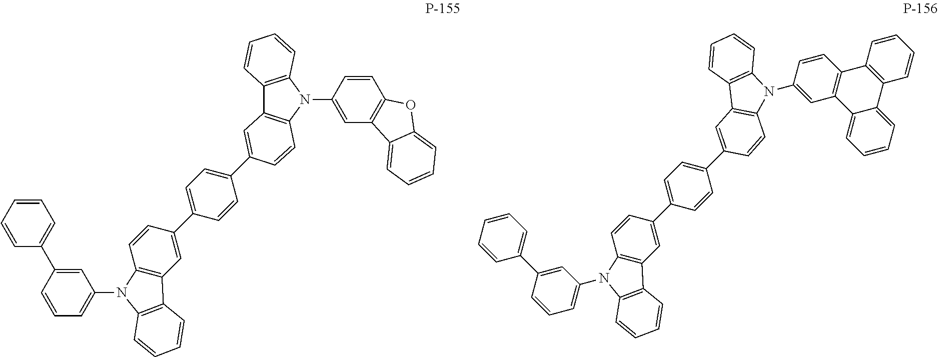 [SMILES: c1ccc(-c2cccc(-n3c4ccccc4c4cc(-c5ccc(-c6ccc7c(c6)c6ccccc6n7-c6ccc7oc8ccccc8c7c6)cc5)ccc43)c2)cc1 ; c1ccc(-c2cccc(-n3c4ccccc4c4cc(-c5ccc(-c6ccc7c(c6)c6ccccc6n7-c6ccc7c8ccccc8c8ccccc8c7c6)cc5)ccc43)c2)cc1]

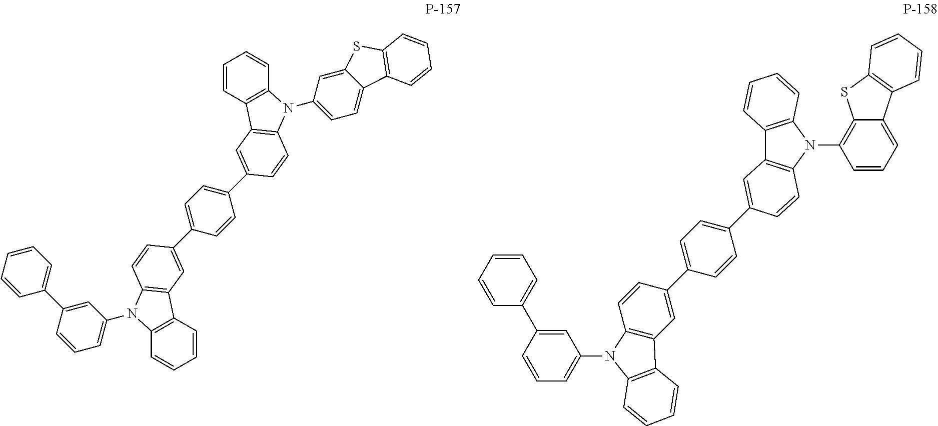 [SMILES: c1ccc(-c2cccc(-n3c4ccccc4c4cc(-c5ccc(-c6ccc7c(c6)c6ccccc6n7-c6ccc7c(c6)sc6ccccc67)cc5)ccc43)c2)cc1 ; c1ccc(-c2cccc(-n3c4ccccc4c4cc(-c5ccc(-c6ccc7c(c6)c6ccccc6n7-c6cccc7c6sc6ccccc67)cc5)ccc43)c2)cc1]

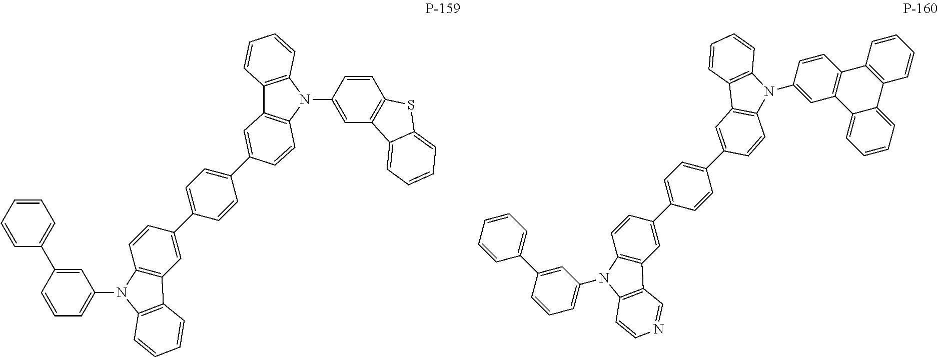 [SMILES: c1ccc(-c2cccc(-n3c4ccccc4c4cc(-c5ccc(-c6ccc7c(c6)c6ccccc6n7-c6ccc7sc8ccccc8c7c6)cc5)ccc43)c2)cc1 ; c1ccc(-c2cccc(-n3c4ccncc4c4cc(-c5ccc(-c6ccc7c(c6)c6ccccc6n7-c6ccc7c8ccccc8c8ccccc8c7c6)cc5)ccc43)c2)cc1]

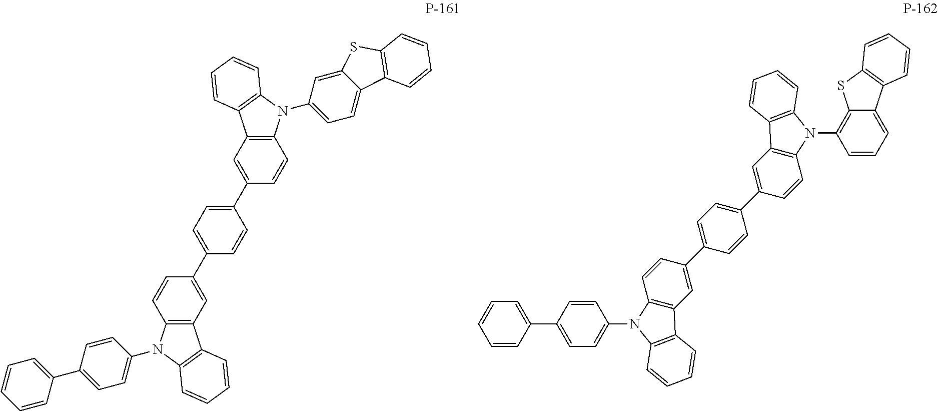 [SMILES: c1ccc(-c2ccc(-n3c4ccccc4c4cc(-c5ccc(-c6ccc7c(c6)c6ccccc6n7-c6ccc7c(c6)sc6ccccc67)cc5)ccc43)cc2)cc1 ; c1ccc(-c2ccc(-n3c4ccccc4c4cc(-c5ccc(-c6ccc7c(c6)c6ccccc6n7-c6cccc7c6sc6ccccc67)cc5)ccc43)cc2)cc1]

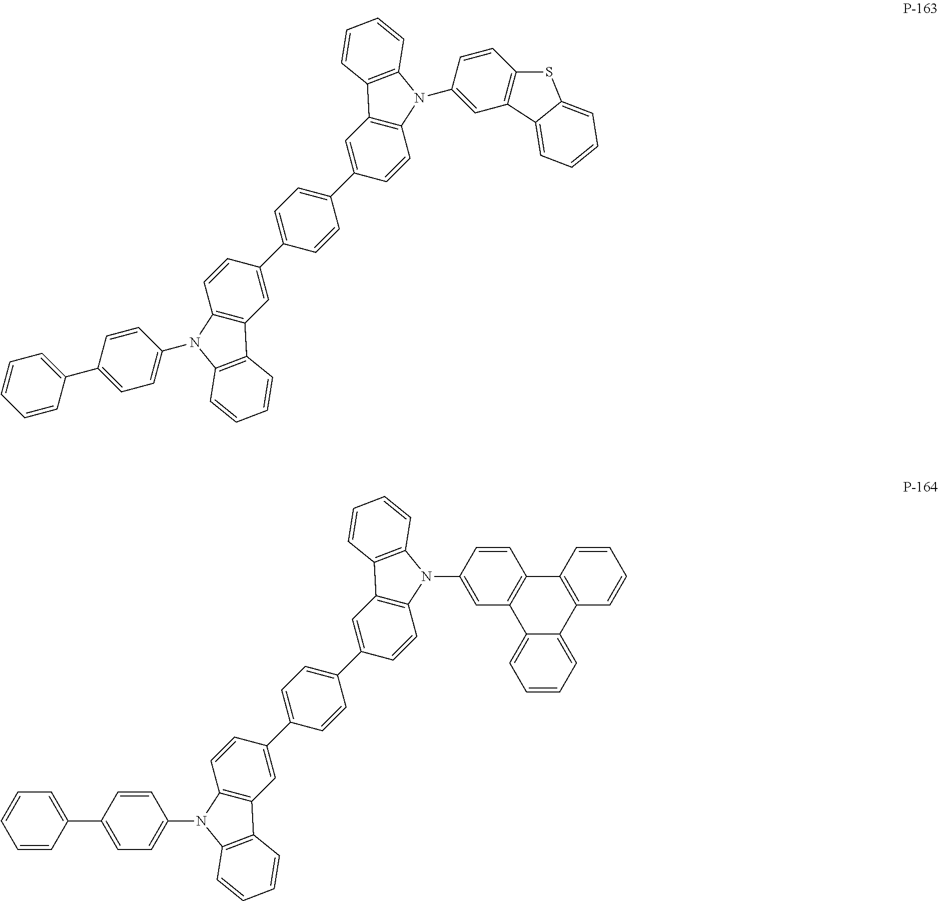 [SMILES: c1ccc(-c2ccc(-n3c4ccccc4c4cc(-c5ccc(-c6ccc7c(c6)c6ccccc6n7-c6ccc7sc8ccccc8c7c6)cc5)ccc43)cc2)cc1 ; c1ccc(-c2ccc(-n3c4ccccc4c4cc(-c5ccc(-c6ccc7c(c6)c6ccccc6n7-c6ccc7c8ccccc8c8ccccc8c7c6)cc5)ccc43)cc2)cc1]

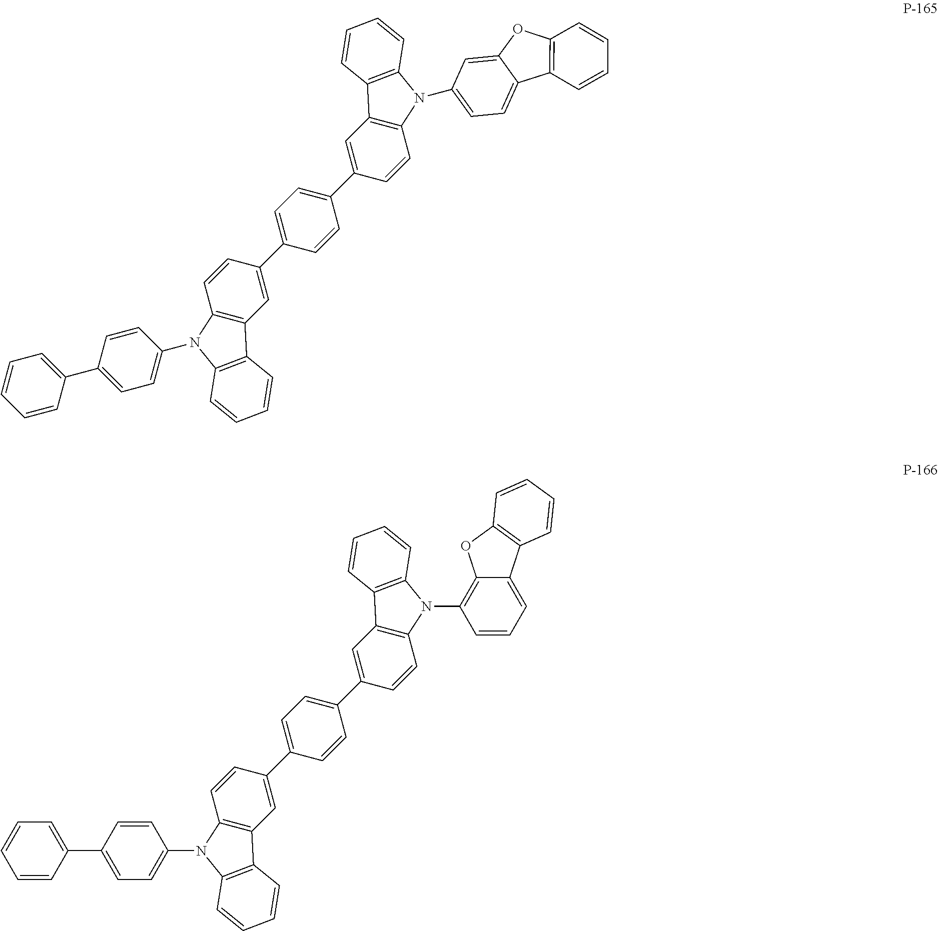 [SMILES: c1ccc(-c2ccc(-n3c4ccccc4c4cc(-c5ccc(-c6ccc7c(c6)c6ccccc6n7-c6ccc7c(c6)oc6ccccc67)cc5)ccc43)cc2)cc1 ; c1ccc(-c2ccc(-n3c4ccccc4c4cc(-c5ccc(-c6ccc7c(c6)c6ccccc6n7-c6cccc7c6oc6ccccc67)cc5)ccc43)cc2)cc1]

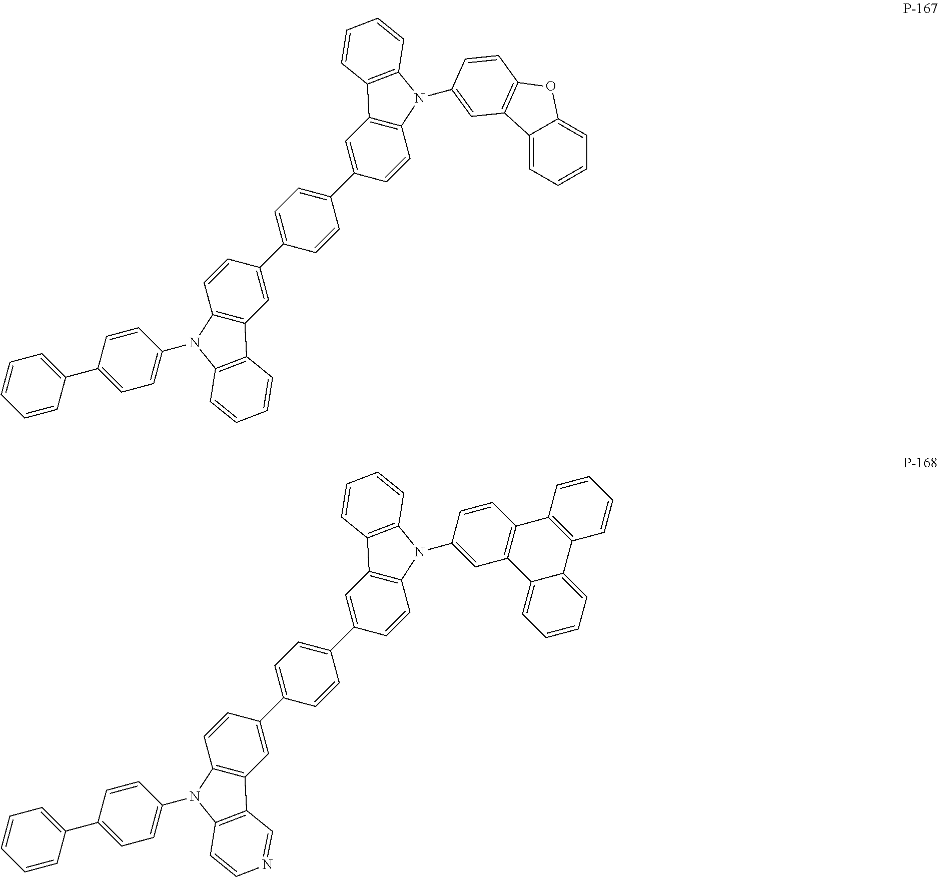 [SMILES: c1ccc(-c2ccc(-n3c4ccccc4c4cc(-c5ccc(-c6ccc7c(c6)c6ccccc6n7-c6ccc7oc8ccccc8c7c6)cc5)ccc43)cc2)cc1 ; c1ccc(-c2ccc(-n3c4ccncc4c4cc(-c5ccc(-c6ccc7c(c6)c6ccccc6n7-c6ccc7c8ccccc8c8ccccc8c7c6)cc5)ccc43)cc2)cc1]

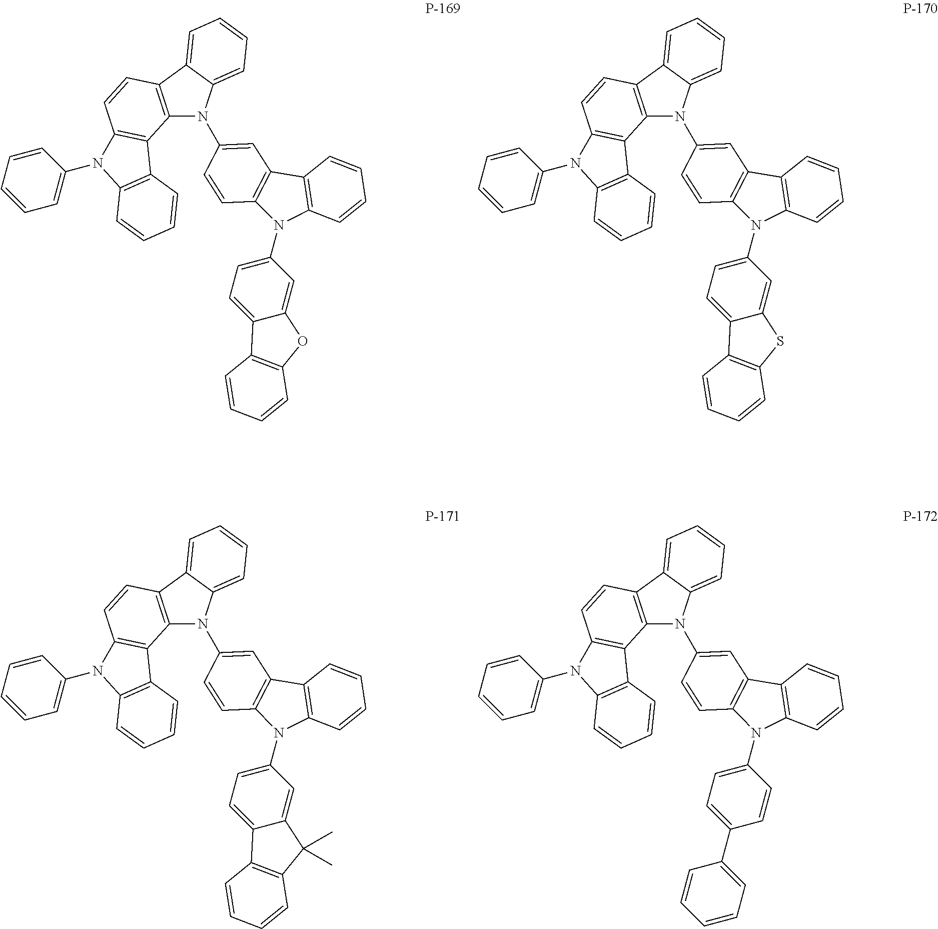 [SMILES: c1ccc(-n2c3ccccc3c3c2ccc2c4ccccc4n(-c4ccc5c(c4)c4ccccc4n5-c4ccc5c(c4)oc4ccccc45)c23)cc1 ; c1ccc(-n2c3ccccc3c3c2ccc2c4ccccc4n(-c4ccc5c(c4)c4ccccc4n5-c4ccc5c(c4)sc4ccccc45)c23)cc1 ; CC1(C)c2ccccc2-c2ccc(-n3c4ccccc4c4cc(-n5c6ccccc6c6ccc7c(c8ccccc8n7-c7ccccc7)c65)ccc43)cc21 ; c1ccc(-c2ccc(-n3c4ccccc4c4cc(-n5c6ccccc6c6ccc7c(c8ccccc8n7-c7ccccc7)c65)ccc43)cc2)cc1]

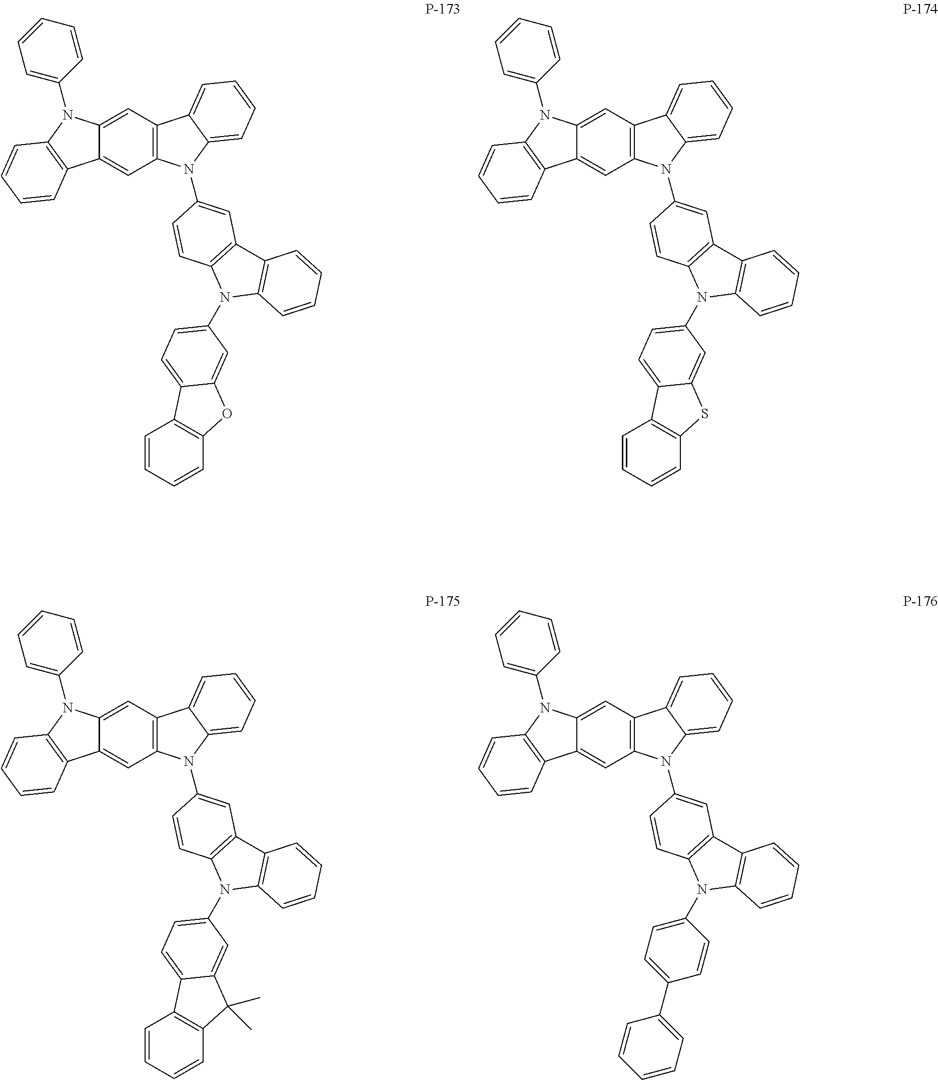 [SMILES: c1ccc(-n2c3ccccc3c3cc4c(cc32)c2ccccc2n4-c2ccc3c(c2)c2ccccc2n3-c2ccc3c(c2)oc2ccccc23)cc1 ; c1ccc(-n2c3ccccc3c3cc4c(cc32)c2ccccc2n4-c2ccc3c(c2)c2ccccc2n3-c2ccc3c(c2)sc2ccccc23)cc1 ; CC1(C)c2ccccc2-c2ccc(-n3c4ccccc4c4cc(-n5c6ccccc6c6cc7c(cc65)c5ccccc5n7-c5ccccc5)ccc43)cc21 ; c1ccc(-c2ccc(-n3c4ccccc4c4cc(-n5c6ccccc6c6cc7c(cc65)c5ccccc5n7-c5ccccc5)ccc43)cc2)cc1]

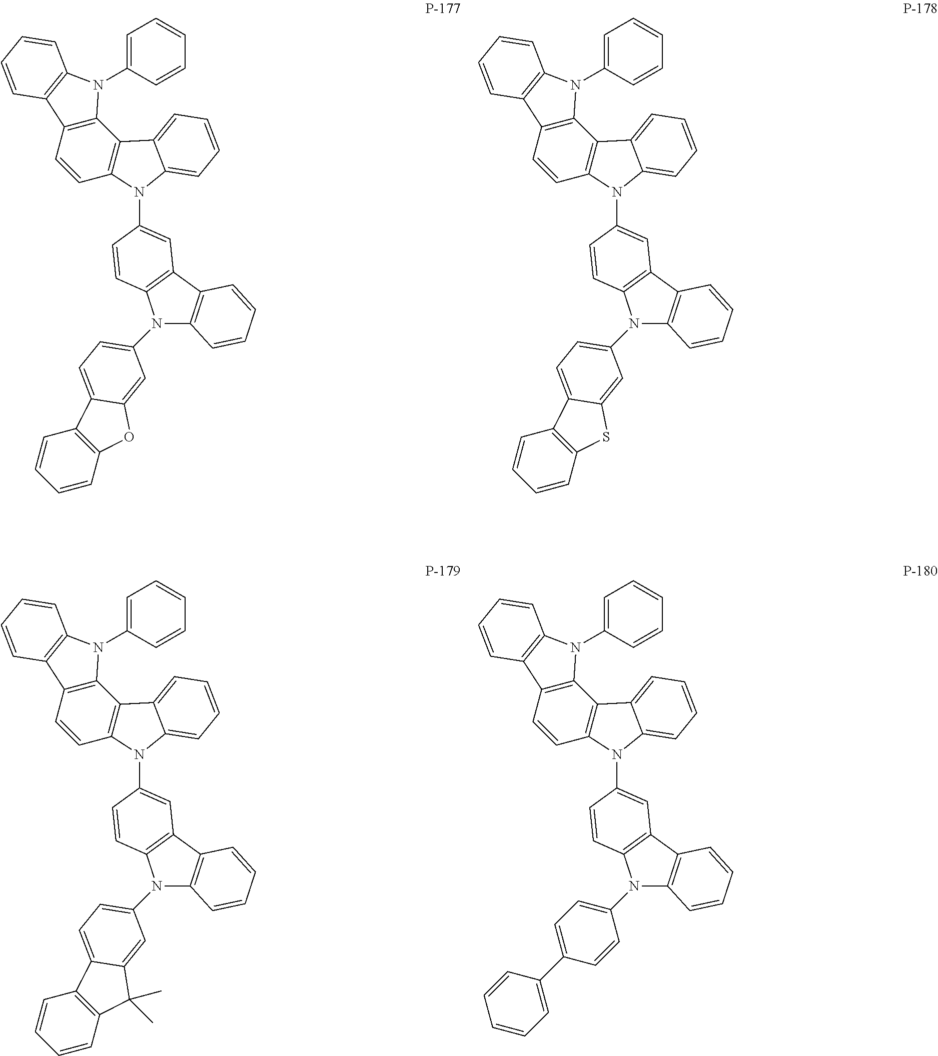 [SMILES: c1ccc(-n2c3ccccc3c3ccc4c(c5ccccc5n4-c4ccc5c(c4)c4ccccc4n5-c4ccc5c(c4)oc4ccccc45)c32)cc1 ; c1ccc(-n2c3ccccc3c3ccc4c(c5ccccc5n4-c4ccc5c(c4)c4ccccc4n5-c4ccc5c(c4)sc4ccccc45)c32)cc1 ; CC1(C)c2ccccc2-c2ccc(-n3c4ccccc4c4cc(-n5c6ccccc6c6c5ccc5c7ccccc7n(-c7ccccc7)c56)ccc43)cc21 ; c1ccc(-c2ccc(-n3c4ccccc4c4cc(-n5c6ccccc6c6c5ccc5c7ccccc7n(-c7ccccc7)c56)ccc43)cc2)cc1]

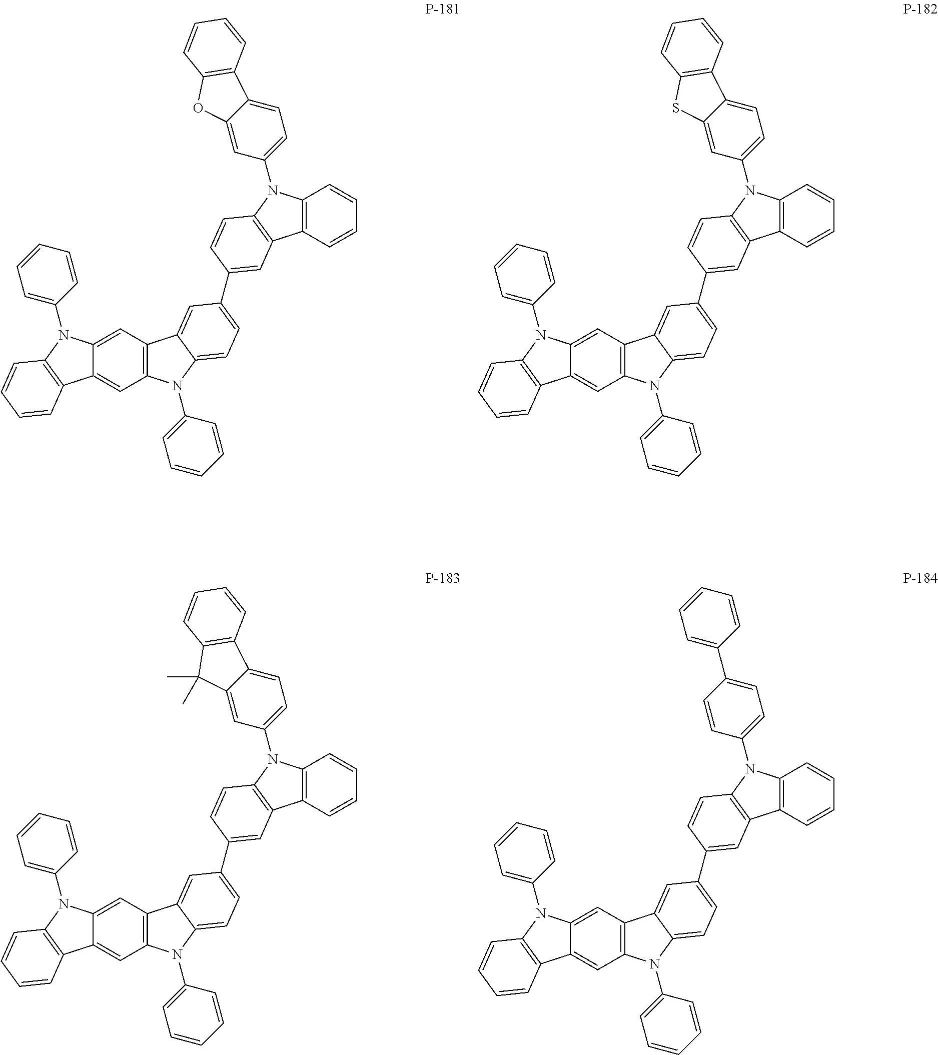 [SMILES: c1ccc(-n2c3ccccc3c3cc4c(cc32)c2cc(-c3ccc5c(c3)c3ccccc3n5-c3ccc5c(c3)oc3ccccc35)ccc2n4-c2ccccc2)cc1 ; c1ccc(-n2c3ccccc3c3cc4c(cc32)c2cc(-c3ccc5c(c3)c3ccccc3n5-c3ccc5c(c3)sc3ccccc35)ccc2n4-c2ccccc2)cc1 ; CC1(C)c2ccccc2-c2ccc(-n3c4ccccc4c4cc(-c5ccc6c(c5)c5cc7c(cc5n6-c5ccccc5)c5ccccc5n7-c5ccccc5)ccc43)cc21 ; c1ccc(-c2ccc(-n3c4ccccc4c4cc(-c5ccc6c(c5)c5cc7c(cc5n6-c5ccccc5)c5ccccc5n7-c5ccccc5)ccc43)cc2)cc1]

In another aspect, the present disclosure also provides an organic compound comprising a structure of formula (VI):

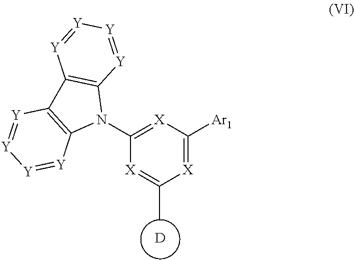

Where each X is independently N or CR₁, and at least one X is N atom; Y at each occurrence is independently CR₂ or N; where no more than two Ys in each ring are N atoms; Ar₁ is independently selected from a substituted/unsubstituted aromatic or heteroaromatic group containing 5 to 40 ring atoms, an aryloxy or heteroaryloxy group containing 5 to 40 ring atoms, or any combination thereof, where one or more Aris may form a monocyclic or polycyclic aliphatic or aromatic ring system with each other and/or with the rings bonded thereto;

D ring is selected from the group consisting of:

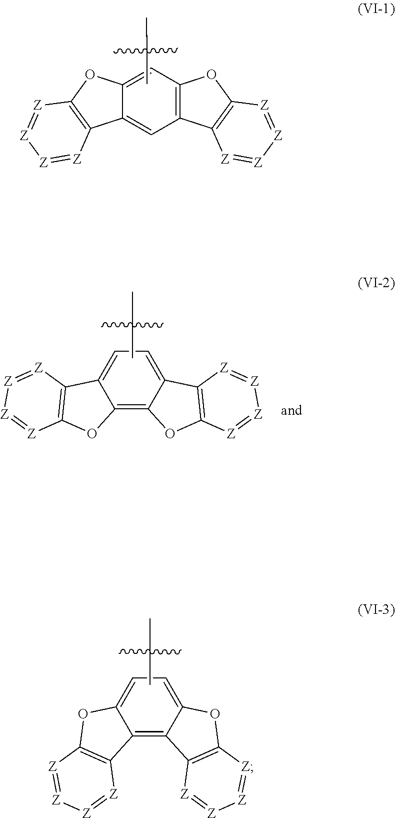

Where each Z is independently N or CR₃; each of R₁ to R₃ is independently selected from —H, -D, a substituted/unsubstituted C₁-C₁₀ alkyl group, a substituted/unsubstituted aromatic or heteroaromatic group containing 5 to 60 ring atoms, or any combination thereof.

In some embodiments, in the organic compound as described herein, each Ar₁ is independently selected from the following groups or any combination thereof:

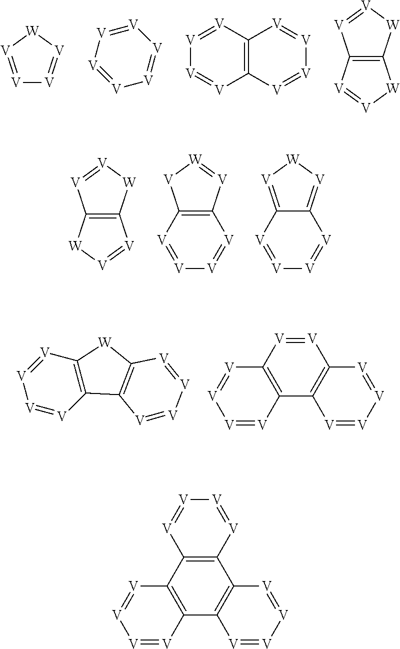

V, in multiple occurrences, is independently selected from CR₄ or N;
W, in multiple occurrences, is independently selected from CR₅R₆, SiR₇R₈, NR₉, C(═O), S, or O;
each of R₄ to R₉ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, where one or more R₄-R₉ may form a monocyclic or polycyclic aliphatic or aromatic ring system with each other and/or with the rings bonded thereto.

In some embodiments, each Ar₁ is independently selected from the following structural formulas and may be substituted with 0, 1, 2, or 3 substituents, and the substituents are each independently selected from -D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group:

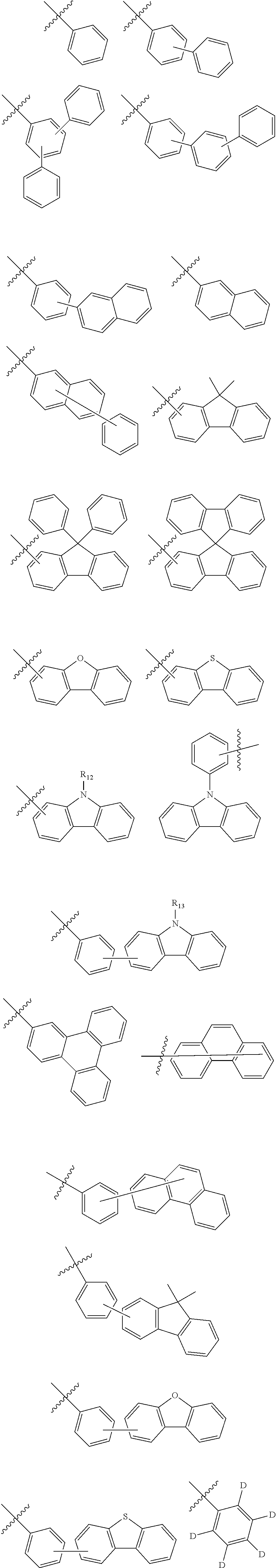

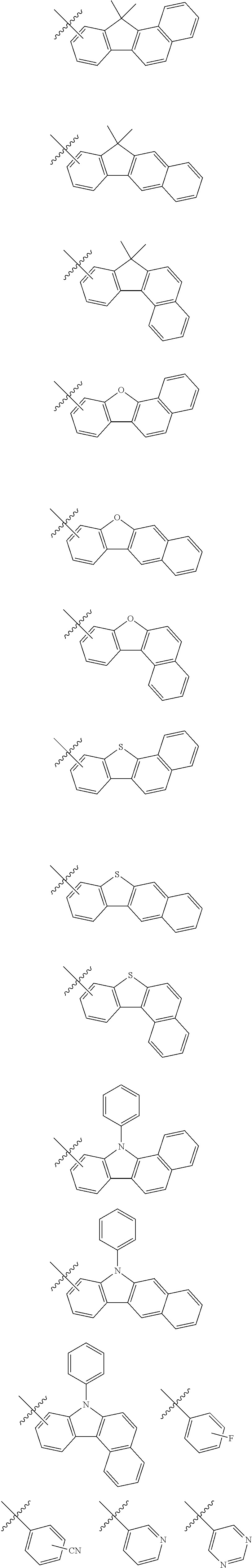

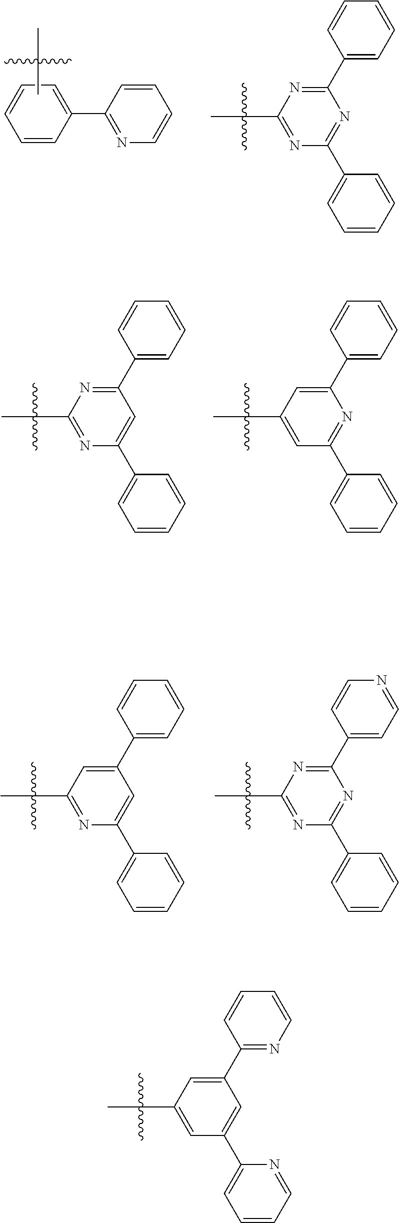

Where R₁₂ and R₁₃ are defined identically as the above-mentioned R₄.

In some embodiments, no more than three Zs in ring D are N atoms, preferably two Zs are N atoms, more preferably one Z is N atom, and most preferably each Z is CH.

The preferred examples of the organic compounds as described herein are shown in the following structures. These structures may be substituted at all possible substitution sites.

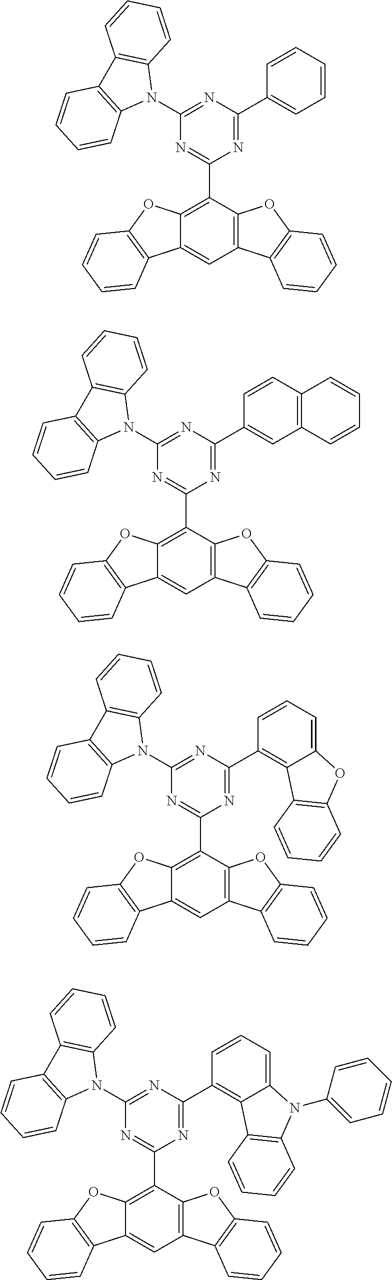

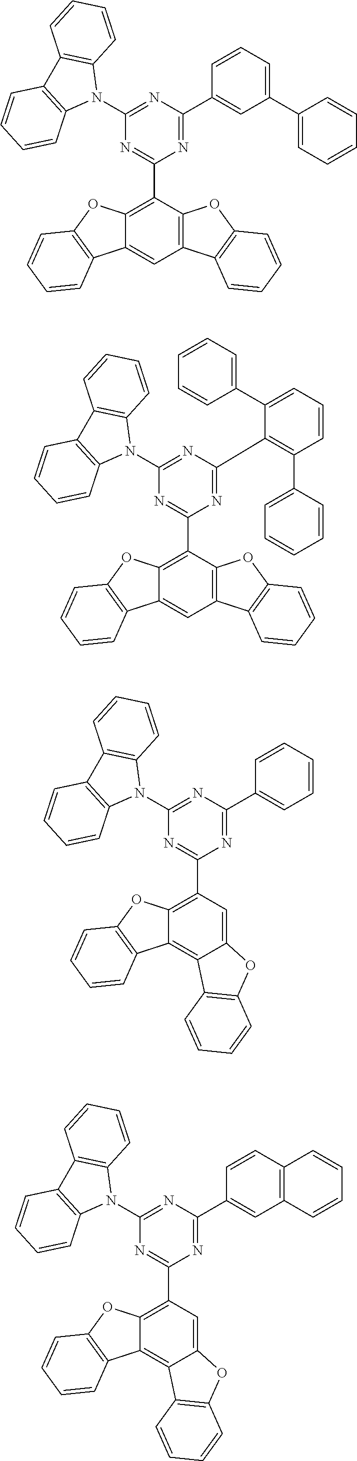 [SMILES: c1ccc(-c2cccc(-c3nc(-c4c5oc6ccccc6c5cc5c4oc4ccccc45)nc(-n4c5ccccc5c5ccccc54)n3)c2)cc1 ; c1ccc(-c2cccc(-c3ccccc3)c2-c2nc(-c3c4oc5ccccc5c4cc4c3oc3ccccc34)nc(-n3c4ccccc4c4ccccc43)n2)cc1 ; c1ccc(-c2nc(-c3cc4oc5ccccc5c4c4c3oc3ccccc34)nc(-n3c4ccccc4c4ccccc43)n2)cc1 ; c1ccc2cc(-c3nc(-c4cc5oc6ccccc6c5c5c4oc4ccccc45)nc(-n4c5ccccc5c5ccccc54)n3)ccc2c1]

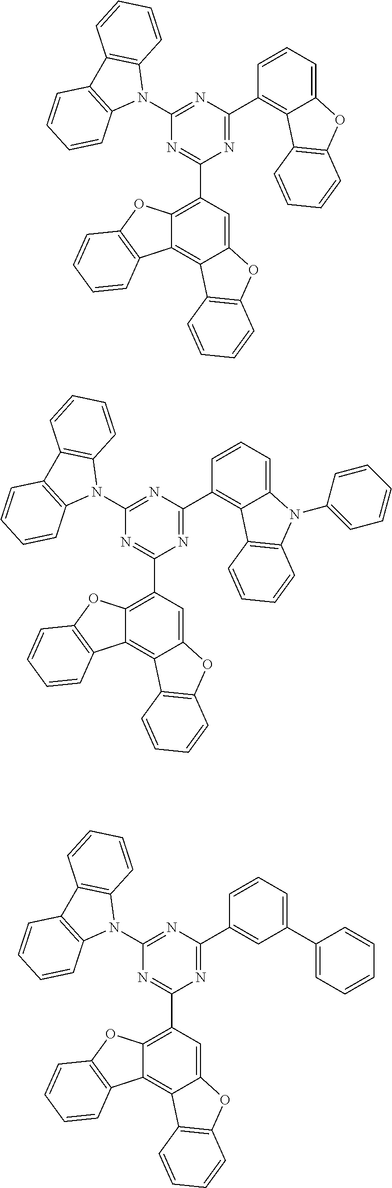 [SMILES: c1ccc2c(c1)oc1cccc(-c3nc(-c4cc5oc6ccccc6c5c5c4oc4ccccc45)nc(-n4c5ccccc5c5ccccc54)n3)c12 ; c1ccc(-n2c3ccccc3c3c(-c4nc(-c5cc6oc7ccccc7c6c6c5oc5ccccc56)nc(-n5c6ccccc6c6ccccc65)n4)cccc32)cc1 ; c1ccc(-c2cccc(-c3nc(-c4cc5oc6ccccc6c5c5c4oc4ccccc45)nc(-n4c5ccccc5c5ccccc54)n3)c2)cc1]

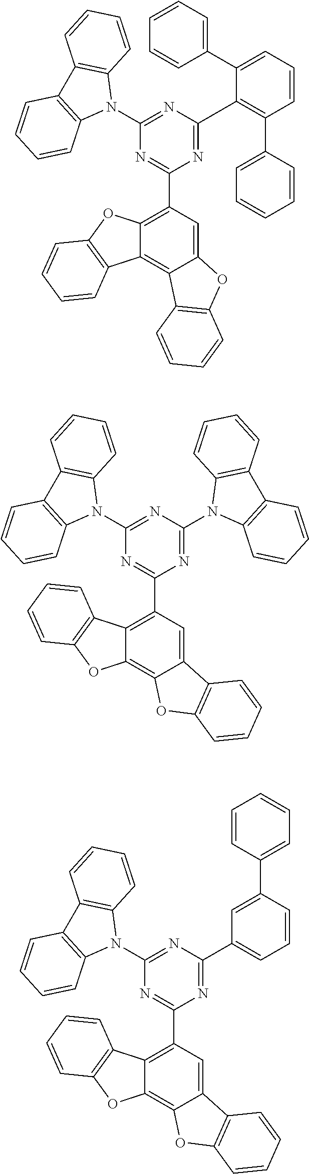 [SMILES: c1ccc(-c2cccc(-c3ccccc3)c2-c2nc(-c3cc4oc5ccccc5c4c4c3oc3ccccc34)nc(-n3c4ccccc4c4ccccc43)n2)cc1 ; c1ccc2c(c1)oc1c2cc(-c2nc(-n3c4ccccc4c4ccccc43)nc(-n3c4ccccc4c4ccccc43)n2)c2c3ccccc3oc12 ; c1ccc(-c2cccc(-c3nc(-c4cc5c6ccccc6oc5c5oc6ccccc6c45)nc(-n4c5ccccc5c5ccccc54)n3)c2)cc1]

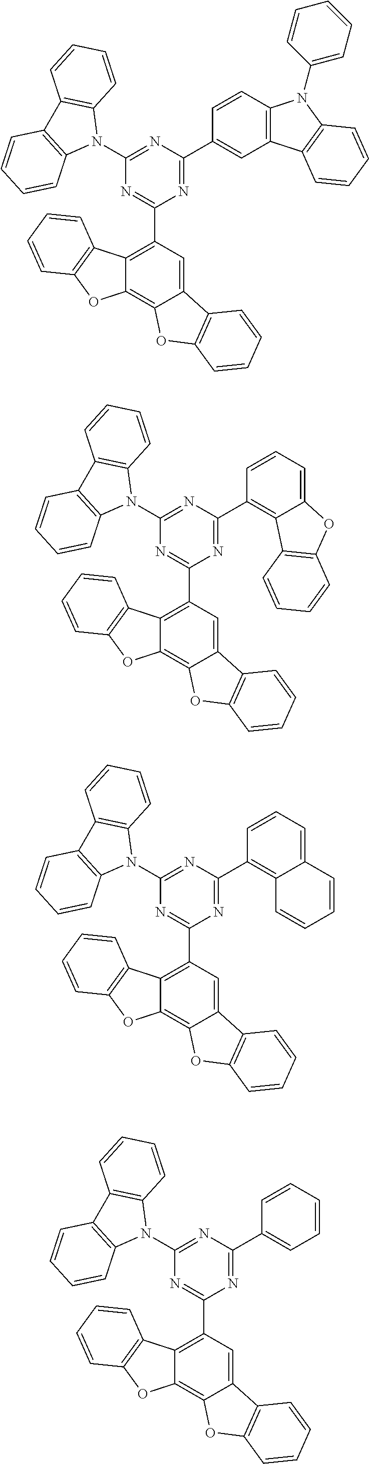 [SMILES: c1ccc(-n2c3ccccc3c3cc(-c4nc(-c5cc6c7ccccc7oc6c6oc7ccccc7c56)nc(-n5c6ccccc6c6ccccc65)n4)ccc32)cc1 ; c1ccc2c(c1)oc1c2cc(-c2nc(-c3cccc4oc5ccccc5c34)nc(-n3c4ccccc4c4ccccc43)n2)c2c3ccccc3oc12 ; c1ccc2c(-c3nc(-c4cc5c6ccccc6oc5c5oc6ccccc6c45)nc(-n4c5ccccc5c5ccccc54)n3)cccc2c1 ; c1ccc(-c2nc(-c3cc4c5ccccc5oc4c4oc5ccccc5c34)nc(-n3c4ccccc4c4ccccc43)n2)cc1]

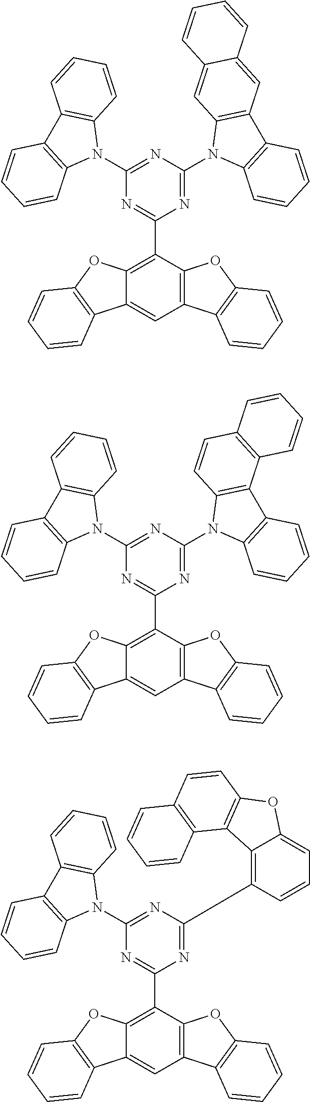 [SMILES: c1ccc2cc3c(cc2c1)c1ccccc1n3-c1nc(-c2c3oc4ccccc4c3cc3c2oc2ccccc23)nc(-n2c3ccccc3c3ccccc32)n1 ; c1ccc2c(c1)ccc1c2c2ccccc2n1-c1nc(-c2c3oc4ccccc4c3cc3c2oc2ccccc23)nc(-n2c3ccccc3c3ccccc32)n1 ; c1ccc2c(c1)ccc1oc3cccc(-c4nc(-c5c6oc7ccccc7c6cc6c5oc5ccccc56)nc(-n5c6ccccc6c6ccccc65)n4)c3c12]

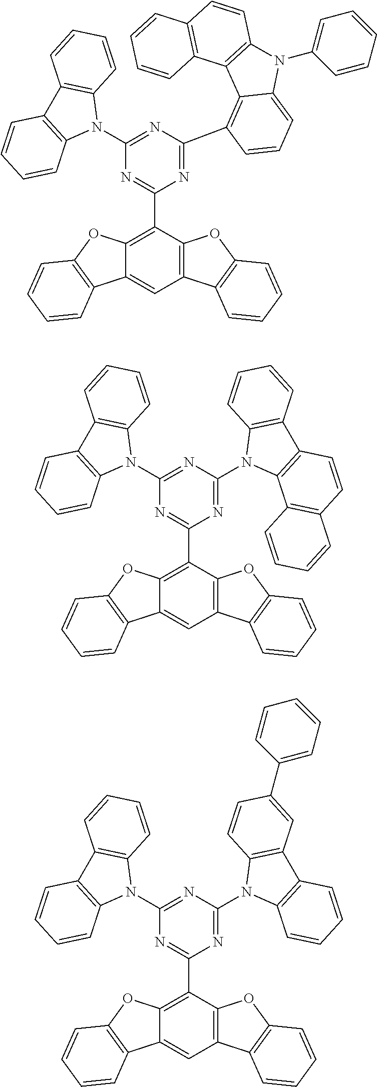

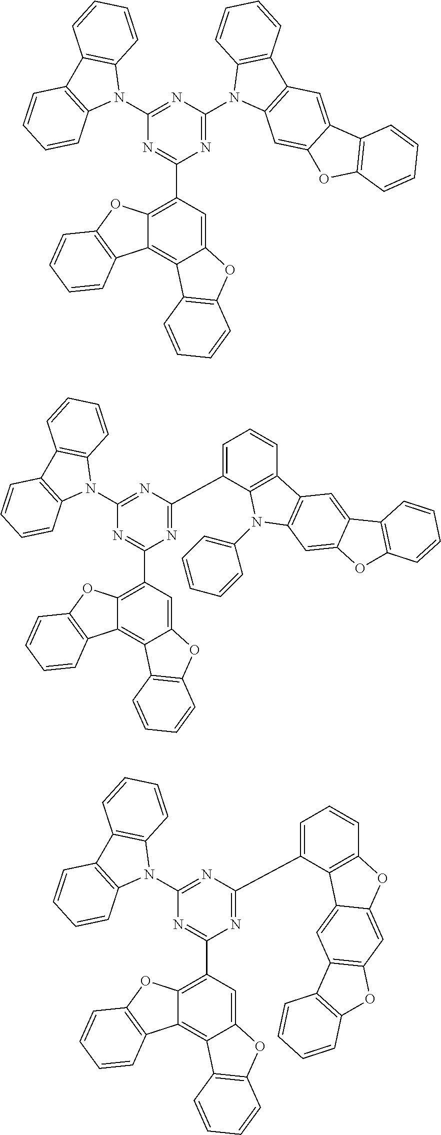 [SMILES: c1ccc2c(c1)oc1cc3c(cc12)c1ccccc1n3-c1nc(-c2cc3oc4ccccc4c3c3c2oc2ccccc23)nc(-n2c3ccccc3c3ccccc32)n1 ; c1ccc(-n2c3cc4oc5ccccc5c4cc3c3cccc(-c4nc(-c5cc6oc7ccccc7c6c6c5oc5ccccc56)nc(-n5c6ccccc6c6ccccc65)n4)c32)cc1 ; c1ccc2c(c1)oc1cc3oc4cccc(-c5nc(-c6cc7oc8ccccc8c7c7c6oc6ccccc67)nc(-n6c7ccccc7c7ccccc76)n5)c4c3cc12]

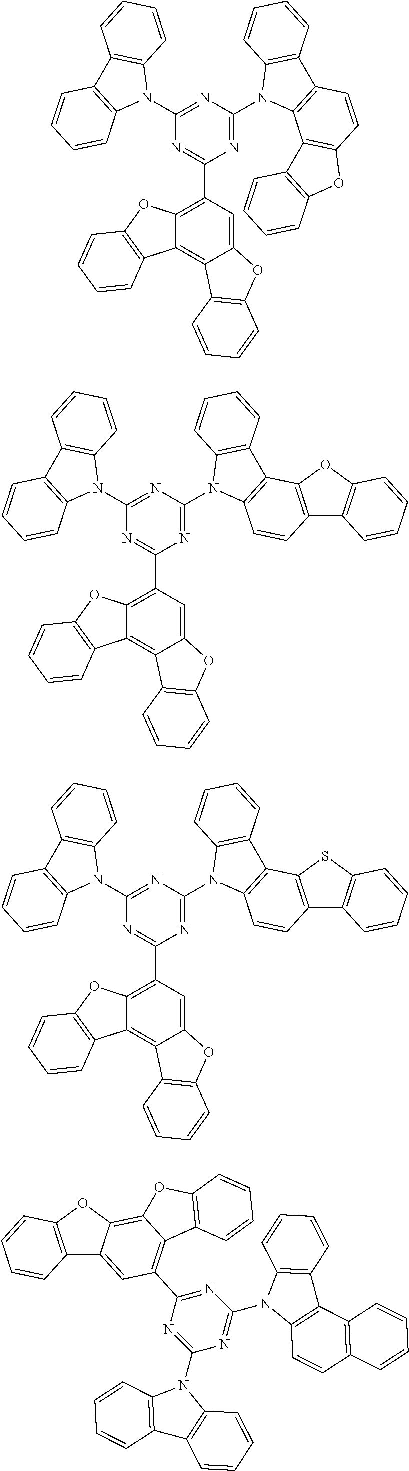

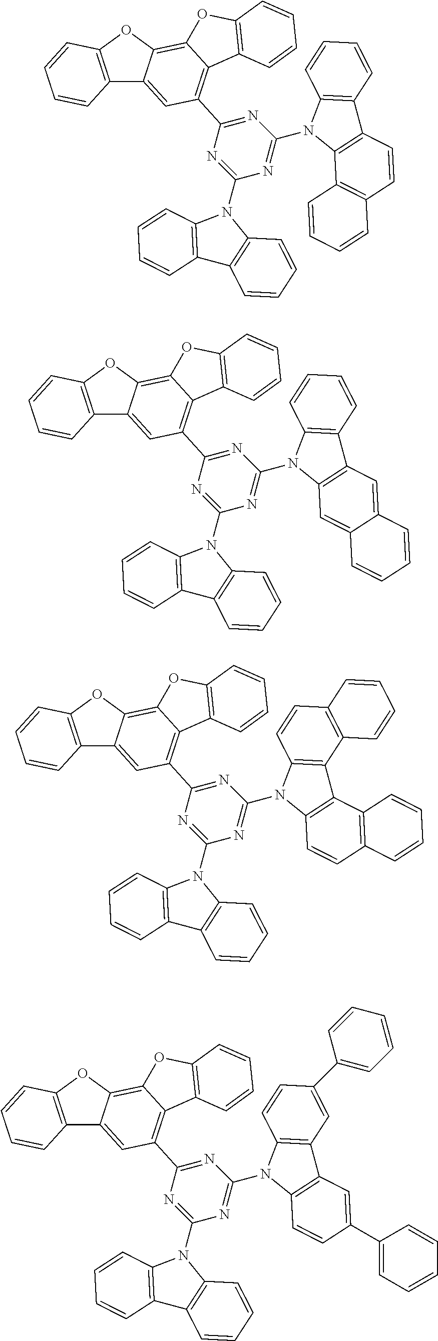

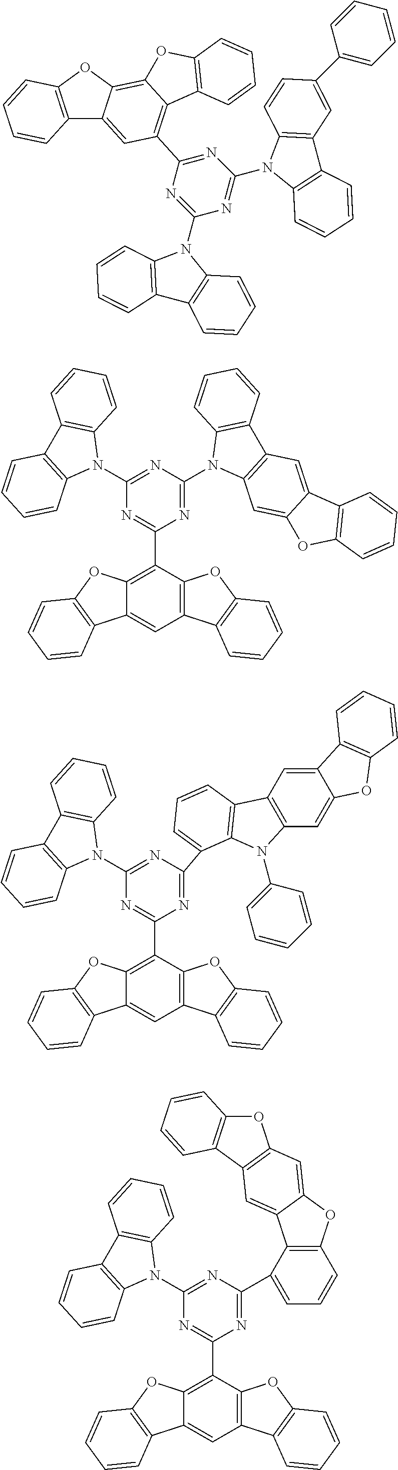

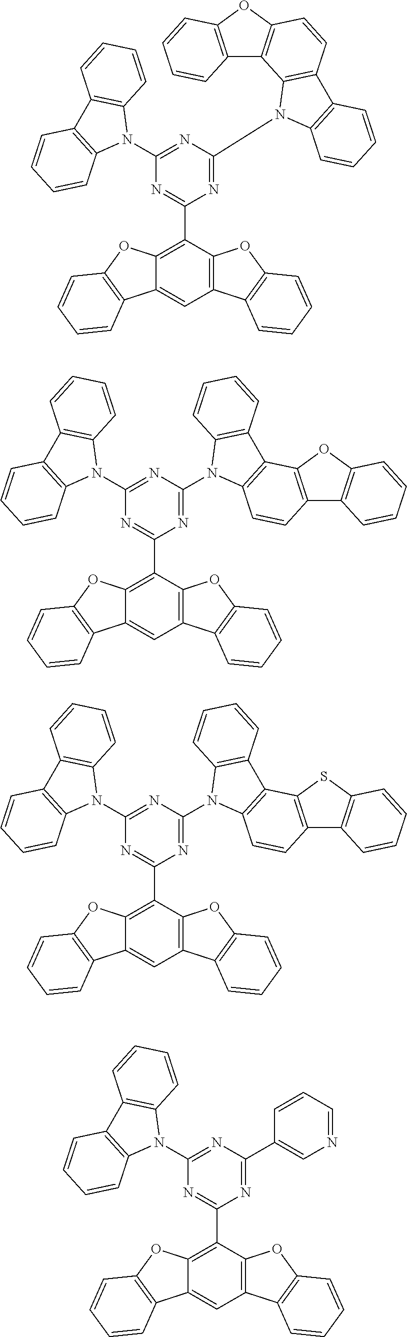

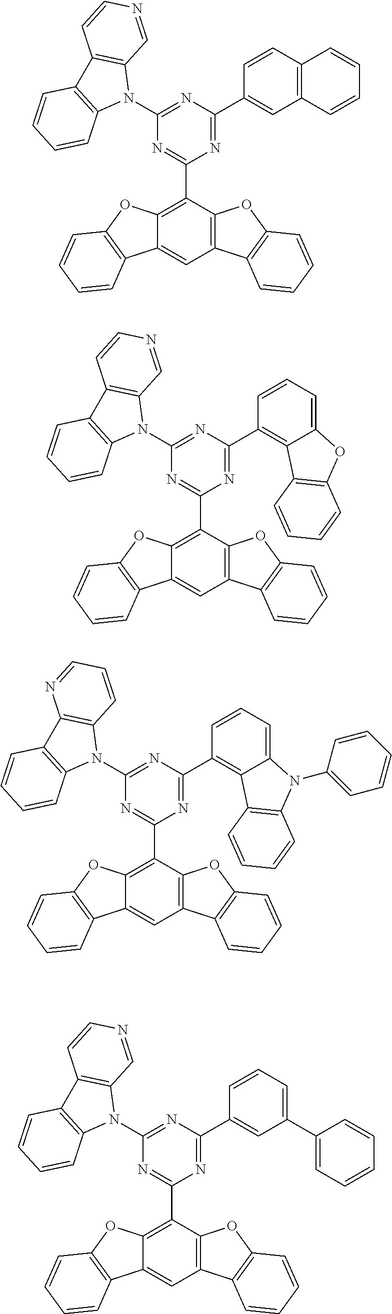

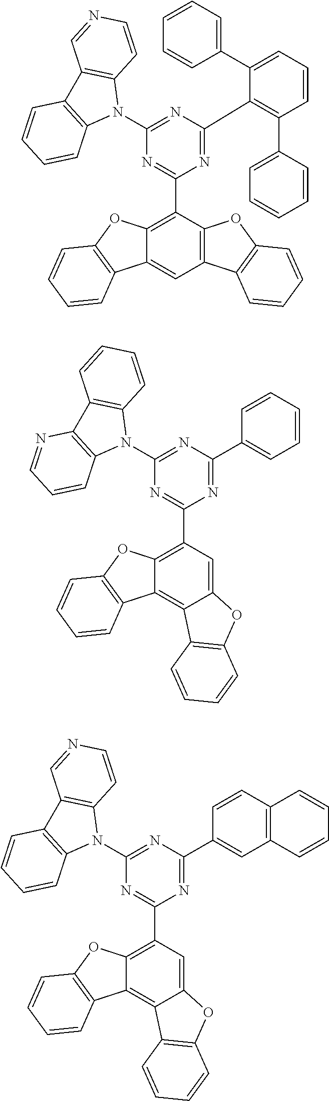

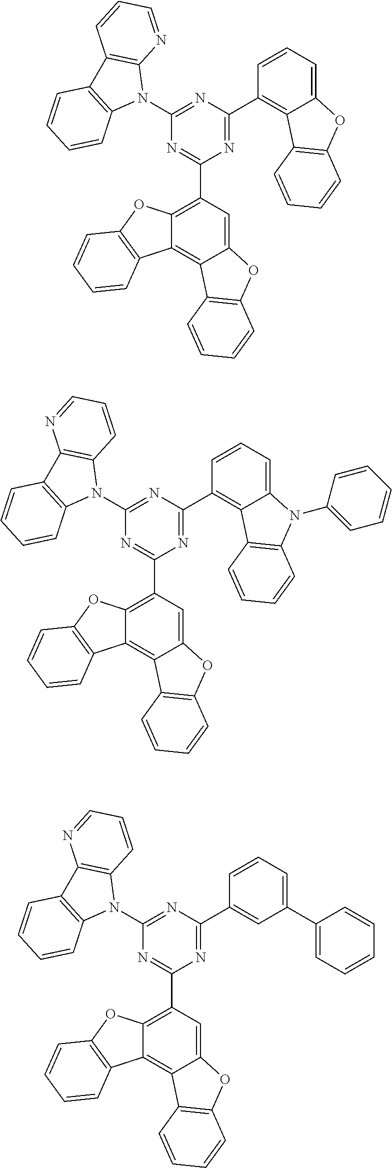 [SMILES: c1ccc2c(c1)oc1cccc(-c3nc(-c4cc5oc6ccccc6c5c5c4oc4ccccc45)nc(-n4c5ccccc5c5cccnc54)n3)c12 ; c1ccc(-n2c3ccccc3c3c(-c4nc(-c5cc6oc7ccccc7c6c6c5oc5ccccc56)nc(-n5c6ccccc6c6ncccc65)n4)cccc32)cc1 ; c1ccc(-c2cccc(-c3nc(-c4cc5oc6ccccc6c5c5c4oc4ccccc45)nc(-n4c5ccccc5c5ncccc54)n3)c2)cc1]

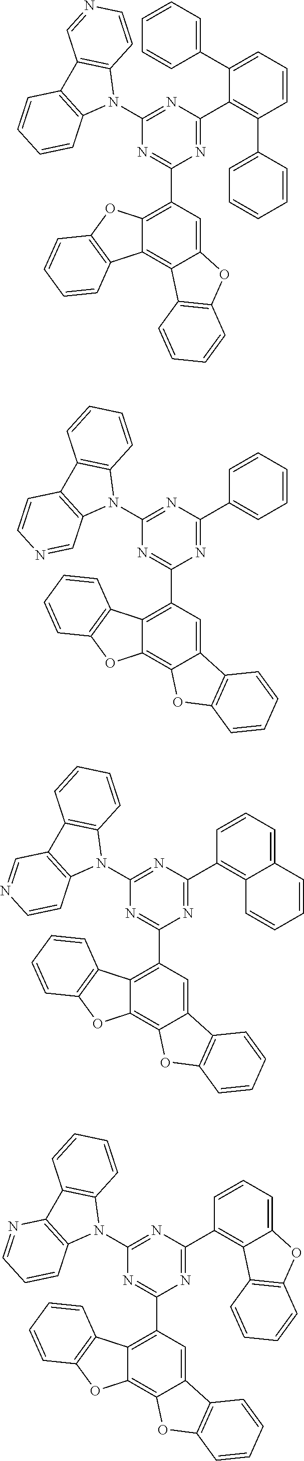 [SMILES: c1ccc(-c2cccc(-c3ccccc3)c2-c2nc(-c3cc4oc5ccccc5c4c4c3oc3ccccc34)nc(-n3c4ccccc4c4cnccc43)n2)cc1 ; c1ccc(-c2nc(-c3cc4c5ccccc5oc4c4oc5ccccc5c34)nc(-n3c4ccccc4c4ccncc43)n2)cc1 ; c1ccc2c(-c3nc(-c4cc5c6ccccc6oc5c5oc6ccccc6c45)nc(-n4c5ccccc5c5cnccc54)n3)cccc2c1 ; c1ccc2c(c1)oc1c2cc(-c2nc(-c3cccc4oc5ccccc5c34)nc(-n3c4ccccc4c4ncccc43)n2)c2c3ccccc3oc12]

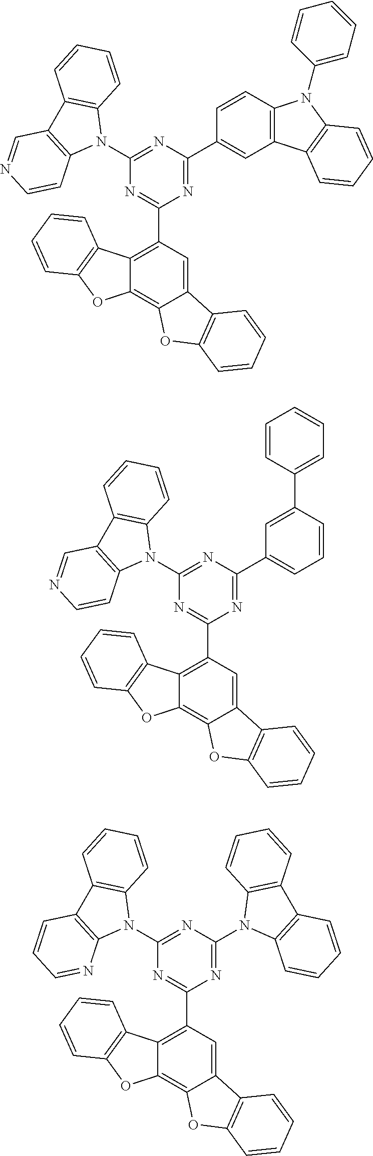

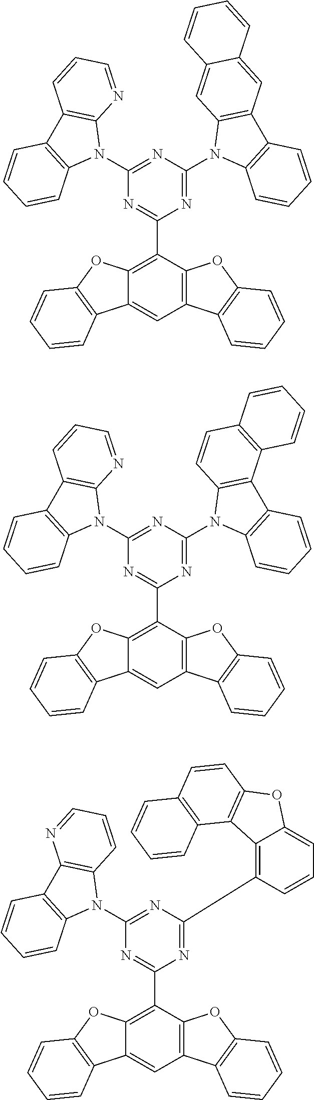

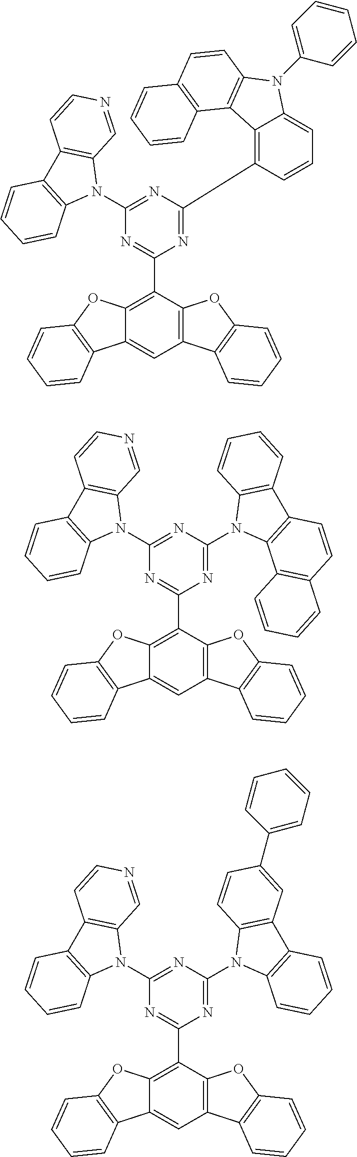

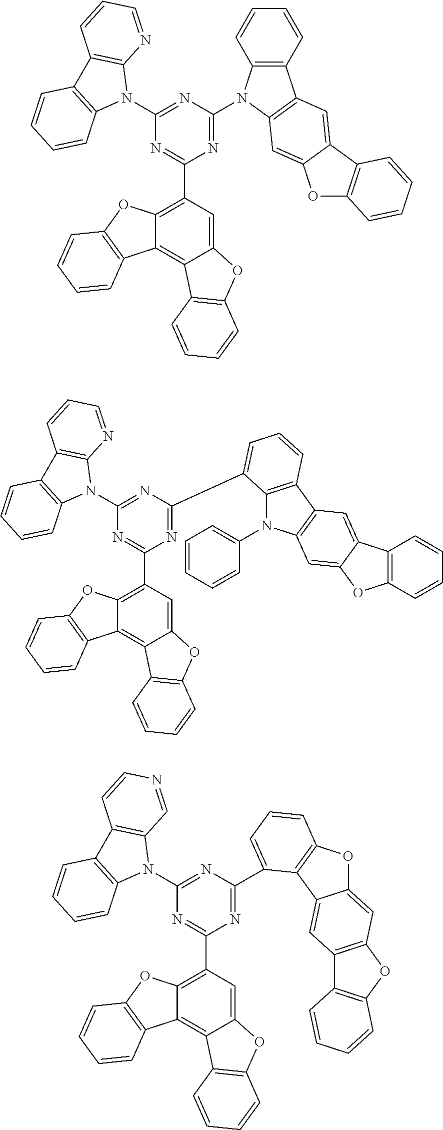

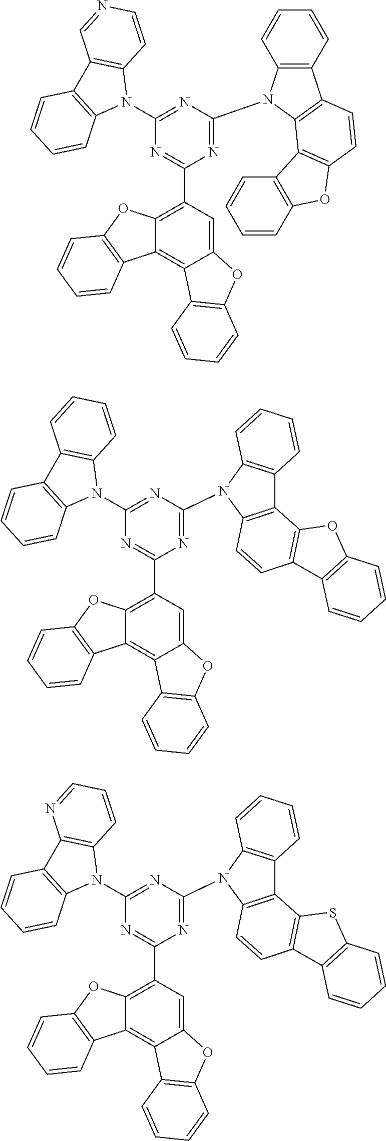 [SMILES: c1ccc2c(c1)oc1cc(-c3nc(-n4c5ccccc5c5cnccc54)nc(-n4c5ccccc5c5ccc6oc7ccccc7c6c54)n3)c3oc4ccccc4c3c12 ; c1ccc2c(c1)oc1c2ccc2c1c1ccccc1n2-c1nc(-c2cc3oc4ccccc4c3c3c2oc2ccccc23)nc(-n2c3ccccc3c3ccccc32)n1 ; c1ccc2c(c1)oc1cc(-c3nc(-n4c5ccccc5c5ncccc54)nc(-n4c5ccccc5c5c6sc7ccccc7c6ccc54)n3)c3oc4ccccc4c3c12]

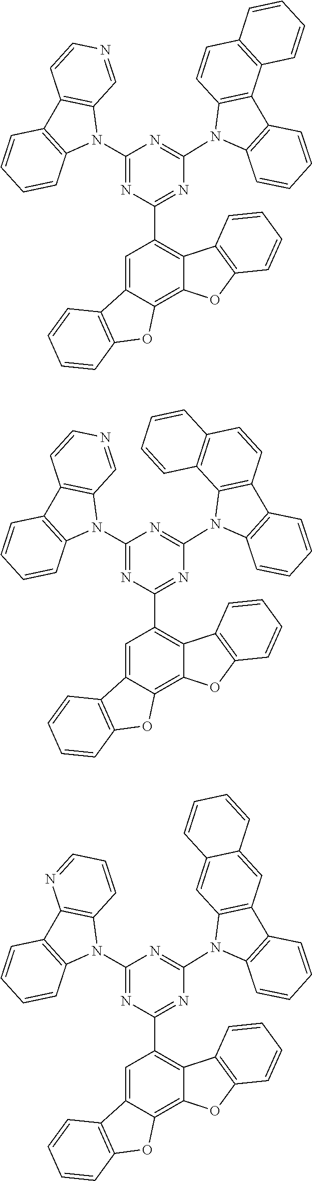 [SMILES: c1ccc2c(c1)ccc1c2c2ccccc2n1-c1nc(-c2cc3c4ccccc4oc3c3oc4ccccc4c23)nc(-n2c3ccccc3c3ccncc32)n1 ; c1ccc2c(c1)ccc1c3ccccc3n(-c3nc(-c4cc5c6ccccc6oc5c5oc6ccccc6c45)nc(-n4c5ccccc5c5ccncc54)n3)c21 ; c1ccc2cc3c(cc2c1)c1ccccc1n3-c1nc(-c2cc3c4ccccc4oc3c3oc4ccccc4c23)nc(-n2c3ccccc3c3ncccc32)n1]

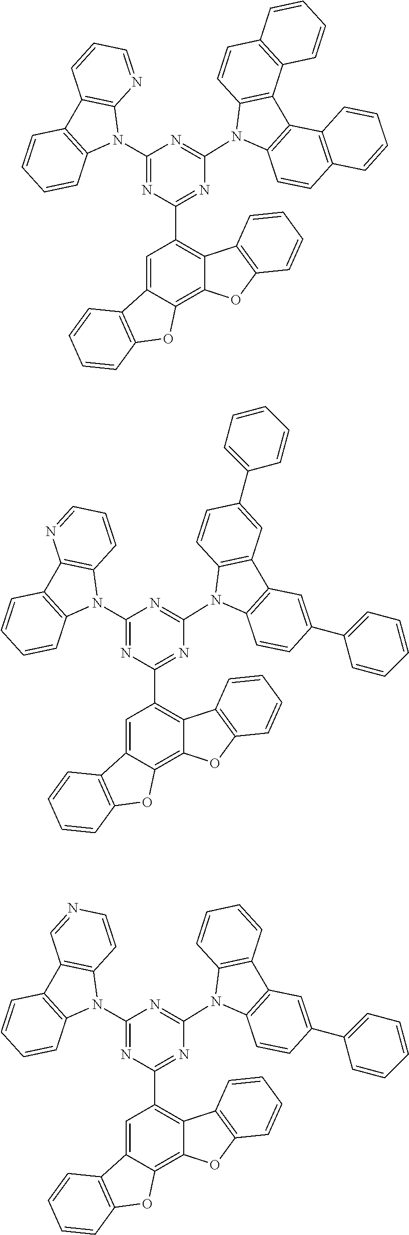 [SMILES: c1ccc2c(c1)ccc1c2c2c3ccccc3ccc2n1-c1nc(-c2cc3c4ccccc4oc3c3oc4ccccc4c23)nc(-n2c3ccccc3c3cccnc32)n1 ; c1ccc(-c2ccc3c(c2)c2cc(-c4ccccc4)ccc2n3-c2nc(-c3cc4c5ccccc5oc4c4oc5ccccc5c34)nc(-n3c4ccccc4c4ncccc43)n2)cc1 ; c1ccc(-c2ccc3c(c2)c2ccccc2n3-c2nc(-c3cc4c5ccccc5oc4c4oc5ccccc5c34)nc(-n3c4ccccc4c4cnccc43)n2)cc1]

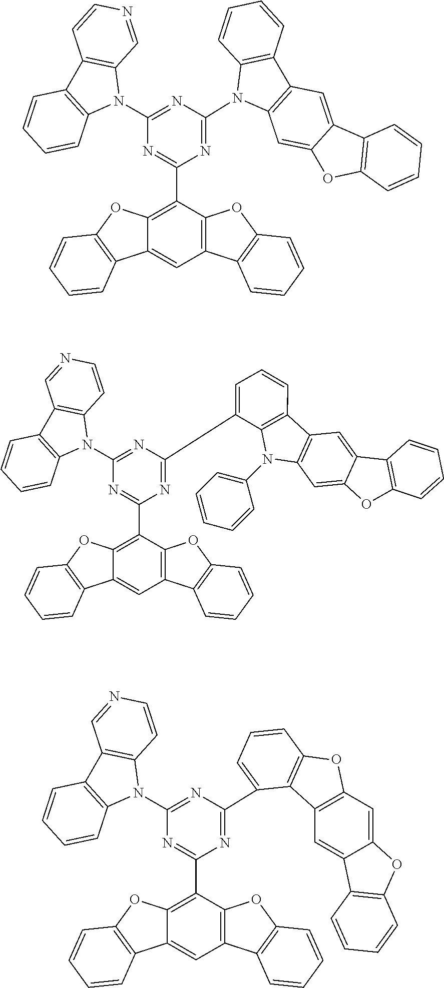 [SMILES: c1ccc2c(c1)oc1cc3c(cc12)c1ccccc1n3-c1nc(-c2c3oc4ccccc4c3cc3c2oc2ccccc23)nc(-n2c3ccccc3c3ccncc32)n1 ; c1ccc(-n2c3cc4oc5ccccc5c4cc3c3cccc(-c4nc(-c5c6oc7ccccc7c6cc6c5oc5ccccc56)nc(-n5c6ccccc6c6cnccc65)n4)c32)cc1 ; c1ccc2c(c1)oc1cc3oc4cccc(-c5nc(-c6c7oc8ccccc8c7cc7c6oc6ccccc67)nc(-n6c7ccccc7c7cnccc76)n5)c4c3cc12]

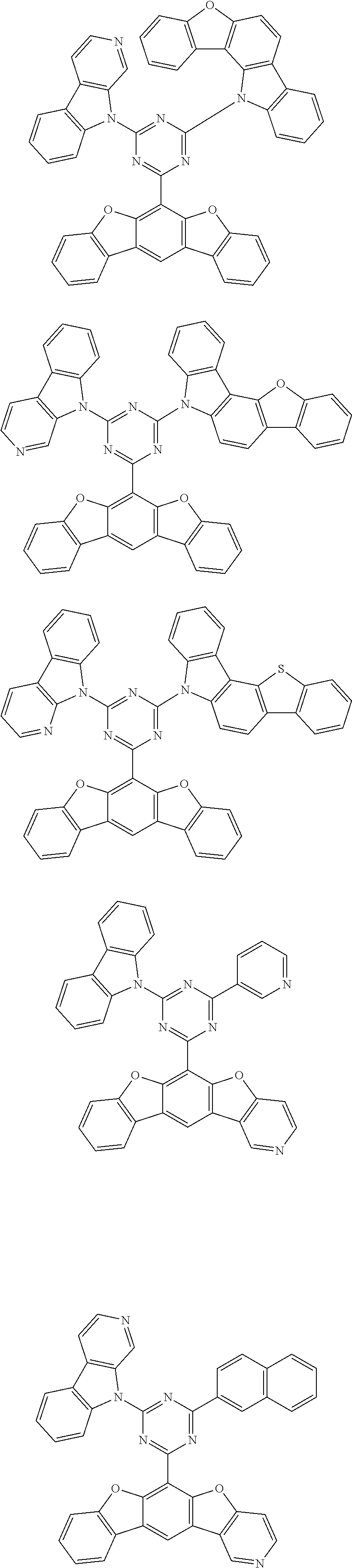

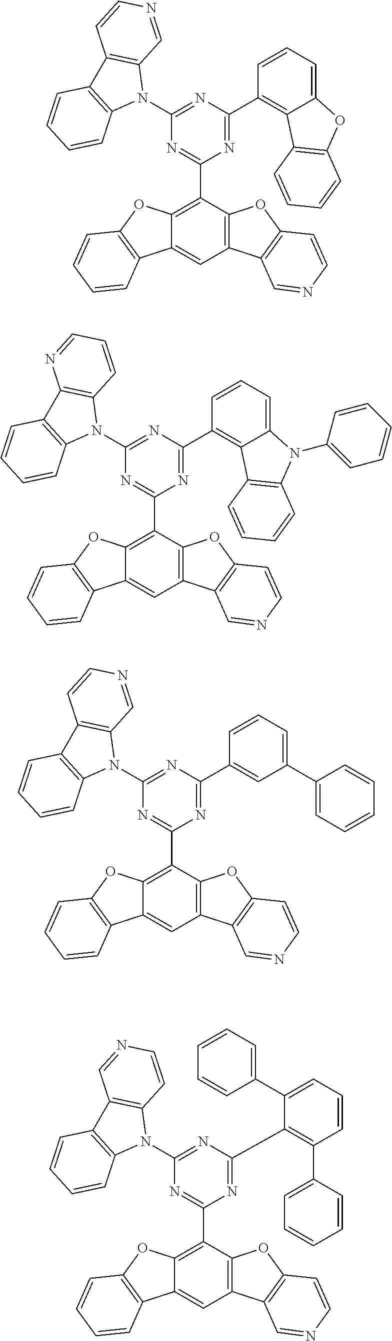 [SMILES: c1ccc2c(c1)oc1c(-c3nc(-c4cccc5oc6ccccc6c45)nc(-n4c5ccccc5c5ccncc54)n3)c3oc4ccncc4c3cc12 ; c1ccc(-n2c3ccccc3c3c(-c4nc(-c5c6oc7ccccc7c6cc6c5oc5ccncc56)nc(-n5c6ccccc6c6ncccc65)n4)cccc32)cc1 ; c1ccc(-c2cccc(-c3nc(-c4c5oc6ccccc6c5cc5c4oc4ccncc45)nc(-n4c5ccccc5c5ccncc54)n3)c2)cc1 ; c1ccc(-c2cccc(-c3ccccc3)c2-c2nc(-c3c4oc5ccccc5c4cc4c3oc3ccncc34)nc(-n3c4ccccc4c4cnccc43)n2)cc1]

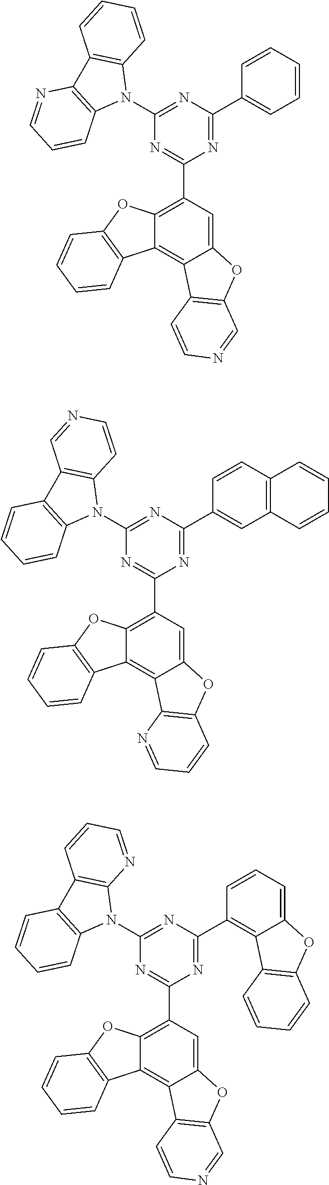 [SMILES: c1ccc(-c2nc(-c3cc4oc5cnccc5c4c4c3oc3ccccc34)nc(-n3c4ccccc4c4ncccc43)n2)cc1 ; c1ccc2cc(-c3nc(-c4cc5oc6cccnc6c5c5c4oc4ccccc45)nc(-n4c5ccccc5c5cnccc54)n3)ccc2c1 ; c1ccc2c(c1)oc1cccc(-c3nc(-c4cc5oc6cnccc6c5c5c4oc4ccccc45)nc(-n4c5ccccc5c5cccnc54)n3)c12]

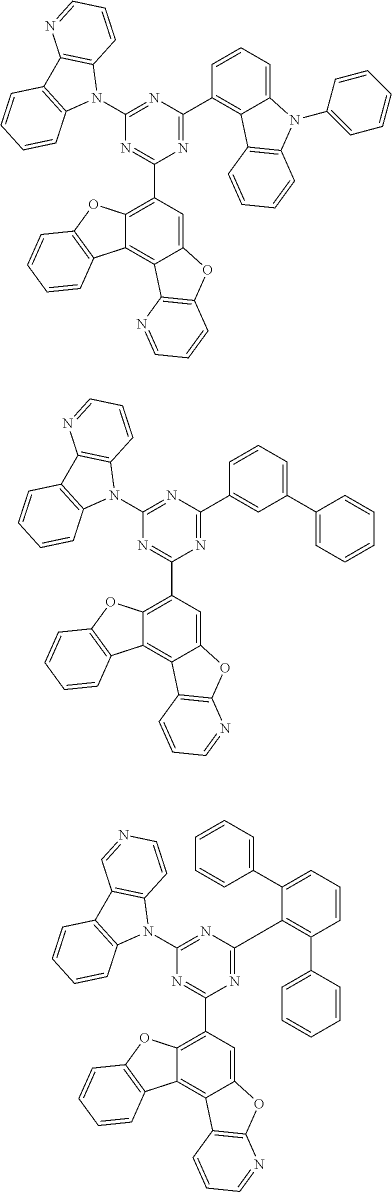 [SMILES: c1ccc(-n2c3ccccc3c3c(-c4nc(-c5cc6oc7cccnc7c6c6c5oc5ccccc56)nc(-n5c6ccccc6c6ncccc65)n4)cccc32)cc1 ; c1ccc(-c2cccc(-c3nc(-c4cc5oc6ncccc6c5c5c4oc4ccccc45)nc(-n4c5ccccc5c5ncccc54)n3)c2)cc1 ; c1ccc(-c2cccc(-c3ccccc3)c2-c2nc(-c3cc4oc5ncccc5c4c4c3oc3ccccc34)nc(-n3c4ccccc4c4cnccc43)n2)cc1]

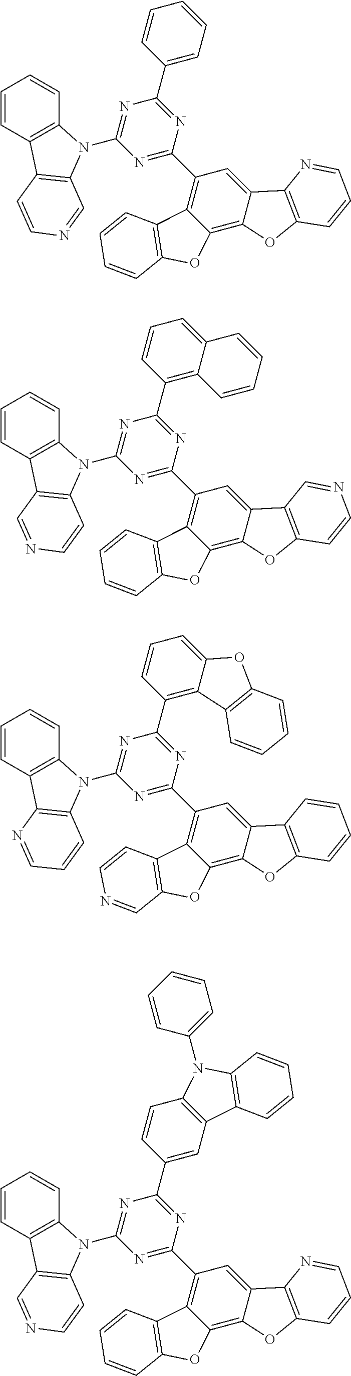 [SMILES: c1ccc(-c2nc(-c3cc4c5ncccc5oc4c4oc5ccccc5c34)nc(-n3c4ccccc4c4ccncc43)n2)cc1 ; c1ccc2c(-c3nc(-c4cc5c6cnccc6oc5c5oc6ccccc6c45)nc(-n4c5ccccc5c5cnccc54)n3)cccc2c1 ; c1ccc2c(c1)oc1c2cc(-c2nc(-c3cccc4oc5ccccc5c34)nc(-n3c4ccccc4c4ncccc43)n2)c2c3ccncc3oc12 ; c1ccc(-n2c3ccccc3c3cc(-c4nc(-c5cc6c7ncccc7oc6c6oc7ccccc7c56)nc(-n5c6ccccc6c6cnccc65)n4)ccc32)cc1]

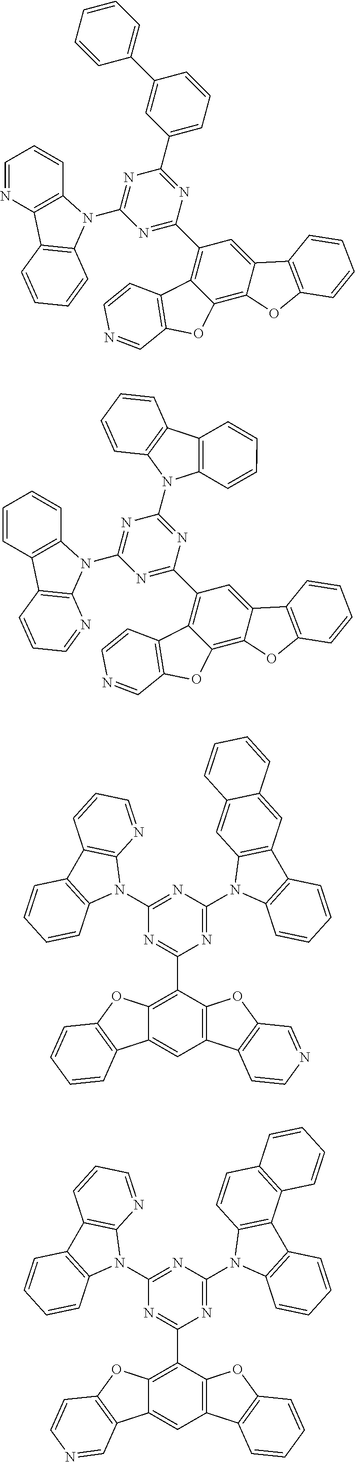

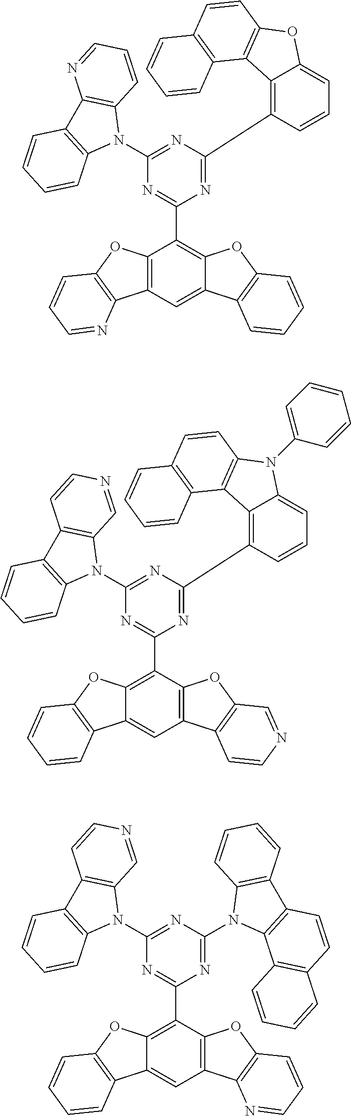 [SMILES: c1ccc2c(c1)ccc1oc3cccc(-c4nc(-c5c6oc7ccccc7c6cc6c5oc5cccnc56)nc(-n5c6ccccc6c6ncccc65)n4)c3c12 ; c1ccc(-n2c3cccc(-c4nc(-c5c6oc7ccccc7c6cc6c5oc5cnccc56)nc(-n5c6ccccc6c6ccncc65)n4)c3c3c4ccccc4ccc32)cc1 ; c1ccc2c(c1)ccc1c3ccccc3n(-c3nc(-c4c5oc6ccccc6c5cc5c4oc4cccnc45)nc(-n4c5ccccc5c5ccncc54)n3)c21]

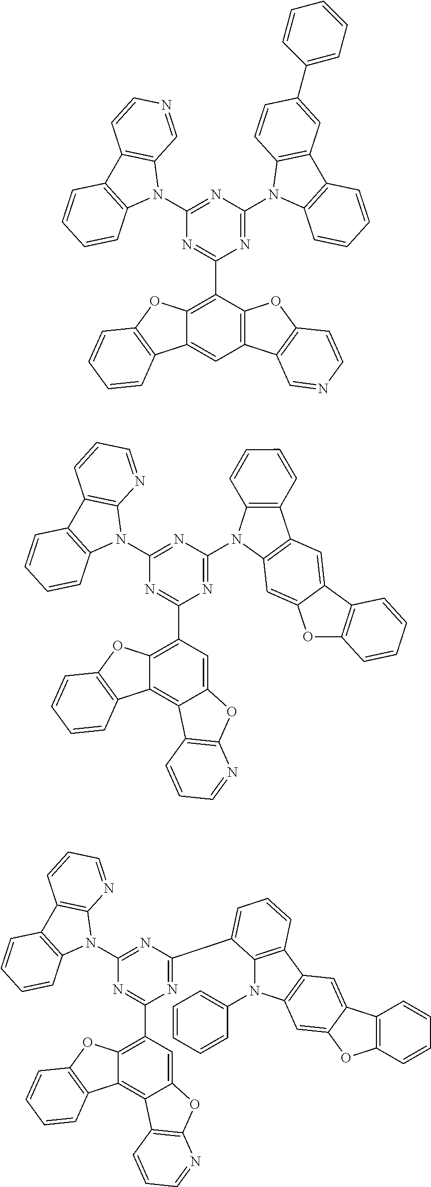 [SMILES: c1ccc(-c2ccc3c(c2)c2ccccc2n3-c2nc(-c3c4oc5ccccc5c4cc4c3oc3ccncc34)nc(-n3c4ccccc4c4ccncc43)n2)cc1 ; c1ccc2c(c1)oc1cc3c(cc12)c1ccccc1n3-c1nc(-c2cc3oc4ncccc4c3c3c2oc2ccccc23)nc(-n2c3ccccc3c3cccnc32)n1 ; c1ccc(-n2c3cc4oc5ccccc5c4cc3c3cccc(-c4nc(-c5cc6oc7ncccc7c6c6c5oc5ccccc56)nc(-n5c6ccccc6c6cccnc65)n4)c32)cc1]

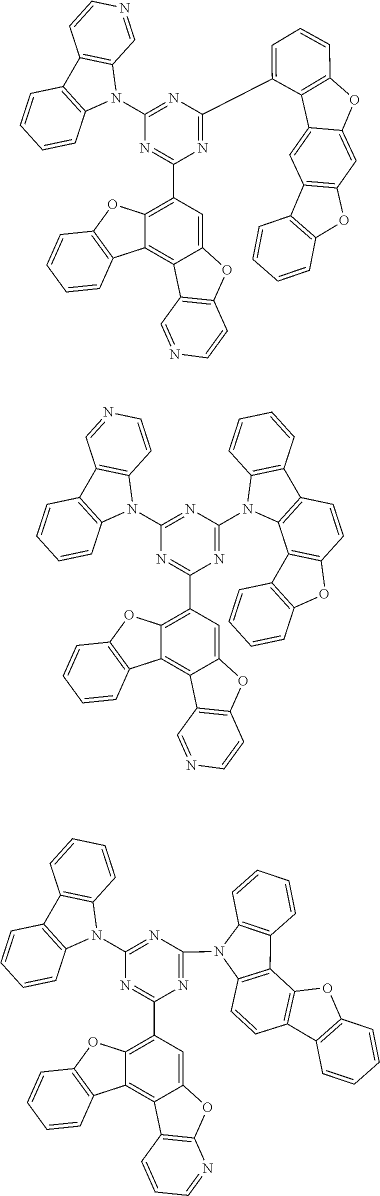 [SMILES: c1ccc2c(c1)oc1cc3oc4cccc(-c5nc(-c6cc7oc8ccncc8c7c7c6oc6ccccc67)nc(-n6c7ccccc7c7ccncc76)n5)c4c3cc12 ; c1ccc2c(c1)oc1c(-c3nc(-n4c5ccccc5c5cnccc54)nc(-n4c5ccccc5c5ccc6oc7ccccc7c6c54)n3)cc3oc4ccncc4c3c12 ; c1ccc2c(c1)oc1c2ccc2c1c1ccccc1n2-c1nc(-c2cc3oc4ncccc4c3c3c2oc2ccccc23)nc(-n2c3ccccc3c3ccccc32)n1]

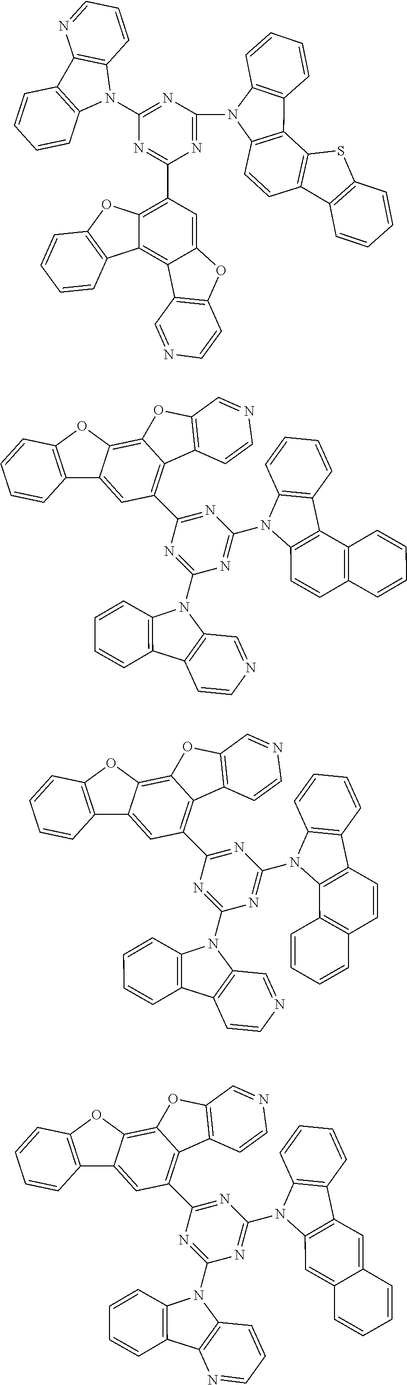

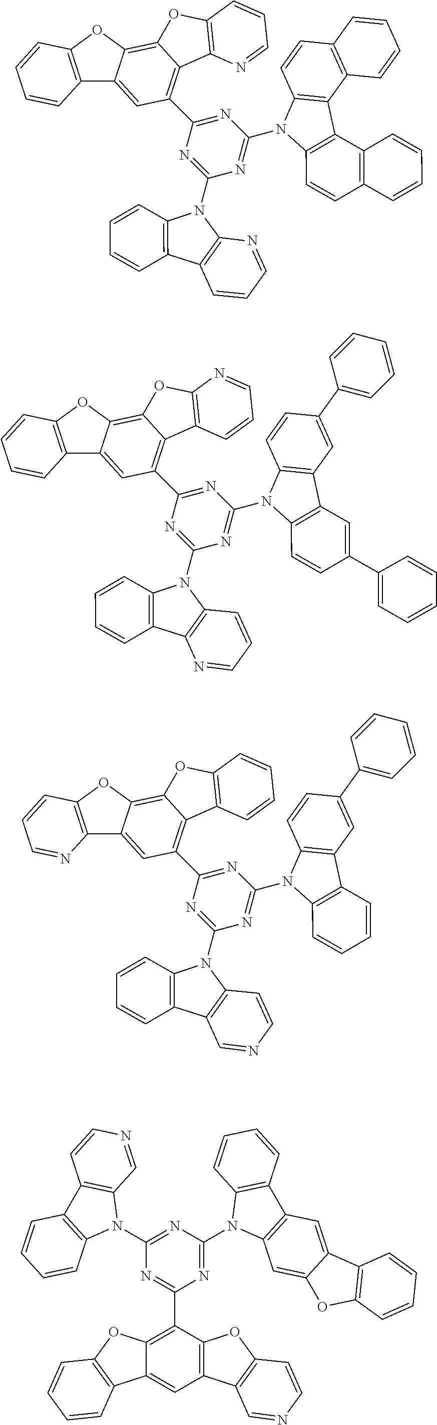 [SMILES: c1ccc2c(c1)ccc1c2c2c3ccccc3ccc2n1-c1nc(-c2cc3c4ccccc4oc3c3oc4cccnc4c23)nc(-n2c3ccccc3c3cccnc32)n1 ; c1ccc(-c2ccc3c(c2)c2cc(-c4ccccc4)ccc2n3-c2nc(-c3cc4c5ccccc5oc4c4oc5ncccc5c34)nc(-n3c4ccccc4c4ncccc43)n2)cc1 ; c1ccc(-c2ccc3c(c2)c2ccccc2n3-c2nc(-c3cc4c5ncccc5oc4c4oc5ccccc5c34)nc(-n3c4ccccc4c4cnccc43)n2)cc1 ; c1ccc2c(c1)oc1cc3c(cc12)c1ccccc1n3-c1nc(-c2c3oc4ccccc4c3cc3c2oc2ccncc23)nc(-n2c3ccccc3c3ccncc32)n1]

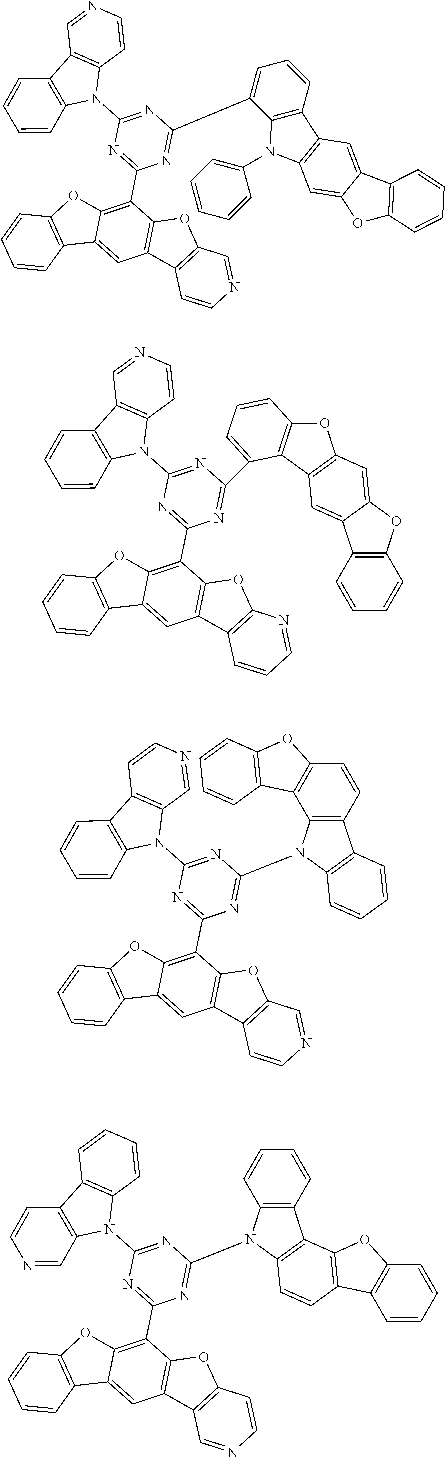 [SMILES: c1ccc(-n2c3cc4oc5ccccc5c4cc3c3cccc(-c4nc(-c5c6oc7ccccc7c6cc6c5oc5cnccc56)nc(-n5c6ccccc6c6cnccc65)n4)c32)cc1 ; c1ccc2c(c1)oc1cc3oc4cccc(-c5nc(-c6c7oc8ccccc8c7cc7c6oc6ncccc67)nc(-n6c7ccccc7c7cnccc76)n5)c4c3cc12 ; c1ccc2c(c1)oc1c(-c3nc(-n4c5ccccc5c5ccncc54)nc(-n4c5ccccc5c5ccc6oc7ccccc7c6c54)n3)c3oc4cnccc4c3cc12 ; c1ccc2c(c1)oc1c(-c3nc(-n4c5ccccc5c5ccncc54)nc(-n4c5ccccc5c5c6oc7ccccc7c6ccc54)n3)c3oc4ccncc4c3cc12]

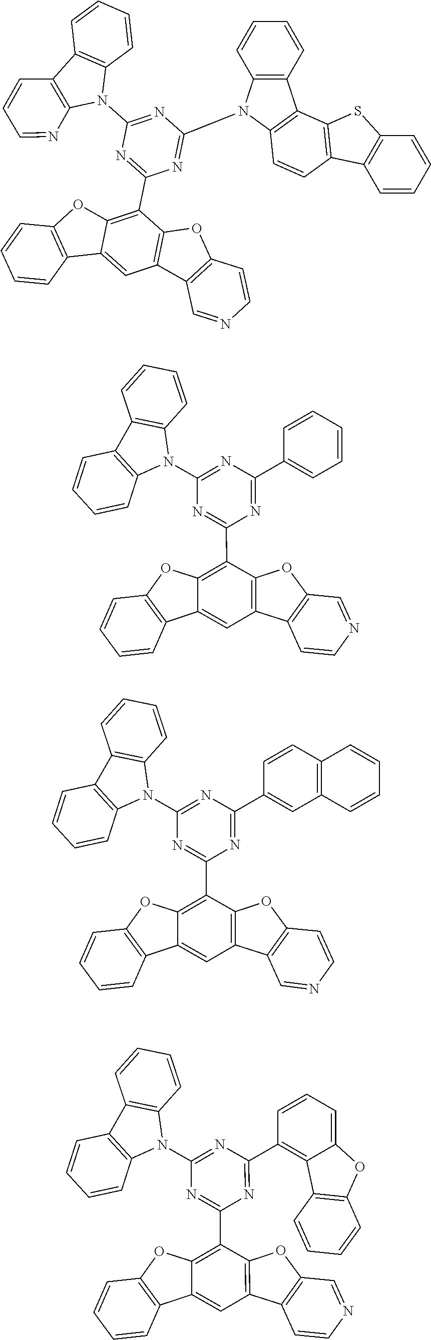

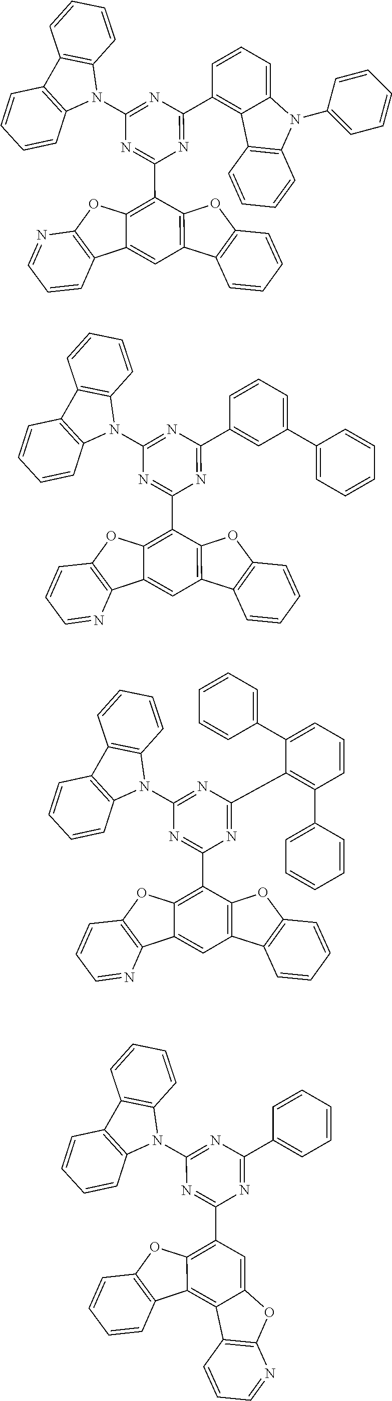

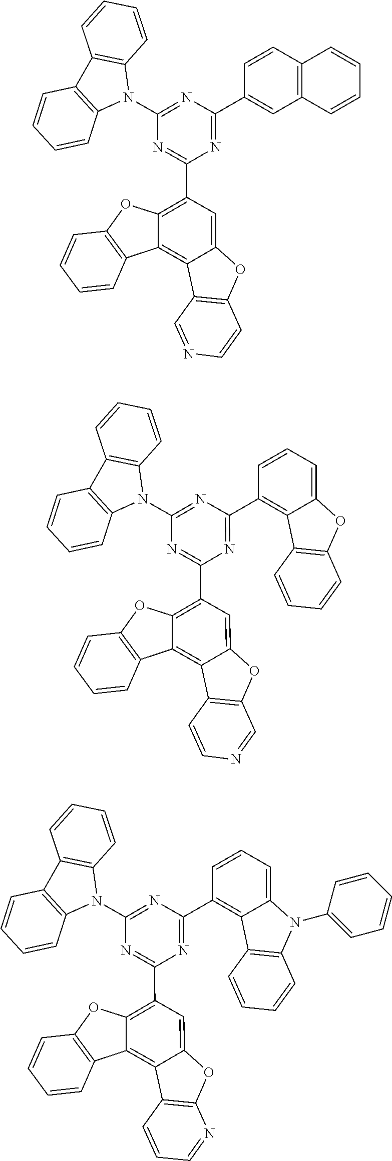 [SMILES: c1ccc2cc(-c3nc(-c4cc5oc6ccncc6c5c5c4oc4ccccc45)nc(-n4c5ccccc5c5ccccc54)n3)ccc2c1 ; c1ccc2c(c1)oc1cccc(-c3nc(-c4cc5oc6cnccc6c5c5c4oc4ccccc45)nc(-n4c5ccccc5c5ccccc54)n3)c12 ; c1ccc(-n2c3ccccc3c3c(-c4nc(-c5cc6oc7ncccc7c6c6c5oc5ccccc56)nc(-n5c6ccccc6c6ccccc65)n4)cccc32)cc1]

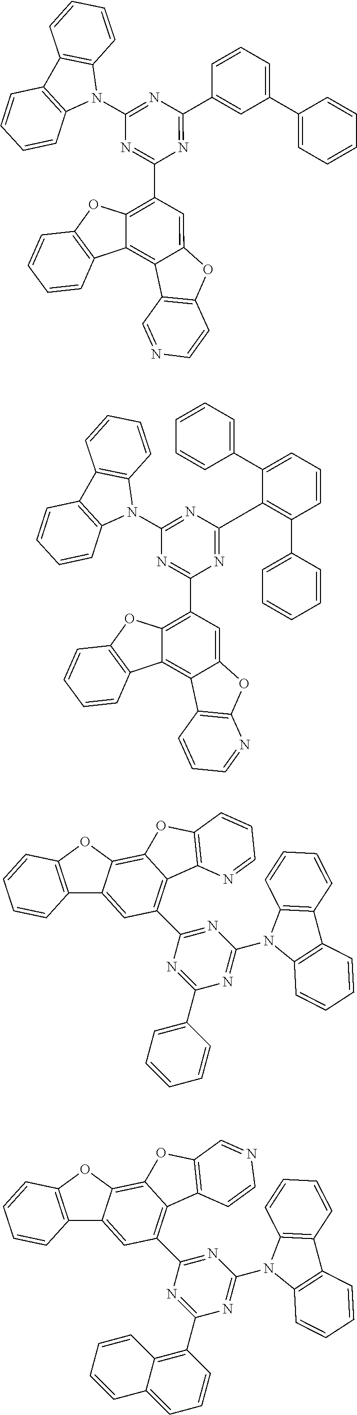

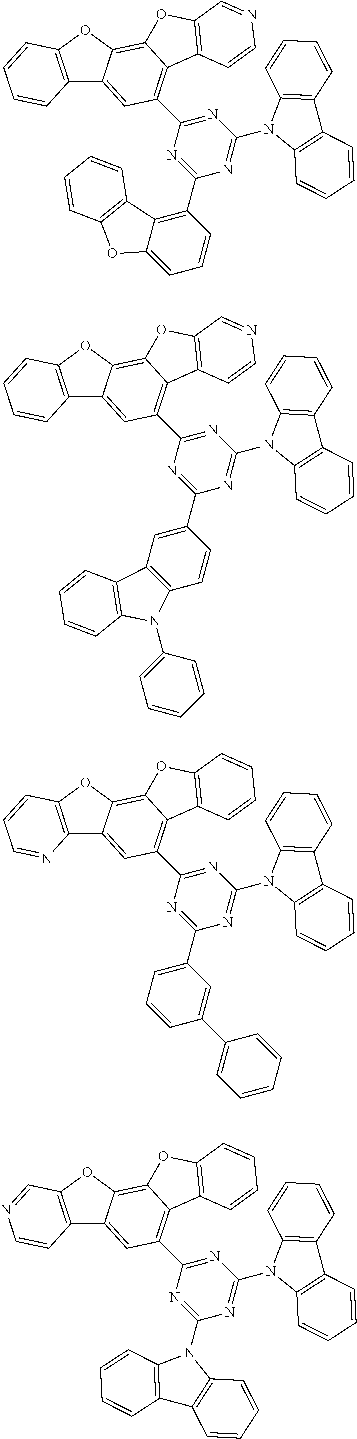 [SMILES: c1ccc2c(c1)oc1c2cc(-c2nc(-c3cccc4oc5ccccc5c34)nc(-n3c4ccccc4c4ccccc43)n2)c2c3ccncc3oc12 ; c1ccc(-n2c3ccccc3c3cc(-c4nc(-c5cc6c7ccccc7oc6c6oc7cnccc7c56)nc(-n5c6ccccc6c6ccccc65)n4)ccc32)cc1 ; c1ccc(-c2cccc(-c3nc(-c4cc5c6ncccc6oc5c5oc6ccccc6c45)nc(-n4c5ccccc5c5ccccc54)n3)c2)cc1 ; c1ccc2c(c1)oc1c3oc4cnccc4c3cc(-c3nc(-n4c5ccccc5c5ccccc54)nc(-n4c5ccccc5c5ccccc54)n3)c21]

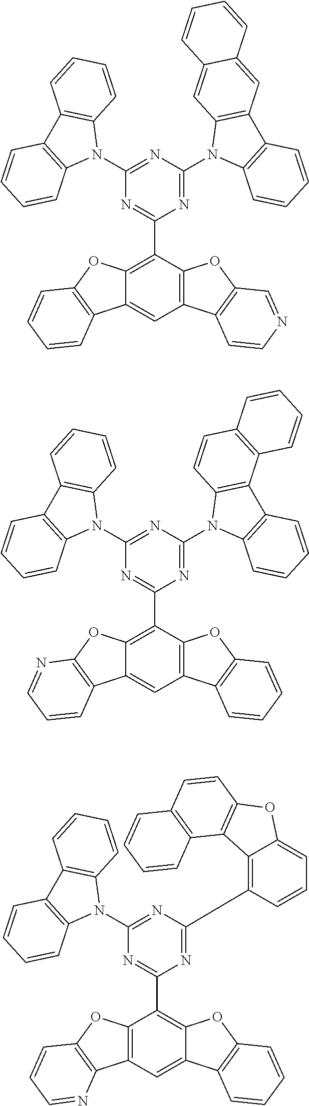

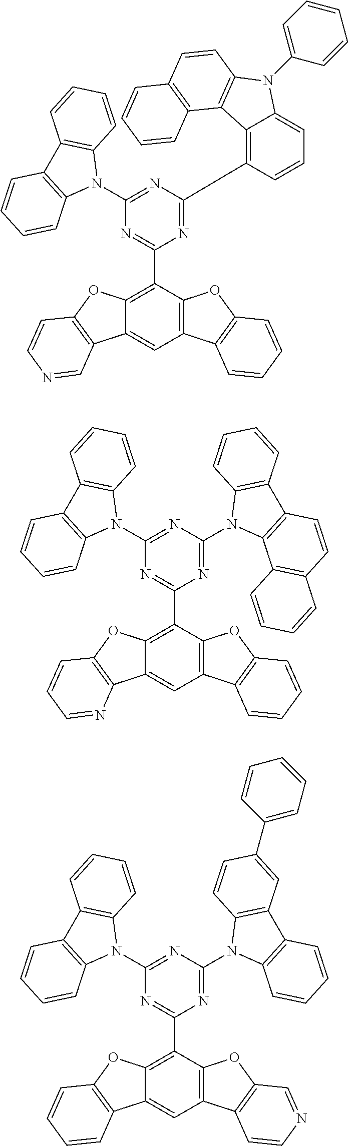

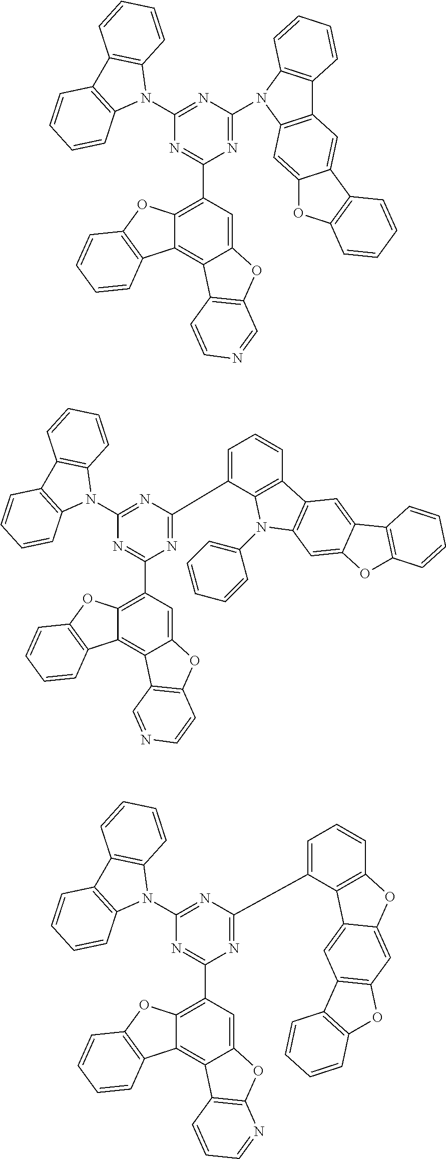 [SMILES: c1ccc2c(c1)oc1cc3c(cc12)c1ccccc1n3-c1nc(-c2cc3oc4cnccc4c3c3c2oc2ccccc23)nc(-n2c3ccccc3c3ccccc32)n1 ; c1ccc(-n2c3cc4oc5ccccc5c4cc3c3cccc(-c4nc(-c5cc6oc7ccncc7c6c6c5oc5ccccc56)nc(-n5c6ccccc6c6ccccc65)n4)c32)cc1 ; c1ccc2c(c1)oc1cc3oc4cccc(-c5nc(-c6cc7oc8ncccc8c7c7c6oc6ccccc67)nc(-n6c7ccccc7c7ccccc76)n5)c4c3cc12]

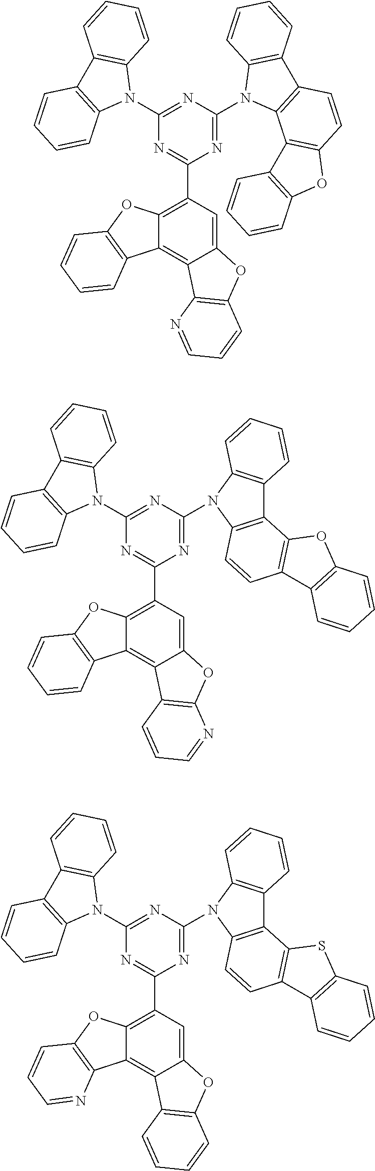 [SMILES: c1ccc2c(c1)oc1c(-c3nc(-n4c5ccccc5c5ccccc54)nc(-n4c5ccccc5c5ccc6oc7ccccc7c6c54)n3)cc3oc4cccnc4c3c12 ; c1ccc2c(c1)oc1c2ccc2c1c1ccccc1n2-c1nc(-c2cc3oc4ncccc4c3c3c2oc2ccccc23)nc(-n2c3ccccc3c3ccccc32)n1 ; c1ccc2c(c1)oc1cc(-c3nc(-n4c5ccccc5c5ccccc54)nc(-n4c5ccccc5c5c6sc7ccccc7c6ccc54)n3)c3oc4cccnc4c3c12]

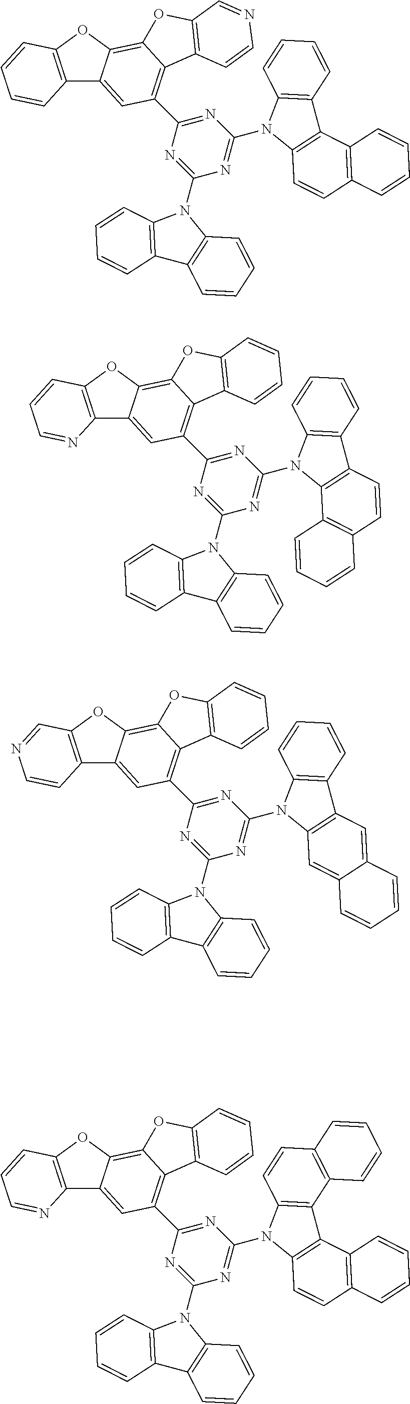

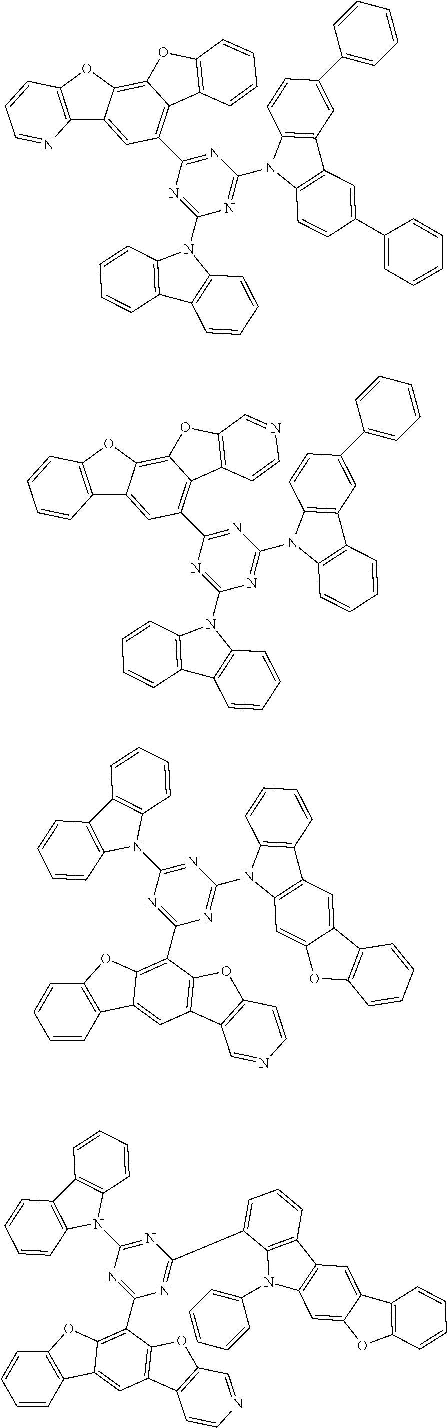 [SMILES: c1ccc(-c2ccc3c(c2)c2cc(-c4ccccc4)ccc2n3-c2nc(-c3cc4c5ncccc5oc4c4oc5ccccc5c34)nc(-n3c4ccccc4c4ccccc43)n2)cc1 ; c1ccc(-c2ccc3c(c2)c2ccccc2n3-c2nc(-c3cc4c5ccccc5oc4c4oc5cnccc5c34)nc(-n3c4ccccc4c4ccccc43)n2)cc1 ; c1ccc2c(c1)oc1cc3c(cc12)c1ccccc1n3-c1nc(-c2c3oc4ccccc4c3cc3c2oc2ccncc23)nc(-n2c3ccccc3c3ccccc32)n1 ; c1ccc(-n2c3cc4oc5ccccc5c4cc3c3cccc(-c4nc(-c5c6oc7ccccc7c6cc6c5oc5cnccc56)nc(-n5c6ccccc6c6ccccc65)n4)c32)cc1]

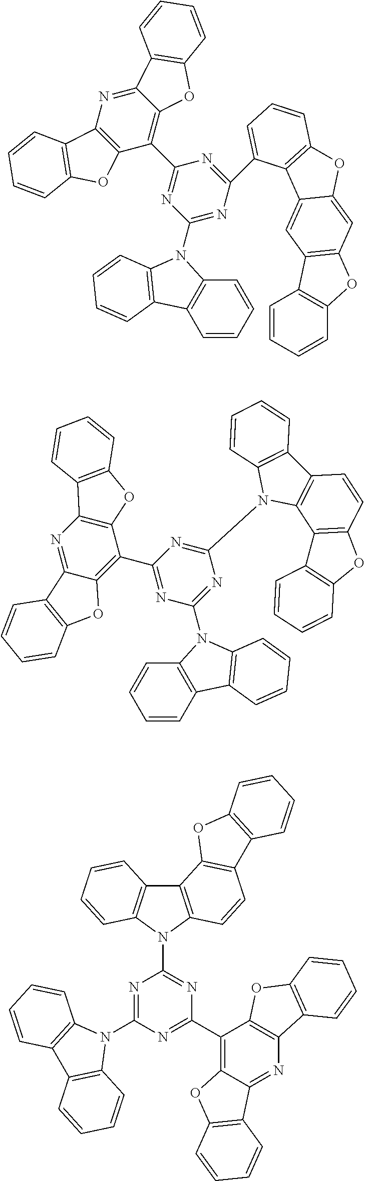 [SMILES: c1ccc2c(c1)oc1cc3oc4cccc(-c5nc(-c6c7oc8ccccc8c7nc7c6oc6ccccc67)nc(-n6c7ccccc7c7ccccc76)n5)c4c3cc12 ; c1ccc2c(c1)oc1c(-c3nc(-n4c5ccccc5c5ccccc54)nc(-n4c5ccccc5c5ccc6oc7ccccc7c6c54)n3)c3oc4ccccc4c3nc12 ; c1ccc2c(c1)oc1c2ccc2c1c1ccccc1n2-c1nc(-c2c3oc4ccccc4c3nc3c2oc2ccccc23)nc(-n2c3ccccc3c3ccccc32)n1]

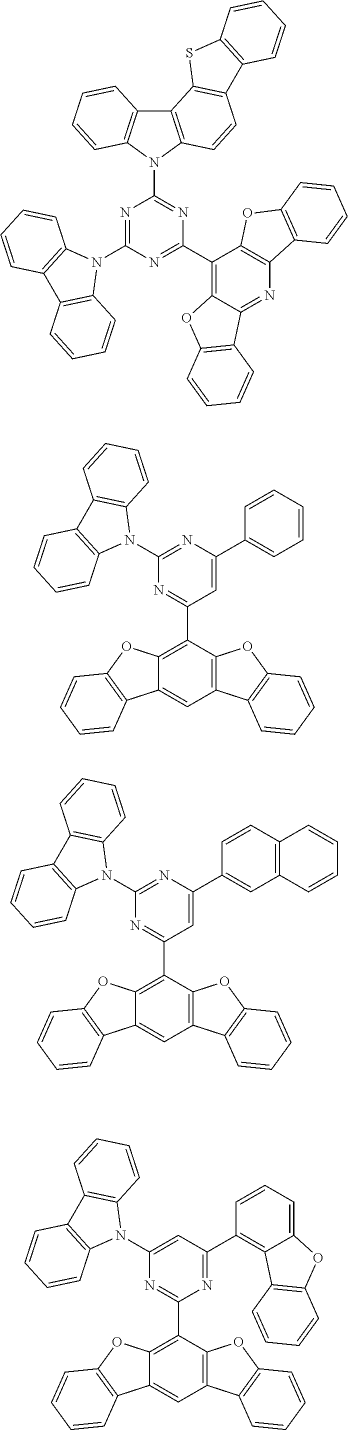

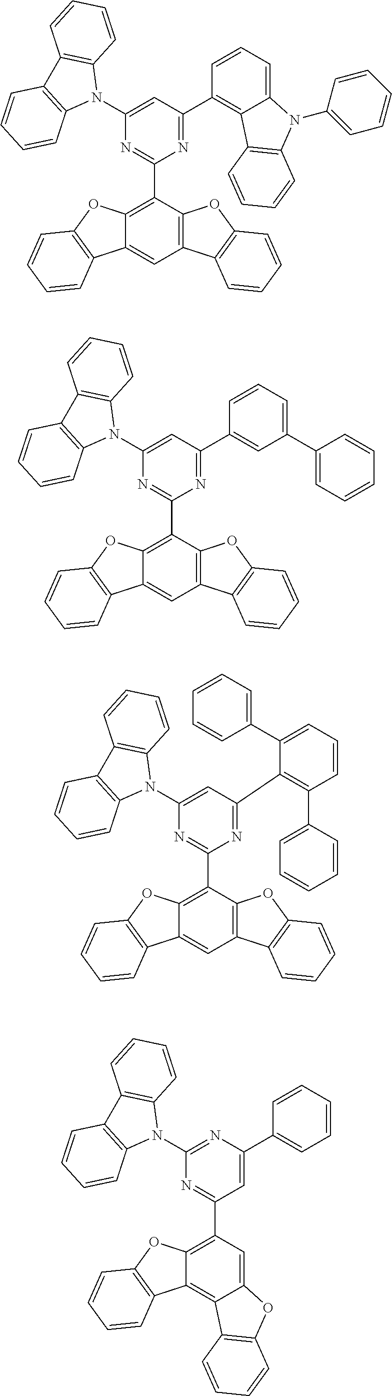 [SMILES: c1ccc(-n2c3ccccc3c3c(-c4cc(-n5c6ccccc6c6ccccc65)nc(-c5c6oc7ccccc7c6cc6c5oc5ccccc56)n4)cccc32)cc1 ; c1ccc(-c2cccc(-c3cc(-n4c5ccccc5c5ccccc54)nc(-c4c5oc6ccccc6c5cc5c4oc4ccccc45)n3)c2)cc1 ; c1ccc(-c2cccc(-c3ccccc3)c2-c2cc(-n3c4ccccc4c4ccccc43)nc(-c3c4oc5ccccc5c4cc4c3oc3ccccc34)n2)cc1 ; c1ccc(-c2cc(-c3cc4oc5ccccc5c4c4c3oc3ccccc34)nc(-n3c4ccccc4c4ccccc43)n2)cc1]

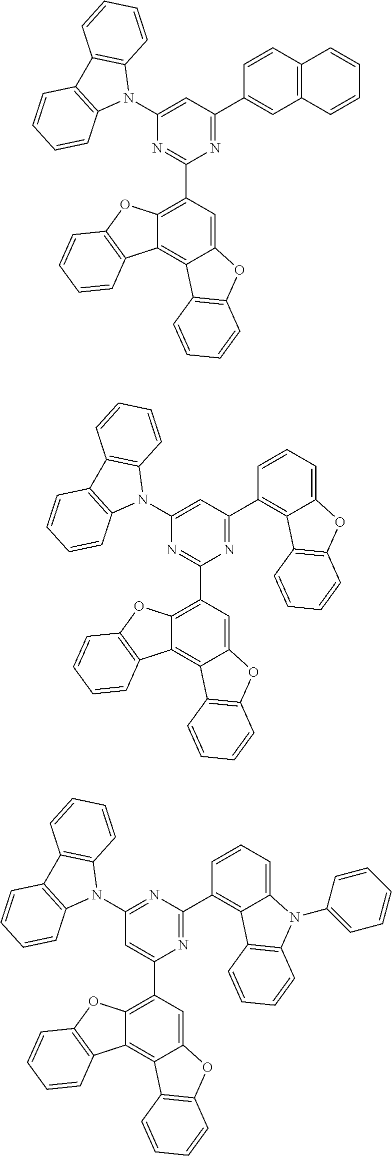 [SMILES: c1ccc2cc(-c3cc(-n4c5ccccc5c5ccccc54)nc(-c4cc5oc6ccccc6c5c5c4oc4ccccc45)n3)ccc2c1 ; c1ccc2c(c1)oc1cccc(-c3cc(-n4c5ccccc5c5ccccc54)nc(-c4cc5oc6ccccc6c5c5c4oc4ccccc45)n3)c12 ; c1ccc(-n2c3ccccc3c3c(-c4nc(-c5cc6oc7ccccc7c6c6c5oc5ccccc56)cc(-n5c6ccccc6c6ccccc65)n4)cccc32)cc1]

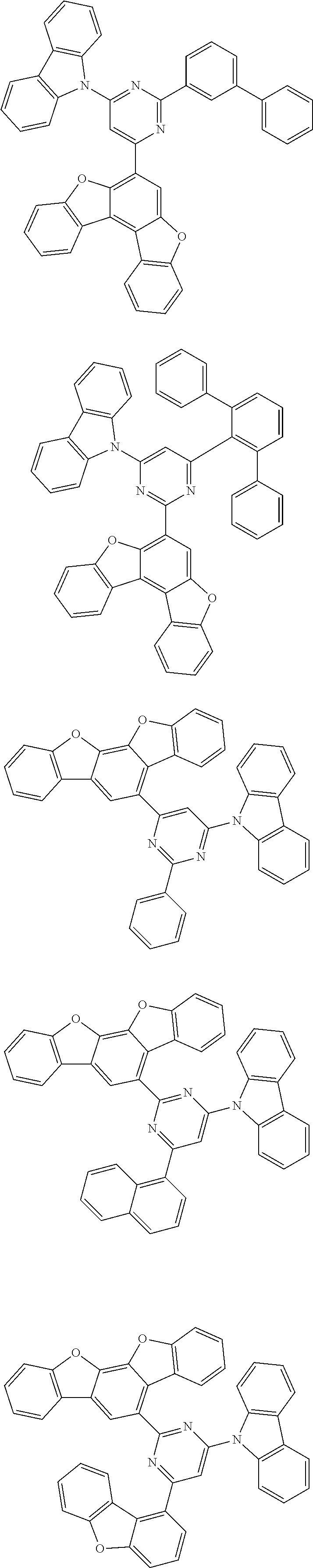

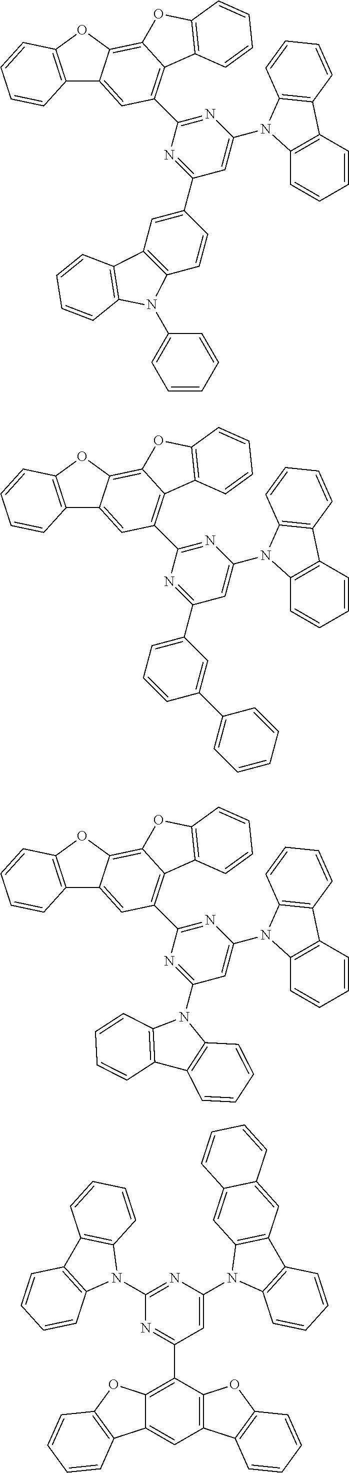

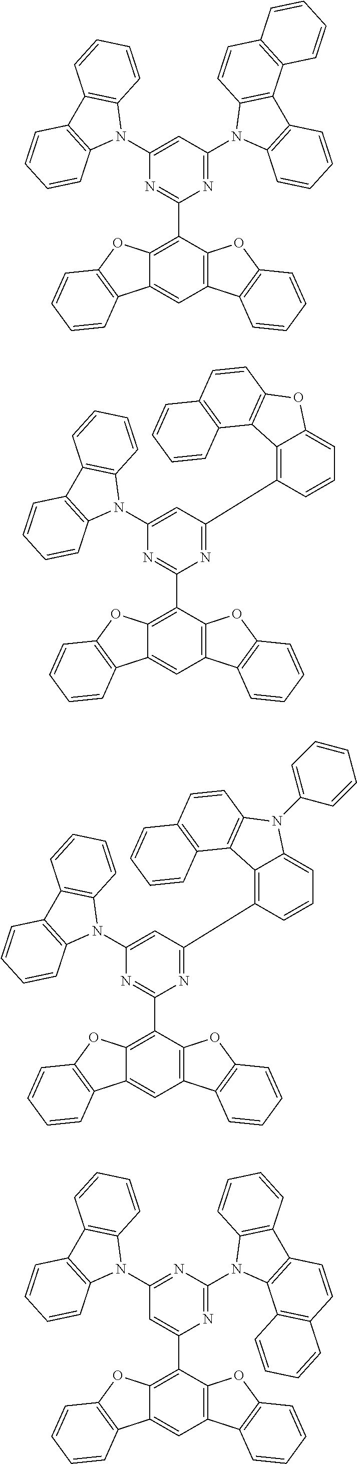

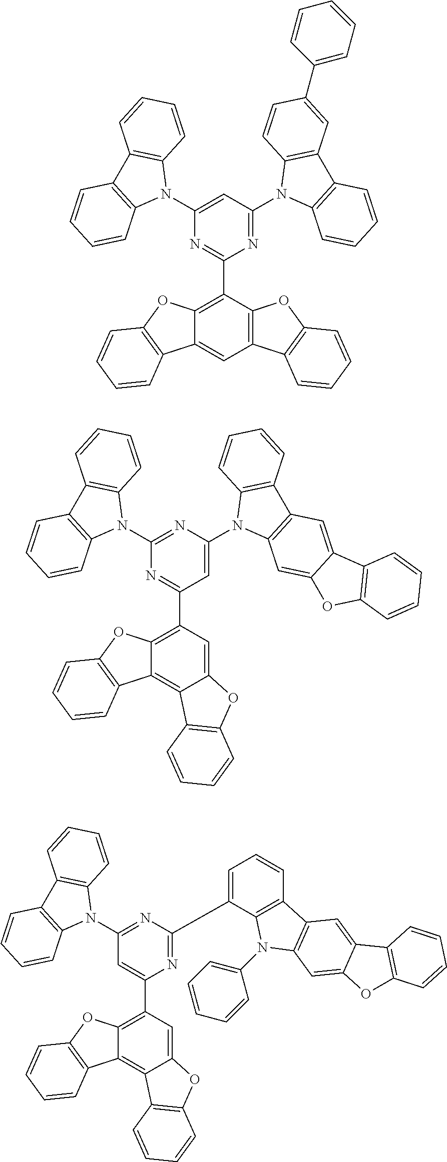

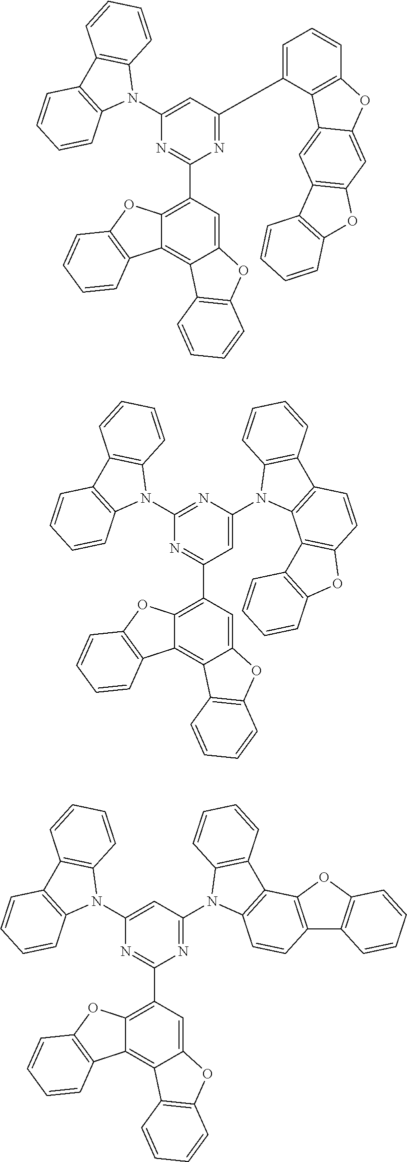

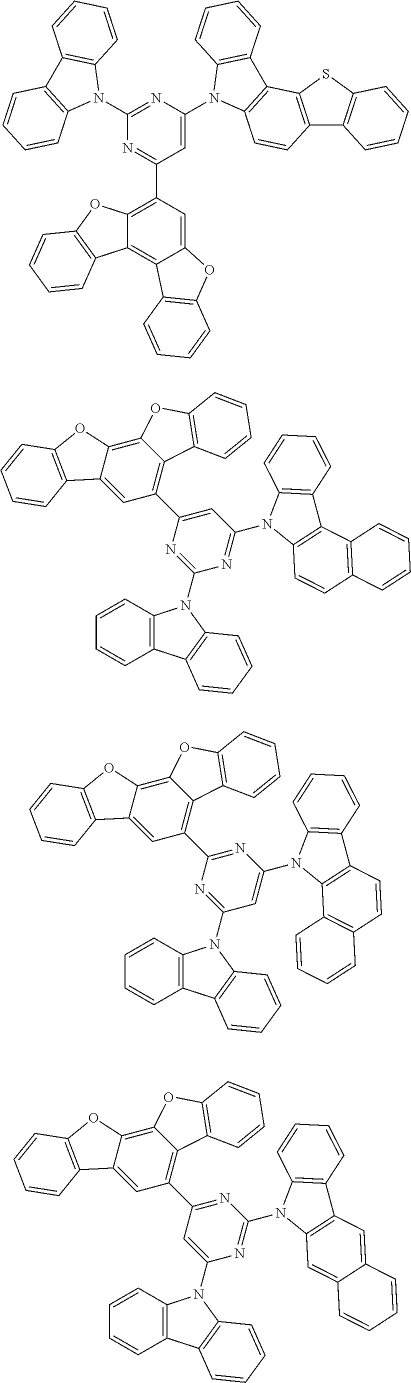

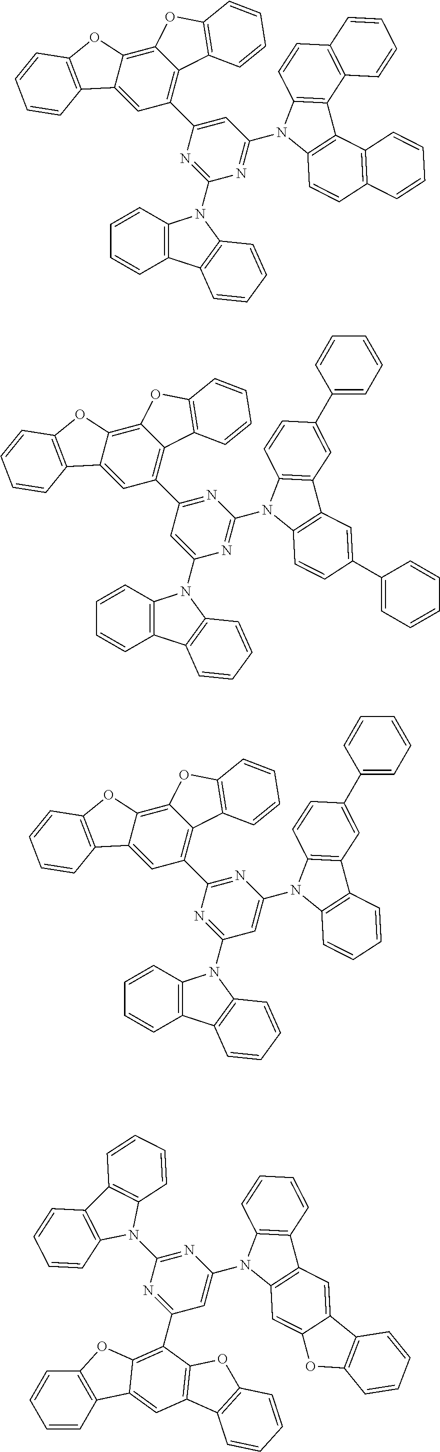 [SMILES: c1ccc2c(c1)ccc1c2c2c3ccccc3ccc2n1-c1cc(-c2cc3c4ccccc4oc3c3oc4ccccc4c23)nc(-n2c3ccccc3c3ccccc32)n1 ; c1ccc(-c2ccc3c(c2)c2cc(-c4ccccc4)ccc2n3-c2nc(-c3cc4c5ccccc5oc4c4oc5ccccc5c34)cc(-n3c4ccccc4c4ccccc43)n2)cc1 ; c1ccc(-c2ccc3c(c2)c2ccccc2n3-c2cc(-n3c4ccccc4c4ccccc43)nc(-c3cc4c5ccccc5oc4c4oc5ccccc5c34)n2)cc1 ; c1ccc2c(c1)oc1cc3c(cc12)c1ccccc1n3-c1cc(-c2c3oc4ccccc4c3cc3c2oc2ccccc23)nc(-n2c3ccccc3c3ccccc32)n1]

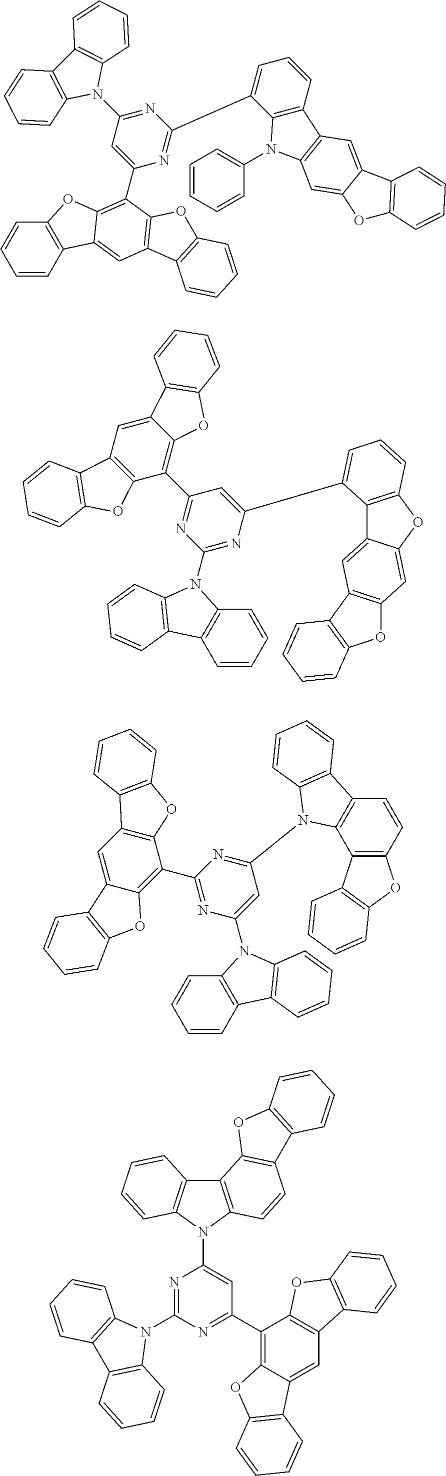

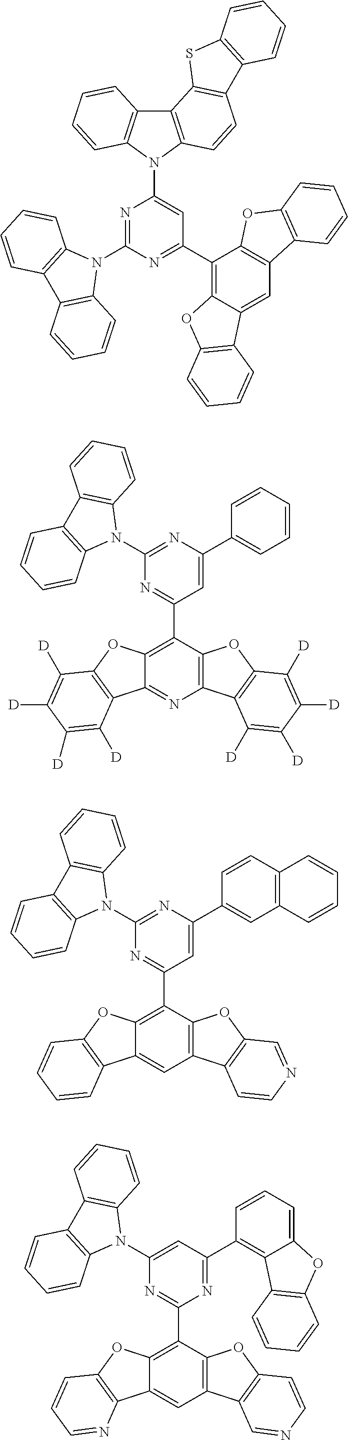 [SMILES: c1ccc2c(c1)oc1c(-c3cc(-n4c5ccccc5c5c6sc7ccccc7c6ccc54)nc(-n4c5ccccc5c5ccccc54)n3)c3oc4ccccc4c3cc12 ; [2H]c1c([2H])c([2H])c2c(oc3c(-c4cc(-c5ccccc5)nc(-n5c6ccccc6c6ccccc65)n4)c4oc5c([2H])c([2H])c([2H])c([2H])c5c4nc32)c1[2H] ; c1ccc2cc(-c3cc(-c4c5oc6ccccc6c5cc5c4oc4cnccc45)nc(-n4c5ccccc5c5ccccc54)n3)ccc2c1 ; c1ccc2c(c1)oc1cccc(-c3cc(-n4c5ccccc5c5ccccc54)nc(-c4c5oc6ccncc6c5cc5c4oc4cccnc45)n3)c12]

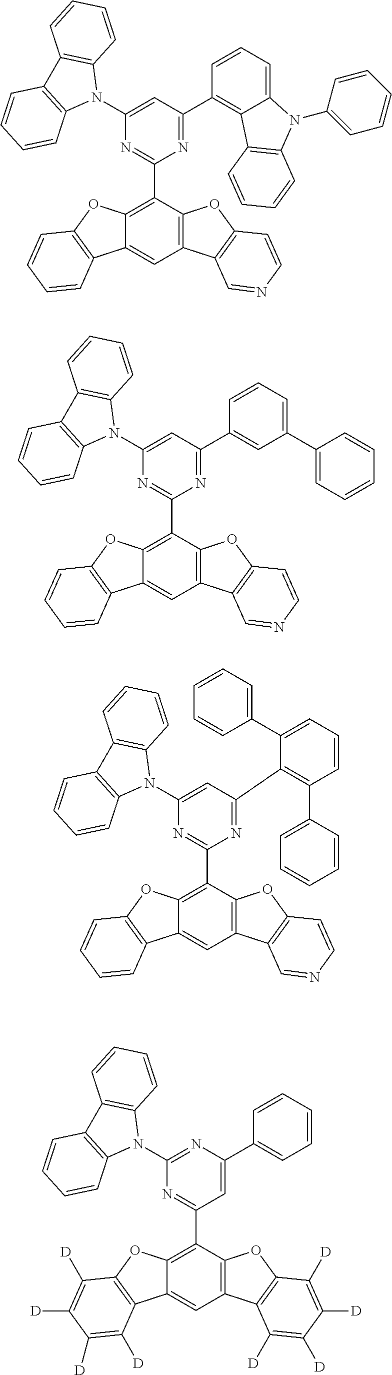 [SMILES: c1ccc(-n2c3ccccc3c3c(-c4cc(-n5c6ccccc6c6ccccc65)nc(-c5c6oc7ccccc7c6cc6c5oc5ccncc56)n4)cccc32)cc1 ; c1ccc(-c2cccc(-c3cc(-n4c5ccccc5c5ccccc54)nc(-c4c5oc6ccccc6c5cc5c4oc4ccncc45)n3)c2)cc1 ; c1ccc(-c2cccc(-c3ccccc3)c2-c2cc(-n3c4ccccc4c4ccccc43)nc(-c3c4oc5ccccc5c4cc4c3oc3ccncc34)n2)cc1 ; [2H]c1c([2H])c([2H])c2c(oc3c(-c4cc(-c5ccccc5)nc(-n5c6ccccc6c6ccccc65)n4)c4oc5c([2H])c([2H])c([2H])c([2H])c5c4cc32)c1[2H]]

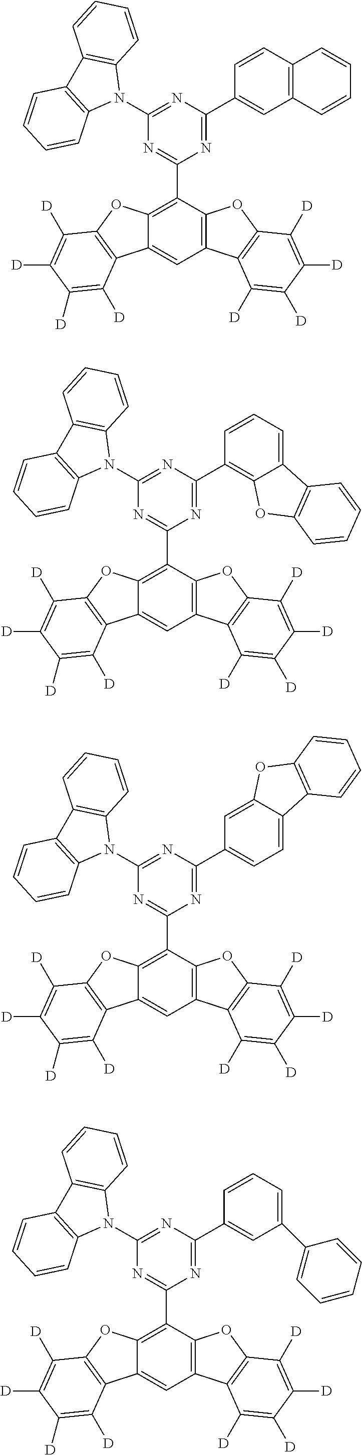

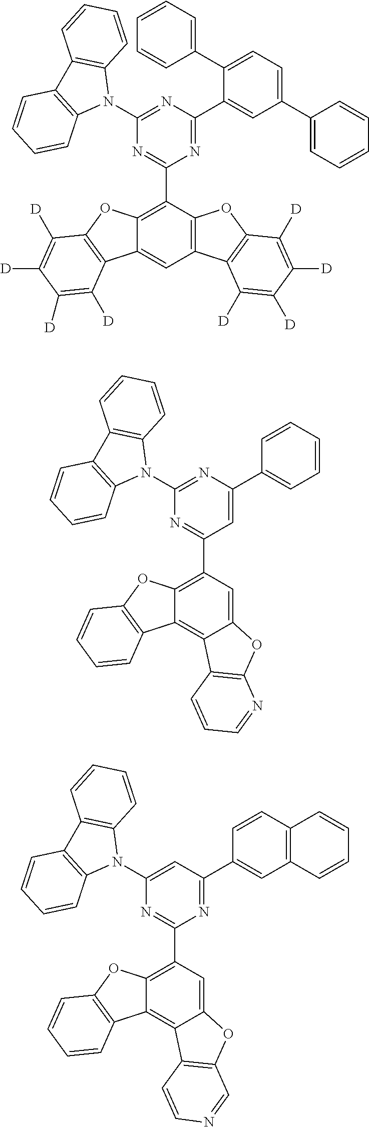 [SMILES: [2H]c1c([2H])c([2H])c2c(oc3c(-c4nc(-c5cc(-c6ccccc6)ccc5-c5ccccc5)nc(-n5c6ccccc6c6ccccc65)n4)c4oc5c([2H])c([2H])c([2H])c([2H])c5c4cc32)c1[2H] ; c1ccc(-c2cc(-c3cc4oc5ncccc5c4c4c3oc3ccccc34)nc(-n3c4ccccc4c4ccccc43)n2)cc1 ; c1ccc2cc(-c3cc(-n4c5ccccc5c5ccccc54)nc(-c4cc5oc6cnccc6c5c5c4oc4ccccc45)n3)ccc2c1]

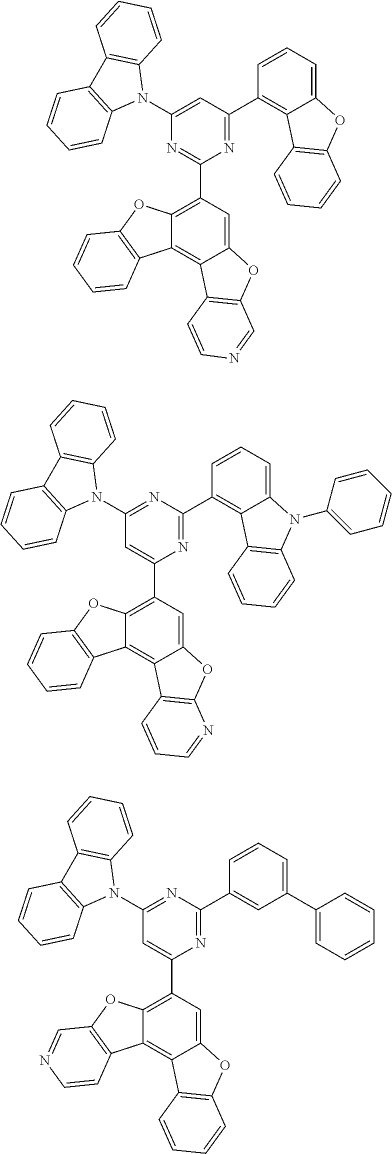 [SMILES: c1ccc2c(c1)oc1cccc(-c3cc(-n4c5ccccc5c5ccccc54)nc(-c4cc5oc6cnccc6c5c5c4oc4ccccc45)n3)c12 ; c1ccc(-n2c3ccccc3c3c(-c4nc(-c5cc6oc7ncccc7c6c6c5oc5ccccc56)cc(-n5c6ccccc6c6ccccc65)n4)cccc32)cc1 ; c1ccc(-c2cccc(-c3nc(-c4cc5oc6ccccc6c5c5c4oc4cnccc45)cc(-n4c5ccccc5c5ccccc54)n3)c2)cc1]

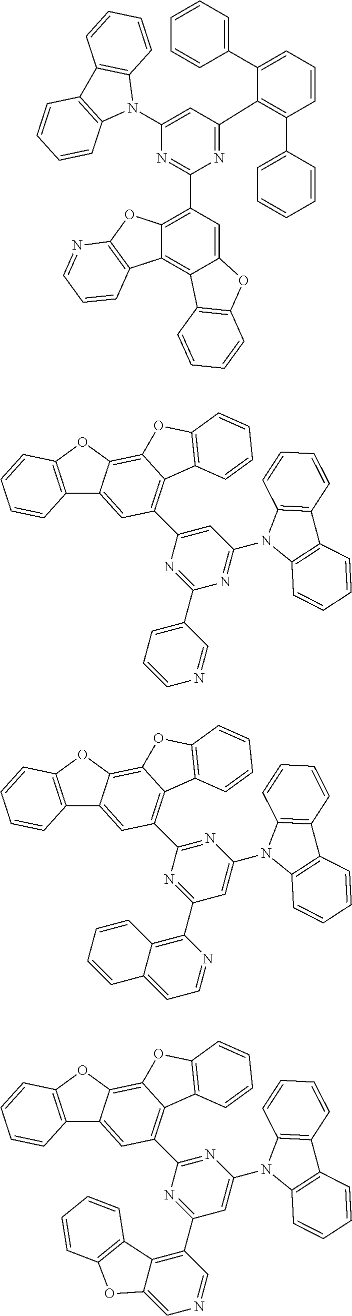

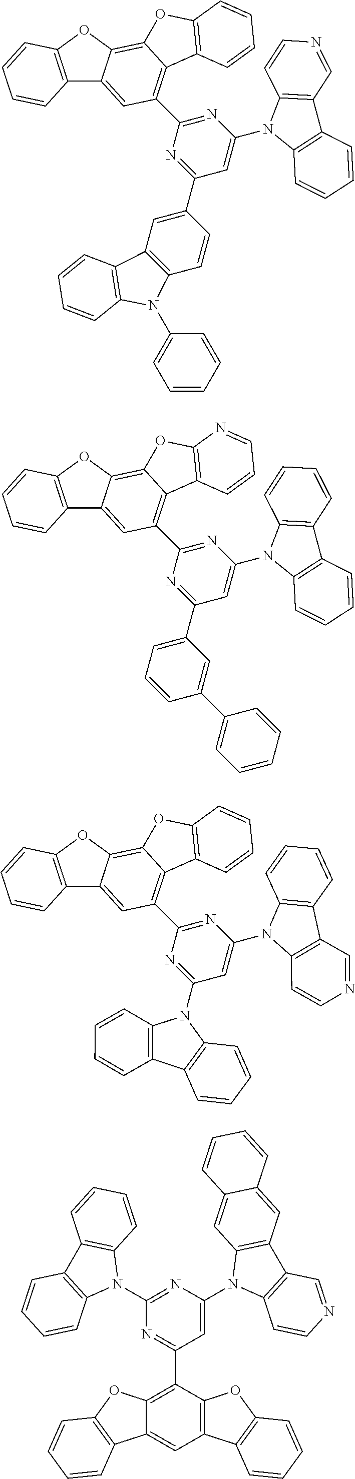

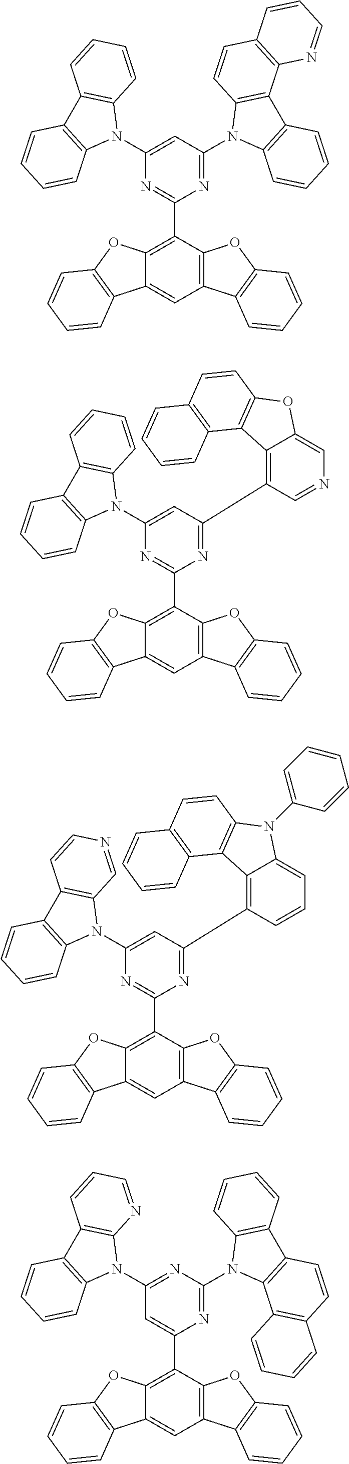

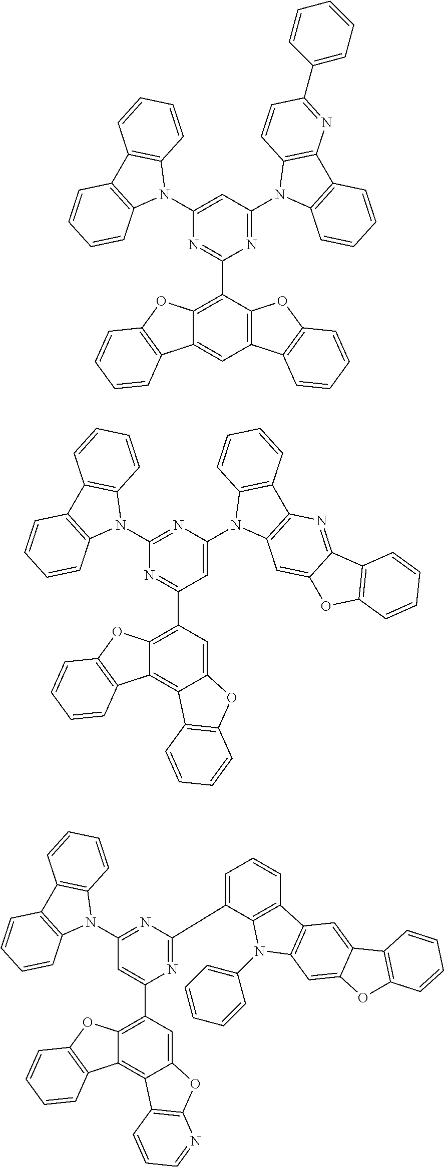

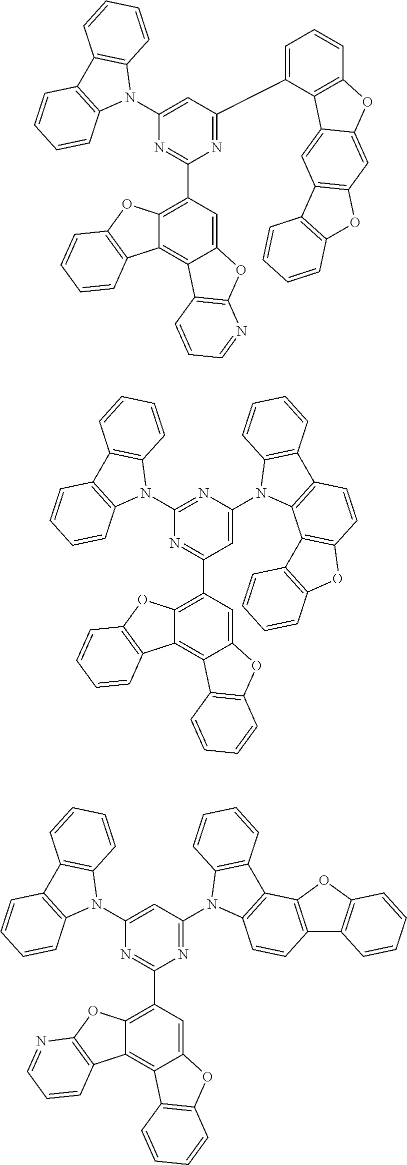 [SMILES: c1ccc2c(c1)oc1cc3oc4cccc(-c5cc(-n6c7ccccc7c7ccccc76)nc(-c6cc7oc8ncccc8c7c7c6oc6ccccc67)n5)c4c3cc12 ; c1ccc2c(c1)oc1cc(-c3cc(-n4c5ccccc5c5ccc6oc7ccccc7c6c54)nc(-n4c5ccccc5c5ccccc54)n3)c3oc4ccccc4c3c12 ; c1ccc2c(c1)oc1c2ccc2c1c1ccccc1n2-c1cc(-n2c3ccccc3c3ccccc32)nc(-c2cc3oc4ccccc4c3c3c2oc2ncccc23)n1]

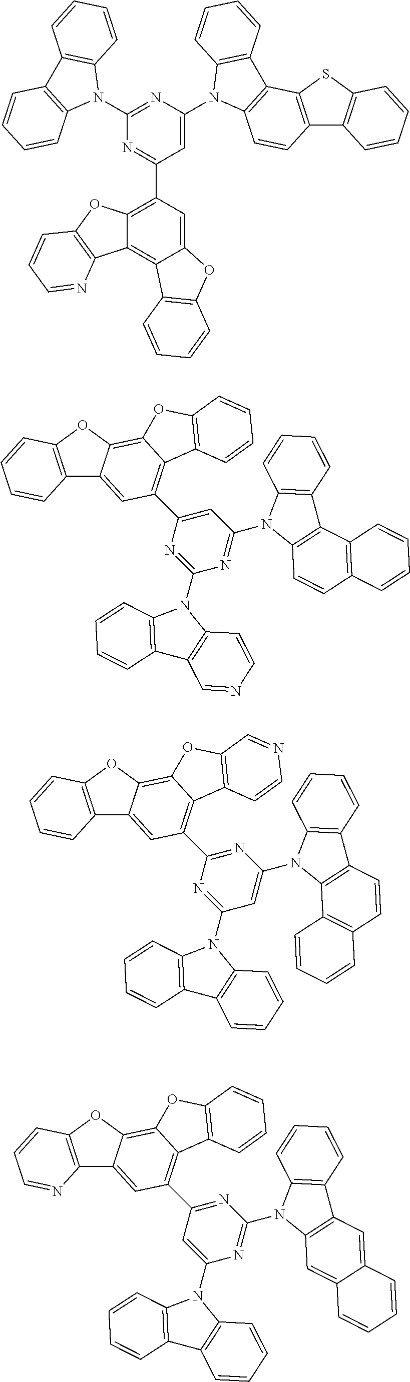

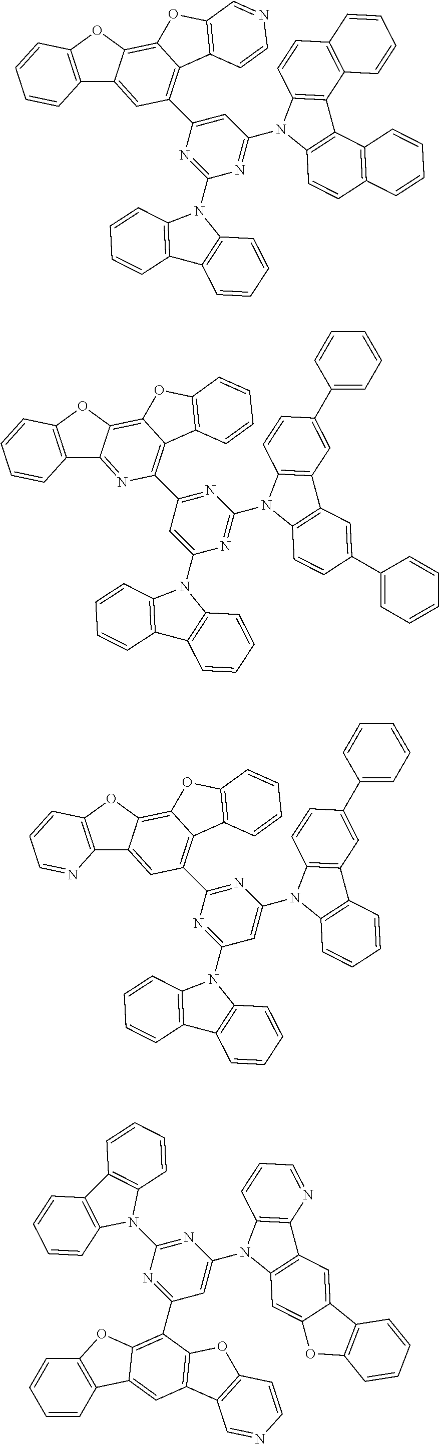 [SMILES: c1ccc2c(c1)ccc1c2c2c3ccccc3ccc2n1-c1cc(-c2cc3c4ccccc4oc3c3oc4cnccc4c23)nc(-n2c3ccccc3c3ccccc32)n1 ; c1ccc(-c2ccc3c(c2)c2cc(-c4ccccc4)ccc2n3-c2nc(-c3nc4c5ccccc5oc4c4oc5ccccc5c34)cc(-n3c4ccccc4c4ccccc43)n2)cc1 ; c1ccc(-c2ccc3c(c2)c2ccccc2n3-c2cc(-n3c4ccccc4c4ccccc43)nc(-c3cc4c5ncccc5oc4c4oc5ccccc5c34)n2)cc1 ; c1ccc2c(c1)oc1cc3c(cc12)c1ncccc1n3-c1cc(-c2c3oc4ccccc4c3cc3c2oc2ccncc23)nc(-n2c3ccccc3c3ccccc32)n1]

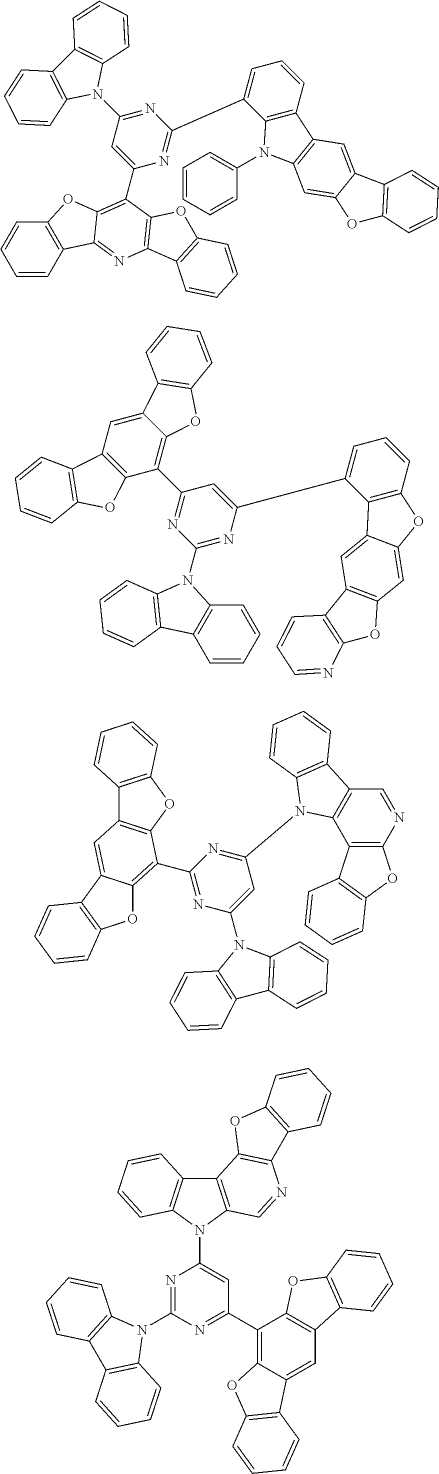

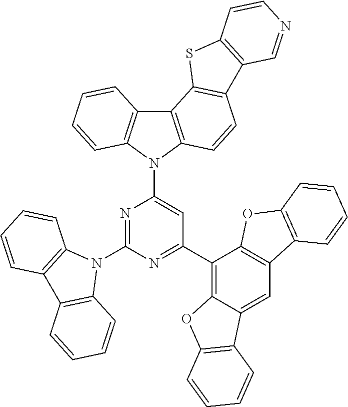 [SMILES: c1ccc2c(c1)oc1c(-c3cc(-n4c5ccccc5c5c6sc7ccncc7c6ccc54)nc(-n4c5ccccc5c5ccccc54)n3)c3oc4ccccc4c3cc12]

In yet another aspect, the present disclosure further provides a polymer comprising at least one repeating unit, where the at least one repeating unit comprises a structure of formula (VI). In some embodiments, the polymer is a non-conjugated polymer in which the structural unit of formula (VI) is on a side chain. In some embodiments, the polymer is a conjugated polymer.

In yet another aspect, the present disclosure further provides another mixture comprising a mixture as described herein, and another organic functional material. The another organic functional material is selected from a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), an organic thermally activated delayed fluorescence material (TADF material), and particularly a luminescent organometallic complex. These organic functional materials are described in detail, for example, in WO2010135519A1, US20090134784A1, and WO2011110277A1. The entire contents of these three documents are hereby incorporated into this document for reference. The another organic functional material can be a small molecule and a polymer.

In some embodiments, the another mixture comprises a mixture as described herein, and a phosphorescent emitter. Herein, the mixture as described herein can be used as a host, and the weight percentage of the phosphorescent emitter ≤20 wt %, preferably ≤15 wt %, more preferably ≤10 wt %.

In some embodiments, the another mixture comprises a mixture as described herein, and a fluorescent emitter. Herein, the mixture as described herein can be used as a fluorescent host material, and the weight percentage of the fluorescent emitter ≤15 wt %, preferably ≤10 wt %, more preferably ≤8 wt %.

In some embodiments, the another mixture comprises a mixture as described herein, a phosphorescent emitter, and a phosphorescent host material. In some embodiments, the mixture as described herein can be used as an auxiliary emitting material, and the weight ratio of the mixture and the phosphorescent emitter ranges from 1:2 to 2:1.

In some embodiments, the another mixture comprises a mixture as described herein, and a TADF material. Herein, the mixture as described herein can be used as a host material of the TADF, and the weight percentage thereof ≤15 wt %, preferably ≤10 wt %, more preferably ≤8 wt %.

The detailed description of the phosphorescent host material, phosphorescent emitting material, fluorescent host material, fluorescent emitting material, and TADF material is described in details in WO2018095395A1. The entire contents of the patent document are hereby incorporated herein for reference.

Another object of the present disclosure is to provide a material for the printed OLEDs.

For this purpose, in the organic compound or the mixture as described herein, the molecular weight of the first compound H1 or the second compound H2≥700 g/mol, preferably ≥800 g/mol, more preferably ≥900 g/mol, further preferably ≥1000 g/mol, and most preferably ≥1100 g/mol.

In some embodiments, the organic compound or mixture as described herein has a solubility of ≥10 mg/mL, preferably ≥15 mg/mL, and most preferably ≥20 mg/mL, in any one solvent selected from toluene, xylene, mesitylene, cyclohexylbenzene, methyl benzoate, or a mixture of two or more of these solvents at 25° C.

In yet another aspect, the present disclosure further provides a formulation or an ink comprising an organic compound or a mixture as described herein, and at least one organic solvent.

The viscosity and surface tension of the ink are important parameters in printing processes. A suitable ink surface tension is required for the specific substrates and the specific printing methods.

In some embodiments, the surface tension of the ink as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In some embodiments, the viscosity of the ink as described herein at 25° C. is in the range of from about 1 cps to 100 cps; preferably in the range of 1 cps to 50 cps; more preferably in the range of 1.5 cps to 20 cps; and most preferably in the range of 4 cps to 20 cps. The resulting formulation will be particularly suitable for ink-jet printing.

The viscosity can be adjusted by different methods, such as by the selection of appropriate solvent and the concentration of the functional materials in the ink. In the ink comprising the mixtures as described herein facilitate the adjustment of the printing ink in the appropriate range according to the printing method used. Generally, in the formulation comprising the functional material as described herein, the weight ratio of the functional material ranges from 0.3 wt % to 30 wt %, preferably in the range of 0.5 wt % to 20 wt %, more preferably in the range of 0.5 wt % to 15 wt %, further preferably in the range of 0.5 wt % to 10 wt %, and most preferably in the range of 1 wt % to 5 wt %.

In some embodiments, the at least one organic solvent of the ink as described herein is selected from aromatic-based or heteroaromatic-based solvents, particular in aliphatic chain/ring substituted aromatic solvents, aromatic ketone solvents, or aromatic ether solvents.

Examples of solvents suitable for the present disclosure include, but not limited to aromatic-based or heteroaromatic-based solvents, such as diisopropylbenzene, phenylpentane, tetraline, cyclohexylbenzene, 1-chloronaphthalene, 1,4-dimethylnaphthalene, 3-isopropylbiphenyl, p-methyl cumene, dipentylbenzene, tripentylbenzene, pentyltoluene, o-xylene, m-xylene, p-xylene, 1,2-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, 1-methoxynaphthalene, 2-methylnaphthalene, 3-isopropylbiphenyl, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine, 4-isopropylbiphenyl, α,α-dichlorodiphenylmethane, 4-(3-phenylpropyl)pyridine, benzyl benzoate, phenyl xylyl ethane, 2-isopropylnaphthalene, benzyl ether, etc; ketone-based solvents, such as 1-tetralone, 2-tetralone, 2-(phenylepoxy)tetralone, 6-(methoxy)tetralone, acetophenone, propiophenone, benzophenone, and derivatives thereof such as 4-methylacetophenone, 3-methylacetophenone, 2-methylacetophenone, 4-methylphenylacetone, 3-methylpropiophenone, 2-methylpropiophenone, isophorone, 2,6,8-trimethyl-4-nonanone, fenchone, 2-nonanone, 3-nonanone, 5-nonanone, 2-decanone, 2,5-hexanedione, phorone, 6-undecanone; aromatic ether solvents, such as 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxan, 1,3-diisopropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene, 1-methoxynaphthalene, diphenyl ether, 2-phenoxymethyl ether, 2-phenoxytetrahydrofuran, 2-ethoxynaphthalene, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc; ester solvent, such as alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc.

Further, in the ink as described herein, the at least one organic solvent can be selected from aliphatic ketones, such as, 2-nonanone, 3-nonanone, 5-nonanone, 2-decanone, 2,5-hexanedione, 2,6,8-trimethyl-4-nonanone, phoron, 6-undecanone, etc; and the at least one organic solvent as described herein can be selected from aliphatic ether, such as, dipentyl ether, hexyl ether, n-octyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, the ink further comprises another organic solvent. Examples of the another organic solvents include, but not limited to: methanol, ethanol, 2-methoxyethanol, dichloromethane, trichloromethane, chlorobenzene, 1,2-dichlorobenzene, tetrahydrofuran, anisole, morpholine, toluene, o-xylene, m-xylene, p-xylene, 1,4-dioxane, acetone, 2-butanone, 1,2-dichloroethane, 3-phenoxytoluene, 1,1,1-trichloroethane, 1,1,2,2-tetrachloroethane, ethyl acetate, butyl acetate, N,N-dimethylformamide, N,N-dimethylacetamide, dimethyl sulfoxide, tetraline, decahydronaphthalene, indene, and/or any combination thereof.

In some embodiments, the formulation as described herein is a solution.

In some embodiments, the formulation as described herein is a dispersion.

The formulation in the embodiments of the present disclosure may comprise the organic compound or the mixture of 0.01 wt % to 20 wt %, preferably 0.1 wt % to 15 wt %, more preferably 0.2 wt % to 10 wt %, and most preferably 0.25 wt % to 5 wt %.

The present disclosure further provides the use of the formulation as coatings or printing inks in the preparation of organic electronic devices, particularly preferably by printing or coating processing methods.

Where suitable printing or coating techniques include, but not limited to, gravure printing, ink-jet printing, nozzle printing, typographic printing, screen printing, dip coating, spin coating, blade coating, roller printing, torsion roll printing, planographic printing, flexographic printing, rotary printing, spray coating, brush coating, pad printing, slit die coating, etc. Preferred techniques are gravure printing, nozzle printing, and ink-jet printing. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information about printing technologies and their requirements for solutions, such as solvent, concentration, and viscosity, and the like, please refer to Handbook of Print Media: Technologies and Production Methods, edited by Helmut Kipphan, ISBN 3-540-67326-1.

In yet another aspect, the present disclosure further provides an application of an organic compound or a mixture as described herein, i.e., the organic compound or the mixture is applied to an organic electronic device, and the organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, an organic plasmon emitting diode (OPED), etc., particularly preferably is an OLED. In the embodiments of the present disclosure, it is preferred to use the mixture or the organic compound for the emissive layer of the OLED device.

In yet another aspect, the present disclosure further provides an organic electronic device comprising an organic compound or a mixture as described herein. In general, such organic electronic device comprises a cathode, an anode, and a functional layer disposed between the cathode and the anode, where the functional layer comprises a mixture as described herein. The organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, an organic plasmon emitting diode (OPED), etc., particularly preferably is an organic electroluminescent device, such as an OLED, an OLEEC, an organic light emitting field effect transistor.

In some embodiments, the organic electronic device is an organic electroluminescent device comprising an emissive layer or an electron-transport layer or a hole-blocking layer. In some embodiments, the organic electroluminescent device comprises an emissive layer, and the emissive layer comprises an organic compound or a mixture as described herein; or comprises an organic compound or a mixture as described herein, and a phosphorescent emitter; or comprises an organic compound or a mixture as described herein, and a host material; or comprises an organic compound or a mixture as described herein, a phosphorescent emitter, and a phosphorescent host material. In some embodiments, the electron-transport layer of the organic electroluminescent device comprises an organic compound or a mixture as described herein. In some embodiments, the hole-blocking layer of the organic electroluminescent device comprises an organic compound or a mixture as described herein.

In the light-emitting device as described herein, in particular an OLED, which comprises a substrate, an anode, at least one emissive layer, and a cathode.

The substrate should be opaque or transparent. A transparent substrate could be used to produce a transparent light-emitting device (for example: Bulovic et al., Nature, 1996, 380, p29, and Gu et al., Appl. Phys. Lett., 1996, 68, p2606). The substrate can be rigid or flexible, e.g. it can be plastic, metal, semiconductor wafer, or glass. Preferably, the substrate has a smooth surface. Particularly ideal are substrates without surface defects. In some embodiments, the substrate is flexible and can be selected from a polymer film or plastic with a glass transition temperature (Tg)>150° C., preferably >200° C., more preferably >250° C., and most preferably >300° C. Examples of the suitable flexible substrates include poly ethylene terephthalate (PET) and polyethylene glycol (2,6-naphthalene) (PEN).

The anode may be a conductive metal, or a metal oxide, or a conductive polymer. The anode should be able to easily inject holes into a hole-injection layer (HIL), a hole-transport layer (HTL), or an emissive layer. In some embodiments, the absolute value of the difference between the work function of the anode and the HOMO energy level/valence band energy level of the emitter of the emissive layer or the p-type semiconductor materials of the hole-injection layer (HIL)/hole-transport layer (HTL)/electron-blocking layer (EBL)<0.5 eV, preferably <0.3 eV, and most preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method including RF magnetron sputtering, vacuum thermal evaporation, e-beam, etc. In some embodiments, the anode is patterned. Patterned conductive ITO substrates are commercially available and can be used to produce the devices as described herein.

The cathode may be a conductive metal or a metal oxide. The cathode should be able to easily inject electrons into the electron-injection layer (EIL), the electron-transport layer (ETL), or the directly into the emissive layer. In some embodiments, the absolute value of the difference between the work function of the cathode and the LUMO energy level/conduction band energy level of the emitter of the emissive layer, or the n-type semiconductor materials of the electron-injection layer (EIL)/electron-transport layer (ETL)/hole-blocking layer (HBL)<0.5 eV, preferably <0.3 eV, and most preferably <0.2 eV. In principle, all materials those can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode materials can be deposited using any suitable technique, such as the suitable physical vapor deposition method including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1. The entire contents of these three documents are hereby incorporated herein for reference.

In some embodiments, the electron-transport layer (ETL) or the hole-blocking layer (HBL) of the organic electroluminescent device as described herein comprises a mixture, an organic compound, or a polymer as described herein. In some embodiments, the emissive layer of the light-emitting device as described herein comprises an emissive layer, which is formed by using the formulation as described herein.

In some embodiments, the emissive layer of the organic electroluminescent device as described herein comprises an organic compound, a mixture, or a polymer as described herein. The emissive layer may preferably be formed by one of the following two methods.

(1) The mixture comprising the first compound H1 and the second compound H2 is deposited as a source. This can be prepared by printing using a formulation as described herein, or by vacuum evaporation deposition using the mixture as a source.

(2) The first compound H1 and the second compound H2 are independently used as two separate sources, and are deposited by vacuum evaporation.

In some embodiments, the molecular weight difference between the first compound H1 and the second compound H2 is small, and its sublimation temperature is also small. In order to simplify the vapor deposition process of the material and reduce the production cost of the OLED display devices, the mixture can be used to prepare the emissive layer by mixing two host materials (i.e., the first compound H1 and the second compound H2) in a certain ratio and then evaporating them through a vapor deposition heat source.

The emission wavelength of the light-emitting device as described herein is between 300 nm and 1000 nm, preferably between 350 nm and 900 nm, more preferably between 400 nm and 800 nm.

In yet another aspect, the present disclosure further provides the applications of organic electronic devices in various electronic equipment, including, but not limited to, display devices, lighting equipments, light sources, sensors, etc.

In yet another aspect, the present disclosure further provides electronic devices comprising organic electronic devices of the present disclosure, including, but not limited to, display devices, lighting equipments, light sources, sensors, etc.

### SPECIFIC EMBODIMENT

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Synthesis of the Second Compound H2 (i.e., the Second Organic Host Material)

### Example 1, Synthesis of Compound 1

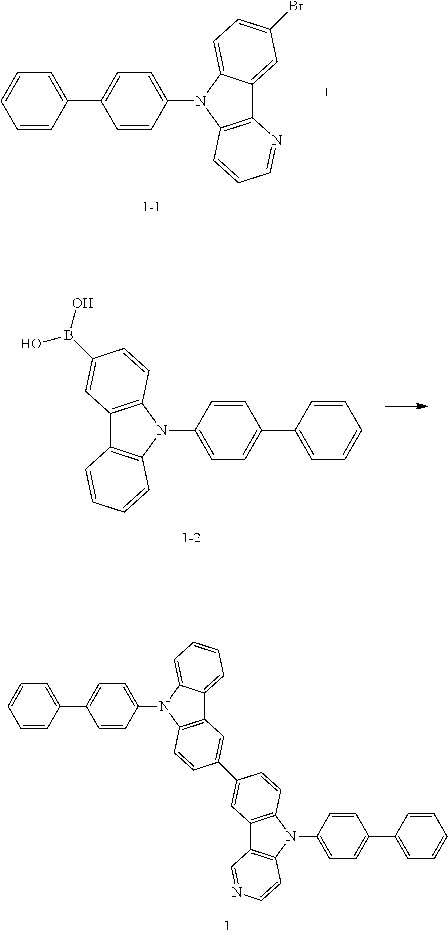 [SMILES: Brc1ccc2c(c1)c1ncccc1n2-c1ccc(-c2ccccc2)cc1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)c5cnccc5n6-c5ccc(-c6ccccc6)cc5)ccc43)cc2)cc1]

1-2 (3.63 g, 10 mmol), 1-1 (3.98 g, 10 mmol), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 5.8 g of compound 1. MS (ASAP)=637.4.

### Example 2, Synthesis of Compound 2

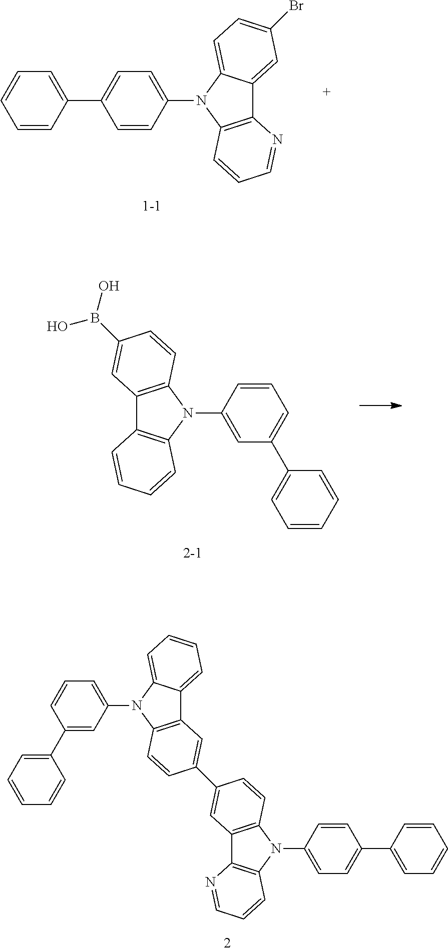 [SMILES: Brc1ccc2c(c1)c1ncccc1n2-c1ccc(-c2ccccc2)cc1 ; OB(O)c1ccc2c(c1)c1ccccc1n2-c1cccc(-c2ccccc2)c1 ; c1ccc(-c2ccc(-n3c4ccc(-c5ccc6c(c5)c5ccccc5n6-c5cccc(-c6ccccc6)c5)cc4c4ncccc43)cc2)cc1]

2-1 (3.63 g, 10 mmol), 1-1 (3.98 g, 10 mmol), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 5.4 g of compound 2. MS (ASAP)=637.8.

### Example 3, Synthesis of Compound 3

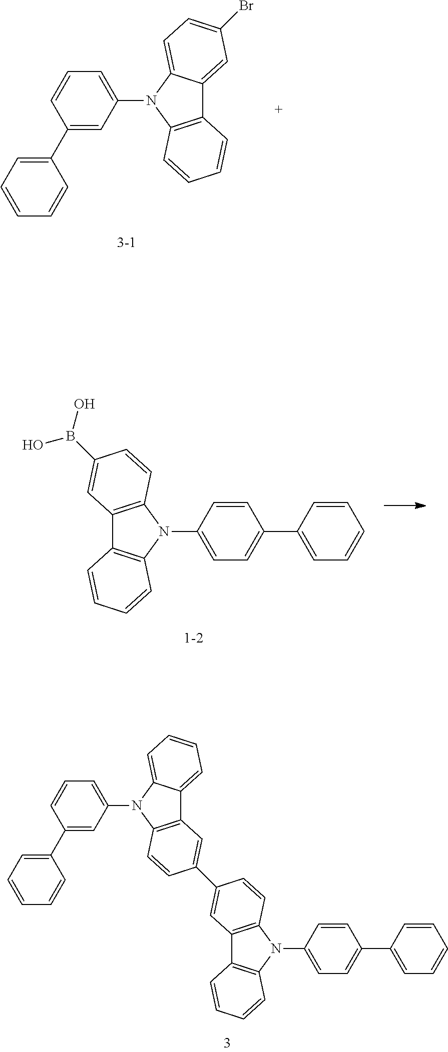 [SMILES: Brc1ccc2c(c1)c1ccccc1n2-c1cccc(-c2ccccc2)c1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

1-2 (3.63 g, 10 mmol), 3-1 (3.98 g, 10 mmol), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 5.4 g of compound 3. MS (ASAP)=636.6.

### Example 4, Synthesis of Compound 4

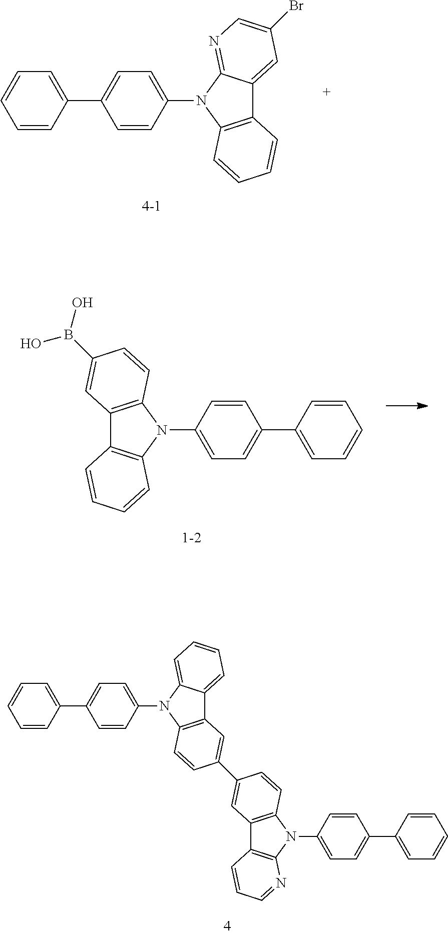 [SMILES: Brc1cnc2c(c1)c1ccccc1n2-c1ccc(-c2ccccc2)cc1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)c5cccnc5n6-c5ccc(-c6ccccc6)cc5)ccc43)cc2)cc1]

1-2 (3.63 g, 10 mmol), 4-1 (3.98 g, 10 mmol), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 5.5 g of compound 4. MS (ASAP)=637.4.

### Example 5, Synthesis of Compound 5

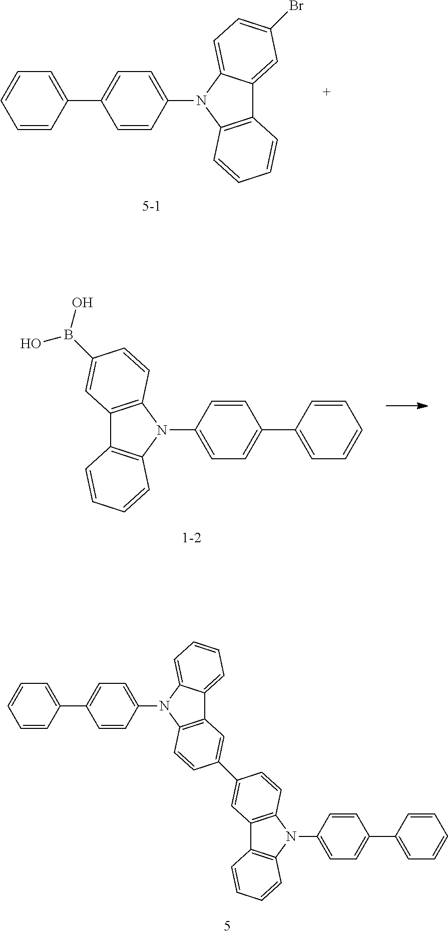 [SMILES: Brc1ccc2c(c1)c1ccccc1n2-c1ccc(-c2ccccc2)cc1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

1-2 (3.63 g, 10 mmol), 5-1 (3.98 g, 10 mmol), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 6.2 g of compound 5. MS (ASAP)=636.2.

### Example 6, Synthesis of Compound 6

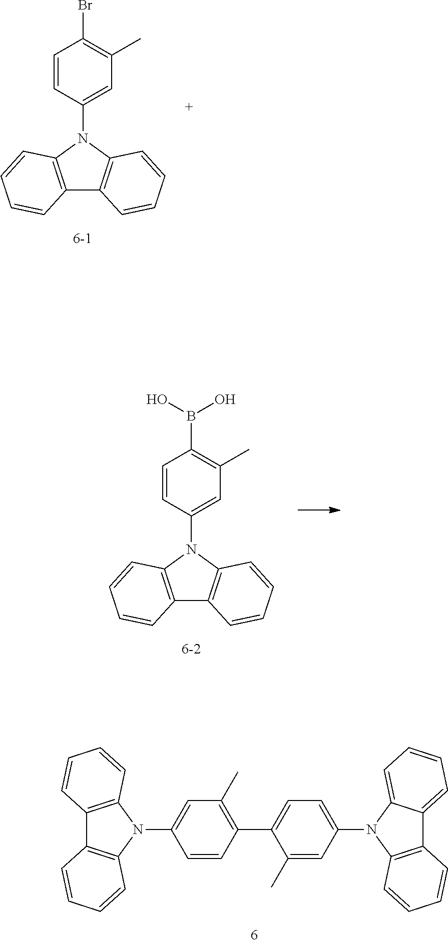 [SMILES: Cc1cc(-n2c3ccccc3c3ccccc32)ccc1Br ; Cc1cc(-n2c3ccccc3c3ccccc32)ccc1B(O)O ; Cc1cc(-n2c3ccccc3c3ccccc32)ccc1-c1ccc(-n2c3ccccc3c3ccccc32)cc1C]

6-1 (3.63 g, 10 mmol), 6-2 (3.98 g, 10 mmol), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 5.2 g of compound 6. MS (ASAP)=484.6.

### Example 7, Synthesis of Compound 7

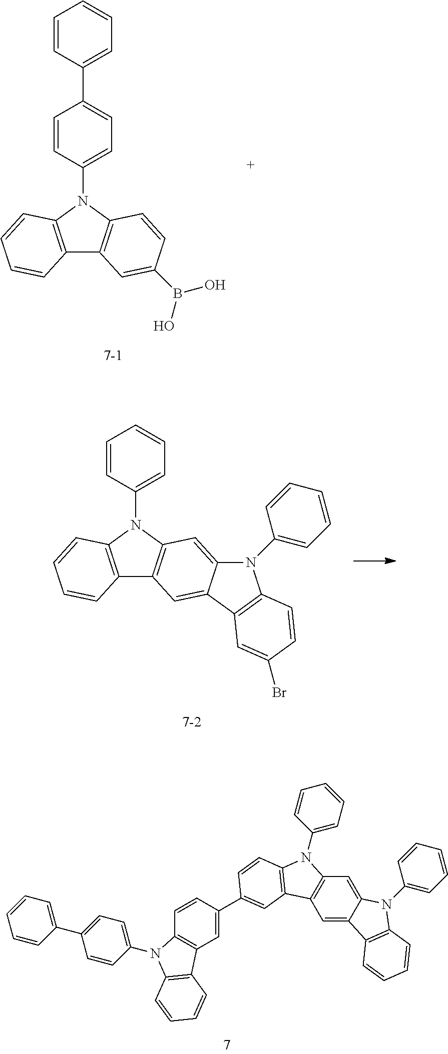 [SMILES: OB(O)c1ccc2c(c1)c1ccccc1n2-c1ccc(-c2ccccc2)cc1 ; Brc1ccc2c(c1)c1cc3c4ccccc4n(-c4ccccc4)c3cc1n2-c1ccccc1 ; c1ccc(-c2ccc(-n3c4ccccc4c4cc(-c5ccc6c(c5)c5cc7c8ccccc8n(-c8ccccc8)c7cc5n6-c5ccccc5)ccc43)cc2)cc1]

7-1 (3.63 g), 7-2 (3.98 g), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 3.2 g of compound 7. MS (ASAP)=725.9.

### Example 8, Synthesis of Compound 8

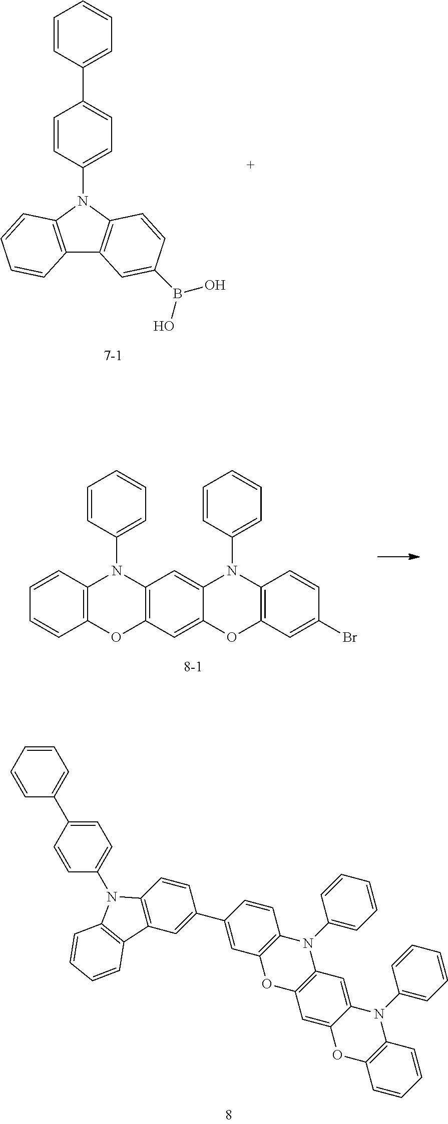 [SMILES: OB(O)c1ccc2c(c1)c1ccccc1n2-c1ccc(-c2ccccc2)cc1 ; Brc1ccc2c(c1)oc1cc3oc4ccccc4n(-c4ccccc4)c3cc1n2-c1ccccc1 ; c1ccc(-c2ccc(-n3c4ccccc4c4cc(-c5ccc6c(c5)oc5cc7oc8ccccc8n(-c8ccccc8)c7cc5n6-c5ccccc5)ccc43)cc2)cc1]

7-1 (3.63 g), 8-1 (3.98 g), potassium carbonate (6.9 g, 50 mmol), Pd(PPh₃)₄ (0.58 g, 0.5 mmol), toluene (100 mL), water (25 mL), and ethanol (25 mL) were added in turn to a 250 mL three-necked flask under a nitrogen atmosphere, then the mixture was heated to 110° C. Monitored by TLC, after the reaction was completed, the reaction solution was cooled to room temperature. The reaction solution was poured into water and washed to remove K₂CO₃. After the filtration, the obtained solid product was washed with dichloromethane, then the obtained crude product was recrystallized with dichloromethane and methanol to yield 4.3 g of compound 8. MS (ASAP)=757.9.

### Synthesis of the First Compound H1 (i.e., the First Organic Host Material)

### Synthesis of Intermediate 1b:

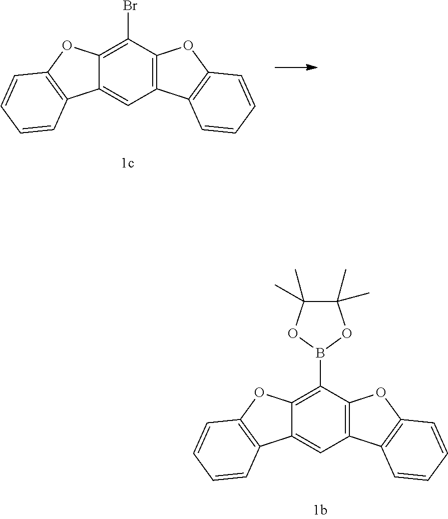 [SMILES: Brc1c2oc3ccccc3c2cc2c1oc1ccccc12 ; CC1(C)OB(c2c3oc4ccccc4c3cc3c2oc2ccccc23)OC1(C)C]

1c (18 g), bis(pinacolato)diboron (20 g), potassium acetate (15 g), catalyst Pd(dppf)Cl₂ (1 g), and toluene (300 mL) were added to a dry three-necked flask under a nitrogen atmosphere. After heating up to 100° C., the resulting mixture was stirred and refluxed for 24 h. After the reaction was completed, the resulting sample was separated by column chromatography (petroleum ether:ethyl acetate=25:1) to yield 19.5 g (yield: 89%) of intermediate 1b. The molecular ion mass was determined to be 384 (calculated value: 384.24) by mass spectrometry.

### Synthesis of Intermediate 4b:

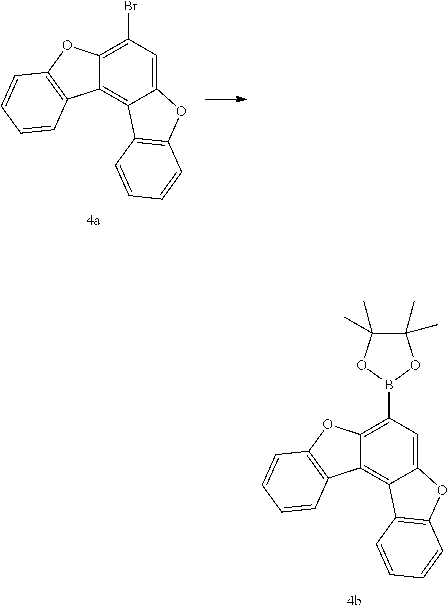 [SMILES: Brc1cc2oc3ccccc3c2c2c1oc1ccccc12 ; CC1(C)OB(c2cc3oc4ccccc4c3c3c2oc2ccccc23)OC1(C)C]

4a (20 g), bis(pinacolato)diboron (20 g), potassium acetate (15 g), catalyst Pd(dppf)Cl₂ (1 g), and toluene (300 mL) were added to a dry three-necked flask under a nitrogen atmosphere. After heating up to 100° C., the resulting mixture was stirred and refluxed for 24 h. After the reaction was completed, the resulting sample was separated by column chromatography (petroleum ether:ethyl acetate=25:1) to yield 16 g (yield: 75%) of intermediate 4b. The molecular ion mass was determined to be 384 (calculated value: 384.24) by mass spectrometry.

### Synthesis of Intermediate 5b:

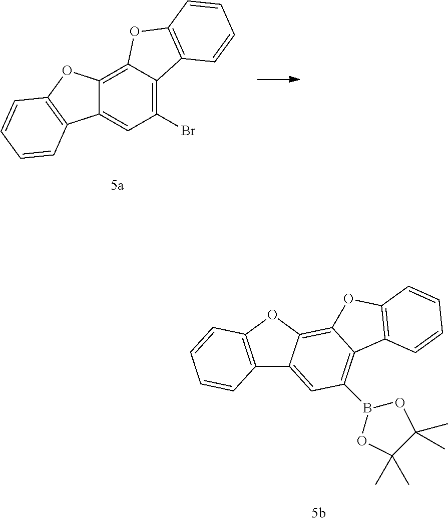 [SMILES: Brc1cc2c3ccccc3oc2c2oc3ccccc3c12 ; CC1(C)OB(c2cc3c4ccccc4oc3c3oc4ccccc4c23)OC1(C)C]

5a (20 g), bis(pinacolato)diboron (20 g), potassium acetate (15 g), catalyst Pd(dppf)Cl₂ (1 g), and toluene (300 mL) were added to a dry three-necked flask under a nitrogen atmosphere. After heating up to 100° C., the resulting mixture was stirred and refluxed for 24 h. After the reaction was completed, the resulting sample was separated by column chromatography (petroleum ether:ethyl acetate=25:1) to yield 14.5 g (yield: 68%) of intermediate 5b. The molecular ion mass was determined to be 384 (calculated value: 384.24) by mass spectrometry.

### Synthesis of Intermediate 13b:

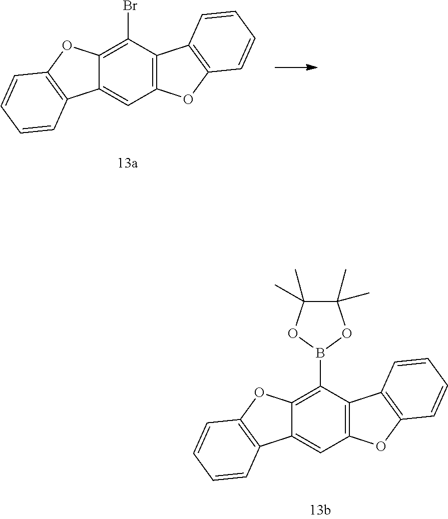 [SMILES: Brc1c2oc3ccccc3c2cc2oc3ccccc3c12 ; CC1(C)OB(c2c3oc4ccccc4c3cc3oc4ccccc4c23)OC1(C)C]

13a (18 g), bis(pinacolato)diboron (20 g), potassium acetate (15 g), catalyst Pd(dppf)Cl₂ (1 g), and toluene (300 mL) were added to a dry three-necked flask under a nitrogen atmosphere. After heating up to 100° C., the resulting mixture was stirred and refluxed for 24 h. After the reaction was completed, the resulting sample was separated by column chromatography (petroleum ether:ethyl acetate=25:1) to yield 20.1 g (yield: 91%) of intermediate 13b. The molecular ion mass was determined to be 384 (calculated value: 384.24) by mass spectrometry.

### Synthesis of Intermediate 19b:

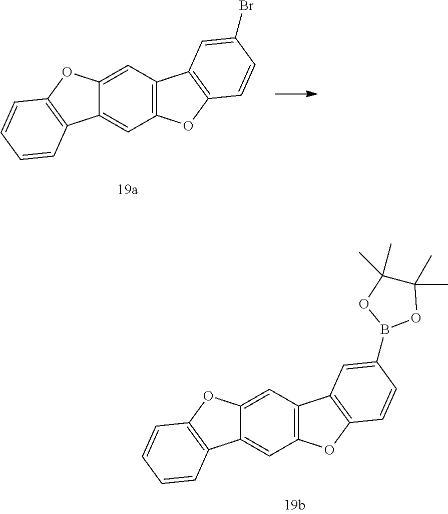 [SMILES: Brc1ccc2oc3cc4c(cc3c2c1)oc1ccccc14 ; CC1(C)OB(c2ccc3oc4cc5c(cc4c3c2)oc2ccccc25)OC1(C)C]

19a (18 g), bis(pinacolato)diboron (20 g), potassium acetate (15 g), catalyst Pd(dppf)Cl₂ (1 g), and toluene (300 mL) were added to a dry three-necked flask under a nitrogen atmosphere. After heating up to 100° C., the resulting mixture was stirred and refluxed for 24 h. After the reaction was completed, the resulting sample was separated by column chromatography (petroleum ether:ethyl acetate=25:1) to yield 20.1 g (yield: 91%) of intermediate 19b. The molecular ion mass was determined to be 384 (calculated value: 384.24) by mass spectrometry.

### Example 9, Synthesis of Compound 9

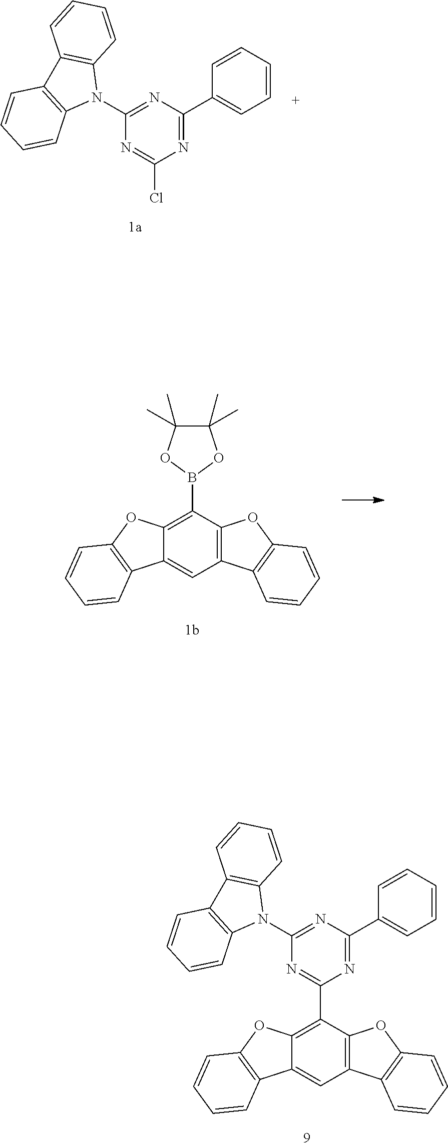 [SMILES: Clc1nc(-c2ccccc2)nc(-n2c3ccccc3c3ccccc32)n1 ; CC1(C)OB(c2c3oc4ccccc4c3cc3c2oc2ccccc23)OC1(C)C ; c1ccc(-c2nc(-c3c4oc5ccccc5c4cc4c3oc3ccccc34)nc(-n3c4ccccc4c4ccccc43)n2)cc1]

1a (30 g), intermediate 1b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 28 g of the crude product (compound 9). After that, the resulting sample was separated by column chromatography (petroleum ether:ethyl acetate=15:1) to yield 25.7 g (yield: 87%) of compound 9. The molecular ion mass was determined to be 579 (calculated value: 578.63) by mass spectrometry.

### Example 10, Synthesis of Compound 10

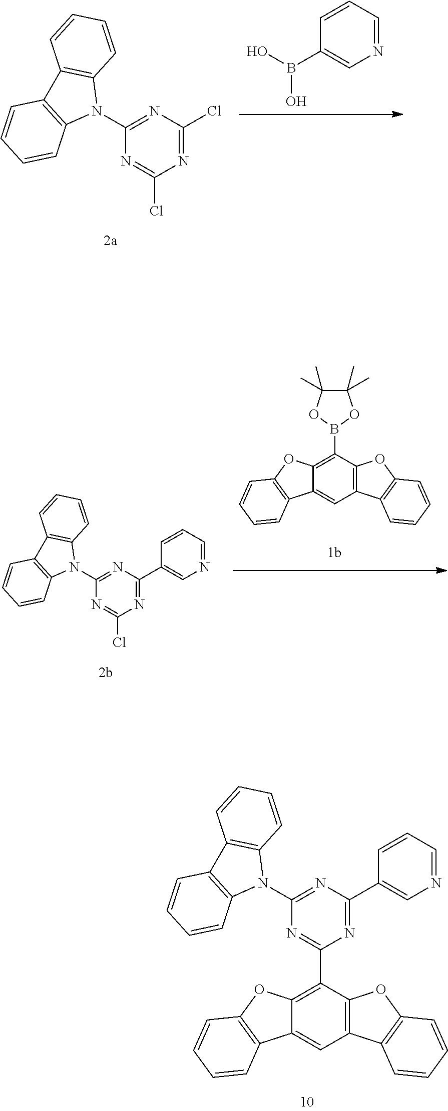 [SMILES: Clc1nc(Cl)nc(-n2c3ccccc3c3ccccc32)n1 ; OB(O)c1cccnc1 ; Clc1nc(-c2cccnc2)nc(-n2c3ccccc3c3ccccc32)n1 ; CC1(C)OB(c2c3oc4ccccc4c3cc3c2oc2ccccc23)OC1(C)C ; c1cncc(-c2nc(-c3c4oc5ccccc5c4cc4c3oc3ccccc34)nc(-n3c4ccccc4c4ccccc43)n2)c1]

2a (30 g), m-pyridineboronic acid (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 25 g of the crude product (2b). After that, the resulting sample was separated by column chromatography (petroleum ether:ethyl acetate=15:1) to yield 22.6 g (yield: 70%) of 2b. The molecular ion mass was determined to be 358 (calculated value: 357.8) by mass spectrometry.

2b (30 g), intermediate 1b (35 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 28 g of the crude product (compound 10). After that, the resulting sample was separated by column chromatography (petroleum ether:ethyl acetate=15:1) to yield 25.8 g (yield: 87%) of compound 10. The molecular ion mass was determined to be 579 (calculated value: 579.6) by mass spectrometry.

### Example 11, Synthesis of Compound 11

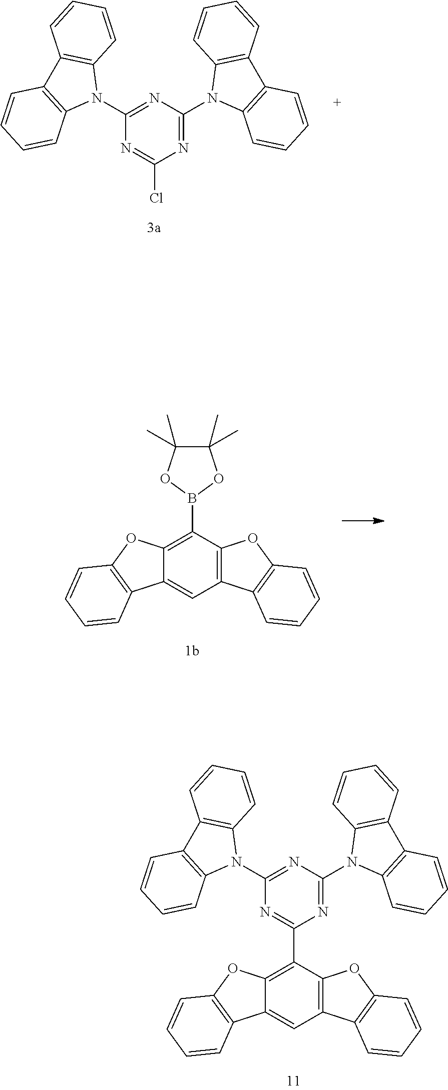 [SMILES: Clc1nc(-n2c3ccccc3c3ccccc32)nc(-n2c3ccccc3c3ccccc32)n1 ; CC1(C)OB(c2c3oc4ccccc4c3cc3c2oc2ccccc23)OC1(C)C ; c1ccc2c(c1)oc1c(-c3nc(-n4c5ccccc5c5ccccc54)nc(-n4c5ccccc5c5ccccc54)n3)c3oc4ccccc4c3cc12]

3a (30 g), intermediate 1b (35 g), potassium (25 g), carbonate tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 27.3 g of the crude product (compound 11). After that, the resulting sample was separated by column chromatography (petroleum ether:ethyl acetate=15:1) to yield 26.5 g (yield: 85%) of compound 11. The molecular ion mass was determined to be 667 (calculated value: 667.73) by mass spectrometry.

### Example 12, Synthesis of Compound 12

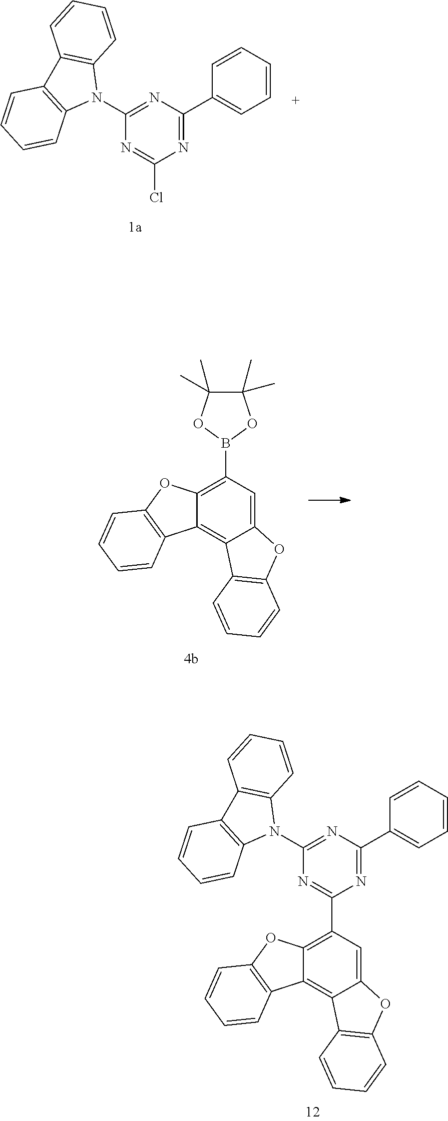 [SMILES: Clc1nc(-c2ccccc2)nc(-n2c3ccccc3c3ccccc32)n1 ; CC1(C)OB(c2cc3oc4ccccc4c3c3c2oc2ccccc23)OC1(C)C ; c1ccc(-c2nc(-c3cc4oc5ccccc5c4c4c3oc3ccccc34)nc(-n3c4ccccc4c4ccccc43)n2)cc1]

1a (30 g), intermediate 4b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 26 g of the crude product (compound 12). After that, the resulting sample was separated by column chromatography (petroleum ether:ethyl acetate=15:1) to yield 23.6 g (yield: 82%) of compound 12. The molecular ion mass was determined to be 579 (calculated value: 578.63) by mass spectrometry.

### Example 13, Synthesis of Compound 13

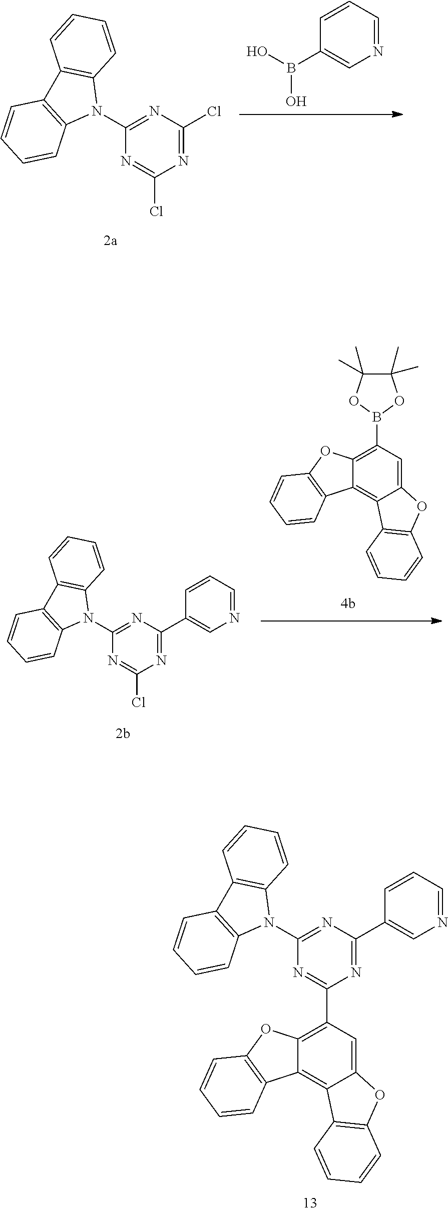 [SMILES: Clc1nc(Cl)nc(-n2c3ccccc3c3ccccc32)n1 ; OB(O)c1cccnc1 ; Clc1nc(-c2cccnc2)nc(-n2c3ccccc3c3ccccc32)n1 ; CC1(C)OB(c2cc3oc4ccccc4c3c3c2oc2ccccc23)OC1(C)C ; c1cncc(-c2nc(-c3cc4oc5ccccc5c4c4c3oc3ccccc34)nc(-n3c4ccccc4c4ccccc43)n2)c1]

2a (30 g), m-pyridineboronic acid (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 25 g of the crude product (2b). After that, the resulting sample was separated by column chromatography (petroleum ether:ethyl acetate=15:1) to yield 22.6 g (yield: 70%) of 2b. The molecular ion mass was determined to be 358 (calculated value: 357.8) by mass spectrometry.

2b (30 g), intermediate 4b (35 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 27 g of the crude product (compound 13). After that, the resulting sample was separated by column chromatography (petroleum ether:ethyl acetate=15:1) to yield 25.4 g (yield: 86%) of compound 13. The molecular ion mass was determined to be 579 (calculated value: 579.6) by mass spectrometry.

### Example 14, Synthesis of Compound 14

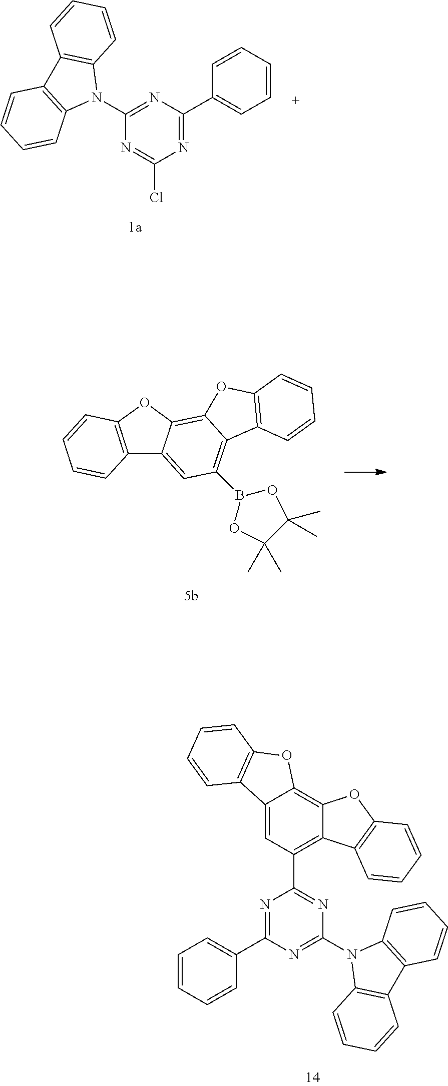 [SMILES: Clc1nc(-c2ccccc2)nc(-n2c3ccccc3c3ccccc32)n1 ; CC1(C)OB(c2cc3c4ccccc4oc3c3oc4ccccc4c23)OC1(C)C ; c1ccc(-c2nc(-c3cc4c5ccccc5oc4c4oc5ccccc5c34)nc(-n3c4ccccc4c4ccccc43)n2)cc1]

1a (30 g), intermediate 5b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 25 g of the crude product (compound 14). After that, the resulting sample was separated by column chromatography (petroleum ether:ethyl acetate=15:1) to yield 23.3 g (yield: 80%) of compound 14. The molecular ion mass was determined to be 579 (calculated value: 578.63) by mass spectrometry.

### Example 15, Synthesis of Compound 15

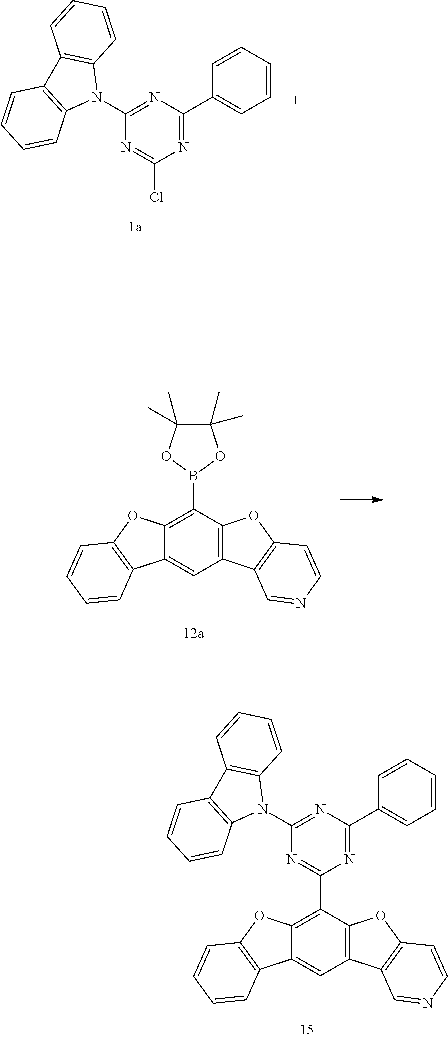 [SMILES: Clc1nc(-c2ccccc2)nc(-n2c3ccccc3c3ccccc32)n1 ; CC1(C)OB(c2c3oc4ccccc4c3cc3c2oc2ccncc23)OC1(C)C ; c1ccc(-c2nc(-c3c4oc5ccccc5c4cc4c3oc3ccncc34)nc(-n3c4ccccc4c4ccccc43)n2)cc1]

1a (30 g), 12a (30 g), potassium carbonate (25 g), tetrakis(triphenylphosphine)palladium (1 g), and 300 mL of toluene/50 mL of ethanol/50 ml of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 22 g of the crude product (compound 15). After that, the resulting sample was separated by column chromatography (petroleum ether:ethyl acetate=15:1) to yield 21.1 g (yield: 72%) of compound 15. The molecular ion mass was determined to be 580 (calculated value: 579.62) by mass spectrometry.

### Example 16, Synthesis of Compound 16

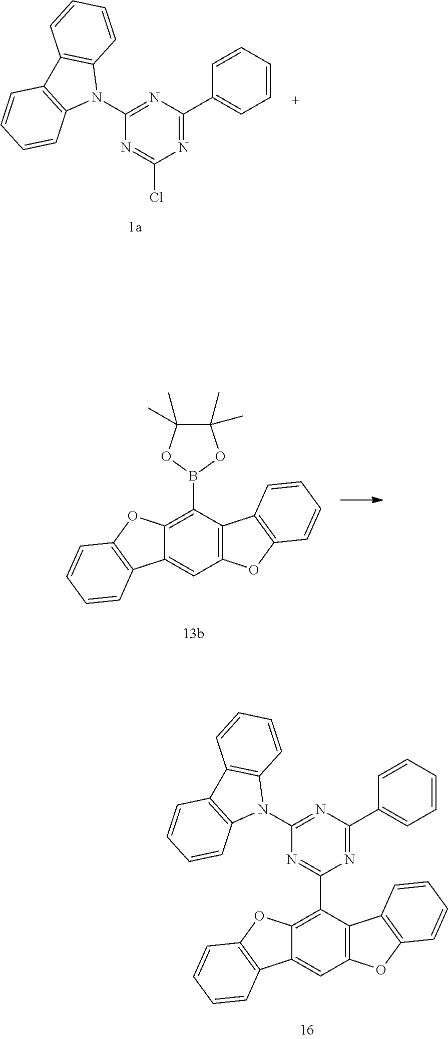 [SMILES: Clc1nc(-c2ccccc2)nc(-n2c3ccccc3c3ccccc32)n1 ; CC1(C)OB(c2c3oc4ccccc4c3cc3oc4ccccc4c23)OC1(C)C ; c1ccc(-c2nc(-c3c4oc5ccccc5c4cc4oc5ccccc5c34)nc(-n3c4ccccc4c4ccccc43)n2)cc1]

1a (30 g), intermediate 13b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 27 g of the crude product (compound 16). After that, the resulting sample was separated by column chromatography (petroleum ether:ethyl acetate=15:1) to yield 24.6 g (yield: 85%) of compound 16. The molecular ion mass was determined to be 579 (calculated value: 578.63) by mass spectrometry.

### Example 17, Synthesis of Compound 17

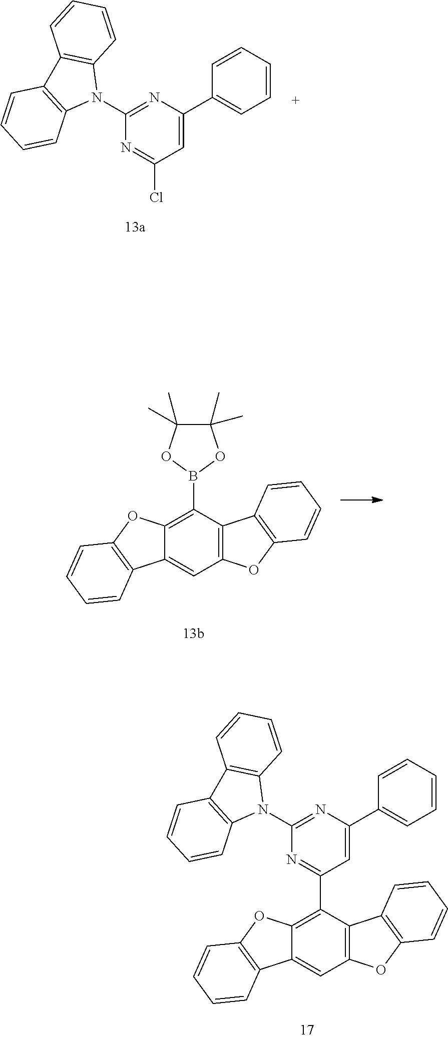 [SMILES: Clc1cc(-c2ccccc2)nc(-n2c3ccccc3c3ccccc32)n1 ; CC1(C)OB(c2c3oc4ccccc4c3cc3oc4ccccc4c23)OC1(C)C ; c1ccc(-c2cc(-c3c4oc5ccccc5c4cc4oc5ccccc5c34)nc(-n3c4ccccc4c4ccccc43)n2)cc1]

13a (30 g), intermediate 13b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 24 g of the crude product (compound 17). After that, the resulting sample was separated by column chromatography (petroleum ether:ethyl acetate=15:1) to yield 23.2 g (yield: 80%) of compound 17. The molecular ion mass was determined to be 578 (calculated value: 577.64) by mass spectrometry.

### Example 18, Synthesis of Compound 18

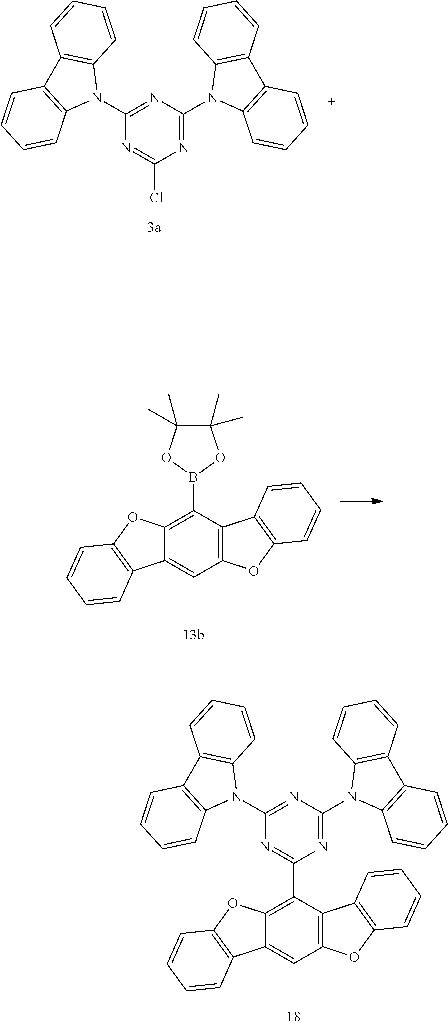 [SMILES: Clc1nc(-n2c3ccccc3c3ccccc32)nc(-n2c3ccccc3c3ccccc32)n1 ; CC1(C)OB(c2c3oc4ccccc4c3cc3oc4ccccc4c23)OC1(C)C ; c1ccc2c(c1)oc1c(-c3nc(-n4c5ccccc5c5ccccc54)nc(-n4c5ccccc5c5ccccc54)n3)c3c(cc12)oc1ccccc13]

3a (30 g), intermediate 13b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 27 g of the crude product (compound 18). After that, the resulting sample was separated by column chromatography (petroleum ether:ethyl acetate=15:1) to yield 21 g (yield: 72%) of compound 18. The molecular ion mass was determined to be 667 (calculated value: 667) by mass spectrometry.

### Example 19, Synthesis of Compound 19

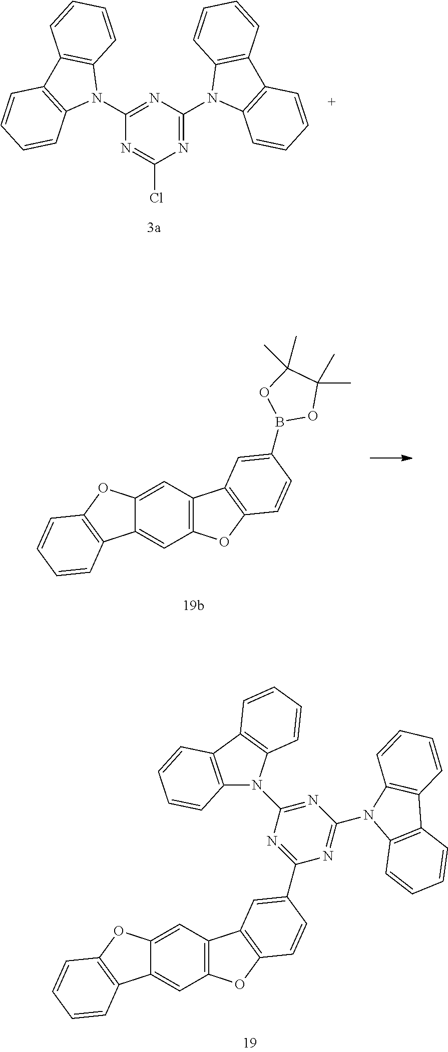 [SMILES: Clc1nc(-n2c3ccccc3c3ccccc32)nc(-n2c3ccccc3c3ccccc32)n1 ; CC1(C)OB(c2ccc3oc4cc5c(cc4c3c2)oc2ccccc25)OC1(C)C ; c1ccc2c(c1)oc1cc3c(cc12)oc1ccc(-c2nc(-n4c5ccccc5c5ccccc54)nc(-n4c5ccccc5c5ccccc54)n2)cc13]

3a (30 g), 19b (30 g), potassium carbonate (25 g), tetrakis(triphenylphosphine)palladium (1 g), and 300 mL of toluene/50 mL of ethanol/50 ml of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 25 g of the crude product (compound 19). After that, the resulting sample was separated by column chromatography (petroleum ether:ethyl acetate=15:1) to yield 20 g (yield: 67%) of compound 19. The molecular ion mass was determined to be 667 (calculated value: 667) by mass spectrometry.

### Example 20, Synthesis of Compound 20

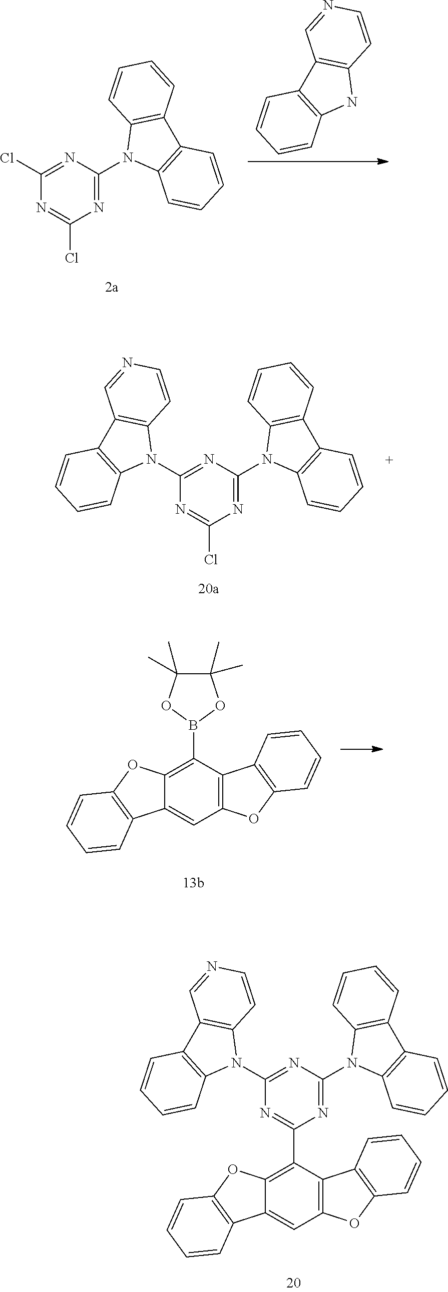 [SMILES: Clc1nc(Cl)nc(-n2c3ccccc3c3ccccc32)n1 ; c1ccc2c(c1)[nH]c1ccncc12 ; Clc1nc(-n2c3ccccc3c3ccccc32)nc(-n2c3ccccc3c3cnccc32)n1 ; CC1(C)OB(c2c3oc4ccccc4c3cc3oc4ccccc4c23)OC1(C)C ; c1ccc2c(c1)oc1c(-c3nc(-n4c5ccccc5c5ccccc54)nc(-n4c5ccccc5c5cnccc54)n3)c3c(cc12)oc1ccccc13]

2a (30 g), aza-carbazole (40 g), sodium tert-butoxide (10 g), and 300 mL of DMF were added to a three-necked flask under a nitrogen atmosphere. After heating up to 100° C., the resulting mixture was reacted for 12 h and then cooled down. After the extraction with ethyl acetate and deionized-water, the organic phase was washed with water two times, dried, filtered, and the solvent was removed to obtain 40 g of the crude product (20a). After that, the resulting sample was separated by column chromatography (petroleum ether:ethyl acetate=15:1) to yield 20 g (yield: 50%) of 20a.

20a (30 g), intermediate 13b (30 g), potassium carbonate (25 g), tetrakis(triphenylphosphine)palladium (1 g), and 300 mL of toluene/50 mL of ethanol/50 mL of water were added to a three-necked flask under a nitrogen atmosphere. After heating up to 100° C., the resulting mixture was refluxed for 12 h and then cooled down. After the extraction with ethyl acetate and deionized-water, the organic phase was washed with water two times, dried, filtered, and the solvent was removed to obtain 27 g of the crude product (compound 20). After that, the resulting sample was separated by column chromatography (petroleum ether:ethyl acetate=15:1) to yield 15 g (yield: 60%) of compound 20. The molecular ion mass was determined to be 668 (calculated value: 668) by mass spectrometry.

Preparation process of the mixture: the first organic host material and the second organic host material were first mixed as uniformly as possible at a mass ratio of 1:1. The resulting mixture was placed in a vacuum environment of ≤10-3 Torr, and the temperature of the vacuum environment was increased until the two host materials completely melted. After thorough mixing, the resulting mixture was cooled to room temperature to allow solidification, then ground into powder with a ball mill for further use.

The energy level of the organic compound can be calculated by quantum computation, for example, using TD-DFT (time-dependent density functional theory) by Gaussian 09W (Gaussian Inc.), the specific simulation methods of which can be found in CN110892543B. Firstly, the molecular geometry is optimized by semi-empirical method “Ground State/Semi-empirical/Default Spin/AM1” (Charge 0/Spin Singlet), then the energy structure of organic molecules is calculated by TD-DFT (time-dependent density functional theory) “TD-SCF/DFT/Default Spin/B3 PW91” and the basis set “6-31G (d)” (Charge 0/Spin Singlet). The HOMO and LUMO energy levels are calculated using the following calibration formula, where E_{S1} and E_{T1} are used directly.

$$HOMO ( eV ) = ( ( HOMO ( G ) \times 27.212 ) - 0.9899 ) / 1.1206$$

$$LUMO ( eV ) = ( ( LUMO ( G ) \times 27.212 ) - 2.0041 ) / 1.385$$

Where HOMO(G) and LUMO(G) are the direct calculation results of Gaussian 09W, in units of Hartree. The results are shown in Table 1 below:

TABLE 1

| | HOMO | LUMO | ΔHOMO | E_{T1} | E_{S1} |
|---|---|---|---|---|---|
| Material | [eV] | [eV] | [eV] | [eV] | [eV] |
| compound 1 | −5.468 | −2.287 | 0.460 | 2.9548 | 3.5456 |
| compound 2 | −5.474 | −2.282 | 0.454 | 2.9638 | 3.5531 |
| compound 3 | −5.412 | −2.219 | 0.454 | 2.9753 | 3.5674 |
| compound 4 | −5.529 | −2.269 | 0.435 | 2.9550 | 3.5495 |
| compound 5 | −5.423 | −2.235 | 0.436 | 2.9652 | 3.5488 |
| compound 6 | −5.774 | −2.021 | 0.002 | 3.3284 | 4.0476 |
| compound 7 | −5.266 | −2.205 | 0.291 | 2.7542 | 3.4985 |
| compound 8 | −4.586 | −2.216 | 0.798 | 2.3193 | 2.8362 |
| compound 9 | −6.104 | −2.888 | 0.056 | 2.7792 | 3.2949 |
| compound 10 | −6.170 | −2.995 | 0.032 | 2.7659 | 3.2158 |
| compound 11 | −6.102 | −2.896 | 0.067 | 2.7418 | 3.3615 |
| compound 12 | −6.124 | −3.061 | 0.081 | 2.5743 | 3.1151 |
| compound 13 | −6.207 | −3.153 | 0.076 | 2.5389 | 3.0705 |
| compound 14 | −6.152 | −2.929 | 0.060 | 2.8113 | 3.2871 |
| compound 15 | −6.178 | −2.981 | 0.048 | 2.829 | 3.255 |
| compound 16 | −6.153 | −2.969 | 0.037 | 2.6046 | 3.2453 |
| compound 17 | −5.916 | −2.875 | 0.135 | 2.653 | 3.1311 |
| compound 18 | −6.158 | −2.947 | 0.038 | 2.5902 | 3.3495 |
| compound 19 | −6.168 | −2.806 | 0.053 | 2.7683 | 3.5153 |
| compound 20 | −6.291 | −2.978 | 0.032 | 2.6423 | 3.4450 |

Among them, compound 1-compound 8 can be used in the second organic host material, while compound 9-compound 20 can be used in the first organic host material. The mixture designations and their corresponding compositions are presented in Table 2, with all mixtures containing the first organic host material and second organic host material in a 1:1 mass ratio.

TABLE 2

| | | | | | | | | | | | | | | | | | | | |
|---|---|---|---|---|---|---|---|---|---|---|---|---|---|---|---|---|---|---|---|
| 1 | 2 | 3 | 4 | 5 | 6 | 7 | 8 | 9 | 10 | 11 | 12 | 13 | 14 | 15 | 16 | 17 | 18 | 19 | 20 |

| | | | | | | | | | | | | | | | | | | | |
|---|---|---|---|---|---|---|---|---|---|---|---|---|---|---|---|---|---|---|---|
| A-1 | ∘ | | | | | | | | | | | | ∘ | | | | | | |
| A-2 | | ∘ | | | | | | | ∘ | | | | | | | | | | |
| A-3 | | ∘ | | | | | | | | ∘ | | | | | | | | | |
| A-4 | | | ∘ | | | | | | ∘ | | | | | | | | | | |
| A-5 | | | | ∘ | | | | | | | ∘ | | | | | | | | |
| A-6 | ∘ | | | | | | | | | | ∘ | | | | | | | | |
| A-7 | | | ∘ | | | | | | | ∘ | | | | | | | | | |
| A-8 | | ∘ | | | | | | | | | ∘ | | | | | | | | |
| A-9 | | | | | ∘ | | | | | | | ∘ | | | | | | | |
| A-10 | | | | | ∘ | | | | | | | | ∘ | | | | | | |
| A-11 | | | | | ∘ | | | | | | | | | ∘ | | | | | |
| A-12 | ∘ | | | | | | | | | | | | ∘ | | | | | | |
| A-13 | ∘ | | | | | | | | | | | | | ∘ | | | | | |
| A-14 | | | | ∘ | | | | | | | | ∘ | | | | | | | |
| A-15 | | | ∘ | | | | | | | | | | ∘ | | | | | | |
| A-16 | | | | ∘ | | | | | | | | | | | ∘ | | | | |
| A-17 | | ∘ | | | | | | | | | | | | | | ∘ | | | |
| A-18 | | | ∘ | | | | | | | | | | | | ∘ | | | | |
| A-19 | | | ∘ | | | | | | | | | | | | | ∘ | | | |
| A-20 | | | | | | ∘ | | | ∘ | | | | | | | | | | |
| A-21 | | | | | | | ∘ | | ∘ | | | | | | | | | | |
| A-22 | | | | | | | | ∘ | ∘ | | | | | | | | | | |
| A-23 | ∘ | | | | | | | | | | | | | | | | ∘ | | |
| A-24 | | | | ∘ | | | | | | | | | | | | | | ∘ | |
| A-25 | | ∘ | | | | | | | | | | | | | | | | | ∘ |

The host material of the commonly used carbazole-based material structure system is labeled as Ref 1 for comparison with the aforementioned mixed phosphorescent host materials.

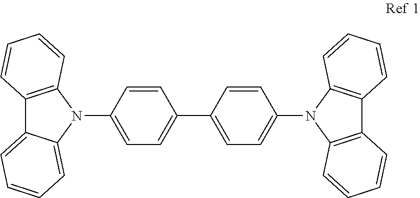 [SMILES: c1ccc2c(c1)c1ccccc1n2-c1ccc(-c2ccc(-n3c4ccccc4c4ccccc43)cc2)cc1]

### Preparation of OLEDs:

The OLEDs were fabricated based on the device structure of ITO/NPB:HATCN(3 wt %) (10 nm)/NPB(50 nm)/TCTA(40 nm)/(A-1)~(A-25):5 wt % Ir(ppy)₃/B3PYMPM(40 nm)/LiQ(2 nm)/Al(100 nm), and the preparation steps are as follows:
Cleaning of the conductive glass substrate: prior to first-time use, the substrates are washed with various solvents (such as chloroform, ketone, or isopropyl alcohol), and then treated with UV and ozone.
b. HIL (10 nm), HTL (50 nm), Prime (40 nm), EML (35 nm), ETL (40 nm): thermal evaporation deposition in high vacuum (1×10⁻⁶ mbar).
c. Cathode: thermal evaporation deposition in high vacuum (1×10⁻⁶ mbar) with LiQ/Al (2 nm/100 nm).
d. Encapsulation: encapsulating the device in a nitrogen-regulated glove box with UV curable resin.

The current-voltage (J-V) characteristics of each OLED were studied. The current efficiency, device lifetime, and the external quantum efficiency were summarized. As shown by testing, the luminescence efficiency and lifetime of OLED4 (corresponding to the mixed host (A-4)) are more than three times that of OLED (corresponding to the mixed host (Ref1)). The luminescence efficiency of OLED10 (corresponding to the mixed host (A-10)) is more than three times that of OLED Ref1, while its lifetime is more than eight times longer. In particular, the maximum external quantum efficiency of OLED10 reaches over 20%. The results clearly show that OLEDs fabricated with the mixture exhibit significantly enhanced luminescence efficiency, extended lifetime, and markedly improved external quantum efficiency.

Furthermore, compound 9, compound 11, and compound 14 were also used as single hosts to fabricate OLEDs, showing over 2.5 times higher efficiency and lifetime than that of OLED Ref1. However, the co-host can achieve better performance in this device structure.

It is to be understood that the application of the present disclosure is not limited to the above-mentioned examples. Those skilled in the art may make improvements or modifications based on the above description, all of which shall fall within the scope of the appended claims of the present disclosure.

What is claimed is:

1. A mixture, comprising a first compound H1 and a second compound H2, wherein
min(LUMO(H1)−HOMO(H2), LUMO(H2)−HOMO(H1))≤min(E_{T1}(H1), E_{T1}(H2))+0.1 eV, LUMO(H1), HOMO(H1), and E_{T1}(H1) respectively represent the lowest unoccupied orbital energy level, the highest occupied orbital energy level, and the triplet energy level of the first compound H1; LUMO(H2), HOMO(H2), and E_{T1}(H2) respectively represent the lowest unoccupied orbital energy level, the highest occupied orbital energy level, and the triplet energy level of the second compound H2;
the first compound H1 comprises a structure of formula (I): 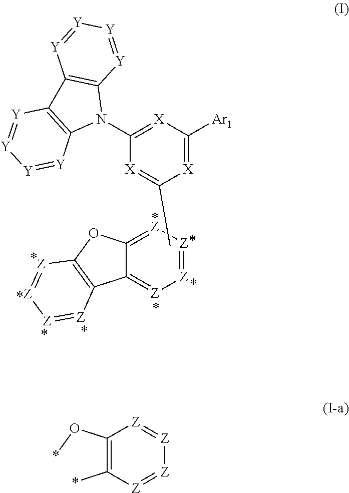
wherein,
two adjacent * in formula (I) are bonded to two * in formula (I-a);
each X is independently N or CR₁, and at least one X is N atom;
Y at each occurrence is independently CR₂ or N; wherein no more than two Ys in each ring are N atoms;
each Z is independently N or CR₃;
Ar₁ is independently selected from a substituted/unsubstituted aromatic or heteroaromatic group containing 5 to 40 ring atoms, an aryloxy or heteroaryloxy group containing 5 to 40 ring atoms, or any combination thereof, wherein one or more Aris form a monocyclic or polycyclic aliphatic or aromatic ring system with each other and/or with the rings bonded thereto; and
each of R₁ to R₃ is independently selected from —H, -D, a substituted/unsubstituted C₁-C₁₀ alkyl group, a substituted/unsubstituted aromatic or heteroaromatic group containing 5 to 60 ring atoms, or any combination thereof.

2. The mixture according to claim 1, wherein the first compound H1 is selected from the group consisting of: 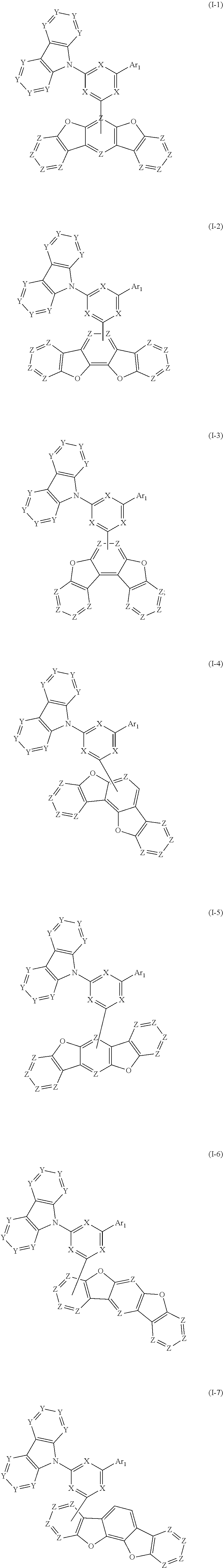 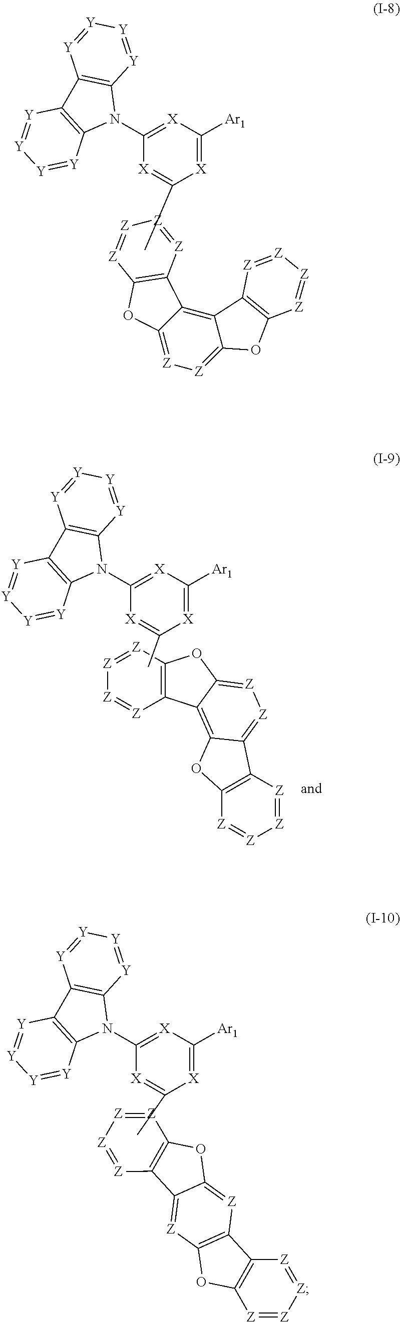
wherein each Ar₁ in formula (I-5) and formula (I-10) is independently selected from the following group:

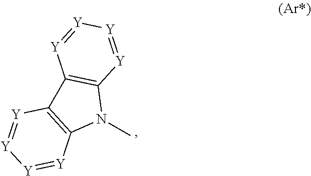

3. The mixture according to claim 1, wherein each Ar₁ is independently selected from one of the following groups or any combination thereof: 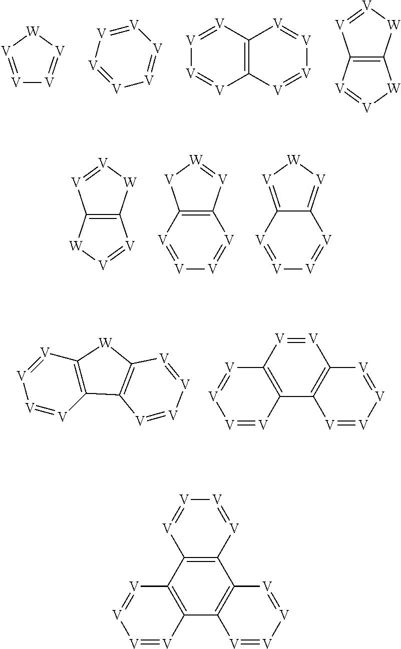
V, in multiple occurrences, is independently selected from CR₄ or N;
W, in multiple occurrences, is independently selected from CR₅R₆, SiR₇R₈, NR₉, C(═O), S, or O; and
each of R₄ to R₉ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, wherein one or more R₄-R₉ form a monocyclic or polycyclic aliphatic or aromatic ring system with each other and/or with the rings bonded thereto.

4. The mixture according to claim 2, wherein each Ar₁ is independently selected from one of the following groups or any combination thereof: 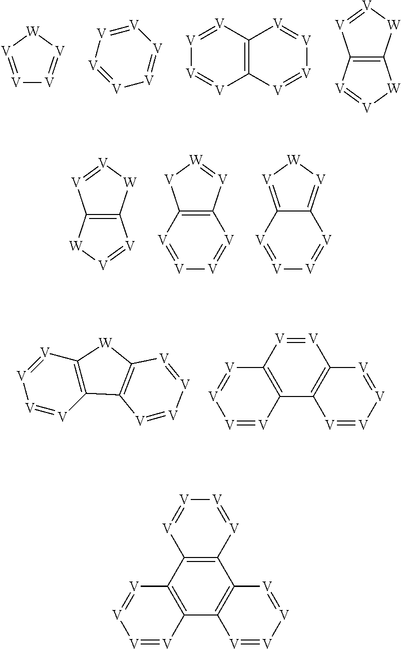
V, in multiple occurrences, is independently selected from CR₄ or N;
W, in multiple occurrences, is independently selected from CR₅R₆, SiR₇R₈, NR₉, C(═O), S, or O; and
each of R₄ to R₉ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, wherein one or more R₄-R₉ form a monocyclic or polycyclic aliphatic or aromatic ring system with each other and/or with the rings bonded thereto.

5. The mixture according to claim 1, wherein each Ar₁ is independently selected from the following structural formulas and is substituted with 0, 1, 2, or 3 substituents, and the substituents are each independently selected from -D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group: 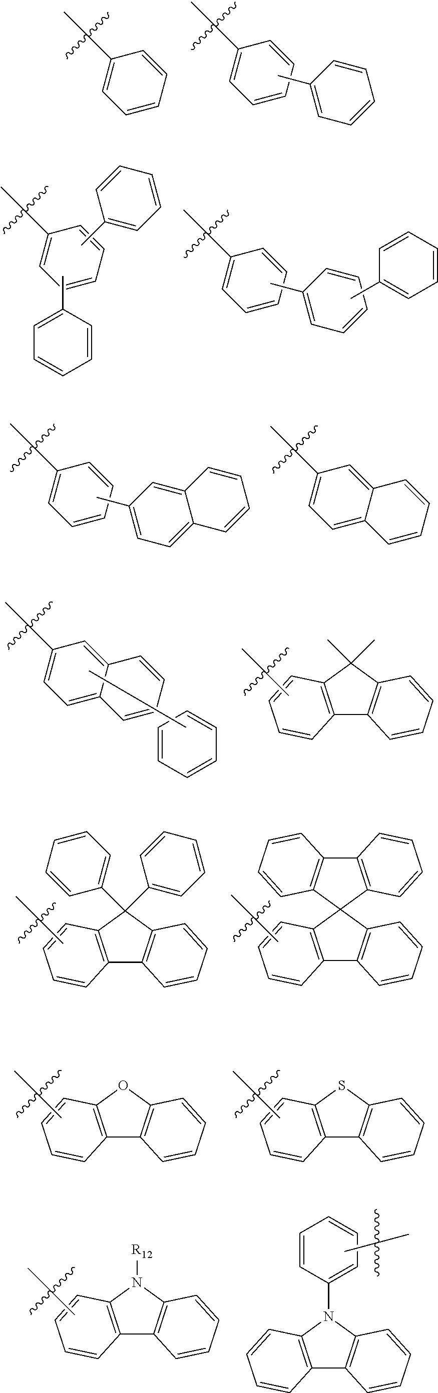 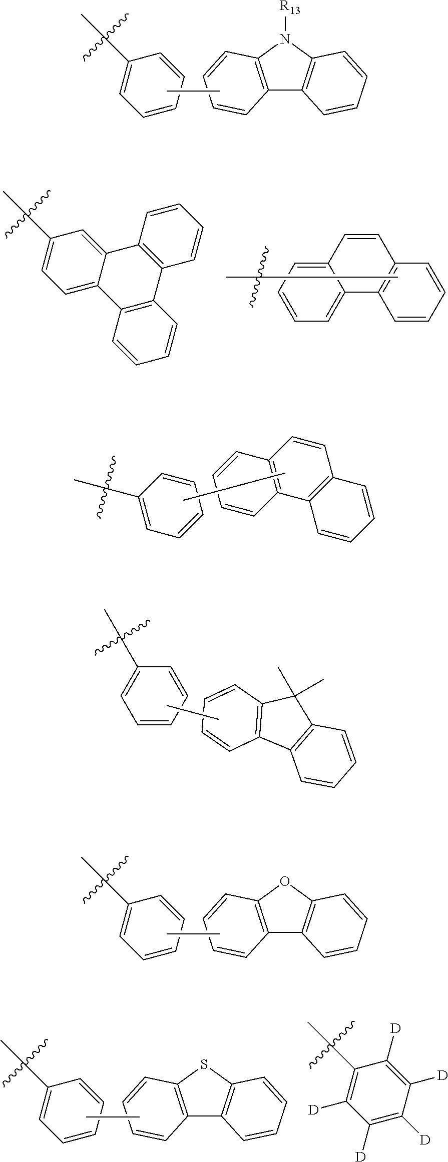 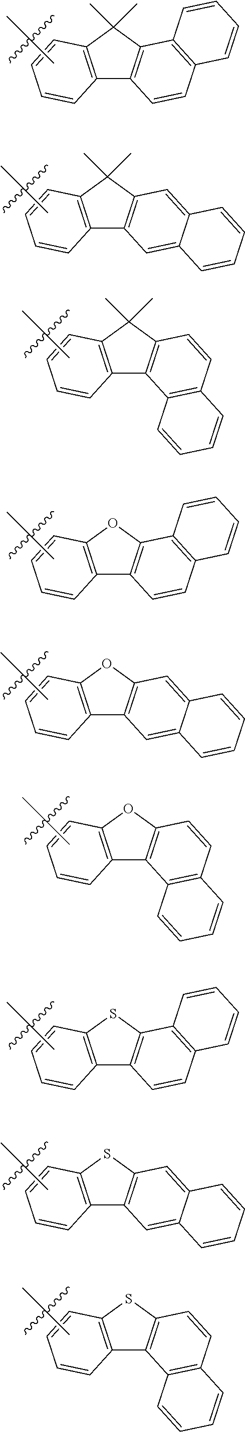 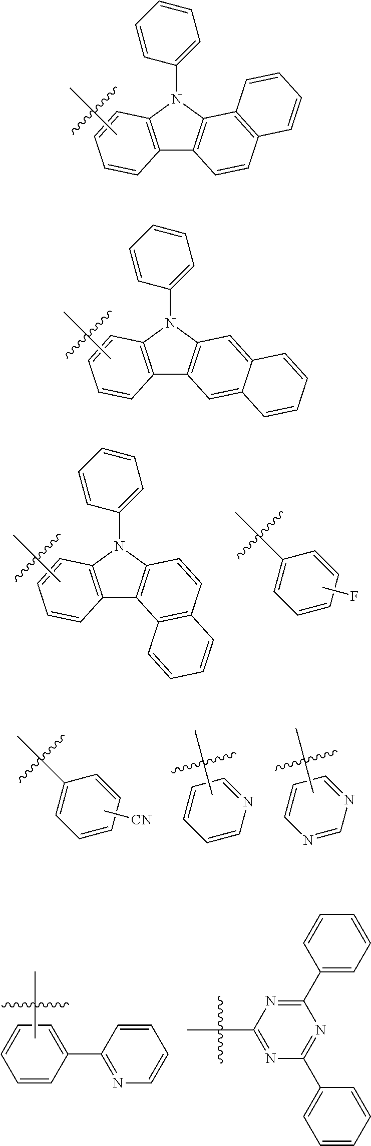 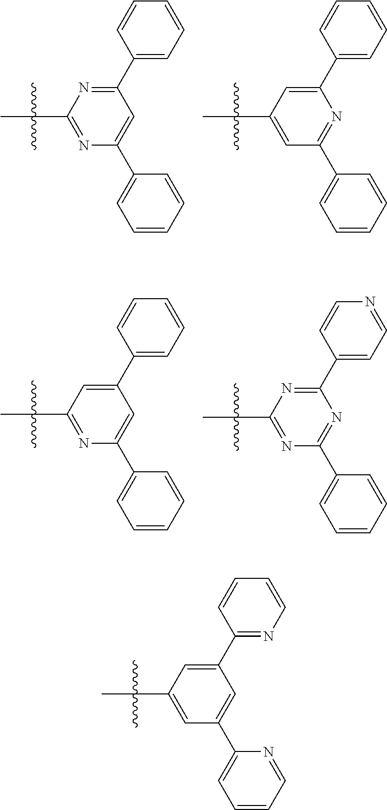
wherein, each of R₁₂ and R₁₃ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, wherein one or more R₁₂-R₁₃ form a monocyclic or polycyclic aliphatic or aromatic ring system with each other and/or with the rings bonded thereto.

6. The mixture according to claim 2, wherein each Ar₁ is independently selected from the following structural formulas and is substituted with 0, 1, 2, or 3 substituents, and the substituents are each independently selected from -D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group: 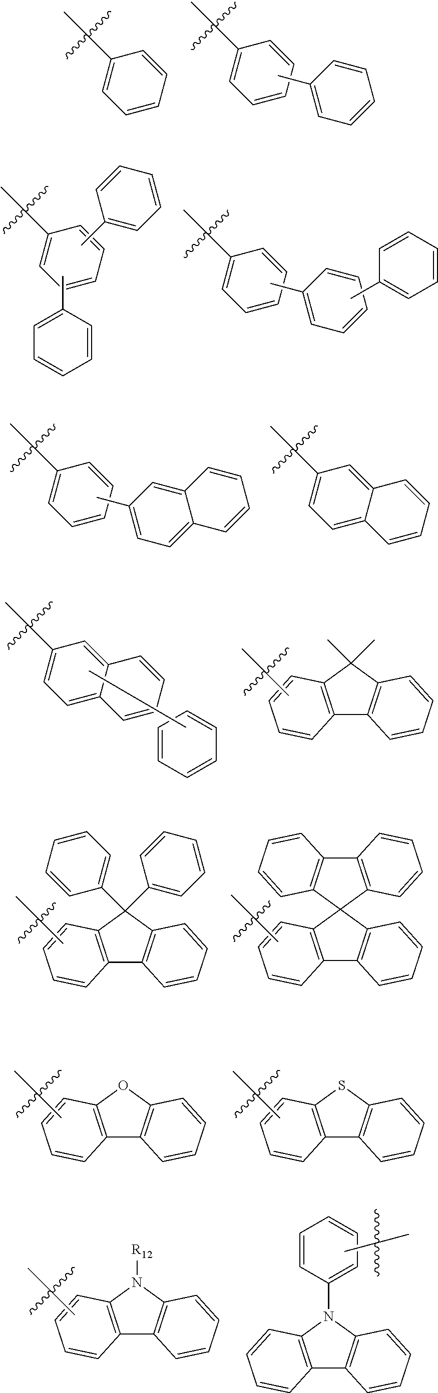 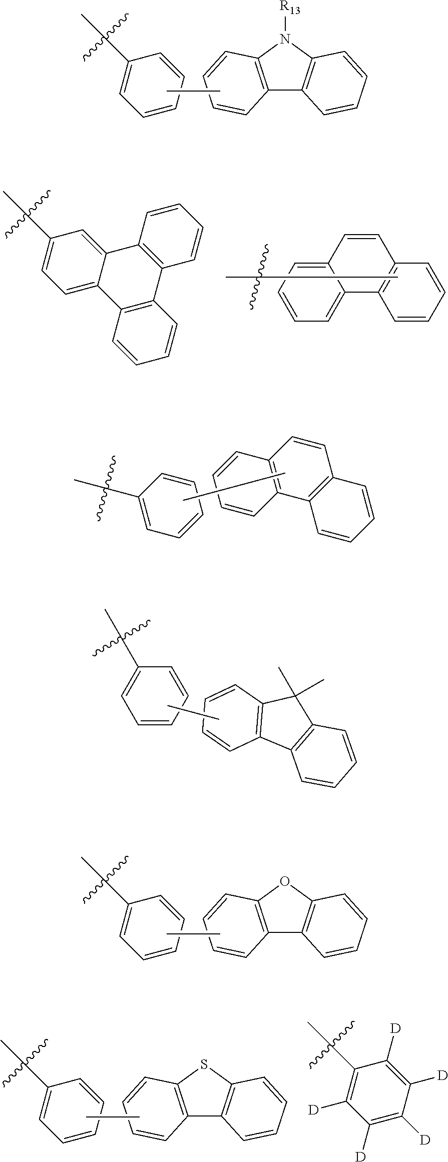 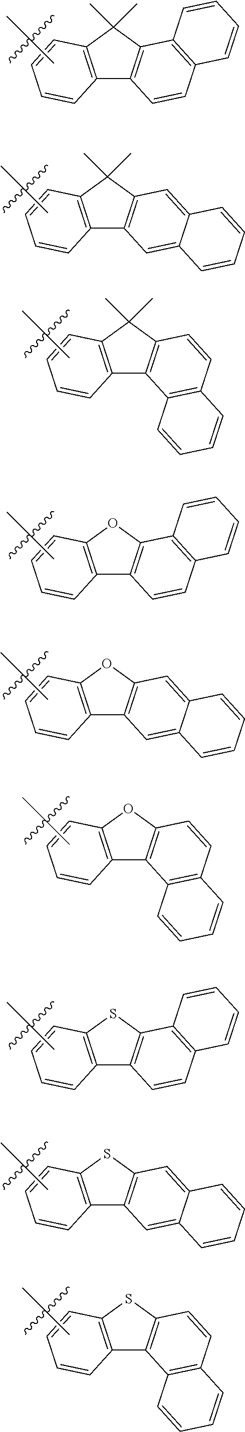 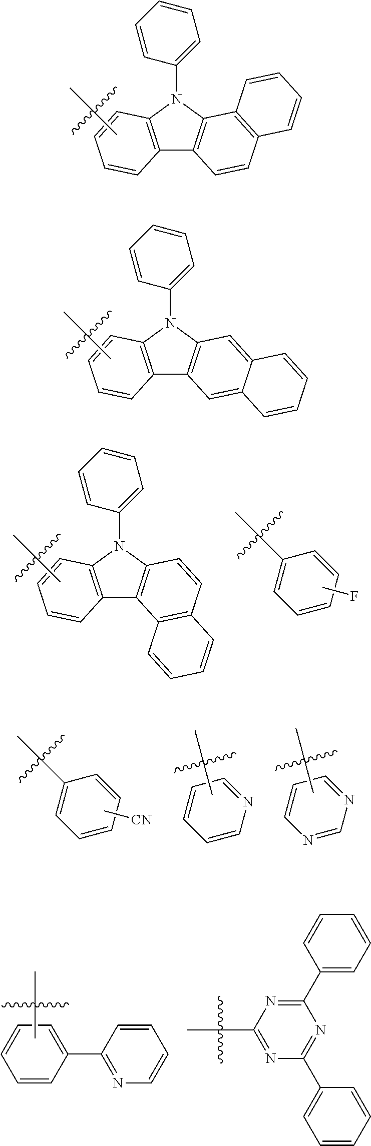 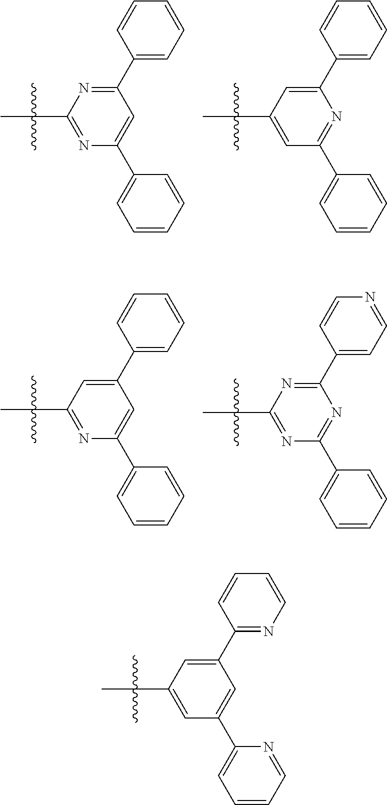
wherein, each of R₁₂ and R₁₃ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, wherein one or more R₁₂-R₁₃ form a monocyclic or polycyclic aliphatic or aromatic ring system with each other and/or with the rings bonded thereto.

7. The mixture according to claim 3, wherein each Ar₁ is independently selected from the following structural formulas and is substituted with 0, 1, 2, or 3 substituents, and the substituents are each independently selected from -D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group: 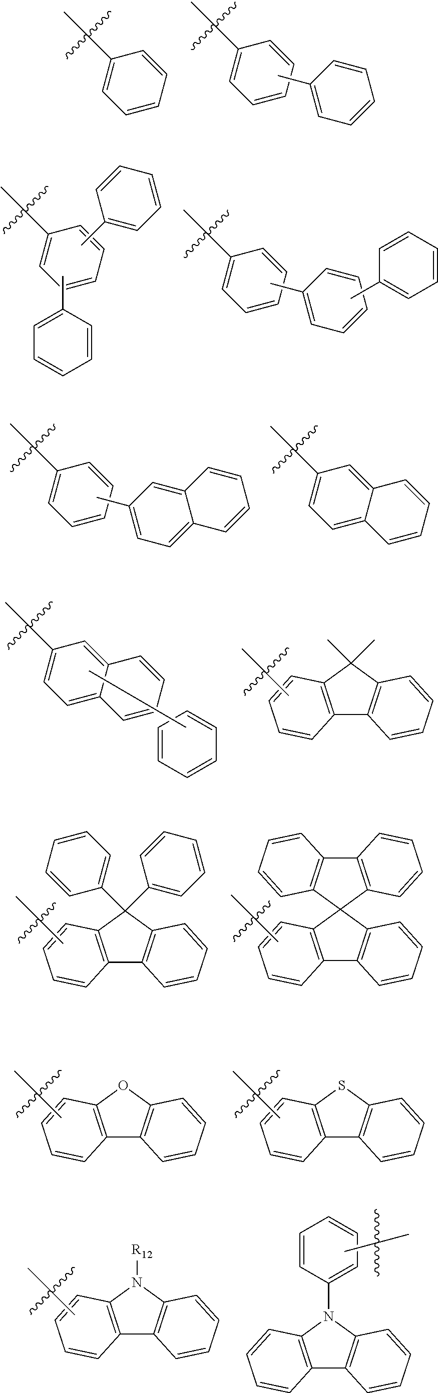 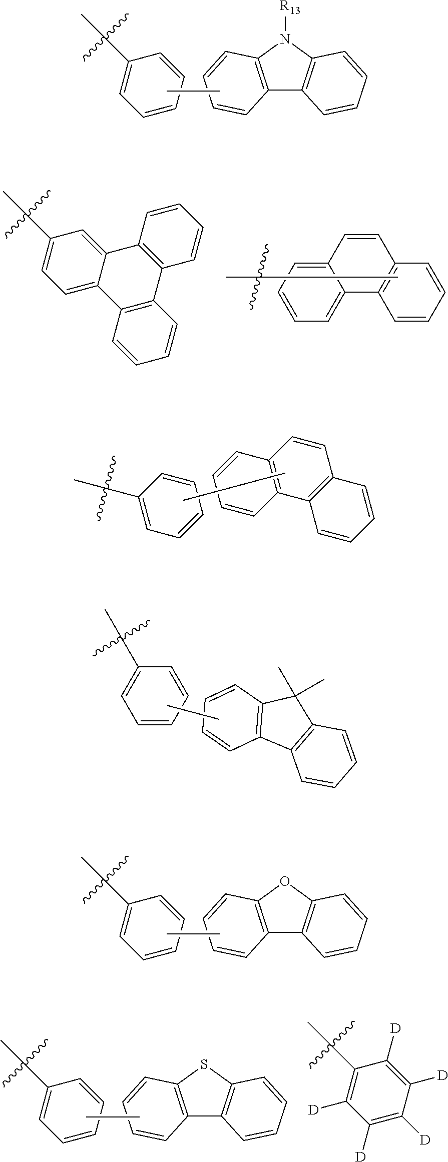 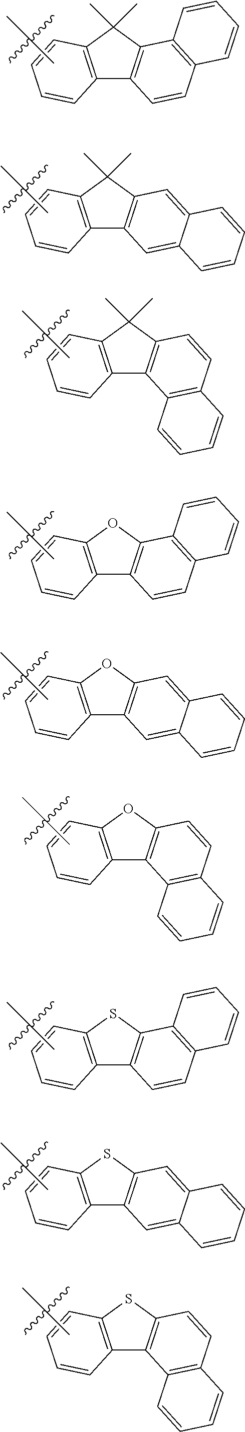 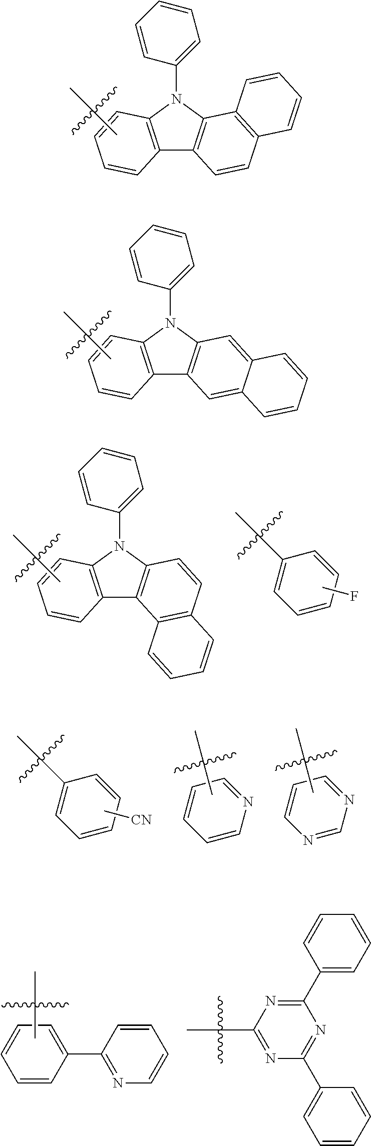 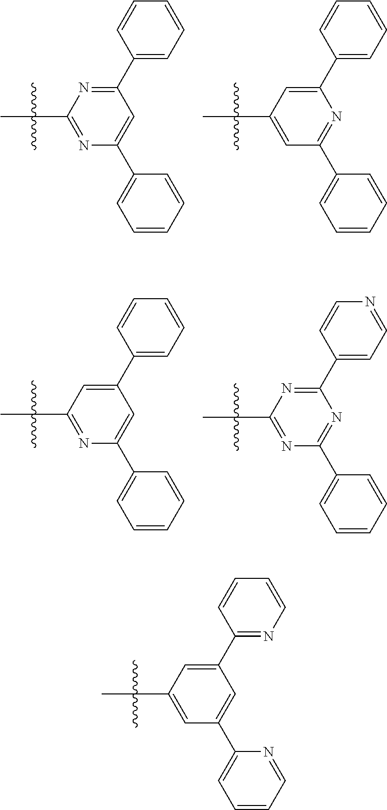
wherein, each of R₁₂ and R₁₃ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, wherein one or more R₁₂-R₁₃ form a monocyclic or polycyclic aliphatic or aromatic ring system with each other and/or with the rings bonded thereto.

8. The mixture according to claim 4, wherein each Ar₁ is independently selected from the following structural formulas and is substituted with 0, 1, 2, or 3 substituents, and the substituents are each independently selected from -D, —F, —Cl, —Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group: 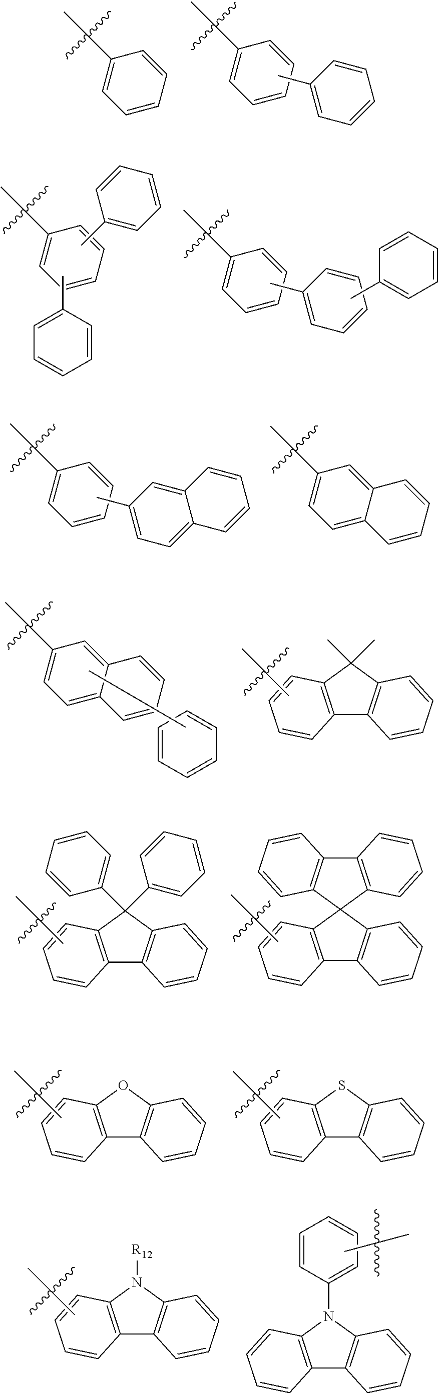 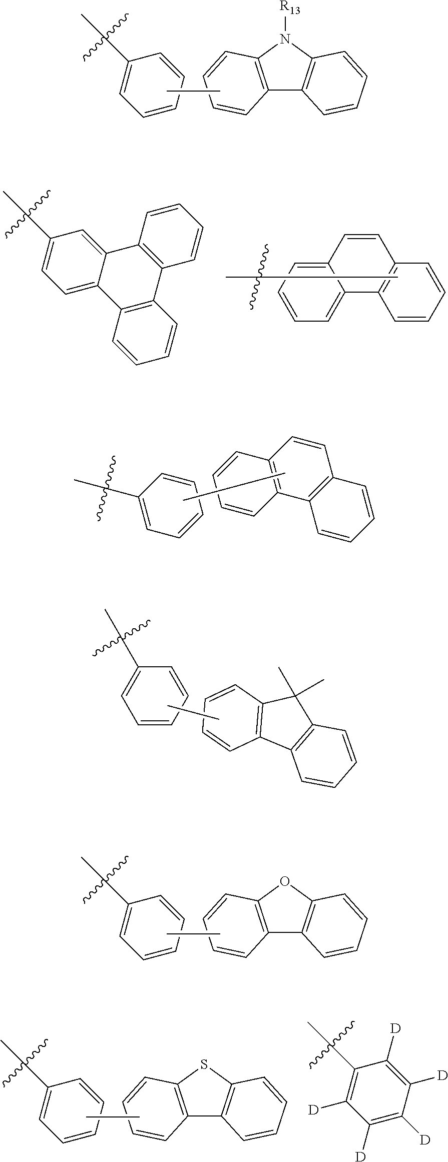 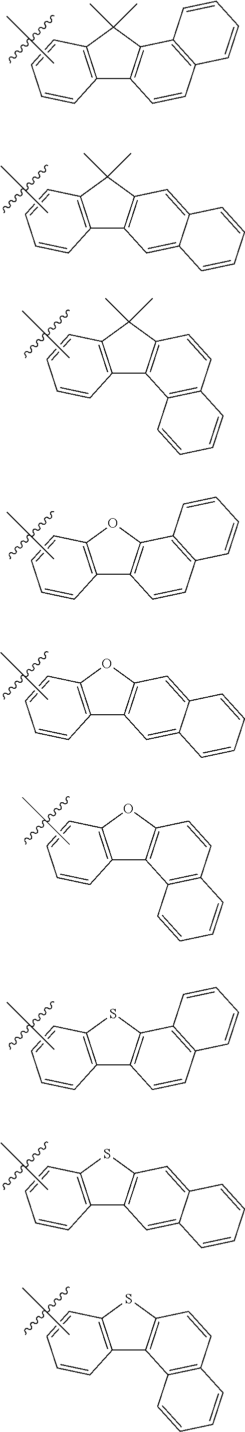 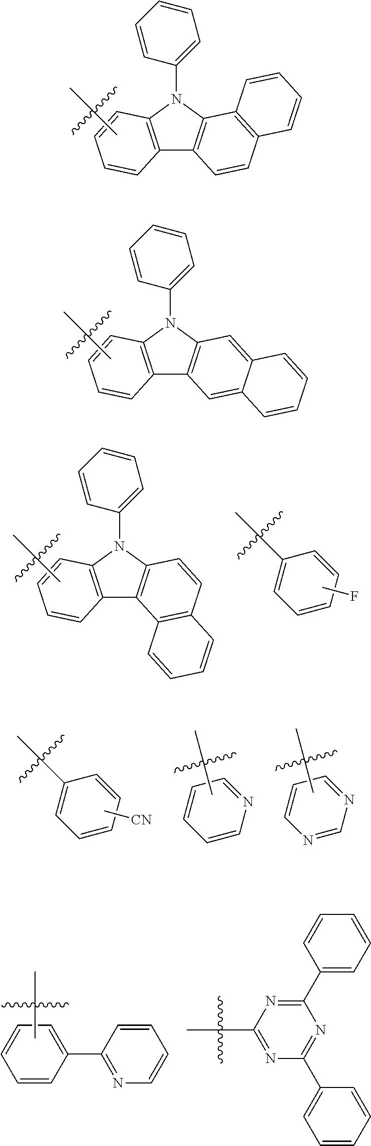 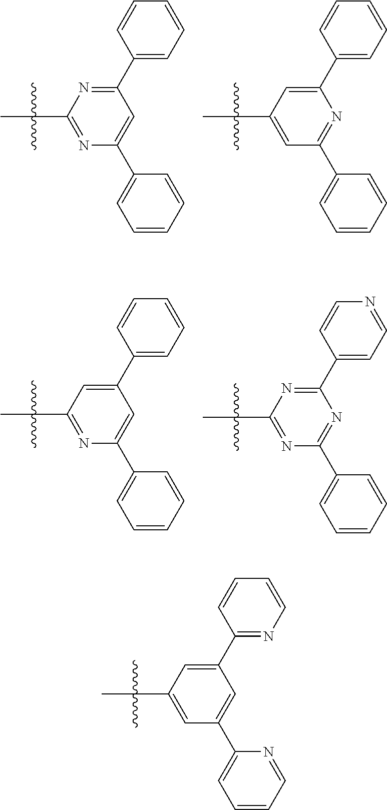
wherein, each of R₁₂ and R₁₃ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, wherein one or more R₁₂-R₁₃ form a monocyclic or polycyclic aliphatic or aromatic ring system with each other and/or with the rings bonded thereto.

9. The mixture according to claim 1, wherein the second compound H2 is selected from the group consisting of: 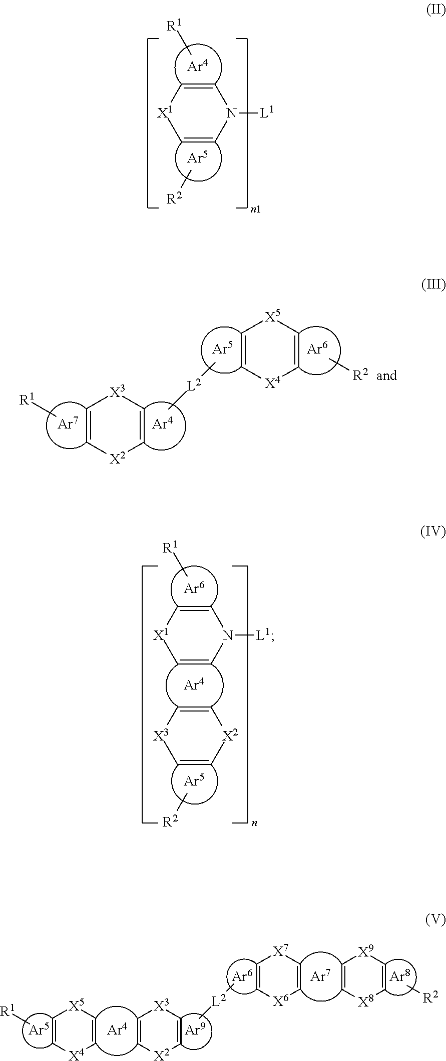
wherein,
each L¹ independently represents an aromatic or a heteroaromatic group containing 5 to 60 ring atoms;
each L² independently represents a single bond, an aromatic or a heteroaromatic group containing 5 to 30 ring atoms, an attachment position of L¹ or L² is on any carbon atom of the benzene ring;
each of Ar⁴ to Ar⁹ is independently selected from an aromatic or a heteroaromatic group containing 5 to 30 ring atoms;
each X¹ independently represents a single bond, N(R), C(R)₂, Si(R)₂, O, C═N(R), C═C(R)₂, P(R), P(═O)R, S, S═O, or SO₂;
each of X² to X⁹ is independently selected from a single bond, N(R), C(R)₂, Si(R)₂, O, C═N(R), C═C(R)₂, P(R), P(═O)R, S, S═O, or SO₂; but X² and X³ are not simultaneously a single bond, X⁴ and X⁵ are not simultaneously a single bond, X⁶ and X⁷ are not simultaneously a single bond, X⁸ and X⁹ are not simultaneously a single bond;
R¹, R², and R are each independently selected from —H, -D, —F, —CN, an alkenyl group, an alkynyl group, a nitrile group, an amine group, a nitro group, an acyl group, an alkoxy group, a carbonyl group, a sulfone group, a C₁-C₃₀ alkyl group, a C₃-C₃₀ cycloalkyl group, an aromatic hydrocarbon group containing 5 to 60 ring atoms, or an aromatic heterocyclic group containing 5 to 60 ring atoms, wherein an attachment position of R¹ or R² is on any carbon atom of the fused ring, and any number of carbon atoms here are substituted with R¹ and R²; and
n1 is an integer from 1 to 4.

10. An organic electronic device, comprising the mixture according to claim 1.

11. The organic electronic device according to claim 10, wherein the organic electronic device is an organic electroluminescent device comprising an emissive layer, and the emissive layer comprises at least one of the mixture.

12. The organic electronic device according to claim 10, wherein the organic electronic device is an organic electroluminescent device comprising an emissive layer or an electron-transport layer or a hole-blocking layer, and the emissive layer or the electron-transport layer or the hole-blocking layer comprises the mixture.

13. An organic compound, comprising a structure of formula (VI): 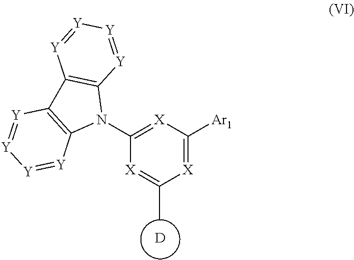
wherein,
each X is independently N or CR₁, and at least one X is N atom;
Y at each occurrence is independently CR₂ or N; wherein no more than two Ys in each ring are N atoms;
Ar₁ is independently selected from a substituted/unsubstituted aromatic or heteroaromatic group containing 5 to 40 ring atoms, an aryloxy or heteroaryloxy group containing 5 to 40 ring atoms, or any combination thereof, wherein one or more Aris form a monocyclic or polycyclic aliphatic or aromatic ring system with each other and/or with the rings bonded thereto;
D ring is selected from the group consisting of: 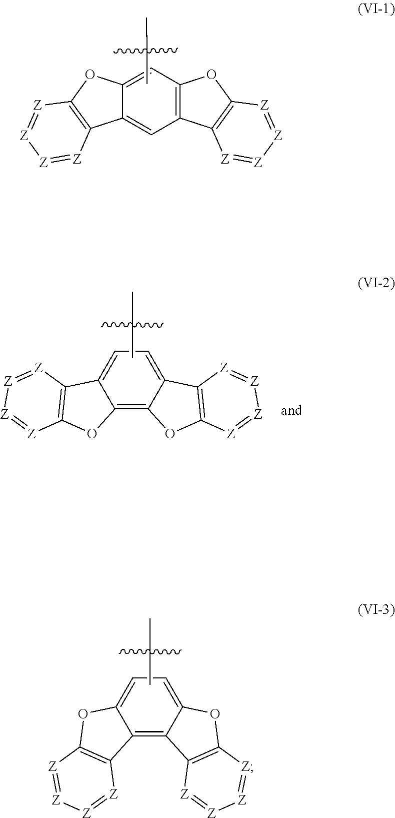
wherein,
each Z is independently N or CR₃; and
each of R₁ to R₃ is independently selected from —H, -D, a substituted/unsubstituted C₁-C₁₀ alkyl group, a substituted/unsubstituted aromatic or heteroaromatic group containing 5 to 60 ring atoms, or any combination thereof.
